(19) United States
(12) Patent Application Publication
(10) Pub. No.: US 2025/0313560 A1
(43) Pub. Date: Oct. 9, 2025

(54) Novel bicyclic benzylamido pyridine derivatives as SOS1 inhibitors

(71) Applicant: Boehringer Ingelheim International GmbH, Ingelheim am Rhein (DE)

(72) Inventors: Christoph HOHN, Freiburg (DE); Riccardo GIOVANNINI, Biberach an der Riss (DE); Niklas Helge HEINE, Biberach an der Riss (DE); Elke LANGKOPF, Biberach an der Riss (DE); Stephan Georg MUELLER, Warthausen (DE)

(21) Appl. No.: 19/098,174

(22) Filed: Apr. 2, 2025

(30) Foreign Application Priority Data
Apr. 3, 2024 (EP) 24168270.7

(51) Int. Cl. C07D 471/04; A61K 31/437; A61K 31/438; A61K 31/4545; A61K 31/5377; A61K 31/55; C07D 519/00

(52) U.S. Cl. CPC C07D 471/04; A61K 31/437; A61K 31/438; A61K 31/4545; A61K 31/5377; A61K 31/55; C07D 519/00

(57) ABSTRACT

The present invention encompasses compounds of formula (I)

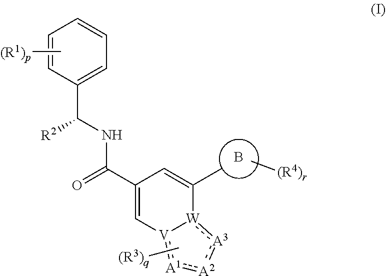

wherein the groups R¹ to R⁴, A¹, A², A³, ring system B, V, W, p, q and r have the meanings given in the claims and specification, their use as inhibitors of SOS1, pharmaceutical compositions which contain compounds of this kind and their use as medicaments/medical uses, especially as agents for treatment and/or prevention of oncological diseases.

### FIELD OF THE INVENTION

The present invention relates to small molecules and their salts capable of inhibiting SOS1 (Son of Sevenless). Specifically, the present invention relates to new substituted bicyclic benzylamido pyridines and derivatives of formula (I)

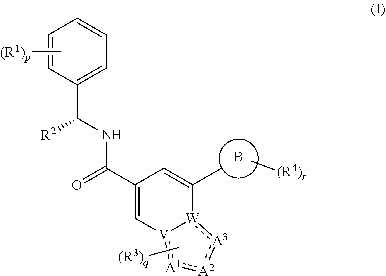

wherein the groups R¹ to R⁴, A¹, A², A³, ring system B, V, W, p, q and r have the meanings given in the claims and specification, as well as the synthesis of these compounds. Furthermore, the invention relates to pharmaceutical compositions and combinations comprising these compounds, as well as their use in methods for the treatment of diseases associated with or modulated by SOS1. Pharmaceutical compositions comprising the compounds of general formula (I) are suitable for the therapy of diseases characterized by excessive or abnormal cell proliferation such as cancer.

### BACKGROUND OF THE INVENTION

RAS-family proteins including KRAS (V-Ki-ras2 Kirsten rat sarcoma viral oncogene homolog), NRAS (neuroblastoma RAS viral oncogene homolog), HRAS (Harvey murine sarcoma virus oncogene) and MRAS (muscle RAS oncogene homolog) and any mutants thereof are small GTPases that exist in cells in either GTP-bound or GDP-bound states and which have a weak intrinsic GTPase activity and slow nucleotide exchange rates (Moore et al., Nat Rev Drug Discov., 2020 August; 19(8):533-552). Binding of GTPase activating proteins (GAPs) such as NF1 increases the GTPase activity of RAS-family proteins. The binding of guanine nucleotide exchange factors (GEFs) such as SOS1 (Son of Sevenless 1) promote release of GDP from RAS-family proteins, enabling GTP binding. When in the GTP-bound state, RAS-family proteins are active and engage effector proteins including C-RAF and phosphoinositide 3-kinase (PI3K) to promote the RAF/mitogen or extracellular signal-regulated kinases (MEK/ERK) pathway, PI3K/AKT/mammalian target of rapamycin (mTOR) pathway and RalGDS (Ral guanine nucleotide dissociation stimulator) pathway. These pathways affect diverse cellular processes such as proliferation, survival, metabolism, motility, angiogenesis, immunity and growth (Moore et al., Nat Rev Drug Discov., 2020 August; 19(8):533-552).

Cancer-associated mutations in RAS-family proteins suppress their intrinsic and GAP-induced GTPase activity leading to an increased population of GTP-bound/active RAS-family proteins. This in turn leads to persistent activation of effector pathways (e.g. MEK/ERK, PI3K/AKT/mTOR, RalGDS pathways) downstream of RAS-family proteins. KRAS mutations (e.g. amino acids G12, G13, Q61, A146) are found in a variety of human cancers including lung cancer, colorectal cancer and pancreatic cancer. Mutations in HRAS (e.g. amino acids G12, G13, Q61) and NRAS (e.g. amino acids G12, G13, Q61, A146) are also found in a variety of human cancer types however typically at a lower frequency compared to KRAS mutations (Moore et al., Nat Rev Drug Discov., 2020 August; 19(8):533-552). MRAS (e.g. amino acid G23V and T681) have been described in Noonan syndrome (Young & Rodriguez-Viciana, Cold Spring Harbor Perspect Med. 2018 Dec. 3; 8(12):a033621). Alterations (e.g. mutation, over-expression, gene amplification) in RAS-family proteins have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Eberlein et al., Cancer Res., 2015, 75(12):2489-500). Resistance mechanisms were also described upon treatment with G12Ci (adagrasib, sotorasib), including the enrichment for secondary KRAS mutations as well as other oncogenic alleles (Awad et al, N Engl J Med 2021; 384:2382-239). Published data furthermore indicate Son of Sevenless 1 (SOS1) inhibitors could overcome acquired resistance to KRAS G12C inhibition mediated by KRAS secondary mutations (Koga T. et al., Journal of Thoracic Oncology 2021, 16, 8, 1321-1332) or upregulation of RAS expression (e.g. MRAS; Thatikonda, et al. Nat Cancer 2024, 5, 1352-1370), therefore highlighting the potential of combination approaches involving combinations including a SOS1 inhibitor.

SOS1 is a multi-domain protein with two binding sites for RAS-family proteins: a catalytic site that binds GDP-bound RAS-family proteins to promote guanine nucleotide exchange and an allosteric site that binds GTP-bound RAS-family proteins, the latter causing further increase in the catalytic GEF function of SOS1. Published data indicate a critical involvement of SOS1 in mutant KRAS activation and oncogenic signaling in cancer (Jeng et al., Nat. Commun., 2012, 3:1168, Hofmann, Gmachl, Ramharter et al, Cancer Discov. 2021, 11(1):142-15). Depleting SOS1 levels decreased the proliferation rate and survival of tumor cells carrying a KRAS mutation whereas no effect was observed in KRAS wild type cell lines and the effect of loss of SOS1 could not be rescued by introduction of a catalytic site mutated SOS1.

Alterations in SOS1 have been implicated in cancer. SOS1 mutations are found in embryonal rhabdomyosarcomas, sertoli cell testis tumors, granular cell tumors of the skin (Denayer et al., Genes Chromosomes Cancer, 2010, 49(3):242-52), lung adenocarcinoma (Cancer Genome Atlas Research Network., Nature. 2014, 511(7511):543-50), bladder cancer (Watanabe et al., IUBMB Life., 2000, 49(4):317-20) and prostate cancer (Timofeeva et al., Int. J. Oncol., 2009, 35(4):751-60). In addition to cancer, hereditary SOS1 mutations are implicated in the pathogenesis of RASopathies e.g. Noonan syndrome (NS) (Pierre et al., Biochem. Pharmacol., 2011, 82(9):1049-56).

SOS1 homolog in mammalian cells, Son of Sevenless 2 (SOS2) also acts as a GEF for the activation of RAS-family proteins. Data from mouse knock-out models suggests a redundant role for SOS1 and SOS2 in homeostasis in the adult mouse and the data suggest that selective targeting of individual SOS isoforms (e.g. selective SOS1 targeting) may be adequately tolerated to achieve a therapeutic index between SOS1/RAS-family protein driven cancers (or other SOS1/RAS-family protein pathologies) and normal cells and tissues.

In publications, small molecules inhibiting SOS1 are for example described in WO 2021/074227, WO 2022/146698, WO 2022/187266 and CN116041344.

Selective pharmacological inhibition of the binding of the catalytic site of SOS1 to RAS-family proteins was shown to prevent SOS1-mediated activation of RAS-family proteins to the GTP-bound form (Hofmann, Gmachl, Ramharter et al, Cancer Discov. 2021, 11(1):142-15). Such SOS1 inhibitor compounds are expected to consequently inhibit signaling in cells downstream of RAS-family proteins (e.g. ERK phosphorylation). In cancer cells associated with dependence on RAS-family proteins (e.g. KRAS mutant cancer cell lines), SOS1 inhibitor compounds are expected to deliver anti-cancer efficacy (e.g. inhibition of proliferation, survival etc.). High potency towards inhibition of SOS1:RAS-family protein binding and ERK phosphorylation are therefore desirable characteristics for a SOS1 inhibitor compound, preferably coupled with a good metabolic stability suitable for oral absorption.

Due to the high potential that combination therapy approaches with SOS1 inhibitors promise (Kessler, et al. Curr Opinion Chem Biol. 2021, 62:109-118), the risks of drug-drug interactions (DDI) need to be evaluated early to prevent adverse effects. In this regard, especially cytochrome P450 (CYP) inhibition is a major DDI concern. The reversible CYP inhibition where the inhibitor binds to the CYP enzyme and is released in a reversible binding scheme is time-independent. Whereas the irreversible binding, also called mechanism-based inhibition, is time-dependent coming from formation of a covalent bond between an inhibitor or its metabolite and the CYP enzyme. Therefore, desirable SOS1 inhibitors show a decreased risk of time-dependent and time-independent inhibition of cytochromes to help prevent adverse effects from drug combinations.

### SUMMARY OF THE INVENTION

Compounds according to the present invention are highly potent inhibitors of SOS1 (see Table 1 for KRAS::SOS1 alphascreen binding assay and Table 2 for Erk phosphorylation assay) which show good stability in hepatocytes (see Table 3) and low risk in CYP inhibition and mechanism based inhibition (see Table 4 and Table 5 for CYP assays).

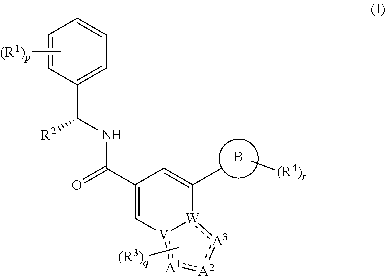

The compounds of formula (I) or the salts thereof as defined herein are particularly suitable for the treatment of pathophysiological processes associated with or modulated by SOS1 inhibition, particularly for the treatment of primary and metastatic tumors associated with dependence on RAS-family protein signaling. Therefore, the compounds of formula (I) or the salts thereof as defined herein are particularly suited for the treatment of cancer associated with dependence on RAS-family protein signaling, including sizeable proportions of NSCLC (non-small cell lung cancer) patients.

In one aspect, the invention relates to compounds of formula (I) in their salt free forms. In another aspect, the invention relates to the method of treatment involving the compounds of formula (I) or the salts thereof. In another aspect, the invention relates to the use of a compound of general formula (I) or a pharmaceutically acceptable salt thereof as a medicament. In another aspect, the invention relates to a pharmaceutical composition comprising at least one compound of general formula (I). In another aspect, the invention relates to compounds of formula (I) or a pharmaceutically acceptable salt thereof and a pharmaceutically acceptable carrier. In another aspect, the invention relates to the use of a compound of general formula (I) in a medicament combination which comprises further active substances. In another embodiment, the invention provides the general synthesis schemes for compounds of general formula (I) including examples and methods.

### DETAILED DESCRIPTION OF THE INVENTION

The present invention therefore relates to compounds of formula (I)

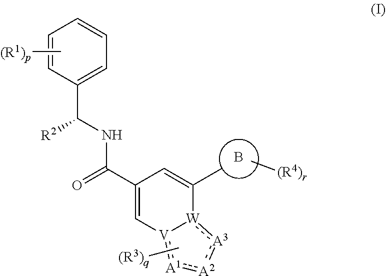

wherein the groups R¹ to R⁴, A¹, A², A³, ring system B, V, W, p, q and r have one of the meanings given hereafter, or salts thereof which are particularly suitable for the treatment of pathophysiological processes associated with or modulated by SOS1-inhibition, particularly for the treatment of cancer, particularly for the treatment of MAPK pathway dependent tumors.

The compounds of the present invention exhibit several advantageous properties, such as high potency shown in vitro by inhibiting the interaction between SOS1 and KRAS alleles G12D and G12C with IC₅₀ values below 300 nM, preferably below 200 nM, more preferably below 100 nM, most preferably below 70 nM (see Table 1). Favorable binding affinity to human SOS1 in combination with favorable cellular activity, as shown by the in vitro ERK phosphorylation assay, and favorable pharmacokinetic properties can enable lower doses for pharmacological efficacy. Lower doses have the advantages of lower “drug load” or “drug burden” (parent drug and metabolites thereof) for the patient causing potentially fewer side effects, and lower production costs for the drug product.

Furthermore, the high cellular potency of the compounds of the present invention is dis-played by IC₅₀ values below 600 nM, preferably below 300 nM, more preferably below 200 nM, most preferably below 100 nM in an in vitro ERK phosphorylation assay (see Table 2).

In addition to the affinity assay demonstrating the binding of the compounds of the invention to the target, the cellular ERK phosphorylation assays are used to examine the potency with which compounds inhibit the SOS1-mediated signal transduction in a KRAS mutant human cancer cell line. This demonstrates the molecular mode of action of compounds by interfering with the RAS-family protein signal transduction cascade. Low IC₅₀ values are indicative of high potency of the SOS1 inhibitor compounds in this assay setting. It is observed that the compounds of the invention demonstrate an inhibitory effect on ERK phosphorylation in a KRAS mutant human cancer cell line, thus confirming the molecular mode of action of the SOS1 inhibitor compounds on RAS-family protein signal transduction.

Further, the compounds of the present invention are metabolically stable in human hepatocytes (metabolically stable in human hepatocytes in this respect is defined as below or equal to 45% QH, preferably below or equal to 35% QH, more preferably below or equal to 25% QH, most preferably below or equal to 20% QH, (see Table 33) and the definition of how to calculate the % QH=hepatic blood flow herein below). Therefore, the compounds of the present invention are expected to have a favorable in vivo clearance and thus the desired duration of action in humans.

In addition, the compounds of the present invention are characterized by a low DDI risk based on the cytochrome P450 (CYP) inhibition. The DDI perpetrator risk can be indicated by the reversible inhibition of CYP3A4 isoform, wherein an IC₅₀>50 μM represents a low inhibition (see Table 4). Another aspect of the perpetrator potential can be evaluated by mechanism-based inhibition (MBI) of CYP3A. Further, the compounds of the present invention show a low risk for mechanism-based inhibition as defined by the remaining CYP3A activities: preferably above or equal to 75% ctrl. after a preincubation with 25 μM compound for 30 min; most preferably above or equal to 90% ctrl. (see Table 5 and the definition of how to calculate the % ctrl. is outlined below).

In summary, the compounds according to the present invention are highly potent inhibitors of the protein-protein interaction between SOS1 and RAS, especially KRAS mutated in position 12 or 13, preferably G12C or G12D mutant KRAS, display high cellular potency as seen in an in vitro ERK phosphorylation assay, are metabolically stable in human hepatocytes and show a low DDI risk.

### Compounds

In one aspect, the invention relates to compounds of formula (I)

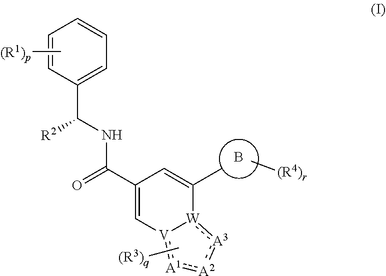

wherein
each R¹ is independently selected from the group consisting of C₁₋₆alkyl, C₁₋₆haloalkyl and halogen;
p denotes 1, 2 or 3;
R² is H, Me or Et;
V is nitrogen (—N═) or carbon

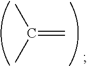

W is nitrogen (—N═) or carbon

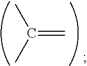

at least one of V and W is nitrogen (—N═);
A¹, A² and A³ are each independently selected from nitrogen (—N═) or carbon (═CH— or ═CR³—)
and [symbol] is a single or double bond;
each R³, if present, is independently selected from the group consisting of C₁₋₆alkyl, C₁₋₆alkoxy, halogen and C₁₋₆haloalkyl;
q denotes 0, 1 or 2;
ring system B is selected from C₃₋₁₀cycloalkyl, C₄₋₁₀cycloalkenyl, 4-13 membered heterocyclyl,
r denotes 0, 1, 2, 3 or 4;
each R⁴, if present, is independently selected from the group consisting of R⁵ and R⁶;
each R⁵ is independently selected from the group consisting of —OR⁶, —NR⁶R⁶, halogen, —CN, —C(═O)R⁶, —C(═O)OR⁶, —C(═O)NR⁶R⁶, —NHC(═O)OR⁶ and the bivalent substituent ═O;
each R⁶ is independently selected from the group consisting of hydrogen, C₁₋₆alkyl, C₁₋₆alkoxy, C₁₋₆haloalkyl, C₃₋₁₀cycloalkyl, C₄₋₁₀cycloalkenyl, 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl and 4-11 membered heterocyclyl, are each independently optionally substituted with one or more, identical or different R⁷ and/or R⁸;
each R⁷ is independently selected from the group consisting of —OH, —NH₂, —NHR⁸, —NR⁸R⁸, halogen, —CN, and C₁₋₆alkoxy;
each R⁸ is independently selected from the group consisting of C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl and 4-11 membered heterocyclyl, are each independently optionally substituted with one or more, identical or different R⁹;
each R⁹ is —OH, halogen or C₁₋₆alkoxy;
or a salt thereof.

In another aspect, the invention relates to the compound of formula (I), or a salt thereof, wherein the ring

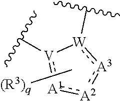

is selected from the group consisting of

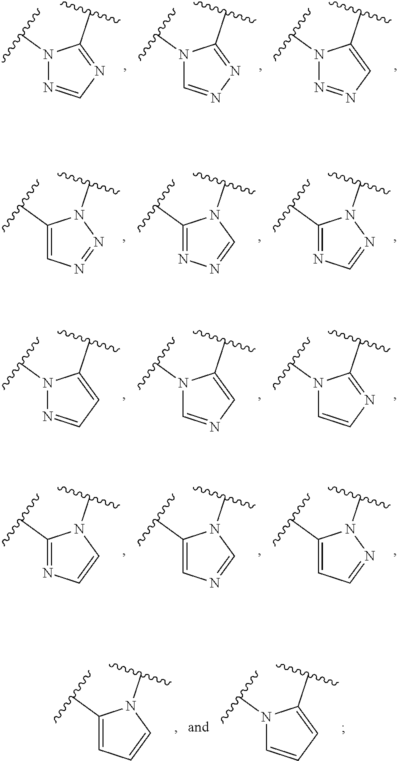

and is optionally substituted with one or two, identical or different R³.

In another aspect, the invention relates to the compound of formula (I), or a salt thereof, wherein A¹, A², A³, V and W form a triazole.

In another aspect, the invention relates to the compound of formula (I), or a salt thereof, wherein the ring

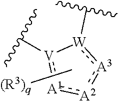

is selected from the group consisting of

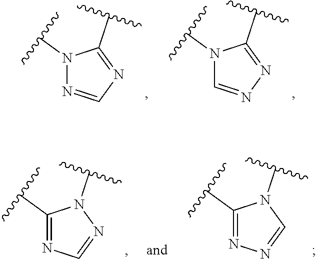 [SMILES: *c1ncnn1* ; *c1nncn1* ; *c1ncnn1* ; *c1nncn1*]

and is optionally substituted with R³.

In another aspect, the invention relates to the compound of formula (I), or a salt thereof, wherein q is 0 to 2 and each R³, if present, is independently selected from the group consisting of C₁₋₆alkyl, C₁₋₆alkoxy, halogen and C₁₋₆haloalkyl.

In another aspect, the invention relates to the compound of formula (I), or a salt thereof, wherein q is 0 to 2 and each R³, if present, is C₁₋₆alkyl.

In another aspect, the invention relates to the compound of formula (I), or a salt thereof, wherein q is 0 to 2 and each R³, if present, is Me, Et or Pr.

In another aspect, the invention relates to the compound of formula (I), or a salt thereof, wherein q is 0 to 2 and each R³, if present, is Me.

In another aspect, the invention relates to the compound of formula (I), or a salt thereof, wherein q is 1 and R³ is Me.

In another aspect, the invention relates to the compound of formula (I), or a salt thereof, wherein q is 0.

In another aspect, the invention relates to the compound of formula (I), or a salt thereof wherein p is 1.

In another aspect, the invention relates to the compound of formula (I), or a salt thereof wherein p is 2.

In another aspect, the invention relates to the compound of formula (I), or a salt thereof wherein p is 2 and each R¹ is independently selected from the group consisting of Me, —CFH₂, —CF₂H, —CF₃, —CFMeH, —CFMe₂, —CF₂Me, F.

In another aspect, the invention relates to the compound of formula (I), or a salt thereof wherein p is 2 and each R¹ is independently selected from the group consisting of Me, —CF₂H, —CF₃, F.

In another aspect, the invention relates to the compound of formula (I), or a salt thereof wherein R² is Me.

In another aspect, the invention relates to the compound of formula (I), or a salt thereof wherein R² is Me, p is 2 and each R¹ is independently selected from the group consisting of Me, —CFH₂, —CF₂H, —CF₃, —CFMeH, —CFMe₂, —CF₂Me, F.

In another aspect, the invention relates to the compound of formula (I), or a salt thereof wherein R² is Me, p is 2 and each R¹ is independently selected from the group consisting of Me, —CFH₂, —CF₃, F.

In another aspect, the invention relates to the compound of the formula (I), or a salt thereof, wherein ring system B is selected from C₃₋₁₀cycloalkyl, and C₄₋₁₀cycloalkenyl; and the C₃₋₁₀cycloalkyl, and C₄₋₁₀cycloalkenyl is optionally and independently substituted with r, identical or different R⁴.

In another aspect, the invention relates to the compound of the formula (I), or a salt thereof, wherein ring system B is selected from C₅₋₇cycloalkyl, and C₅₋₇cycloalkenyl; and the C₅₋₇cycloalkyl, and C₅₋₇cycloalkenyl is optionally and independently substituted with r, identical or different R⁴.

In another aspect, the invention relates to the compound of the formula (I), or a salt thereof, wherein ring system B is selected from cyclohexyl, and cyclohexenyl; and the cyclohexyl, and cyclohexenyl is optionally and independently substituted with r, identical or different R⁴.

In another aspect, the invention relates to the compound of the formula (I), or a salt thereof, wherein ring system B is cyclohexyl; and the cyclohexyl is optionally and independently substituted with r, identical or different R⁴.

In another aspect, the invention relates to the compound of the formula (I), or a salt thereof, wherein ring system B is cyclohexenyl; and the cyclohexenyl is optionally and independently substituted with r, identical or different R⁴.

In another aspect, the invention relates to the compound of the formula (I), or a salt thereof, wherein ring system B is a 4-13 membered heterocyclyl; and the 4-13 membered heterocyclyl is optionally and independently substituted with r, identical or different R⁴.

In another aspect, the invention relates to the compound of the formula (I), or a salt thereof, wherein ring system B is a piperidine.

In another aspect, the invention relates to the compound of the formula (I), or a salt thereof, wherein ring system B is selected from the group consisting of

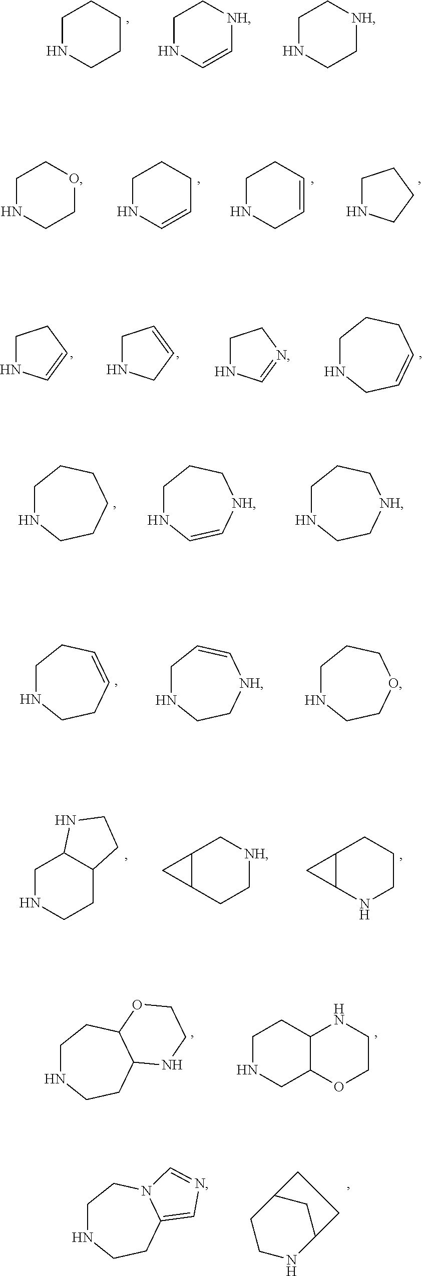

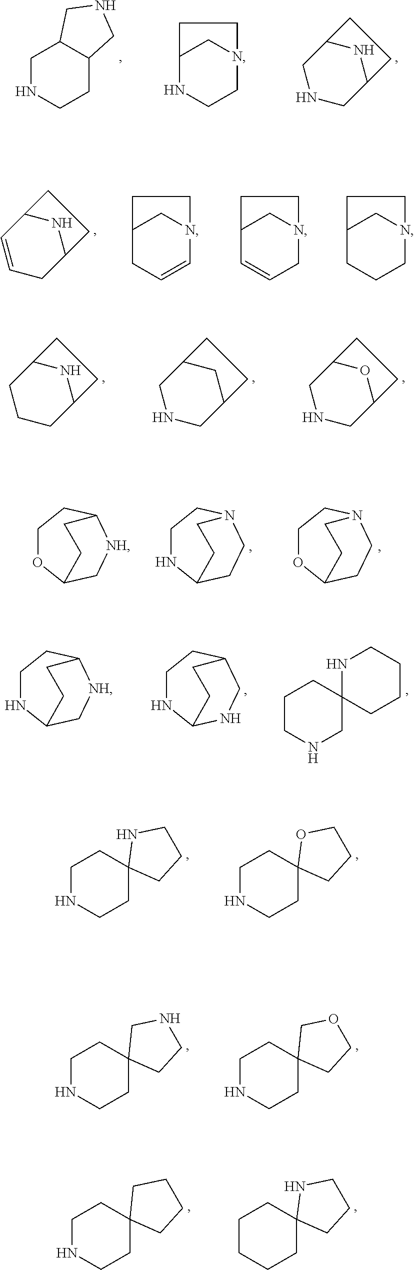

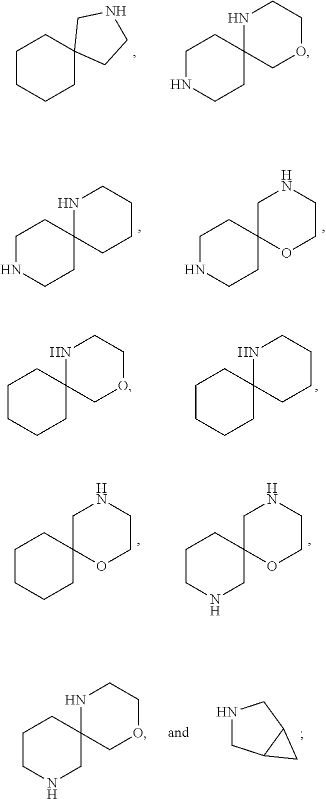

wherein ring system B can be attached to the compound of formula (I) and R⁴, if present, at any ring position by removal of a hydrogen atom.

In another aspect, the invention relates to the compound of the formula (I), or a salt thereof, wherein ring system B is selected from the group consisting of

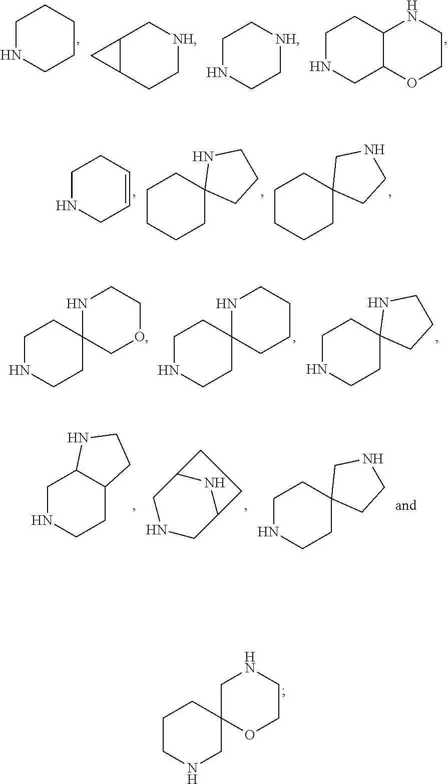

wherein ring system B can be attached to the compound of formula (I) and R⁴, if present, at any ring position by removal of a hydrogen atom.

In another aspect, the invention relates to the compound of formula (I), or a salt thereof, wherein r is 0, 1, 2, or 3.

In another aspect, the invention relates to the compound of formula (I), or a salt thereof, wherein r is 0, 1, or 2.

In another aspect, the invention relates to the compound of formula (I), or a salt thereof, wherein r is 0 or 1.

In another aspect, the invention relates to the compound of formula (I), or a salt thereof, wherein r is 1.

In another aspect, the invention relates to the compound of formula (I), or a salt thereof, wherein r is 0.

In another aspect, the invention relates to the compound of formula (I), or a salt thereof
wherein each R⁴, if present, is independently selected from the group consisting of R⁵ and R⁶;
   each R⁵ is independently selected from the group consisting of —OR⁶, —NR⁶R⁶, halogen, and —CN;
   each R⁶ is independently selected from the group consisting of hydrogen, C₁₋₆alkyl, C₁₋₆alkoxy, C₁₋₆haloalkyl, C₃₋₁₀cycloalkyl, C₄₋₁₀cycloalkenyl, 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl and 4-11 membered heterocyclyl, are each independently optionally substituted with one or more, identical or different R⁷ and/or R⁸;
each R⁷ is independently selected from the group consisting of —OH, —NH₂, —NHR⁸, —NR⁸R⁸, halogen, —CN, and C₁₋₆alkoxy;
each R⁸ is independently selected from the group consisting of C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, are each independently optionally substituted with one or more, identical or different R⁹;
each R⁹ is —OH, halogen or C₁₋₆alkoxy.

In another aspect, the invention relates to the compound of formula (I), or a salt thereof
wherein each R⁴, if present, is independently selected from the group consisting of R⁵ and R⁶;
   each R⁵ is independently selected from the group consisting of —OR⁶, —NR⁶R⁶, halogen, and —CN;
   each R⁶ is independently selected from the group consisting of hydrogen, C₁₋₆alkyl, C₃₋₁₀cycloalkyl, and 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, —NH₂, —NHR⁸, —NR⁸R⁸, halogen, —CN, and C₁₋₆alkoxy;
each R⁸ is independently selected from the group consisting of C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, are each independently optionally substituted with one or more, identical or different R⁹;
each R⁹ is —OH, halogen or C₁₋₆alkoxy.

In another aspect, the invention relates to the compound of formula (I), or a salt thereof
wherein each R⁴, if present, is independently selected from the group consisting of R⁵ and R⁶;
   each R⁵ is independently selected from the group consisting of —OR⁶, —NR⁶R⁶, halogen, and —CN;
   each R⁶ is independently selected from the group consisting of hydrogen, C₁₋₆alkyl, C₃₋₁₀cycloalkyl, 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH.

In another aspect, the invention relates to the compound of the formula (I), or a salt thereof, wherein each R⁴, if present, is independently selected from the group consisting of

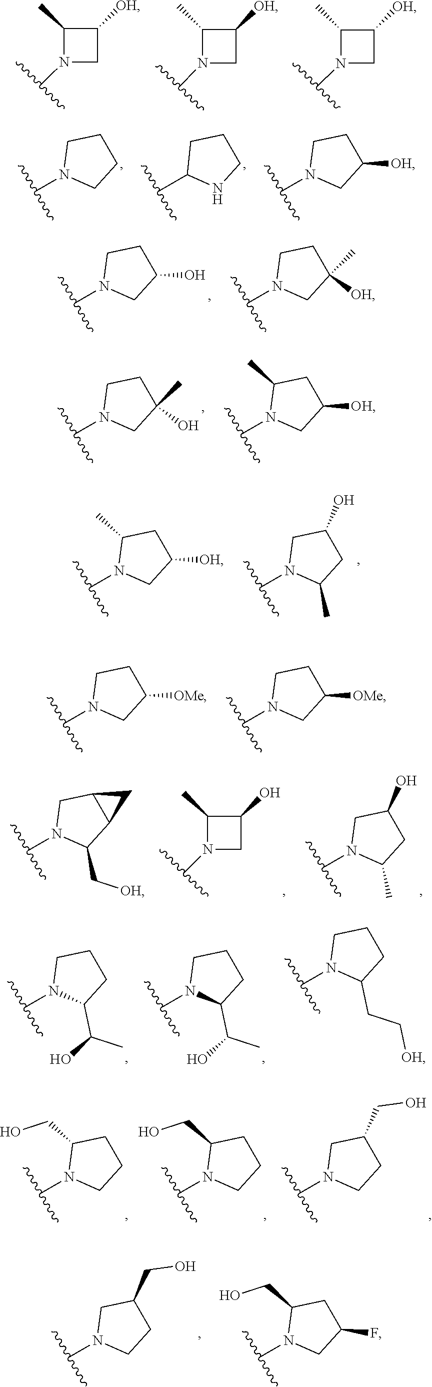

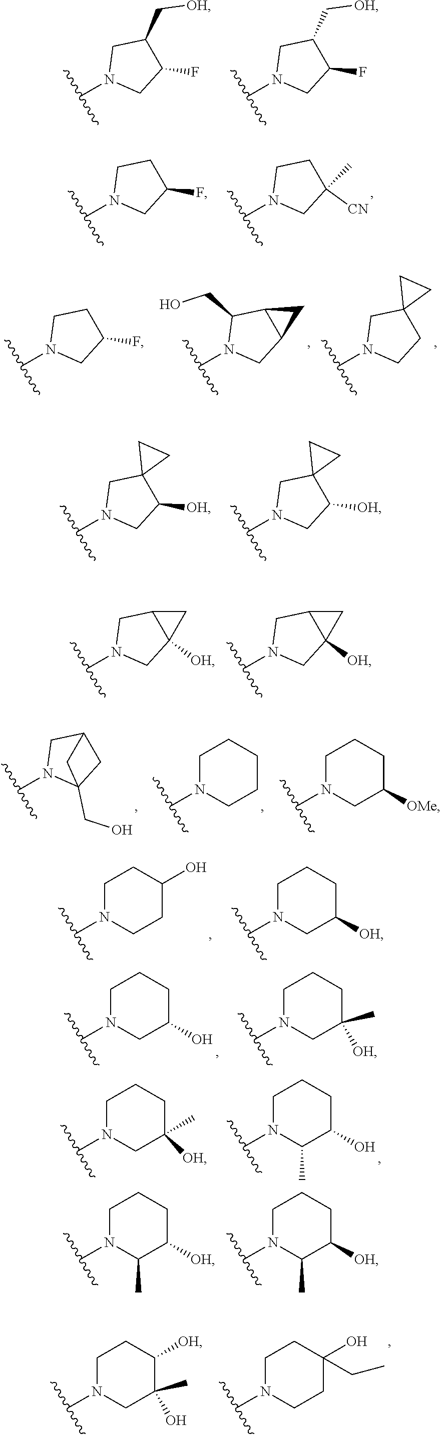

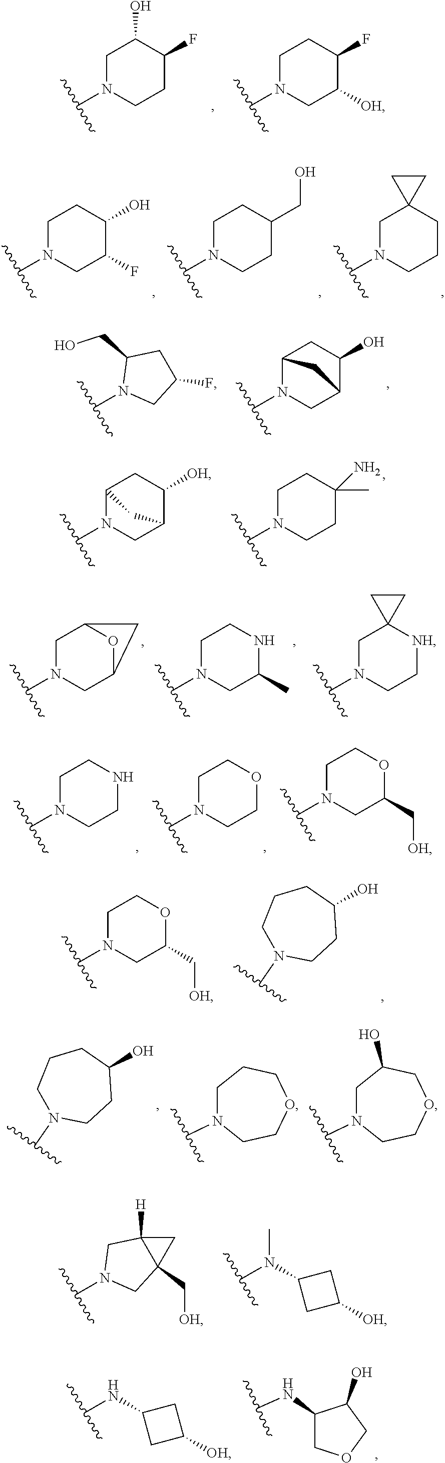

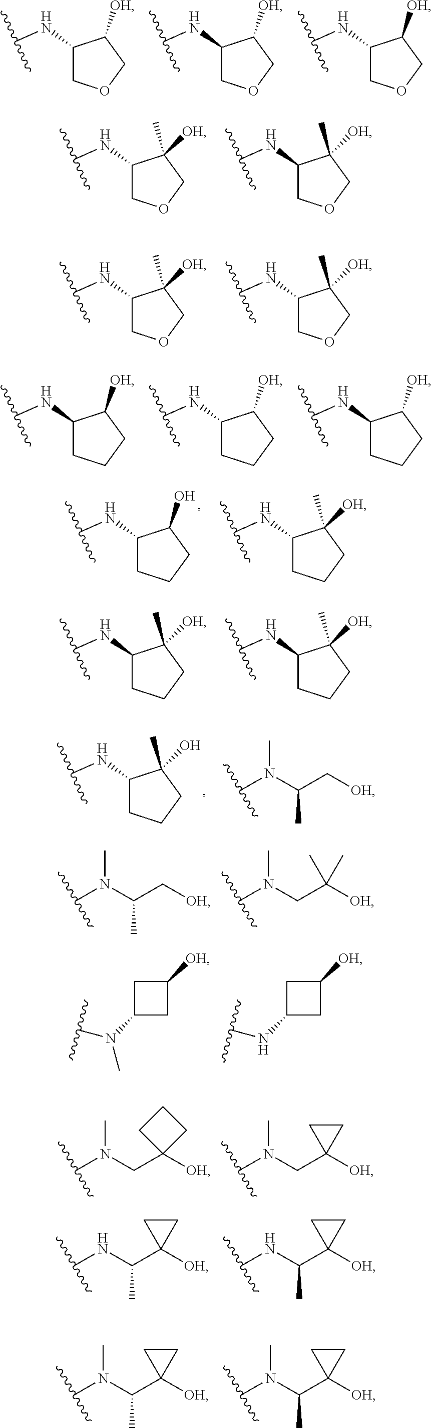

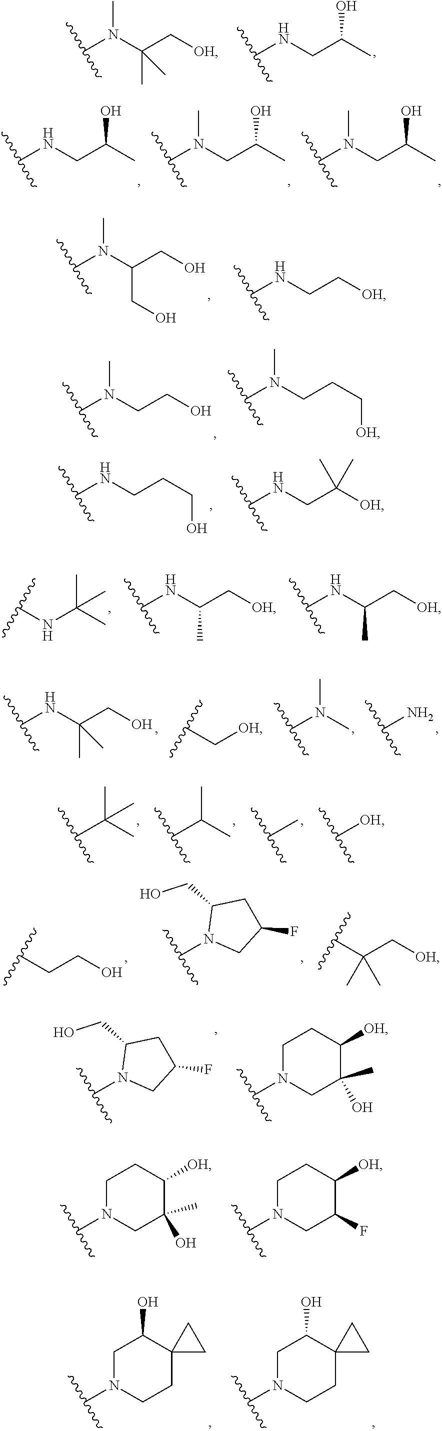

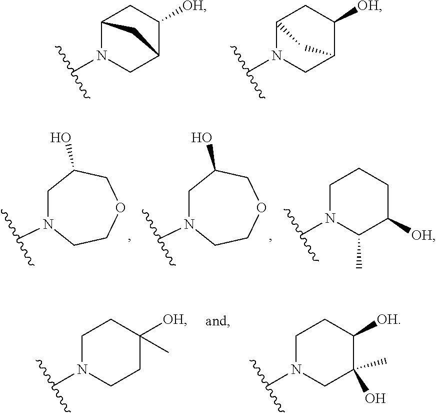

In one embodiment, the invention relates to compounds of formula (I) wherein
each R¹ is independently selected from the group consisting of Me, —CFH₂, —CF₂H, —CF₃, —CFMeH, —CFMe₂, —CF₂Me, F;
p is 2;
R² is Me;
V is nitrogen (—N═) or carbon

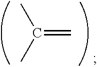

W is nitrogen (—N═) or carbon

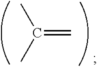

at least one of V and W is a nitrogen (—N═);
A¹, A² and A³ are each independently selected from nitrogen (—N═) or carbon (═CH—); at least one of A¹, A² and A³ is a nitrogen (—N═)
and [symbol] is a single or double bond;
q is 0,
ring system B is a 4-13 membered heterocyclyl,
r denotes 0, 1, 2, or 3
each R⁴, if present, is independently selected from the group consisting of R⁵ and R⁶;
   each R⁵ is independently selected from the group consisting of —OR⁶, and —NR⁶R⁶;
   each R⁶ is independently selected from the group consisting of hydrogen, C₁₋₆alkyl, C₃₋₁₀cycloalkyl, 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of Me, —CFH₂, —CF₂H, —CF₃, —CFMeH, —CFMe₂, —CF₂Me, F;
p is 2;
R² is Me;
V is nitrogen (—N═) or carbon

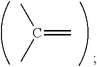

W is nitrogen (—N═) or carbon

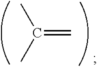

one of V and W is a nitrogen (—N═)
A¹, A² and A³ are each independently selected from nitrogen (—N═) or carbon (═CH—); at least two of A¹, A² and A³ are a nitrogen (—N═)
and [symbol] is a single or double bond;
q is 0,
ring system B is a 4-13 membered heterocyclyl,
r denotes 0, 1, 2, or 3
each R⁴, if present, is independently selected from the group consisting of R¹ and R;
   each R⁵ is independently selected from the group consisting of —OR⁶, and —NR⁶R⁶;
   each R⁶ is independently selected from the group consisting of hydrogen, C₁₋₆alkyl, C₃₋₁₀cycloalkyl, and 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of Me, —CFH₂, —CF₂H, —CF₃, —CFMeH, —CFMe₂, —CF₂Me, F;
p is 2;
R² is Me;
V is nitrogen (—N═);
W is carbon

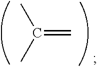

A¹ and A² are independently nitrogen (—N═) or carbon (═CH—);
A³ is nitrogen (—N═);
q is 0,
ring system B is a 4-13 membered heterocyclyl,
r denotes 0, 1, 2, or 3
each R⁴, if present, is independently selected from the group consisting of R⁵ and R⁶;
   each R⁵ is independently selected from the group consisting of —OR⁶, and —NR⁶R⁶;
   each R⁶ is independently selected from the group consisting of hydrogen, C₁₋₆alkyl, C₃₋₁₀cycloalkyl, and 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of —CF₂H, —CF₃, — and F;
p is 2;
R² is Me;
V is nitrogen (—N═);
W is carbon

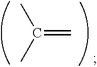

A¹ and A² are independently nitrogen (—N═) or carbon (═CH—);
A³ is nitrogen (—N═);
q is 0,
ring system B is a 5-6 membered heterocyclyl,
r denotes 1 or 2
each R⁴, is independently selected from the group consisting of R⁵ and R⁶;
   each R⁵ is independently selected from the group consisting of —OR⁶, and —NR⁶R⁶;
   each R⁶ is independently selected from the group consisting of hydrogen, C₁₋₆alkyl, C₃₋₁₀cycloalkyl, and 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of —CF₂H, —CF₃, — and F;
p is 2;
R² is Me;
V is nitrogen (—N═);
W is carbon

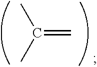

A¹ and A² are independently nitrogen (—N═) or carbon (═CH—);
A³ is nitrogen (—N═);
q is 0,
ring system B is a 5-6 membered heterocyclyl,
r is 1
R⁴ is R⁶;
   R⁶ is a 4-11 membered heterocyclyl, wherein the 4-11 membered heterocyclyl, is optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of —CF₂H, —CF₃, — and F;
p is 2;
R² is Me;
V is nitrogen (—N═);
W is carbon

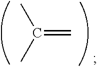

A¹ and A² are independently nitrogen (—N═) or carbon (═CH—);
A³ is nitrogen (—N═);
q is 0,
ring system B is a 5-6 membered heterocyclyl,
r is 1
R⁴ is R⁶;
   R⁶ is a 4-11 membered heterocyclyl, wherein the 4-11 membered heterocyclyl, is optionally substituted with one or more, identical or different R⁷ and/or R⁸;
each R⁷ is —OH;
each R⁸ is independently selected from the group consisting of C₁₋₆alkyl, and C₃₋₁₀cycloalkyl; or a salt thereof.

In a further embodiment, the invention relates to compounds of formula (I) wherein
each R¹ is independently selected from the group consisting of —CF₂H, —CF₃, — and F;
p is 2;
R² is Me;
V is nitrogen (—N═);
W is carbon

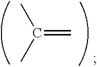

A¹ and A² are independently nitrogen (—N═) or carbon (═CH—);
A³ is nitrogen (—N═);
q is 0,
ring system B is a C₃₋₁₀cycloalkyl,
r is 1
R⁴ is R⁶;
   R⁶ is a 4-11 membered heterocyclyl, wherein the 4-11 membered heterocyclyl, is optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH;
or a salt thereof.

In a further embodiment, the invention relates to compounds of formula (I) wherein
each R¹ is independently selected from the group consisting of —CF₂H, —CF₃, — and F;
p is 2;
R² is Me;
V is nitrogen (—N═);
W is carbon

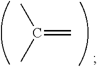

A¹ and A² are independently nitrogen (—N═) or carbon (═CH—);
A³ is nitrogen (—N═);
q is 0,
ring system B is a C₃₋₁₀cycloalkyl,
r is 1
R⁴ is R⁶;
   R⁶ is a 4-11 membered heterocyclyl, wherein the 4-11 membered heterocyclyl, is optionally substituted with one or more, identical or different R⁷ and/or R⁸;
each R⁷ is —OH;
each R⁸ is independently selected from the group consisting of C₁₋₆alkyl, and C₃₋₁₀cycloalkyl;
or a salt thereof.

In a preferred embodiment the compound of formula (I) is selected from

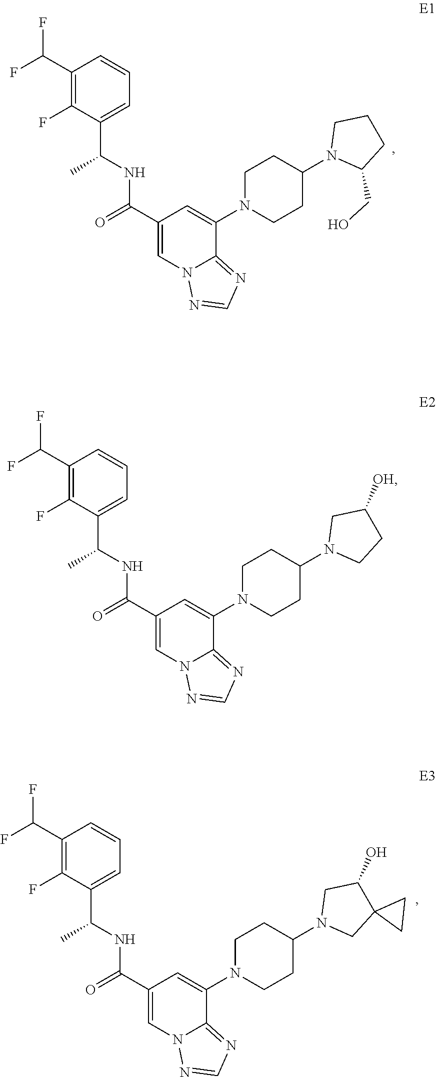 [SMILES: C[C@@H](NC(=O)c1cc(N2CCC(N3CCC[C@@H]3CO)CC2)c2ncnn2c1)c1cccc(C(F)F)c1F ; C[C@@H](NC(=O)c1cc(N2CCC(N3CC[C@@H](O)C3)CC2)c2ncnn2c1)c1cccc(C(F)F)c1F ; C[C@@H](NC(=O)c1cc(N2CCC(N3C[C@H](O)C4(CC4)C3)CC2)c2ncnn2c1)c1cccc(C(F)F)c1F]

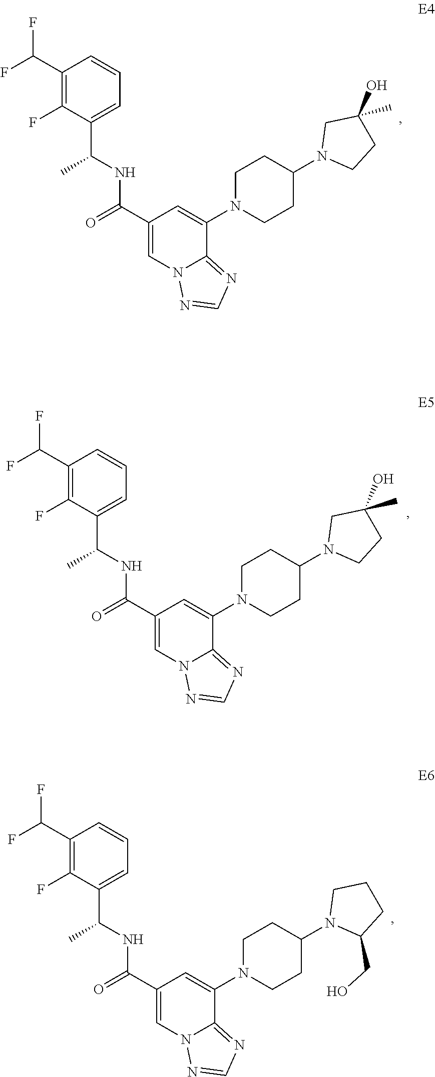 [SMILES: C[C@@H](NC(=O)c1cc(N2CCC(N3CC[C@](C)(O)C3)CC2)c2ncnn2c1)c1cccc(C(F)F)c1F ; C[C@@H](NC(=O)c1cc(N2CCC(N3CC[C@@](C)(O)C3)CC2)c2ncnn2c1)c1cccc(C(F)F)c1F ; C[C@@H](NC(=O)c1cc(N2CCC(N3CCC[C@H]3CO)CC2)c2ncnn2c1)c1cccc(C(F)F)c1F]

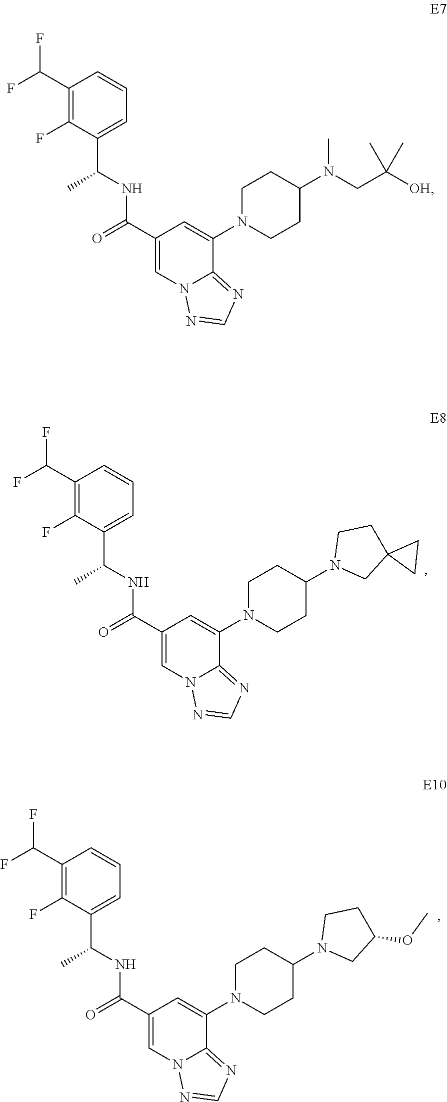 [SMILES: C[C@@H](NC(=O)c1cc(N2CCC(N(C)CC(C)(C)O)CC2)c2ncnn2c1)c1cccc(C(F)F)c1F ; C[C@@H](NC(=O)c1cc(N2CCC(N3CCC4(CC4)C3)CC2)c2ncnn2c1)c1cccc(C(F)F)c1F ; CO[C@H]1CCN(C2CCN(c3cc(C(=O)N[C@H](C)c4cccc(C(F)F)c4F)cn4ncnc34)CC2)C1]

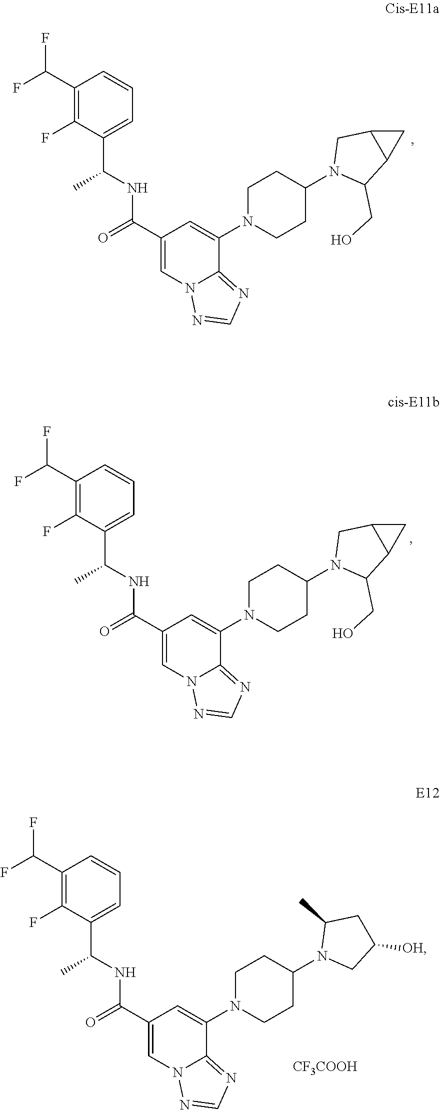 [SMILES: C[C@@H](NC(=O)c1cc(N2CCC(N3CC4CC4C3CO)CC2)c2ncnn2c1)c1cccc(C(F)F)c1F ; C[C@@H](NC(=O)c1cc(N2CCC(N3CC4CC4C3CO)CC2)c2ncnn2c1)c1cccc(C(F)F)c1F ; C[C@@H](NC(=O)c1cc(N2CCC(N3C[C@@H](O)C[C@@H]3C)CC2)c2ncnn2c1)c1cccc(C(F)F)c1F ; "CF3COOH"]

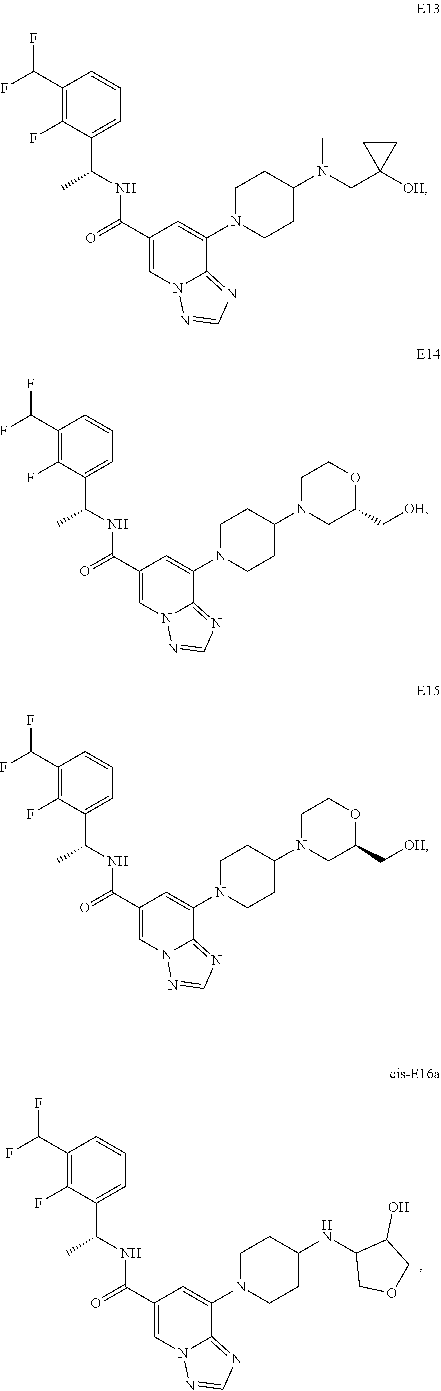 [SMILES: C[C@@H](NC(=O)c1cc(N2CCC(N(C)CC3(O)CC3)CC2)c2ncnn2c1)c1cccc(C(F)F)c1F ; C[C@@H](NC(=O)c1cc(N2CCC(N3CCO[C@H](CO)C3)CC2)c2ncnn2c1)c1cccc(C(F)F)c1F ; C[C@@H](NC(=O)c1cc(N2CCC(N3CCO[C@@H](CO)C3)CC2)c2ncnn2c1)c1cccc(C(F)F)c1F ; C[C@@H](NC(=O)c1cc(N2CCC(NC3COCC3O)CC2)c2ncnn2c1)c1cccc(C(F)F)c1F]

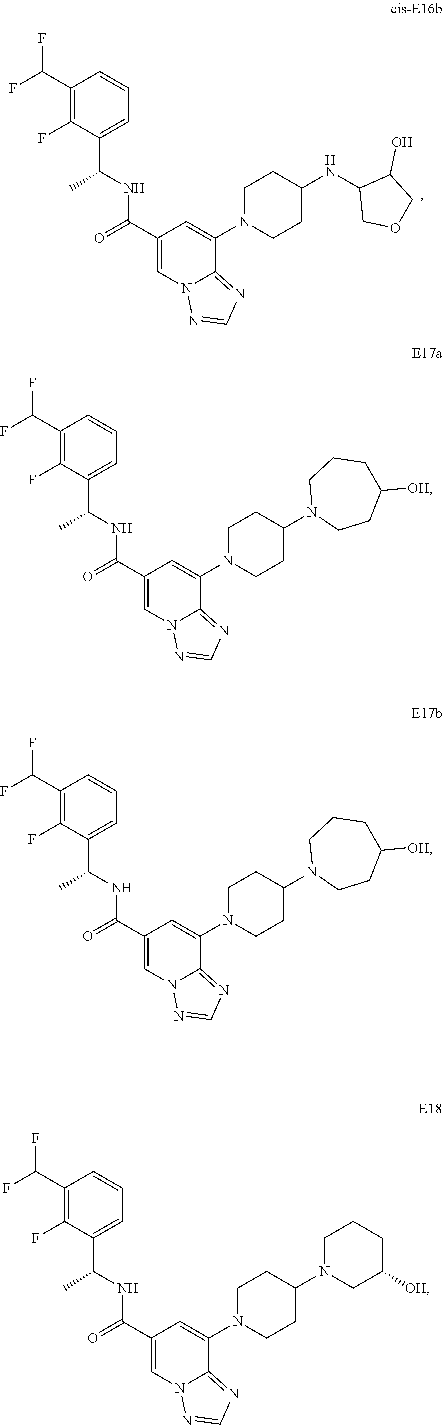 [SMILES: C[C@@H](NC(=O)c1cc(N2CCC(NC3COCC3O)CC2)c2ncnn2c1)c1cccc(C(F)F)c1F ; C[C@@H](NC(=O)c1cc(N2CCC(N3CCCC(O)CC3)CC2)c2ncnn2c1)c1cccc(C(F)F)c1F ; C[C@@H](NC(=O)c1cc(N2CCC(N3CCCC(O)CC3)CC2)c2ncnn2c1)c1cccc(C(F)F)c1F ; C[C@@H](NC(=O)c1cc(N2CCC(N3CCC[C@H](O)C3)CC2)c2ncnn2c1)c1cccc(C(F)F)c1F]

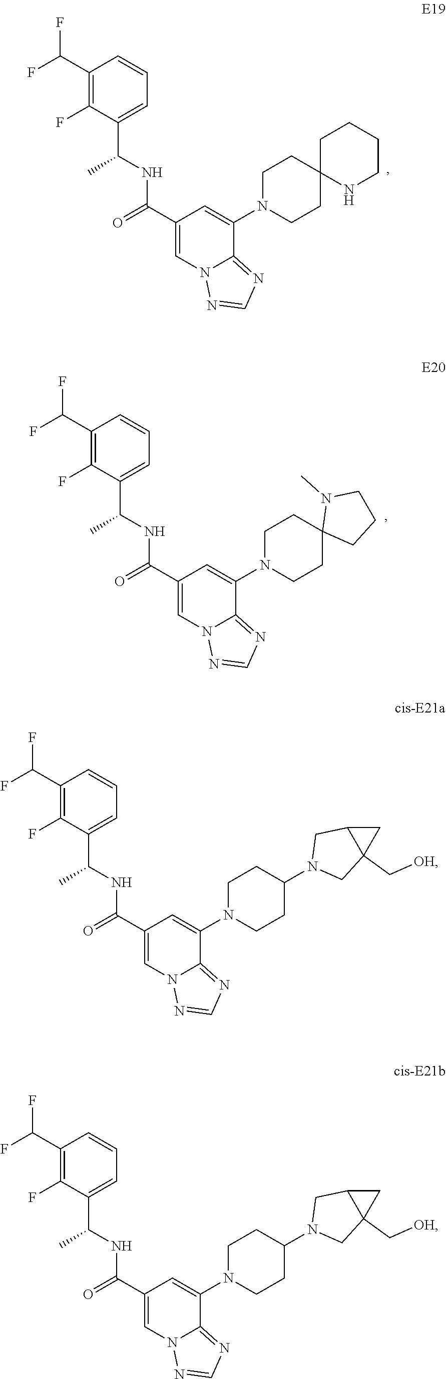 [SMILES: C[C@@H](NC(=O)c1cc(N2CCC3(CCCCN3)CC2)c2ncnn2c1)c1cccc(C(F)F)c1F ; C[C@@H](NC(=O)c1cc(N2CCC3(CCCN3C)CC2)c2ncnn2c1)c1cccc(C(F)F)c1F ; C[C@@H](NC(=O)c1cc(N2CCC(N3CC4CC4(CO)C3)CC2)c2ncnn2c1)c1cccc(C(F)F)c1F ; C[C@@H](NC(=O)c1cc(N2CCC(N3CC4CC4(CO)C3)CC2)c2ncnn2c1)c1cccc(C(F)F)c1F]

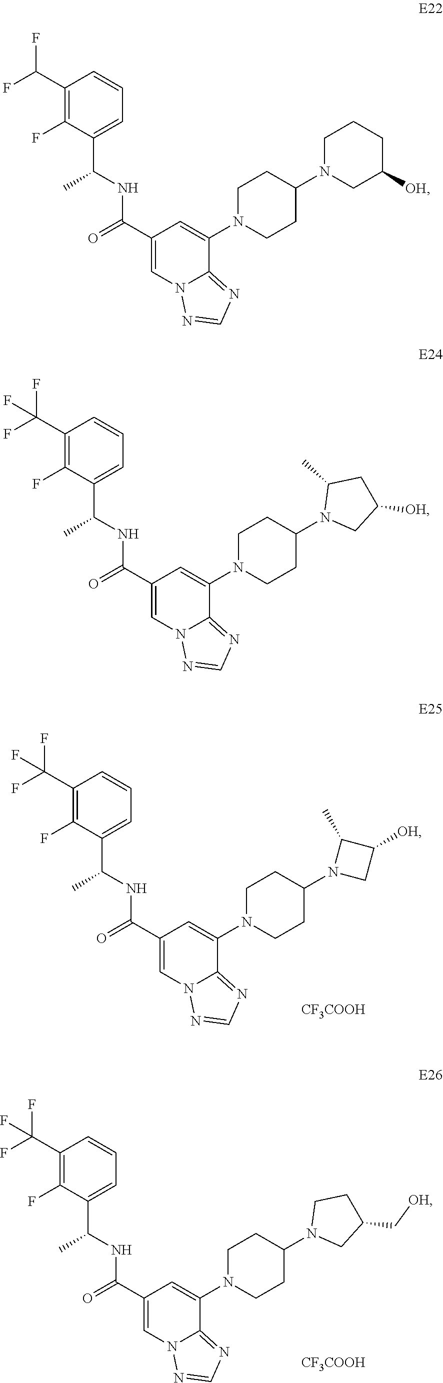 [SMILES: C[C@@H](NC(=O)c1cc(N2CCC(N3CCC[C@@H](O)C3)CC2)c2ncnn2c1)c1cccc(C(F)F)c1F ; C[C@@H]1C[C@H](O)CN1C1CCN(c2cc(C(=O)N[C@H](C)c3cccc(C(F)(F)F)c3F)cn3ncnc23)CC1 ; C[C@@H]1[C@H](O)CN1C1CCN(c2cc(C(=O)N[C@H](C)c3cccc(C(F)(F)F)c3F)cn3ncnc23)CC1 ; "CF3COOH" ; C[C@@H](NC(=O)c1cc(N2CCC(N3CC[C@H](CO)C3)CC2)c2ncnn2c1)c1cccc(C(F)(F)F)c1F ; "CF3COOH"]

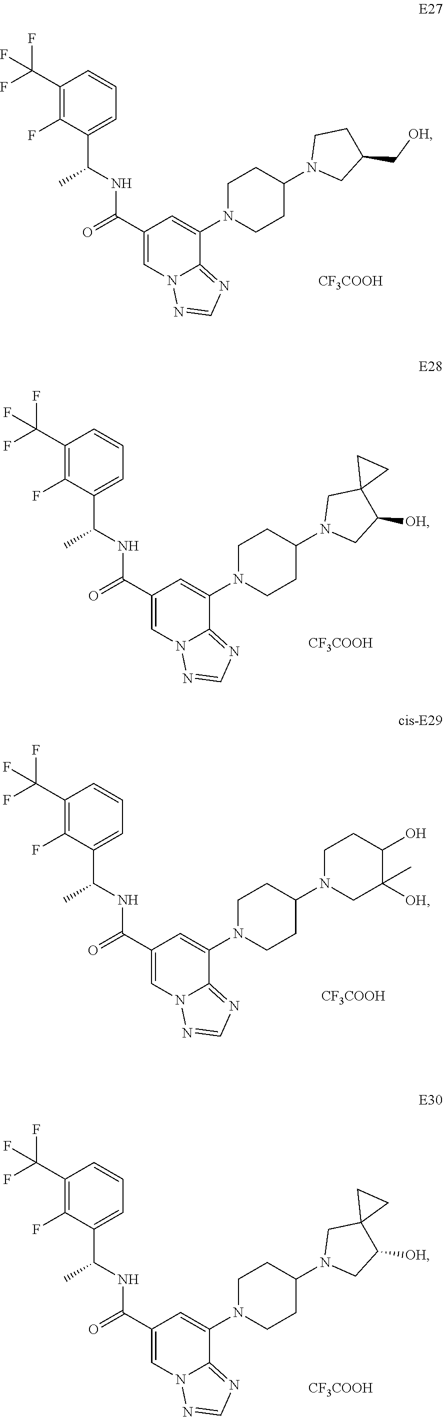

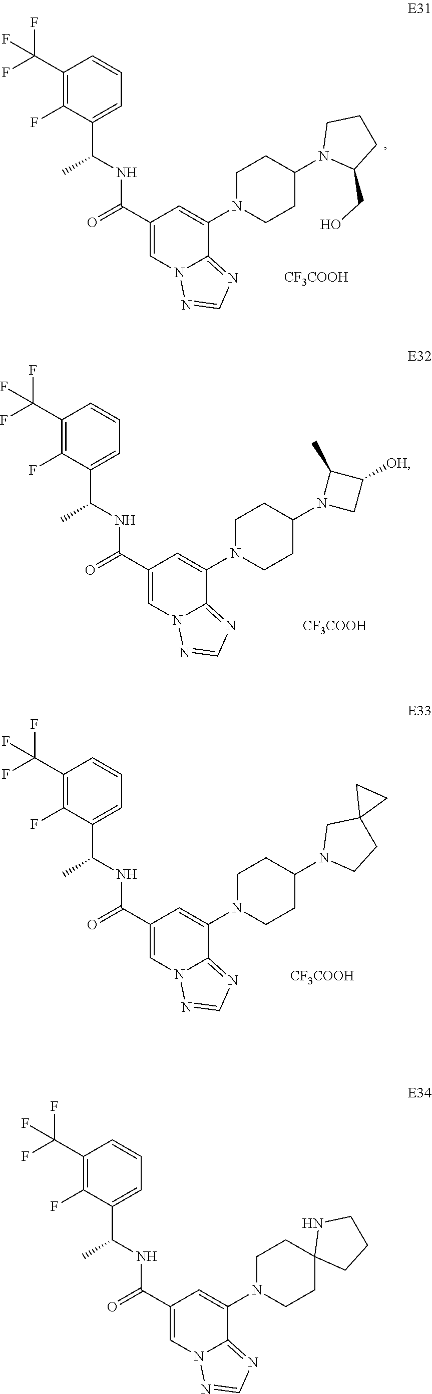

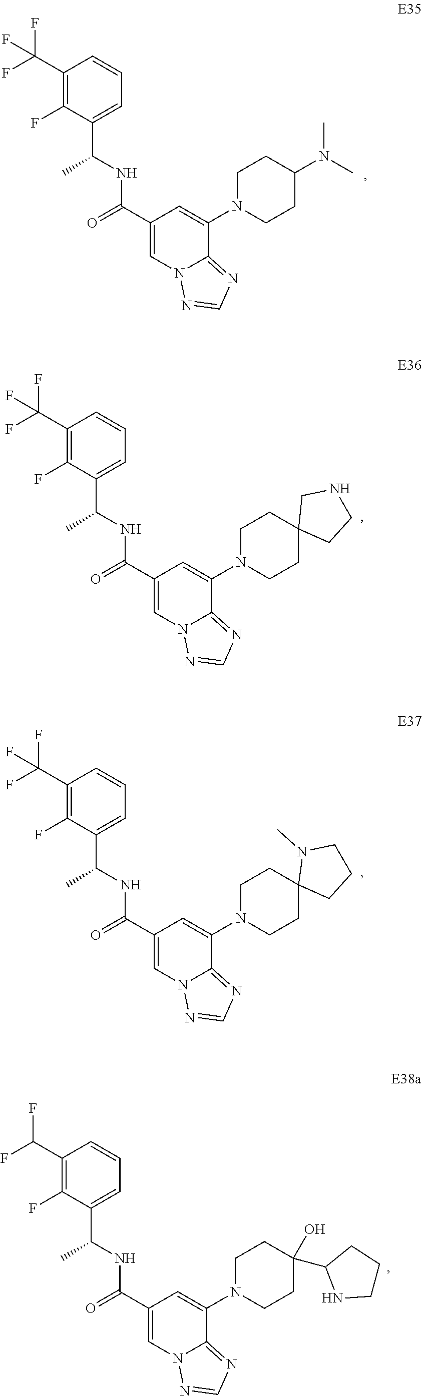 [SMILES: C[C@@H](NC(=O)c1cc(N2CCC(N(C)C)CC2)c2ncnn2c1)c1cccc(C(F)(F)F)c1F ; C[C@@H](NC(=O)c1cc(N2CCC3(CCNC3)CC2)c2ncnn2c1)c1cccc(C(F)(F)F)c1F ; C[C@@H](NC(=O)c1cc(N2CCC3(CCCN3C)CC2)c2ncnn2c1)c1cccc(C(F)(F)F)c1F ; C[C@@H](NC(=O)c1cc(N2CCC(O)(C3CCCN3)CC2)c2ncnn2c1)c1cccc(C(F)F)c1F]

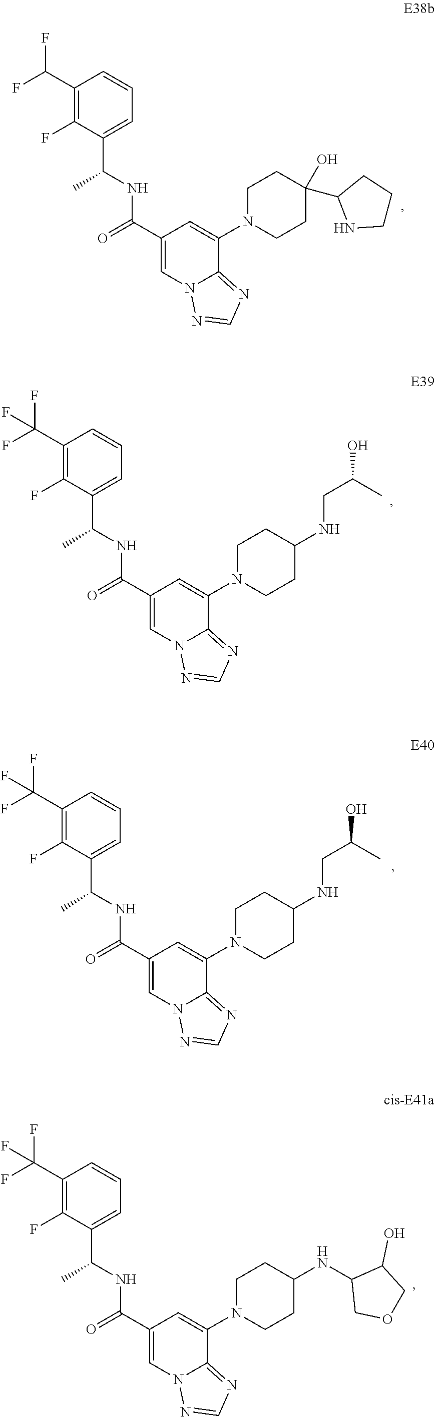 [SMILES: C[C@@H](NC(=O)c1cc(N2CCC(O)(C3CCCN3)CC2)c2ncnn2c1)c1cccc(C(F)F)c1F ; C[C@@H](O)CNC1CCN(c2cc(C(=O)N[C@H](C)c3cccc(C(F)(F)F)c3F)cn3ncnc23)CC1 ; C[C@H](O)CNC1CCN(c2cc(C(=O)N[C@H](C)c3cccc(C(F)(F)F)c3F)cn3ncnc23)CC1 ; C[C@@H](NC(=O)c1cc(N2CCC(NC3COCC3O)CC2)c2ncnn2c1)c1cccc(C(F)(F)F)c1F]

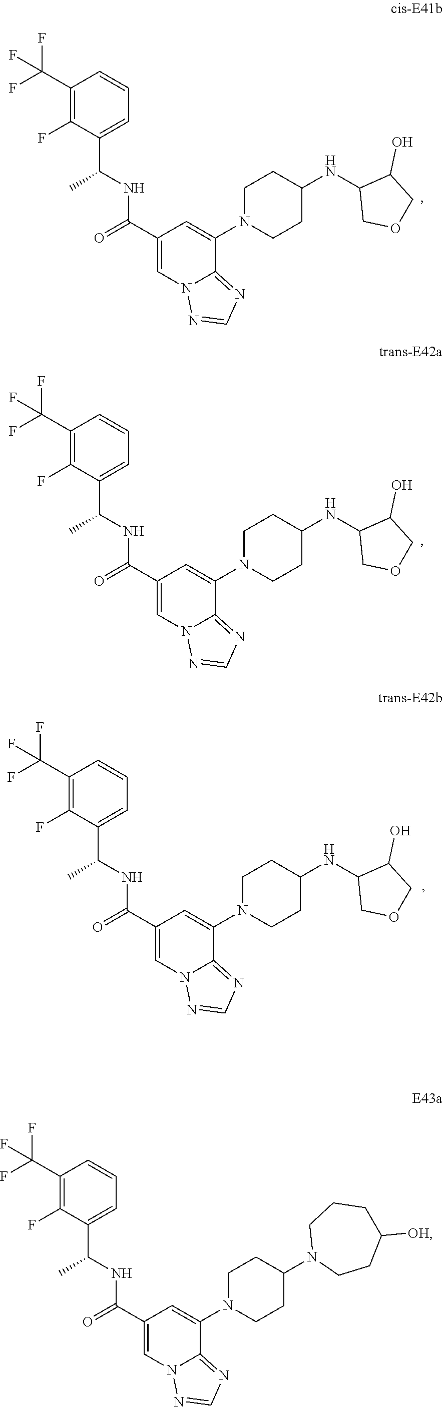 [SMILES: C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(N3CCCC(O)CC3)CC2)c2ncnn2c1)c1cccc(C(F)(F)F)c1F]

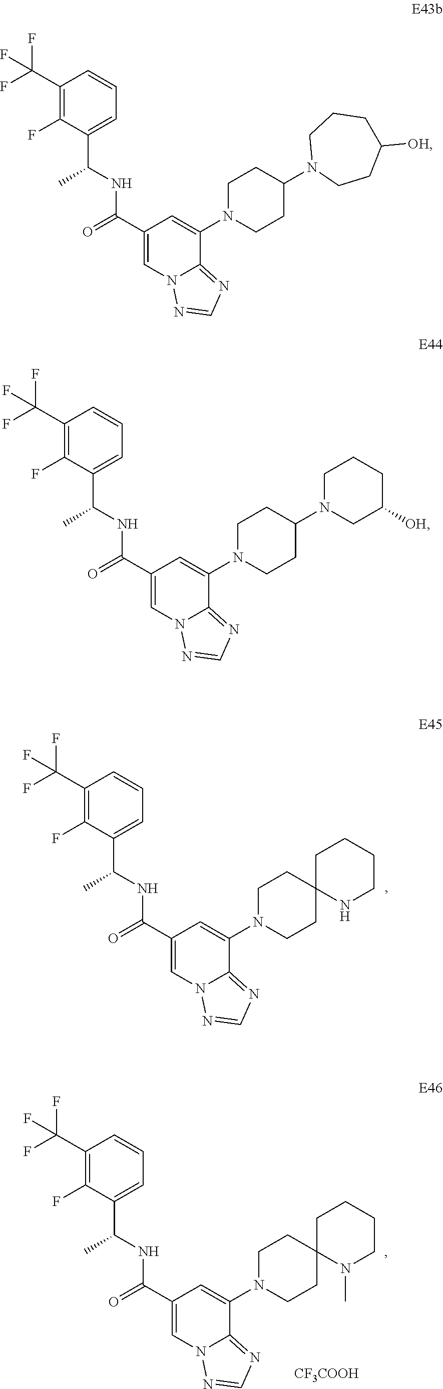 [SMILES: C[C@@H](NC(=O)c1cc(N2CCC(N3CCCC(O)CC3)CC2)c2ncnn2c1)c1cccc(C(F)(F)F)c1F ; C[C@@H](NC(=O)c1cc(N2CCC(N3CCC[C@H](O)C3)CC2)c2ncnn2c1)c1cccc(C(F)(F)F)c1F ; C[C@@H](NC(=O)c1cc(N2CCC3(CCCCN3)CC2)c2ncnn2c1)c1cccc(C(F)(F)F)c1F ; C[C@@H](NC(=O)c1cc(N2CCC3(CCCCN3C)CC2)c2ncnn2c1)c1cccc(C(F)(F)F)c1F ; "CF3COOH"]

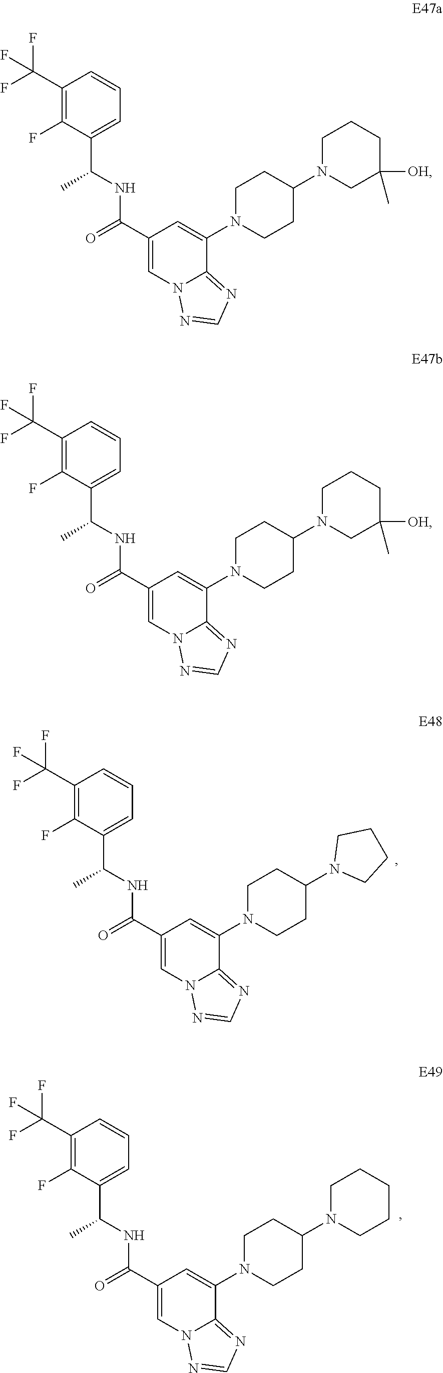 [SMILES: C[C@@H](NC(=O)c1cc(N2CCC(N3CCCC(C)(O)C3)CC2)c2ncnn2c1)c1cccc(C(F)(F)F)c1F ; C[C@@H](NC(=O)c1cc(N2CCC(N3CCCC(C)(O)C3)CC2)c2ncnn2c1)c1cccc(C(F)(F)F)c1F ; C[C@@H](NC(=O)c1cc(N2CCC(N3CCCC3)CC2)c2ncnn2c1)c1cccc(C(F)(F)F)c1F ; C[C@@H](NC(=O)c1cc(N2CCC(N3CCCCC3)CC2)c2ncnn2c1)c1cccc(C(F)(F)F)c1F]

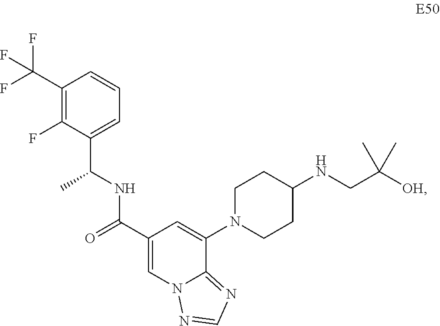 [SMILES: C[C@@H](NC(=O)c1cc(N2CCC(NCC(C)(C)O)CC2)c2ncnn2c1)c1cccc(C(F)(F)F)c1F]

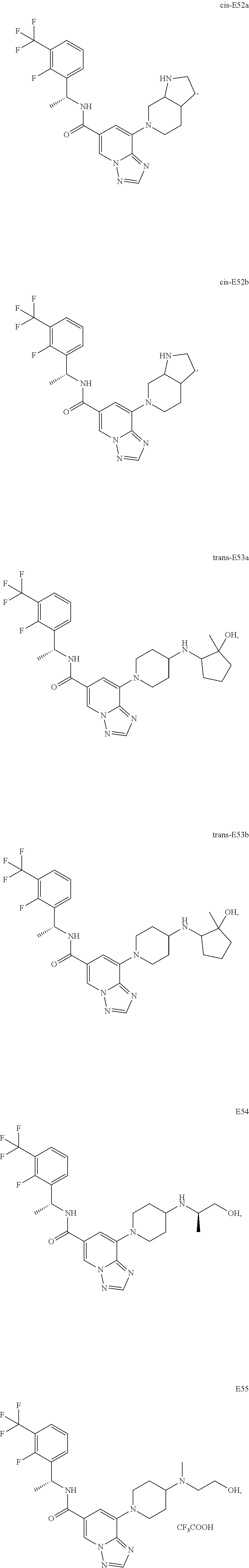

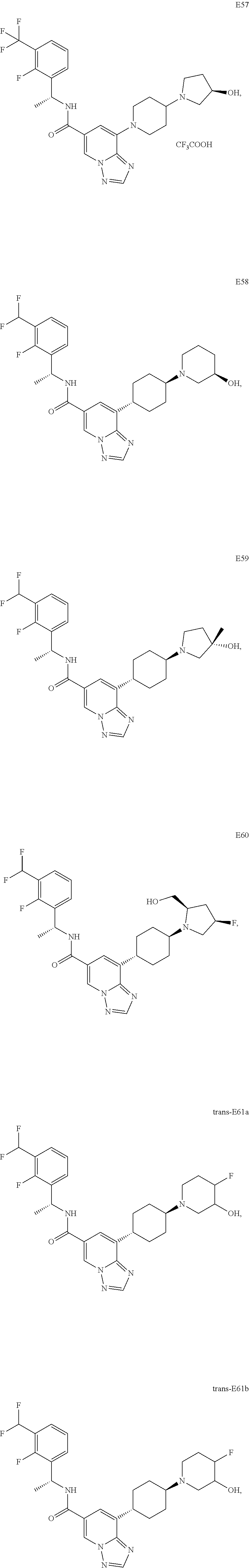

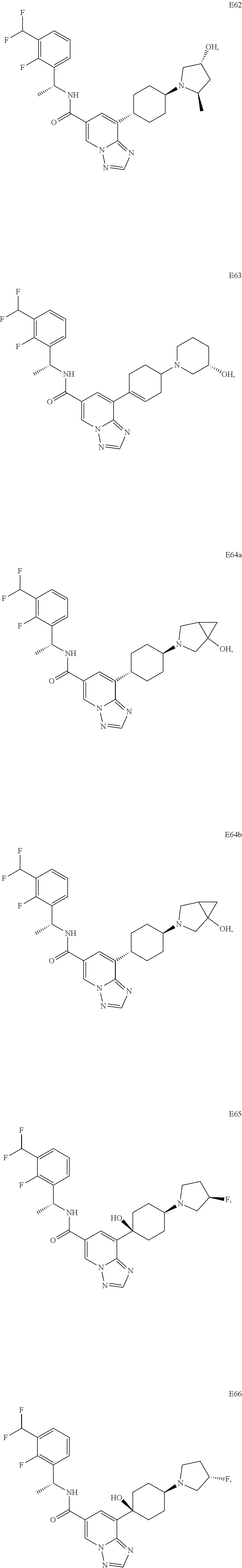 [SMILES: C[C@@H]1C[C@@H](O)CN1[C@H]1CC[C@H](c2cc(C(=O)N[C@H](C)c3cccc(C(F)F)c3F)cn3ncnc23)CC1 ; C[C@@H](NC(=O)c1cc(C2=CCC(N3CCC[C@H](O)C3)CC2)c2ncnn2c1)c1cccc(C(F)F)c1F ; C[C@@H](NC(=O)c1cc([C@H]2CC[C@H](N3CC4CC4(O)C3)CC2)c2ncnn2c1)c1cccc(C(F)F)c1F ; C[C@@H](NC(=O)c1cc([C@H]2CC[C@H](N3CC4CC4(O)C3)CC2)c2ncnn2c1)c1cccc(C(F)F)c1F ; C[C@@H](NC(=O)c1cc([C@]2(O)CC[C@@H](N3CC[C@@H](F)C3)CC2)c2ncnn2c1)c1cccc(C(F)F)c1F ; C[C@@H](NC(=O)c1cc([C@]2(O)CC[C@@H](N3CC[C@H](F)C3)CC2)c2ncnn2c1)c1cccc(C(F)F)c1F]

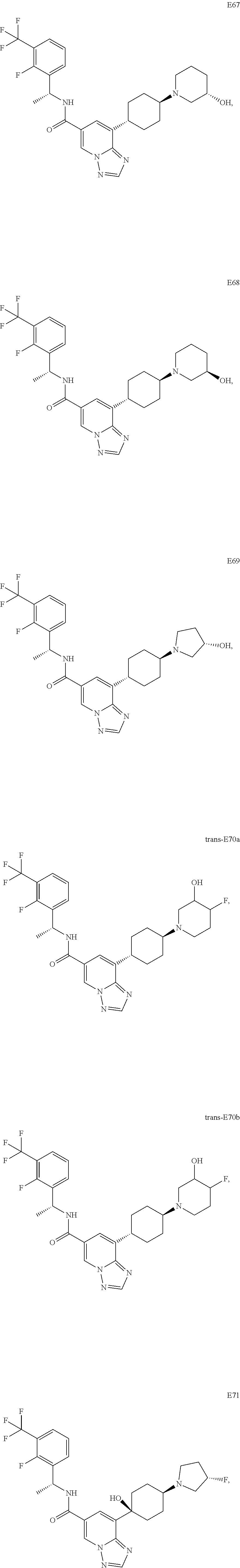

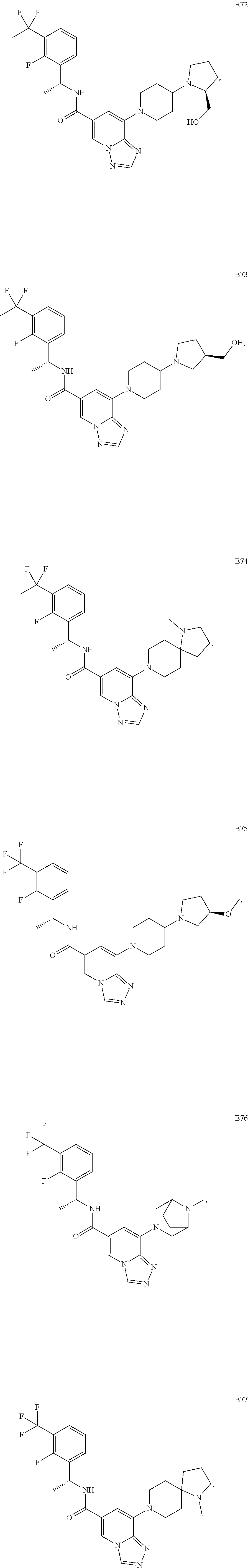 [SMILES: C[C@@H](NC(=O)c1cc(N2CCC(N3CCC[C@H]3CO)CC2)c2ncnn2c1)c1cccc(C(C)(F)F)c1F ; C[C@@H](NC(=O)c1cc(N2CCC(N3CC[C@@H](CO)C3)CC2)c2ncnn2c1)c1cccc(C(C)(F)F)c1F ; C[C@@H](NC(=O)c1cc(N2CCC3(CCCN3C)CC2)c2ncnn2c1)c1cccc(C(C)(F)F)c1F ; CO[C@@H]1CCN(C2CCN(c3cc(C(=O)N[C@H](C)c4cccc(C(F)(F)F)c4F)cn4cnnc34)CC2)C1 ; C[C@@H](NC(=O)c1cc(N2CC3CCC(C2)N3C)c2nncn2c1)c1cccc(C(F)(F)F)c1F ; C[C@@H](NC(=O)c1cc(N2CCC3(CCCN3C)CC2)c2nncn2c1)c1cccc(C(F)(F)F)c1F]

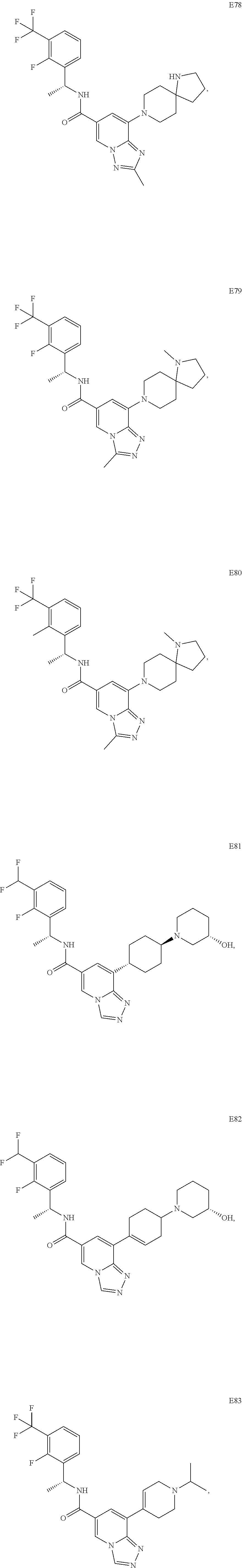 [SMILES: Cc1nc2c(N3CCC4(CCCN4)CC3)cc(C(=O)N[C@H](C)c3cccc(C(F)(F)F)c3F)cn2n1 ; Cc1nnc2c(N3CCC4(CCCN4C)CC3)cc(C(=O)N[C@H](C)c3cccc(C(F)(F)F)c3F)cn12 ; Cc1c([C@@H](C)NC(=O)c2cc(N3CCC4(CCCN4C)CC3)c3nnc(C)n3c2)cccc1C(F)(F)F ; C[C@@H](NC(=O)c1cc([C@H]2CC[C@H](N3CCC[C@H](O)C3)CC2)c2nncn2c1)c1cccc(C(F)F)c1F ; C[C@@H](NC(=O)c1cc(C2=CCC(N3CCC[C@H](O)C3)CC2)c2nncn2c1)c1cccc(C(F)F)c1F ; CC(C)N1CC=C(c2cc(C(=O)N[C@H](C)c3cccc(C(F)(F)F)c3F)cn3cnnc23)CC1]

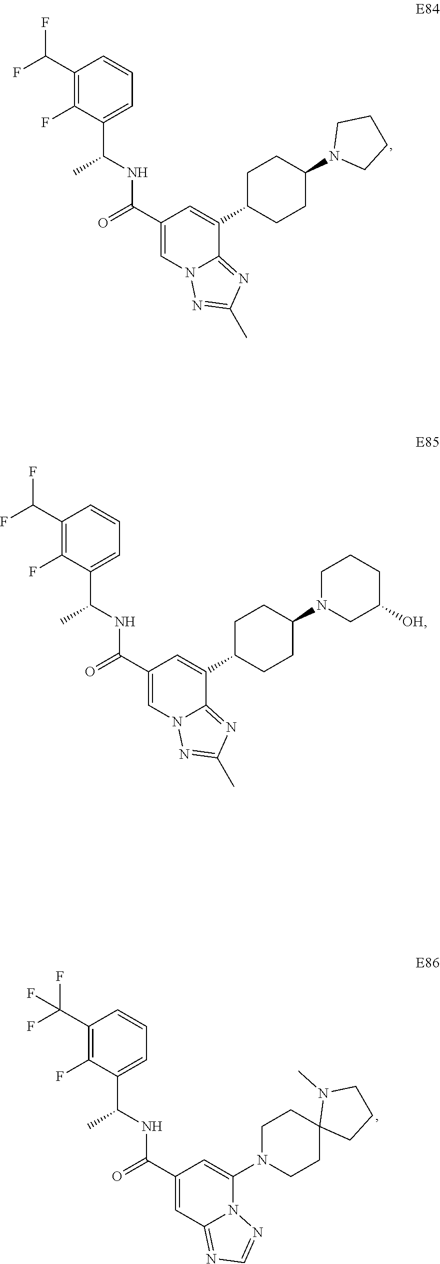 [SMILES: Cc1nc2c([C@H]3CC[C@H](N4CCCC4)CC3)cc(C(=O)N[C@H](C)c3cccc(C(F)F)c3F)cn2n1 ; Cc1nc2c([C@H]3CC[C@H](N4CCC[C@H](O)C4)CC3)cc(C(=O)N[C@H](C)c3cccc(C(F)F)c3F)cn2n1 ; C[C@@H](NC(=O)c1cc(N2CCC3(CCCN3C)CC2)n2ncnc2c1)c1cccc(C(F)(F)F)c1F]

and
or a pharmaceutically acceptable salt thereof.

It is to be understood that any two or more aspects and/or preferred embodiments of formula (I)—or subformulas thereof—may be combined in any way leading to a chemically stable structure to obtain further aspects of formula (I)—or subformulas thereof.

Preferred embodiments of the invention are example compounds E1 to E86.

Preferred embodiments of the invention are example compounds E1 to E86 and/or the pharmaceutically acceptable salts thereof.

In an aspect, the present invention further relates to hydrates, solvates, polymorphs, metabolites, derivatives, stereoisomers and prodrugs of a compound of formula (I) (including all embodiments thereof).

In another aspect, the invention further relates to a hydrate of a compound of formula (I) (including all aspects thereof).

In another aspect, the present invention further relates to a solvate of a compound of formula (I) (including all embodiments thereof).

Compounds of formula (I) (including all embodiments thereof) which e.g. bear ester groups are potential prodrugs the ester being cleaved under physiological conditions and are also part of the invention.

In another aspect, the present invention further relates to a pharmaceutically acceptable salt of a compound of formula (I) (including all embodiments thereof).

In another aspect, the present invention further relates to a pharmaceutically acceptable salt of a compound of formula (I) (including all embodiments thereof) with anorganic or organic acids or bases.

Accordingly, in another aspect the present invention further relates to compounds of formula (I) as defined herein or pharmaceutically acceptable salts thereof for use as a medicament.

Other aspects of the present invention will become apparent to the person skilled in the art directly from the foregoing and following description and examples.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to:
The use of the prefix C_{x-y}, wherein x and y each represent a positive integer (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (e.g. heteroaryl, heteroarylalkyl, heterocyclyl, heterocycylalkyl) relates to the total number of atoms of all the ring members or the total of all the ring and carbon chain members. Obviously, a ring structure has at least three members.

In general, for groups comprising two or more subgroups (e.g. heteroarylalkyl, heterocycylalkyl, cycloalkylalkyl, arylalkyl) the last named subgroup is the radical attachment point, for example, the substituent aryl-C₁₋₆alkyl means an aryl group which is bound to a C₁₋₆alkyl group, the latter of which is bound to the core or to the group to which the substituent is attached.

In groups like HO, H₂N, (O)S, (O)₂S, NC (cyano), HOOC, F₃C or the like, the skilled artisan can see the radical attachment point(s) to the molecule from the free valences of the group itself.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}alkylamino or C_{x-y}alkyloxy.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

By heteroatoms are meant oxygen, nitrogen and sulphur atoms.

Haloalkyl is derived from the previously defined alkyl by replacing one or more hydrogen atoms of the hydrocarbon chain independently of one another by halogen atoms, which may be identical or different. If a haloalkyl is to be further substituted, the substitutions may take place independently of one another, in the form of mono- or polysubstitutions in each case, on all the hydrogen-carrying carbon atoms.

Examples of haloalkyl are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CHFCH₂CH₃, —CHFCH₂CF₃ etc.

Halogen relates to fluorine, chlorine, bromine and/or iodine atoms.

Cycloalkyl is made up of the subgroups monocyclic hydrocarbon rings, bicyclic hydrocarbon rings and spiro-hydrocarbon rings. The systems are saturated. In bicyclic hydrocarbon rings two rings are joined together so that they have at least two carbon atoms in common. In spiro-hydrocarbon rings one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

If the free valency of a cycloalkyl is saturated, then an alicycle is obtained.

Cycloalkenyl is also made up of the subgroups monocyclic hydrocarbon rings, bicyclic hydrocarbon rings and spiro-hydrocarbon rings. However, the systems are unsaturated, i.e. there is at least one C—C double bond but no aromatic system. If in a cycloalkyl as hereinbefore defined two hydrogen atoms at adjacent cyclic carbon atoms are formally removed and the free valencies are saturated to form a second bond, the corresponding cycloalkenyl is obtained.

If a cycloalkenyl is to be substituted, the substitutions may take place independently of one another, in the form of mono- or polysubstitutions in each case, on all the hydrogen-carrying carbon atoms. Cycloalkenyl itself may be linked as a substituent to the molecule via every suitable position of the ring system.

Examples of cycloalkenyl are cycloprop-1-enyl, cycloprop-2-enyl, cyclobut-1-enyl, cyclobut-2-enyl, cyclopent-1-enyl, cyclopent-2-enyl, cyclopent-3-enyl, cyclohex-1-enyl, cyclohex-2-enyl, cyclohex-3-enyl, cyclohept-1-enyl, cyclohept-2-enyl, cyclohept-3-enyl, cyclohept-4-enyl, cyclobuta-1,3-dienyl, cyclopenta-1,4-dienyl, cyclopenta-1,3-dienyl, cyclopenta-2,4-dienyl, cyclohexa-1,3-dienyl, cyclohexa-1,5-dienyl, cyclohexa-2,4-dienyl, cyclohexa-1,4-dienyl, cyclohexa-2,5-dienyl, bicyclo[2.2.1]hepta-2,5-dienyl (norborna-2,5-dienyl), bicyclo[2.2.1]hept-2-enyl (norbornenyl), spiro[4,5]dec-2-enyl etc.

If the free valency of a cycloalkenyl is saturated, then an unsaturated alicycle is obtained.

Heterocyclyl denotes ring systems, which are derived from the previously defined cycloalkyl and cycloalkenyl by replacing one or more of the groups —CH₂— independently of one another in the hydrocarbon rings by the groups —O—, —S— or —NH— or by replacing one or more of the groups ═CH— by the group ═N—, wherein a total of not more than five heteroatoms may be present, at least one carbon atom must be present between two oxygen atoms and between two sulphur atoms or between an oxygen and a sulphur atom and the ring as a whole must have chemical stability. Heteroatoms may optionally be present in all the possible oxidation stages (sulphur→sulphoxide —SO—, sulphone —SO₂-nitrogen→N-oxide). In a heterocyclyl there is no heteroaromatic ring, i.e. no heteroatom is part of an aromatic system.

A direct result of the derivation from cycloalkyl and cycloalkenyl is that heterocyclyl is made up of the subgroups monocyclic heterorings, bicyclic heterorings, tricyclic heterorings and spiro-heterorings, which may be present in saturated or unsaturated form.

By unsaturated is meant that there is at least one double bond in the ring system in question, but no heteroaromatic system is formed. In bicyclic heterorings two rings are linked together so that they have at least two (hetero)atoms in common. In spiro-heterorings one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system. Substituents on heterocyclyl do not count for the number of members of a heterocyclyl.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diaza-spiro[4,5]decyl etc.

Further examples are the structures illustrated below, which may be attached via each hydrogen-carrying atom (exchanged for hydrogen):

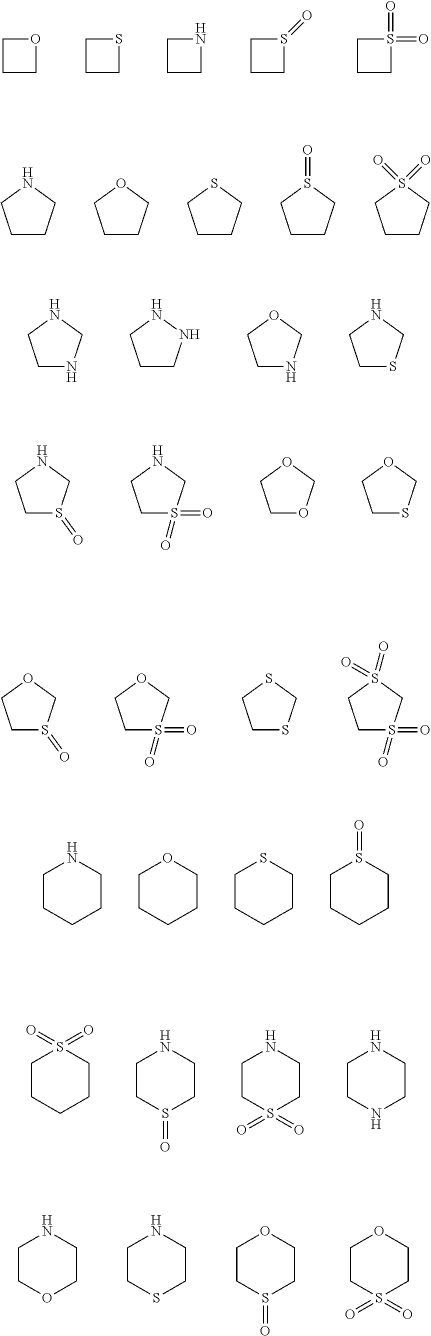

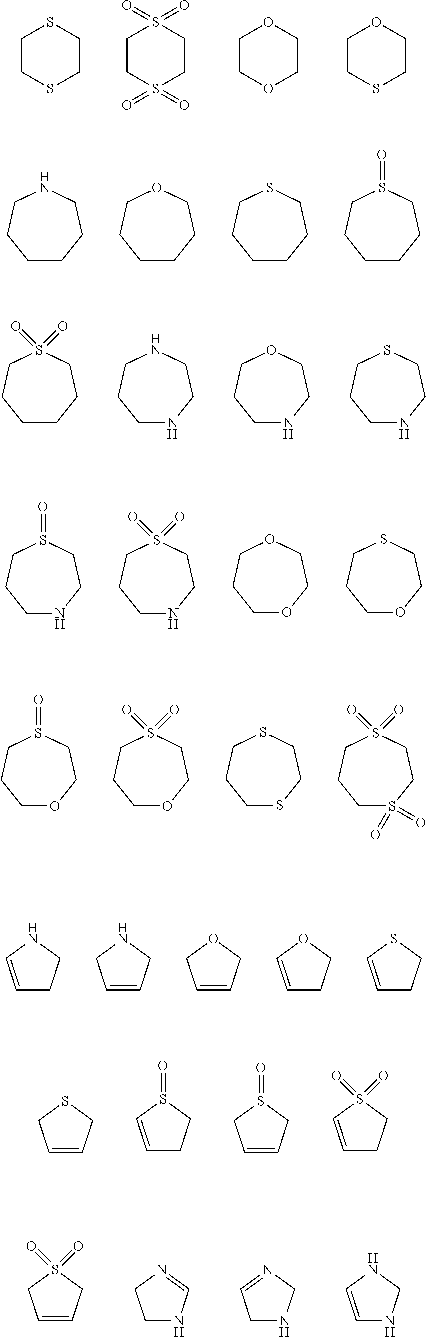

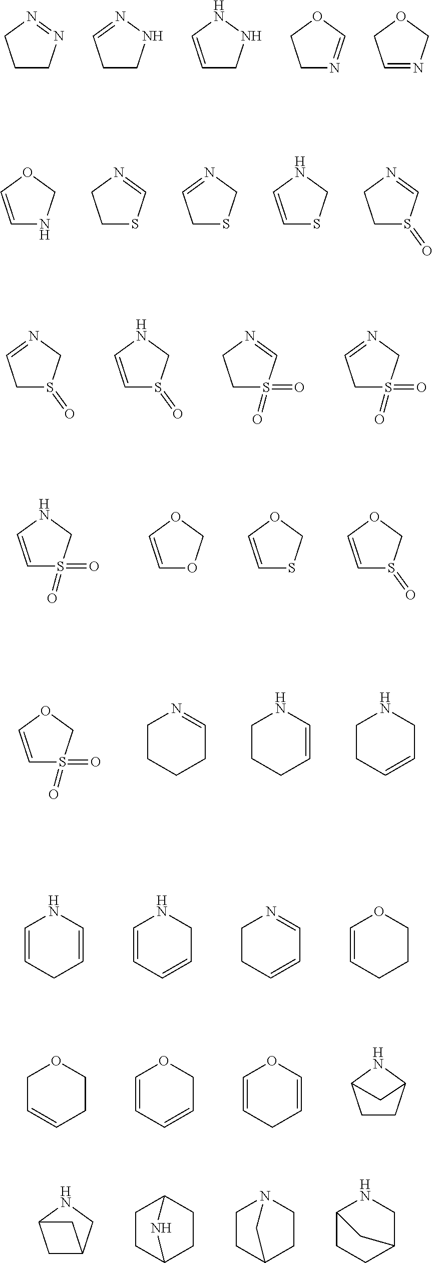

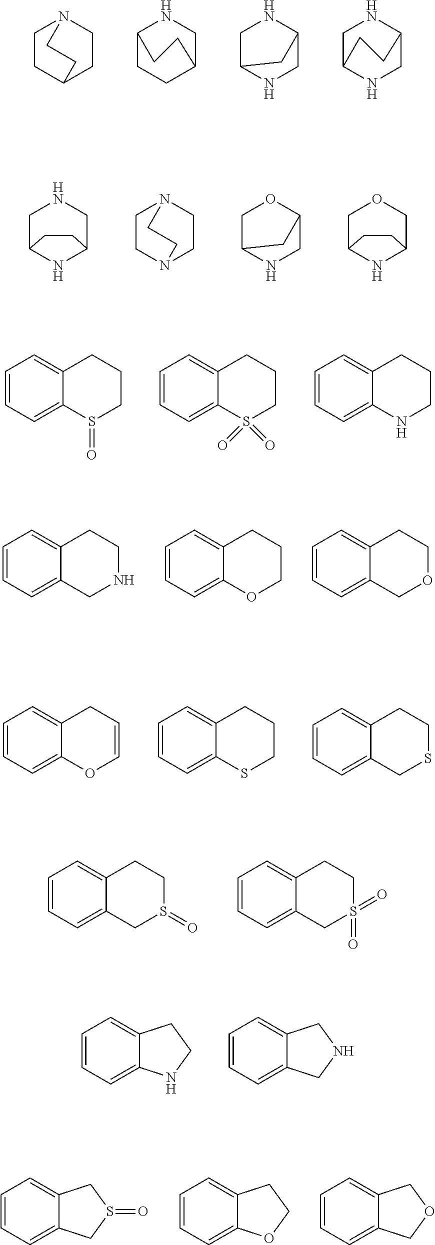

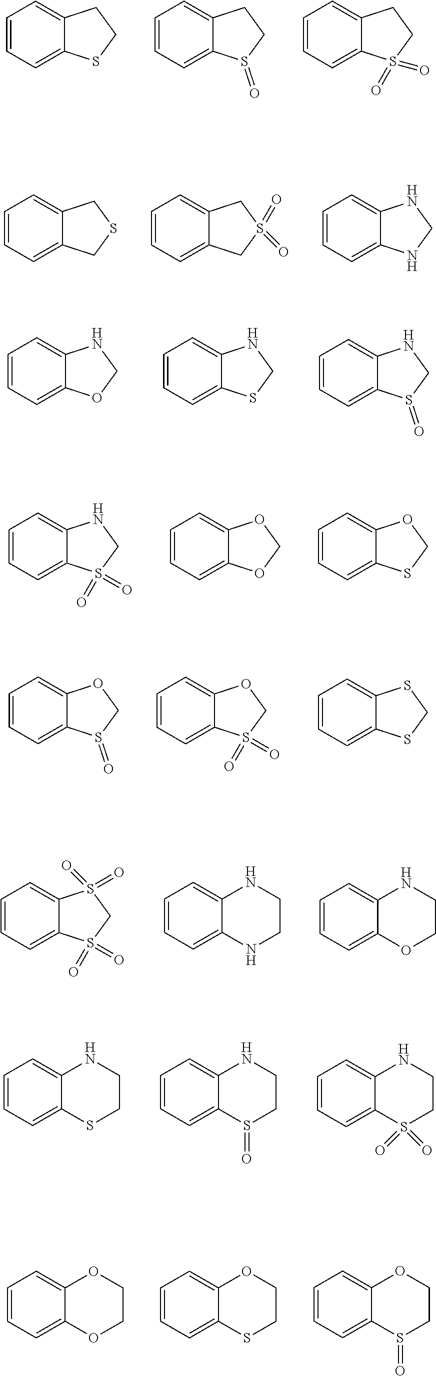

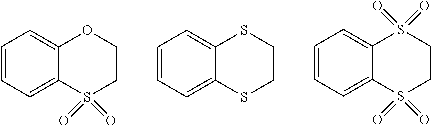

If the free valency of a heterocyclyl is saturated, then a heterocycle is obtained.

Heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. The prerequisite for the presence of heteroaryl is a heteroatom and a heteroaromatic system.

If a heteroaryl is to be substituted, the substitutions may take place independently of one another, in the form of mono- or polysubstitutions in each case, on all the hydrogen-carrying carbon and/or nitrogen atoms. Heteroaryl itself may be linked as a substituent to the molecule via every suitable position of the ring system, both carbon and nitrogen. Substituents on heteroaryl do not count for the number of members of a heteroaryl.

Examples of heteroaryl are furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, pyridyl-N-oxide, pyrrolyl-N-oxide, pyrimidinyl-N-oxide, pyridazinyl-N-oxide, pyrazinyl-N-oxide, imidazolyl-N-oxide, isoxazolyl-N-oxide, oxazolyl-N-oxide, thiazolyl-N-oxide, oxadiazolyl-N-oxide, thiadiazolyl-N-oxide, triazolyl-N-oxide, tetrazolyl-N-oxide, indolyl, isoindolyl, benzofuryl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl, benzotriazinyl, indolizinyl, oxazolopyridyl, imidazopyridyl, naphthyridinyl, benzoxazolyl, pyridopyridyl, pyrimidopyridyl, purinyl, pteridinyl, benzothiazolyl, imidazopyridyl, imidazothiazolyl, quinolinyl-N-oxide, indolyl-N-oxide, isoquinolyl-N-oxide, quinazolinyl-N-oxide, quinoxalinyl-N-oxide, phthalazinyl-N-oxide, indolizinyl-N-oxide, indazolyl-N-oxide, benzothiazolyl-N-oxide, benzimidazolyl-N-oxide etc.

Further examples are the structures illustrated below, which may be attached via each hydrogen-carrying atom (exchanged for hydrogen):

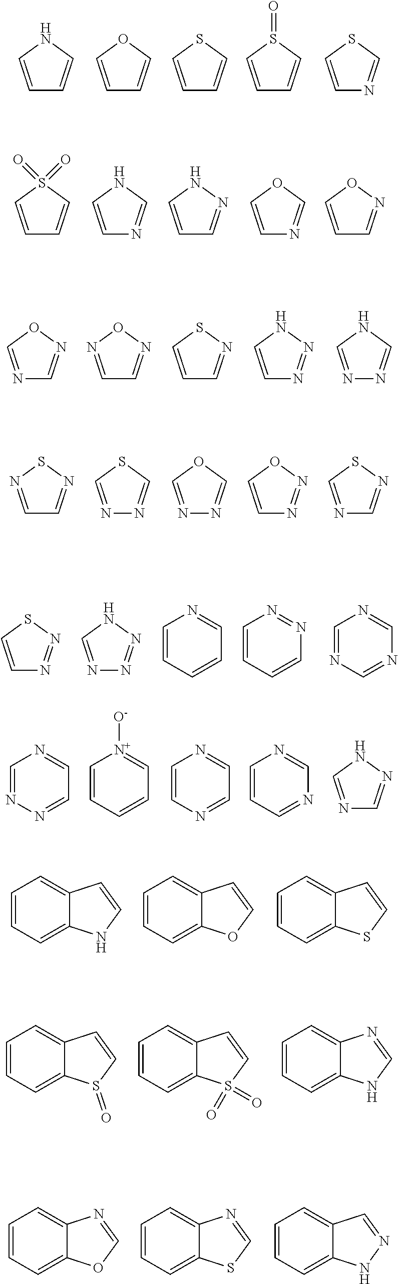

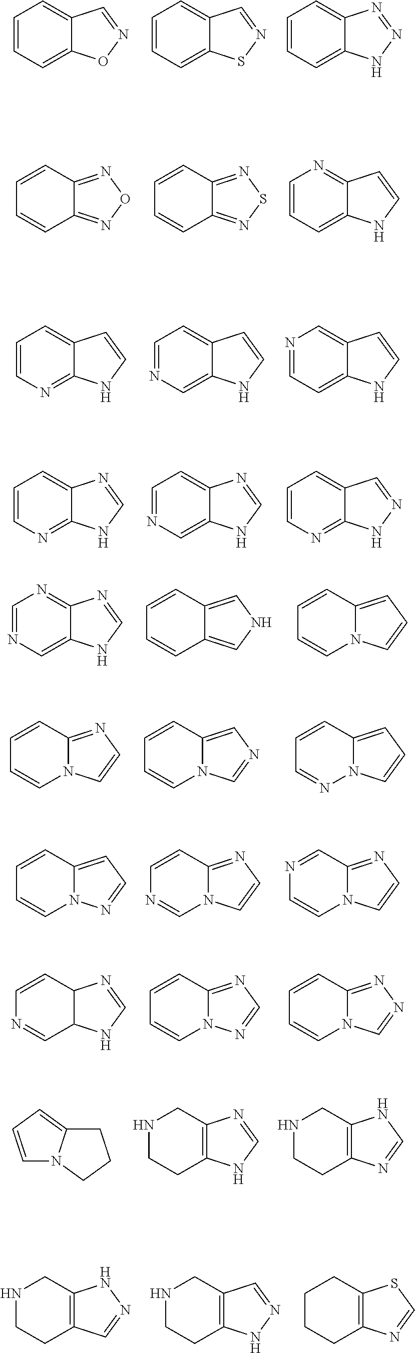

Preferably, heteroaryls are 5-6 membered monocyclic or 9-10 membered bicyclic, each with 1 to 4 heteroatoms independently selected from oxygen, nitrogen and sulfur.

If the free valency of a heteroaryl is saturated, a heteroarene is obtained.

[symbol] is a single or double bond.

By substituted is meant that a hydrogen atom which is bound directly to the atom under consideration, is replaced by another atom or another group of atoms (substituent). Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents on carbon atoms, whereas the bivalent substituents ═O and ═NR may also be a substituent on sulphur. Generally, substitution may be carried out by a bivalent substituent only at ring systems and requires replacement of two geminal hydrogen atoms, i.e. hydrogen atoms that are bound to the same carbon atom that is saturated prior to the substitution. Substitution by a bivalent substituent is therefore only possible at the group —CH₂— or sulphur atoms (═O group or ═NR group only, one or two ═O groups possible or, e.g., one ═O group and one ═NR group, each group replacing a free electron pair) of a ring system.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, cis/trans/Zisomers, etc.) and racemates thereof as well as mixtures in different proportions of the separate enantiomers, mixtures of diastereomers, or mixtures of any of the foregoing forms where such isomers and enantiomers exist, and solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

By a therapeutically effective amount for the purposes of this invention is meant a quantity of substance that is capable of obviating symptoms of illness or of preventing or alleviating these symptoms, or which prolong the survival of a treated patient.

RAS-family proteins are meant to include KRAS (V-Ki-ras2 Kirsten rat sarcoma viral oncogene homolog), NRAS (neuroblastoma RAS viral oncogene homolog), HRAS (Harvey murine sarcoma virus oncogene) and MRAS (muscle RAS oncogene homolog) and any mutants thereof.

A SOS1 inhibitor compound is a compound, which binds to SOS1 and thereby prevents the SOS1 mediated nucleotide exchange and subsequently reduces the levels of RAS in its GTP bound form. More specifically, a SOS1 inhibitor compound shows a pharmacological inhibition of the binding of the catalytic site of SOS1 to RAS-family proteins. Thus, such a compound interacts with SOS1, e.g. the catalytic site on SOS1, and reduces the level of binding to the RAS-family protein in relation to said binding without addition of a SOS1 inhibitor compound. Accordingly, it is envisaged that a SOS1 inhibitor compound at least reduces the level of binding to the RAS-family protein about 5%, 10%, 20%, 30%, 40%, 50%, 60%, 70%, 80%, 90% or even 100% when compared to the binding that is achieved without the addition of said inhibitor compound. Suitable test systems to measure the binding to the catalytic site of SOS1 are disclosed herein. Said compound may be chemically synthesized (e.g. a small molecule) or microbiologically produced (e.g. a monoclonal antibody) and/or comprised in, for example, samples, e.g., cell extracts from, e.g., plants, animals or microorganisms. Preferably, the SOS1 inhibitor compound is a small molecule.

### Pharmaceutical Composition

A further object of the invention is a pharmaceutical composition comprising a compound of formula (I)—or a pharmaceutically acceptable salt thereof—and one or more pharmaceutically acceptable excipient(s).

In one aspect, said pharmaceutical composition optionally comprises one or more other pharmacologically active substance(s). Said one or more other pharmacologically active substance(s) may be the pharmacologically active substances or combination partners as herein defined.

Suitable pharmaceutical compositions for administering the compounds of formula (I) according to the invention will be apparent to those with ordinary skill in the art and include for example tablets, pills, capsules, suppositories, lozenges, troches, solutions, suspensions—particularly solutions, suspensions or other mixtures for injection (s.c., i.v., i.m.) and infusion (injectables)—elixirs, syrups, sachets, emulsions, inhalatives or dispersible powders The content of the compounds of formula (I) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the compounds of formula (I) with known pharmaceutically acceptable excipients, for example inert diluents, carriers, disintegrants, adjuvants, surfactants, binders and/or lubricants. The tablets may also comprise several layers.

Coated tablets may be prepared accordingly by coating cores produced analogously to the tablets with excipients normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups or elixirs containing one or more compounds of formula (I) or combinations with one or more other pharmaceutically active substance(s) may additionally contain excipients like a sweetener such as saccharine, cyclamate, glycerol or sugar and a flavour enhancer, e.g. a flavouring such as vanillin or orange extract They may also contain excipients like suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of excipients like isotonic agents, preservatives such as p-hydroxybenzoates, or stabilisers such as alkali metal salts of ethylenediamine tetra acetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more compounds of formula (I) or combinations with one or more other pharmaceutically active substance(s) may for example be prepared by mixing the compounds with inert excipients such as lactose or sorbitol and packing them into gelatine capsules.

Suitable suppositories may be made for example by mixing with excipients provided for this purpose such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulfate).

The pharmaceutical compositions are administered by the usual methods, preferably by oral or transdermal route, most preferably by oral route. For oral administration the tablets may of course contain, apart from the above-mentioned excipients, additional excipients such as sodium citrate, calcium carbonate and dicalcium phosphate together with various excipients such as starch, preferably potato starch, gelatine and the like. Moreover, lubricants such as magnesium stearate, sodium lauryl sulfate and talc may be used at the same time for the tableting process. In the case of aqueous suspensions, the active substances may be combined with various flavour enhancers or colourings in addition to the excipients mentioned above.

For parenteral use, solutions of the compounds of formula (I) with suitable liquid excipients may be used.

The dosage range of the compounds of formula (I) applicable per day is usually from 1 mg to 2000 mg, preferably from 1 mg to 1500 mg, more preferably from 1 mg to 1000 mg, most preferably from 1 mg to 750 mg.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts, it may be advisable to divide them up into a number of smaller doses spread over the day.

Thus, in a further aspect the invention relates to a pharmaceutical composition comprising at least one (preferably one) compound of formula (I)—or a pharmaceutically acceptable salt thereof—and one or more pharmaceutically acceptable excipient(s).

The compounds of formula (I)—or the pharmaceutically acceptable salts thereof—and the pharmaceutical compositions comprising such compound and salts may also be co-administered with other pharmacologically active substances, e.g. with other anti-neoplastic compounds (e.g. chemotherapy), i.e. used in combination (see combination treatment further below).

The elements of such combinations may be administered (whether dependently or independently) by methods customary to the skilled person and as they are used in monotherapy, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable excipients appropriate for each route of administration.

The combinations may be administered at therapeutically effective single or divided daily doses. The active components of the combinations may be administered in such doses which are therapeutically effective in monotherapy, or in such doses which are lower than the doses used in monotherapy, but when combined result in a desired (joint) therapeutically effective amount.

However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side effects that are associated with the use of one or more of the substances or principles when they are used in their usual amounts, while still obtaining the desired pharmacological or therapeutic effect.

Thus, in a further aspect the invention also relates to a pharmaceutical composition comprising a compound of formula (I)—or a pharmaceutically acceptable salt thereof—and one or more (preferably one or two, most preferably one) other pharmacologically active substance(s).

In a further aspect the invention also relates to a pharmaceutical preparation comprising a compound of formula (I)—or a pharmaceutically acceptable salt thereof—and one or more (preferably one or two, most preferably one) other pharmacologically active substance(s).

Pharmaceutical compositions to be co-administered or used in combination can also be provided in the form of a kit.

Thus, in a further aspect the invention also relates to a kit comprising
a first pharmaceutical composition or dosage form comprising a compound of formula (I) and, optionally, one or more pharmaceutically acceptable excipient(s), and
a second pharmaceutical composition or dosage form comprising another pharmacologically active substance and, optionally, one or more pharmaceutically acceptable excipient(s).

In one aspect such kit comprises a third pharmaceutical composition or dosage form comprising still another pharmacologically active substance and, optionally, one or more pharmaceutically acceptable excipient(s).

### Medical Uses—Methods of Treatment

The present invention is directed to compounds of formula (I) (including all its embodiments), which are useful in the treatment and/or prevention of a disease and/or condition associated with or modulated by SOS1, especially wherein the inhibition of the interaction of SOS1 and a RAS-family protein and/or RAC1 is of therapeutic benefit, including but not limited to the treatment and/or prevention of cancer.

In another aspect the invention relates to a compound of formula (I)—or a pharmaceutically acceptable salt thereof—for use as a medicament.

In another aspect the invention relates to a compound of formula (I)—or a pharmaceutically acceptable salt thereof—for use in a method of treatment of the human or animal body.

In another aspect the invention relates to a compound of formula (I)—or a pharmaceutically acceptable salt thereof—for use in the treatment and/or prevention of a disease and/or condition wherein the inhibition of the interaction of SOS1 and a RAS-family protein and/or RAC1 is of therapeutic benefit, including but not limited to the treatment and/or prevention of cancer.

In another aspect the invention relates to a compound of formula (I)—or a pharmaceutically acceptable salt thereof—for use in the treatment and/or prevention of cancer.

In another aspect the invention relates to a compound of formula (I)—or a pharmaceutically acceptable salt thereof—for use in a method of treatment and/or prevention of cancer in the human or animal body.

In another aspect the invention relates to a compound of formula (I)—or a pharmaceutically acceptable salt thereof—for use as hereinbefore defined wherein said compound is administered before, after, or together with at least one other pharmacologically active substance.

In another aspect the invention relates to a pharmacologically active substance prepared for being administered before, after, or together with a SOS1 inhibitor compound—or a pharmaceutically acceptable salt thereof—for use as hereinbefore defined for the use of the compound of formula (I).

In another aspect the invention relates to a pharmacologically active substance prepared for being administered before, after, or together with a compound of formula (I)—or a pharmaceutically acceptable salt thereof—for use as hereinbefore defined for the use of the compound of formula (I).

In another aspect the invention relates to a compound of formula (I), —or a pharmaceutically acceptable salt thereof—for use in the treatment or in a method of treatment as hereinbefore defined.

In another aspect the invention relates to the use of a compound of formula (I), —or a pharmaceutically acceptable salt thereof—for preparing a pharmaceutical composition for the treatment and/or prevention of cancer.

In another aspect the invention relates to the use of a compound of formula (I)—or a pharmaceutically acceptable salt thereof—as hereinbefore defined wherein said compound is administered before, after, or together with at least one other pharmacologically active substance.

In another aspect the invention relates to the use of a compound of formula (I), —or a pharmaceutically acceptable salt thereof—as hereinbefore defined for the treatment.

In another aspect the invention relates to a method for the treatment and/or prevention of a disease and/or condition wherein the inhibition of the interaction of SOS1 and a RAS-family protein or RAC1 is of therapeutic benefit comprising administering a therapeutically effective amount of a compound of formula (I), —or a pharmaceutically acceptable salt thereof—to a human being.

In another aspect the invention relates to a method for the treatment and/or prevention of cancer comprising administering a therapeutically effective amount of a compound of formula (I), —or a pharmaceutically acceptable salt thereof—to a human being.

In another aspect the invention relates to a method as hereinbefore defined wherein the compound of formula (I)—or a pharmaceutically acceptable salt thereof—is administered before, after, or together with at least one other pharmacologically active substance.

In another aspect the invention relates to a method as hereinbefore defined wherein the compound of formula (I)—or a pharmaceutically acceptable salt thereof—is administered in combination with a therapeutically effective amount of at least one other pharmacologically active substance.

In another aspect the invention relates to a method for the treatment as hereinbefore defined.

In another aspect the invention relates to a pharmaceutical composition comprising at least one (preferably one) compound of formula (I)—or a pharmaceutically acceptable salt thereof—and one or more pharmaceutically acceptable excipient(s).

In another aspect the invention relates to a pharmaceutical preparation comprising a compound of formula (I)—or a pharmaceutically acceptable salt thereof—and at least one (preferably one) other pharmacologically active substance.

In another aspect the pharmacologically active substance to be used together/in combination with the compound of formula (I) (including all individual embodiments or generic subsets of compounds (I)), or in the medical uses, uses, methods of treatment and/or prevention as herein (above and below) defined can be selected from any one or more of the following (preferably there is only one additional pharmacologically active substance used in all these embodiments):
1. an inhibitor of EGFR and/or of mutants thereof
   a. e.g. afatinib, erlotinib, gefitinib, lapatinib, cetuximab, panitumumab, osimertinib, olmutinib, EGF-816;
   b. preferred are cetuximab, osimertinib and sunvozertinib;
   c. most preferred is cetuximab
2. an inhibitor of ErbB2 (Her2) and/or of mutants thereof
   a. e.g. zongertinib, tucatinib, lapatinib, trastuzumab, pertuzumab;
   b. preferred are zongertinib and trastuzumab;
   c. most preferred is zongertinib;
3. an inhibitor of ALK and/or of mutants thereof
   a. e.g. crizotinib, alectinib, entrectinib, brigatinib;
   b. preferred are crizotinib and alectinib;
   c. most preferred is crizotinib;
4. an inhibitor of BCR-ABL and/or of mutants thereof
   a. e.g. imatinib, dasatinib, nilotinib;
   b. preferred are imatinib and nilotinib;
   c. most preferred is imatinib;
5. an inhibitor of FGFR1 and/or FGFR2 and/or FGFR3 and/or of mutants thereof
   a. e.g. erdafitinib, infigratinib, pemigatinib, futibatinib;
   b. preferred are pemigatinib and futibatinib
6. an inhibitor of ROS1 and/or of mutants thereof
   a. e.g. crizotinib, entrectinib, lorlatinib, ceritinib, merestinib;
   b. preferred are crizotinib and entrectinib;
   c. most preferred is crizotinib;
7. an inhibitor of c-MET and/or of mutants thereof
   e.g. capmatinib, amivantamab
8. an inhibitor of AXL and/or of mutants thereof
9. an inhibitor of NTRK1 and/or of mutants thereof
10. an inhibitor of RET and/or of mutants thereof
11. an inhibitor of MEK and/or of mutants thereof
   a. e.g. trametinib, cobimetinib, binimetinib, selumetinib, refametinib;
   b. preferred are trametinib and cobimetinib;
   c. most preferred is trametinib;
12. an inhibitor of GDP-bound KRAS and/or of mutants thereof
   a. an irreversible inhibitor of KRAS G12C
      i. e.g. adagrasib, sotorasib
   b. a reversible inhibitor of GDP-bound KRAS and/or of mutants thereof;
      a. e.g. BI-3706674
13. an inhibitor of GTP-bound KRAS and/or mutants thereof
   a. e.g. RMC-6236 RAS^{MULTI} (ON) Inhibitor
   b. RAS(ON) Inhibitors targeting specific alleles e.g. RMC-6291 (KRAS^{G12C}), RMC-9805 (KRAS^{G12D}) RMC-8839 (KRAS^{G13C})
14. an inhibitor of A-Raf and/or B-Raf and/or C-Raf and/or of mutants thereof
   a. e.g. RAF-709 (=example 131 in WO 2014/151616), LY-3009120 (=example 1 in WO 2013/134243);
15. an inhibitor of ERK and/or of mutants thereof
   a. e.g. ulixertinib;
16. an inhibitor of RAS GEFs and/or of mutants thereof
   e.g. an inhibitor of SOS2 and/or of mutants thereof
17. an inhibitor of PI3K and/or of mutants thereof
18. an inhibitor of mTOR
   a. e.g. rapamycin, temsirolimus, everolimus, ridaforolimus;
19. a taxane
   a. e.g. paclitaxel, nab-paclitaxel, docetaxel;
   b. preferred is paclitaxel;
20. a platinum-containing compound
   a. e.g. cisplatin, carboplatin, oxaliplatin;
21. an anti-metabolite
   a. e.g. 5-fluorouracil, capecitabine, floxuridine, cytarabine, gemcitabine, combination of trifluridine and tipiracil (=TAS102);
   b. preferred is gemcitabine;
22. mitotic kinase inhibitor
   a. e.g. CDK4/6 inhibitor
      i. e.g. palbociclib, ribociclib, abemaciclib;
      ii. preferred are palbociclib and abemaciclib;
      iii. most preferred is abemaciclib;
23. an immunotherapeutic agent
   a. e.g. an immune checkpoint inhibitor
      i. e.g. an anti-CTLA4 mAb, anti-PD1 mAb, anti-PD-L1 mAb, anti-PD-L2 mAb, anti-LAG3 mAb, anti-TIM3 mAb;
      ii. preferred is an anti-PD1 mAb;
      iii. e.g. ipilimumab, nivolumab, pembrolizumab, atezolizumab, avelumab, durvalumab, pidilizumab, PDR-001 (=spartalizumab);
      iv. preferred are nivolumab, pembrolizumab and PDR-001 (=spartalizumab);
      v. most preferred is pembrolizumab;
24. an anti-angiogenic drug
   a. e.g. bevacizumab, nintedanib;
   b. most preferred is bevacizumab;
25. a topoisomerase inhibitor
   a. e.g. irinotecan, liposomal irinotecan, topotecan;
   b. most preferred is irinotecan;
26. an apoptosis regulator
   a. e.g. an inhibitor of the interaction between p53 (preferably functional p53, most preferably wt p53) and MDM2 (a “MDM2 inhibitor”);
      i. e.g. HDM-201, NVP-CGM097, RG-7112, MK-8242, RG-7388, SAR405838, AMG-232, DS-3032, RG-7775, APG-115, Brigimadlin;
      ii. preferred are HDM-201, RG-7388 and AMG-232
   b. e.g. a PARP inhibitor;
   c. e.g. a MCL-1 inhibitor;
27. an epigenetic regulator
   a. e.g. a BET inhibitor
      i. e.g. JQ-1, GSK 525762, OTX 015 (=MK8628), CPI 0610, TEN-010 (=RO6870810);
   b. e.g. a CDK9 inhibitor;
28. an inhibitor of IGF1/2 and/or of IGF1-R
   a. e.g. xentuzumab (antibody 60833 in WO 2010/066868), MEDI-573 (=dusigitumab);

Within this invention it is to be understood that the combinations, compositions, kits, methods, uses or compounds for use according to this invention may envisage the simultaneous, concurrent, sequential, successive, alternate, or separate administration of the active ingredients or components. It will be appreciated that the compound of formula (I) and the at least one other pharmacologically active substance can be administered formulated either dependently or independently, such as e.g. the compound of formula (I) and the at least one other pharmacologically active substance may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently, or sequentially with no specific time limits, wherein such administration provides therapeutically effective levels of the two compounds in the body of the patient.

The administration of the compound of formula (I) and the at least one other pharmacologically active substance may take place by co-administering the active components or ingredients, such as e.g. by administering them simultaneously or concurrently in one single or in two or more separate formulations or dosage forms. Alternatively, the administration of the compound of formula (I) and the at least one other pharmacologically active substance may take place by administering the active components or ingredients sequentially or in alternation, such as e.g. in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect of the invention the invention provides a method for the treatment and/or prevention of cancer comprising administering to a patient in need thereof a therapeutically effective amount of a compound of formula (I) and a therapeutically effective amount of at least one other pharmacologically active substance, wherein the compound of formula (I) is administered simultaneously, concurrently, sequentially, successively, alternately or separately with the at least one other pharmacologically active substance.

In another aspect the invention provides a compound of formula (I) for use in the treatment and/or prevention of cancer, wherein the compound of formula (I) is administered simultaneously, concurrently, sequentially, successively, alternately or separately with the at least one other pharmacologically active substance.

In another aspect the invention provides a kit comprising
a first pharmaceutical composition or dosage form comprising a compound of formula (I), and, optionally, one or more pharmaceutically acceptable carriers, excipients and/or vehicles, and
at least a second pharmaceutical composition or dosage form comprising another pharmacologically active substance, and, optionally, one or more pharmaceutically acceptable carriers, excipients and/or vehicles,
for use in the treatment and/or prevention of cancer, wherein the first pharmaceutical composition is to be administered simultaneously, concurrently, sequentially, successively, alternately, or separately with the second and/or additional pharmaceutical composition or dosage form.

In a further embodiment of the invention the components (i.e. the combination partners) of the combinations, kits, uses, methods and compounds for use according to the invention (including all embodiments) are administered simultaneously.

In a further embodiment of the invention the components (i.e. the combination partners) of the combinations, kits, uses, methods and compounds for use according to the invention (including all embodiments) are administered concurrently.

In a further embodiment of the invention the components (i.e. the combination partners) of the combinations, kits, uses, methods and compounds for use according to the invention (including all embodiments) are administered sequentially.

In a further embodiment of the invention the components (i.e. the combination partners) of the combinations, kits, uses, methods and compounds for use according to the invention (including all embodiments) are administered successively.

In a further embodiment of the invention the components (i.e. the combination partners) of the combinations, kits, uses, methods and compounds for use according to the invention (including all embodiments) are administered alternately.

In a further embodiment of the invention the components (i.e. the combination partners) of the combinations, kits, uses, methods and compounds for use according to the invention (including all embodiments) are administered separately.

The “therapeutically effective amount” of the active compound(s) to be administered is the minimum amount necessary to prevent, ameliorate, or treat a disease or disorder.

The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (joint) therapeutically effective amount.

In another aspect the disease/condition/cancer to be treated/prevented with the compound of formula (I), compound of formula (I) for use, use for preparing and method for the treatment and/or prevention as herein (above and below) defined is selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, gastroesophageal/gastroesophageal junction cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer, skin squamous cell carcinoma and sarcomas.

In another aspect the disease/condition/cancer to be treated/prevented with the compound of formula (I), compound of formula (I) for use, use for preparing and method for the treatment and/or prevention as herein (above and below) defined is selected from the group consisting of lung cancer (preferably non-small cell lung cancer (NSCLC)), head and neck squamous cell carcinoma, cholangiocarcinoma, bladder cancer and colorectal cancer.

In another aspect the disease/condition to be treated/prevented with the compound of formula (I), compound of formula (I) for use, use for preparing and method for the treatment and/or prevention as herein (above and below) defined is a RASopathy, preferably selected from the group consisting of Neurofibromatosis type 1 (NF1), NF1-associated Plexiform Neurofibroma, Malignant Peripheral Nerve Sheath Tumors (MPNST), Cutaneous Neurofibromas, Noonan Syndrome (NS), NS-associated congenital heart defects, Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

In another aspect the disease/condition to be treated/prevented with the compound of formula (I), compound of formula (I) for use, use for preparing and method for the treatment for diabetes, preferably type I diabetes.

In another aspect the disease/condition/cancer to be treated/prevented with the compound of formula (I), compound of formula (I) for use, use for preparing and method for the treatment and/or prevention as herein (above and below) defined is a disease/condition/cancer defined as exhibiting one or more of the following molecular features:
1. KRAS alterations:
   a. KRAS amplification (wt or mutant);
   b. KRAS overexpression (wt or mutant);
   c. KRAS mutation(s):
      i. G12 mutations (e.g. G12C, G12V, G12S, G12A, G12R, G12F, G12D);
      ii. G13 mutations (e.g. G13C, G13D, G13R, G13V, G13S, G13A)
      iii. T35 mutation (e.g. T351);
      iv. 136 mutation (e.g. 136L, 136M);
      v. E49 mutation (e.g. E49K);
      vi. Q61 mutation (e.g. Q61H, Q61R, Q61P, Q61E, Q61K, Q61 L;
      vii. K117 mutation (e.g. K117N);
      viii. A146 mutation (e.g. A146T, A146V);
2. NRAS alterations:
   a. NRAS amplification (wt or mutant);
   b. NRAS overexpression (wt or mutant);
   c. NRAS mutation(s):
      i. G12 mutations (e.g. G12A, G12V, G12D, G12C, G12S, G12R);
      ii. G13 mutation (e.g. G13V, G13D, G13R, G13S, G13C, G13A);
      iii. Q61 mutation (e.g. Q61K, Q61 L, Q61H, Q61P, Q61R);
      iv. A146 mutation (e.g. A146T, A146V);
3. HRAS alterations:
   a. HRAS amplification (wt or mutant);
   b. HRAS overexpression (wt or mutant);
   c. HRAS mutation(s);
      i. G12 mutation (e.g. G12C, G12V, G12S, G12A, G12R, G12F, G12D);
      ii. G13 mutation (e.g. G13C, G13D, G13R, G13V, G13S, G13A);
      iii. Q61 mutation (e.g. Q61K, Q61 L, Q61H, Q61P, Q61R);
4. MRAS alterations:
   a. G23V
   b. T681
4. EGFR alterations:
   a. EGFR amplification (wt or mutant);
   b. EGFR overexpression (wt or mutant);
   c. EGFR mutation(s)
      i. e.g. exon 20 insertion, exon 19 deletion (Del19), G719X (e.g. G719A, G719C, G719S), T790M, C797S, T854A, L858R, L861Q, or any combination thereof;
5. ErbB2 (Her2) alterations:
   a. ErbB2 amplification;
   b. ErbB2 overexpression;
   c. ErbB2 mutation(s)
      i. e.g. R678, G309, L755, D769, V777, P780, V842, R⁸⁹⁶, c.2264_2278del (L755_T759del), c.2339_2340ins (G778_P780dup), S310;
6. c-MET alterations:
   a. c-MET amplification;
   b. c-MET overexpression;
   c. c-MET mutation(s)
      i. e.g. E168, N375, Q648, A887, E908, T1010, V1088, H1112, R1166, R1188, Y1248, Y1253, M1268, D1304, A1357, P1382;
7. AXL alterations:
   a. AXL amplification;
   b. AXL overexpression;
8. BCR-ABL alterations:
   a. chromosomal rearrangements involving the ABL gene;
9. ALK alterations:
   a. ALK amplification;
   b. ALK overexpression;
   c. ALK mutation(s)
      1. e.g. 1151Tins, L1152R, C1156Y, F1174L, L1196M, L1198F, G1202R, S1206Y, G1269A;
   d. chromosomal rearrangements involving the ALK gene;
10. FGFR1 alterations:
   a. FGFR1 amplification;
   b. FGFR1 overexpression;
11. FGFR2 alterations:
   a. FGFR2 amplification;
   b. FGFR2 overexpression;
   c. FGFR2 mutation;
   d. chromosomal rearrangement involving the FGFR2 gene
12. FGFR3 alterations:
   a. FGFR3 amplification;
   b. FGFR3 overexpression;
   c. chromosomal rearrangement involving the FGFR3 gene;
   d. FGFR3 mutation e.g. R248C
13. FGFR4 alterations:
   a. FGFR4 mutations
14. NTRK1 alterations:
   a. chromosomal rearrangements involving the NTRK1 gene;
15. NF1 alterations:
   a. NF1 mutation(s);
16. RET alterations:
   a. RET amplification;
   b. RET overexpression;
   c. chromosomal rearrangements involving the RET gene
17. ROS1 alterations:
   a. ROS1 amplification;
   b. ROS1 overexpression;
   c. ROS1 mutation(s)
      i. e.g. G2032R, D2033N, L2155S;
   d. chromosomal rearrangements involving the ROS1 gene;
18. SOS1 alterations
   a. SOS1 amplification;
   b. SOS1 overexpression;
   c. SOS1 mutation(s); e.g. N233Y
19. RAC1 alterations
   a. RAC1 amplification;
   b. RAC1 overexpression;
   c. RAC1 mutation(s);
20. MDM2 alterations
   a. MDM2 amplification
   b. MDM2 overexpression
   c. MDM2 amplification in combination with functional p53
   d. MDM2 amplification in combination with wild-type p53
21. RAS wild-type
   a. KRAS wild-type
   b. HRAS wild-type
   c. NRAS wild-type
22. B-Raf mutation(s), including V600E but also other mutations e.g. class II and class III

In another aspect the disease/condition/cancer to be treated/prevented with compound of formula (I), compound of formula (I) for use, use for preparing and method for the treatment and/or prevention as herein (above and below) defined is a cancer that is resistant to a treatment with a KRAS G12C inhibitor. For example, RAS amplification (KRAS or MRAS) was published as a resistant mechanism following treatment with a KRAS G12C inhibitor that can be addressed by a combination with a SOS1i (Thatikonda, et al. Nat Cancer 2024, 5, 1352-1370).

In another aspect the combined treatment of compound of formula (I), and a KRAS G12C inhibitor is administered following treatment with a KRAS G12C inhibitor. Preferably, the patients were treated with adagrasib (or MRTX 849) or sotorasib (or AMG 510), but also including next generation inhibitors such as Divarisib.

Any disease/condition/cancer, medical use, use, method of treatment and/or prevention as disclosed or defined herein (including molecular/genetic features) may be treated/performed with any compound of formula (I) as disclosed or defined herein (including all individual embodiments or generic subsets of compounds (I)).

### Therapeutic Use

Due to their biological properties the compounds of the invention, their tautomers, racemates, enantiomers, diastereomers, mixtures thereof and the salts of all the above-mentioned forms may be suitable for treating diseases characterised by excessive or abnormal cell proliferation such as cancer.

For example, the following cancers, tumors and other proliferative diseases may be treated with compounds of the invention, without being restricted thereto:

Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas,
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);
All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithelial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas;

The compounds of the invention may be used in therapeutic regimens in the context of first line, second line, or any further line treatments.

The compounds of the invention may be used for the prevention, short-term or long-term treatment of the above-mentioned diseases, optionally also in combination with radiotherapy and/or surgery.

Of course, the above also includes the use of the compounds of the invention in various methods of treating the above diseases by administering a therapeutically effective dose to a patient in need thereof, as well as the use of these compounds for the manufacture of medicaments for the treatment of such diseases, as well as pharmaceutical compositions including such compounds of the invention, as well as the preparation and/or manufacture of medicaments including such compounds of the invention, and the like.

### Preparation of the Compounds According to the Invention

The compounds according to the invention and intermediates are prepared by the methods of synthesis described hereinafter in which the substituents of the general formula have the meanings given hereinbefore. These methods are intended as an illustration of the invention without restricting its subject matter and the scope of the compounds claimed to these examples. Where the preparation of starting compounds is not described, they are commercially obtainable or their synthesis is described in the prior art or they may be prepared analogously to known prior art compounds or methods described herein, i.e. it is within the skills of an organic chemist to synthesize these compounds. Substances described in the literature can be prepared according to the published methods of synthesis.

The general processes for preparing the compounds according to the invention will become apparent to the one skilled in the art studying the following schemes. Starting materials may be either commercially available or may be prepared by methods that are described in the literature or herein or may be prepared in an analogous or similar manner. Any functional groups in the starting materials or intermediates may be protected using conventional protecting groups. These protecting groups may be cleaved again at a suitable stage within the reaction sequence using methods familiar to the one skilled in the art.

The scheme below (Scheme 1) illustrates the possible synthesis of the compounds of general formula (I) and its intermediates. The first step can be an amide coupling using carboxylic acid derivatives of structure B and benzylic amines of structure A as reaction partners to form bicyclic intermediates of structure C containing an aromatic bicyclic 5-6 fused ring system with an attached functional group 1 (FG1). The functional group 1 enables use of cross coupling reactions such as Suzuki reactions, Buchwald couplings, or alike in the next reaction step the. This allows either to form directly compounds of general formula (I) or the intermediate structures D. The functional group 2 (FG2) in intermediate D can be further derivatized using for example reductive amination reactions, nucleophilic substitution reactions or alike with reagents known to the people skilled in the art to form compounds of general formula (I).

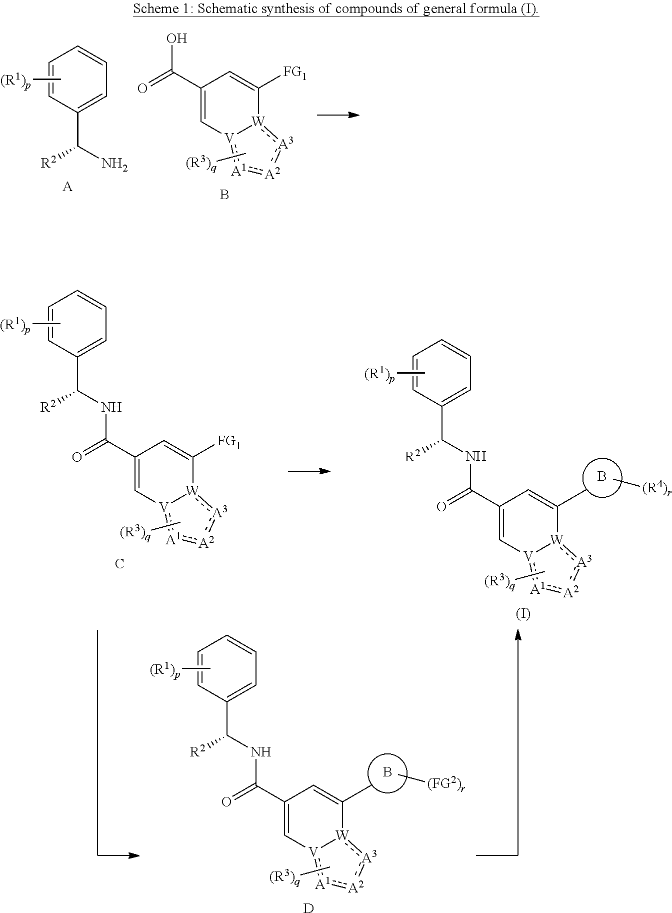

As described for some exemplified compounds, a reaction called Dimroth rearrangement (described for example herein: ACS Med. Chem. Lett. 2017, 8, 12, 1320-1325) can also be used to form intermediates of structure C, which can be used in the synthesis of compound of general formula (I).

Abbreviations

| | |
|---|---|
| ° | degree symbol |
| Å | angstrom |
| ACN | acetonitrile |
| AcOH | acetic acid |
| A.M. | analytical method |
| catalyst I | [1,3-bis[2,6-bis(1-ethylpropyl)phenyl]-4,5-dichloro- |
| | imidazol-2-yl]-dichloro-(2-methyl-1-pyridyl)palladium |
| | (CAS: 1612891-29-8) |
| catalyst II | 1,1′-bis(diphenylphosphino)ferrocene- |
| | palladium(II)dichloride dichloromethane complex (CAS: |
| | 95464-05-4) |
| C. | Celsius |
| Cs₂CO₃ | cesium carbonate |
| d.e. | diastereomeric excess |
| DAD | diode Array Detector |
| δ | delta (chemical shift) |
| DCM | dichloromethane |
| DCE | dichloroethane |
| DMEM | Dulbecco's Modified Eagle's Medium |
| DMF | N,N-dimethylformamide |
| DMSO-d₆ | deuterated dimethyl sulfoxide |
| e.e. | enantiomeric excess |
| e.g. | exempli gratia; anglicised: example given |
| EGTA | (ethylene glycol-bis(β-aminoethyl ether)-N,N,N′,N′ |
| | tetraacetic acid), also known as egtazic acid |
| Et | ethyl |
| EtOAc | ethyl acetate |
| EtOH | ethanol |
| ESI | electrospray ionization |
| FBS | fetal Bovine Serum |
| FG | functional group |
| FLIPR | fluorometric imaging plate reader |
| g | gram(s) |
| h | hour(s) |
| H₂O | water |
| HCl | hydrogen chloride |
| HATU | hexafluorophosphate azabenzotriazole tetramethyl |
| | uronium |
| HEK293 | cell line derived from human embryonic kidney cells |
| HEPES | hydroxyethyl-piperazineethane-sulfonic acid buffer |
| HPLC | high performance liquid chromatography |
| Hz | hertz |
| IC₅₀ | half maximal inhibitory concentration |
| KHSO₄ | potassium bisulfate |
| IPA | isopropanol |
| IUPAC | International Union of Pure and Applied Chemistry |
| L | liter(s) |
| LDA | lithium diisopropylamide |
| μ | micro |
| μL | microliter (s) |
| μmol | micromole (s) |
| μM | micromolar |
| M | molar; mega |
| MHz | megahertz |
| m | milli |
| mg | milligram (s) |
| MgSO₄ | magnesium sulfate |
| mL | milliliter (s) |
| mmol | millimole (s) |
| m/z | mass-to-charge ratio |
| Me | methyl |
| MeOH | methanol |
| MDCK | Madin-Darby canine kidney |
| MDR1 | Multi drug resistance protein 1 |
| min | minute(s) |
| mol | mole |
| MS | mass spectrometry |
| MTBE | methyl tert-butyl ether |
| N | normal |
| NaOH | sodium hydoxide |
| NaHCO₃ | sodium bicarbonate |
| NMR | nuclear magnetic resonance spectroscopy |
| P-gp | p-glycoprotein |
| PPA | Polyphosphoric acid |
| ppm | part(s) per million |
| prep | preperative |
| PSI | pound-force per square inch |
| Rₜ | retention time |
| RP | reversed phase |
| RPMI | Roswell Park Memorial Institute |
| rt | room temperature |
| scCO2 | supercritical carbon dioxide |
| SEM | standard error of the mean |
| SFC | supercritical fluid chromatography |
| SiO₂ | silicon dioxide |
| TEV | tobacco etch virus |
| TFA | trifluoroacetic acid |
| THF | tetrahydrofuran |
| TLC | thin layer chromatography |
| v/v | volume ratio |
| wt % | weight percentage |
| % | percentage |
| % w/w | percentage composition by weight |

### Experimental Part—Chemical Synthesis

Other features and advantages of the present invention will become apparent from the following more detailed examples which exemplarily illustrate the principles of the invention without restricting its scope.

### General

The terms “cis” and “trans” are used in accordance with the IUPAC Gold Book's guidelines to denote the stereochemical information of the substituents, which varies based on the positions of atoms (or groups) relative to a reference plane in a ring system. In the cis-isomer, the atoms are located on the same side, while in the trans-isomer, they are on opposing sides. This follows the traditional order of priority for substituents/ligands. (PAC, 1996, 68, 2193. (Basic terminology of stereochemistry (IUPAC Recommendations 1996)) on page 2203).

Unless stated otherwise, all the reactions are carried out in commercially obtainable apparatuses using methods that are commonly used in chemical laboratories. Starting materials that are sensitive to air and/or moisture are stored under protective gas and corresponding reactions and manipulations therewith are carried out under inert gas (nitrogen or argon).

Room temperature in the following schemes means the temperature ranging from 19° C. to 24° C.

If a compound is to be represented both by a structural formula and by its nomenclature, in the event of a conflict the structural formula is decisive.

Some compounds according to the exemplified preparation are filtered through carbonate functionalized MP resin (carbonate cartridge) by Agilent Technologies (PL-HCO₃ MP SPE; Part. No. PL3540-C603) as indicated.

Some compounds according to the exemplified preparation are filtered through thiol functionalized SPEmedia (thiol resin) by Agilent Technologies (PL-Thiol MP SPE Part. No. 3582-CM89) prior to chromatography, if indicated.

The following catalyst, termed catalyst I, is used for some exemplified coupling reactions of this invention [1,3-bis[2,6-bis(1-ethylpropyl)phenyl]-4,5-dichloro-imidazol-2-yl]-dichloro-(2-methyl-1-pyridyl)palladium (catalyst I; CAS: 1612891-29-8).

The following catalyst, termed catalyst II, is used for some exemplified coupling reactions of this invention [1,1′-bis(diphenylphosphino)ferrocene-palladium(II)dichloride dichloromethane complex (CAS: 95464-05-4).

### NMR Method

NMR spectra were recorded on a Bruker AVANCE IIIHD 400 MHz instrument using TopSpin 3.2 pl6 software. Chemical shifts are given in parts per million (ppm) downfield from an internal reference like trimethylsilane and/or water and/or solvent (eg. d⁶-DMSO) in δ units. Selected data are reported in the following manner: chemical shift (number of hydrogens).

### Chromatography

Thin layer chromatography is carried out on ready-made TLC plates of silica gel 60 on glass (with fluorescence indicator F-254) made by Merck.

Prep. RP-HPLC is carried out with columns made by Waters (Sunfire C18, 10 μm, 30×100 mm Part. No. 186003971 or X-Bridge C18, 10 μm, 30×100 mm Part. No. 186003930). The compounds are eluted using either different gradients of H₂O/ACN or H₂O/MeOH, where 0.1% TFA is added to the water, or with different gradients utilizing a basic aqueous buffer solution (1 L water contains 5 mL of an ammonium hydrogencarbonate solution (158 g per 1 L H₂O) and 2 mL NH₃ (7 mol/L solution in MeOH)) instead of the water-TFA-mixture.

### Mass Spectroscopy

Low resolution mass spectra are obtained using a high performance liquid chromatography coupled to a quadrupole mass spectrometer (HPLC-MS; electrospray positive ionization). The reported mass spectrometry (MS) data correspond to the observed mass peaks of the monoisotopic masses of the respective compound ([M+H]+) or a fragment thereof (e.g., [M-Boc+H]⁺, [M-NH₃+H]⁺). Analytical HPLC Methods (A.M.)

| | |
|---|---|
| Method Name: | A |
| Device description: | Waters Acquity, QDa Detector |
| Column: | XBridge C18_3.0 × 30 mm_2.5 μm |
| Column producer: | Waters |

| Gradient/Solvent | % Sol [Water | % Sol | Flow | Temp |
|---|---|---|---|---|
| Time [min] | 0.1% NH3] | [ACN] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |
| 1.6 | 95.0 | 5.0 | 1.5 | 60.0 |

| | |
|---|---|
| Method Name: | D |
| Device description: | Agilent 1200 with DA- and MS-Detector |
| Column: | XBridge C18_3.0 × 30 mm_2.5 μm |
| Column producer: | Waters |

| Gradient/Solvent | % Sol [Water | % Sol | Flow | Temp |
|---|---|---|---|---|
| Time [min] | 0.1% NH3] | [ACN] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | E |
| Device description: | Agilent 1200 with DA- and MS-Detector |
| Column: | Sunfire C18_3.0 × 30 mm_2.5 μm |
| Column producer: | Waters |

| Gradient/Solvent | % Sol [Water | % Sol | Flow | Temp |
|---|---|---|---|---|
| Time [min] | 0.1% TFA (v/v)] | [ACN] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | M |
| Device description: | Agilent 1200 with DA- and MS-Detector |
| Column: | Sunfire C18_3.0 × 30 mm_2.5 μm |
| Column producer: | Waters |

| Gradient/Solvent | % Sol [Water | % Sol | Flow | Temp |
|---|---|---|---|---|
| Time [min] | 0.1% NH3] | [ACN] | [ml/min] | [° C.] |
| 0.0 | 50.0 | 50.0 | 2.2 | 60.0 |
| 0.2 | 50.0 | 50.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

### Mobile Phase Preparations

### EXAMPLES

The mobile phase “Water 0.1% TFA (v/v)” is prepared by adding 1 mL of a commercially available TFA solution to 999 mL water.

The mobile phase “Water 0.1% NH₃” is prepared by adding 4 mL ofa commercially available concentrated ammonium hydroxide solution (25 wt %) to 996 mL water.

### Analytical SEC Methods (A.M.)

| | |
|---|---|
| Method Name: | F |
| Device description: | Agilent 1260 Infinity II SFC with DAD and MS |
| Column: | Lux(R) Cellulose-2_3 × 100 mm_3 μm |
| Column producer: | Phenomenex |

| | | | | | Back |
|---|---|---|---|---|---|
| Gradient/Solvent | % Sol | % Sol [MEOH | Flow | Temp | pressure |
| Time [min] | [scCO2] | 20 mM NH3] | [ml/min] | [° C.] | [PSI] |
| 0.0 | 60.0 | 40.0 | 2.0 | 40.0 | 2175.0 |
| 4.0 | 60.0 | 40.0 | 2.0 | 40.0 | 2175.0 |

| | |
|---|---|
| Method Name: | G |
| Device description: | Agilent 1260 Infinity II SFC with DAD and MS |
| Column: | Chiralpak ® IH_4.6 × 250 mm_5 μm |
| Column producer: | Daicel |

| | | | | | Back |
|---|---|---|---|---|---|
| Gradient/Solvent | % Sol | % Sol [IPA | Flow | Temp | pressure |
| Time [min] | [scCO2] | 20 mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

| | |
|---|---|
| Method Name: | H |
| Device description: | Agilent 1260 Infinity II SFC with DAD and MS |
| Column: | Chiralpak ® IH_4.6 × 250 mm_5 μm |
| Column producer: | Daicel |

| | | | | | Back |
|---|---|---|---|---|---|
| Gradient/Solvent | % Sol | % Sol [MEOH | Flow | Temp | pressure |
| Time [min] | [scCO2] | 20 mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | I |
| Device | Agilent 1260 Infinity II SFC with DAD and MS |
| description: | |
| Column: | CHIRAL ART ® Amylose-C Neo_3 × 100 mm_3 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 95.0 | 5.0 | 2.0 | 40.0 | 2175.0 |
| 3.6 | 40.0 | 60.0 | 2.0 | 40.0 | 2175.0 |
| 4.0 | 40.0 | 60.0 | 2.0 | 40.0 | 2175.0 |

| | |
|---|---|
| Method Name: | J |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [ETOH 20 | Flow | Temp | pressure |
| Time [min] | [scCO2] | mM NH3] | [ml/min] | [° C.] | [PSI] |
| 0.0 | 70.0 | 30.0 | 4.0 | 40.0 | 2175.0 |
| 10.0 | 70.0 | 30.0 | 4.0 | 40.0 | 2175.0 |

| Method Name: | K | | | | |
|---|---|---|---|---|---|
| Device | Agilent 1260 Infinity II SFC with DAD and MS | | | | |
| description: | | | | | |
| Column: | Chiralpak ® IG_4.6 × 250 mm_5 μm | | | | |
| Column | Daicel | | | | |
| producer: | | | | | |
| | | % Sol | | | Back |
| Gradient/Solvent | % Sol | [MEOH 20 | Flow | Temp | pressure |
| Time [min] | [scCO2] | mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| Method Name: | L | | | | |
|---|---|---|---|---|---|
| Device | Agilent 1260 Infinity II SFC with DAD and MS | | | | |
| description: | | | | | |
| Column: | Chiralcel ® OD-3_3 × 100 mm_3 μm | | | | |
| Column | Daicel | | | | |
| producer: | | | | | |
| | | % Sol | | | Back |
| Gradient/Solvent | % Sol | [MEOH 20 | Flow | Temp | pressure |
| Time [min] | [scCO2] | mM NH3] | [ml/min] | [° C.] | [PSI] |
| 0.0 | 95.0 | 5.0 | 2.0 | 40.0 | 2175.0 |
| 3.6 | 40.0 | 60.0 | 2.0 | 40.0 | 2175.0 |
| 4.0 | 40.0 | 60.0 | 2.0 | 40.0 | 2175.0 |

| | |
|---|---|
| Method Name: | N |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 60.0 | 40.0 | 4.0 | 40.0 | 2175.0 |
| 10.0 | 60.0 | 40.0 | 4.0 | 40.0 | 2175.0 |

| | |
|---|---|
| Method Name: | O |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

| | |
|---|---|
| Method Name: | P |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 65.0 | 35.0 | 4.0 | 40.0 | 2175.0 |
| 10.0 | 65.0 | 35.0 | 4.0 | 40.0 | 2175.0 |

| | |
|---|---|
| Method Name: | Q |
| Device | Agilent 1260 Infinity II SFC with DAD and MS |
| description: | |
| Column: | CHIRAL ART ® Cellulose-SB_3 × 100 mm_3 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 80.0 | 20.0 | 2.0 | 40.0 | 2175.0 |
| 4.0 | 80.0 | 20.0 | 2.0 | 40.0 | 2175.0 |

| Method Name: | R | | | | |
|---|---|---|---|---|---|
| Device | Agilent 1260 Infinity II SFC with DAD and MS | | | | |
| description: | | | | | |
| Column: | Chiralpak ® IG_4.6 × 250 mm_5 μm | | | | |
| Column | Daicel | | | | |
| producer: | | | | | |
| | | % Sol | | | Back |
| Gradient/Solvent | % Sol | [IPA 20 | Flow | Temp | pressure |
| Time [min] | [scCO2] | mM NH3] | [ml/min] | [° C.] | [PSI] |
| 0.0 | 70.0 | 30.0 | 4.0 | 40.0 | 2175.0 |
| 9.0 | 70.0 | 30.0 | 4.0 | 40.0 | 2175.0 |

| | |
|---|---|
| Method Name: | S |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | T |
| Device | Agilent 1260 Infinity II SFC with DAD and MS |
| description: | |
| Column: | CHIRAL ART ® Cellulose SZ_4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 70.0 | 30.0 | 4.0 | 40.0 | 2175.0 |
| 10.0 | 70.0 | 30.0 | 4.0 | 40.0 | 2175.0 |

| | |
|---|---|
| Method Name: | U |
| Device | Agilent 1260 Infinity II SFC with DAD and MS |
| description: | |
| Column: | CHIRAL ART ® Cellulose SB 4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

| | |
|---|---|
| Method Name: | V |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | X |
| Device | Agilent 1260 Infinity II SFC with DAD and MS |
| description: | |
| Column: | CHIRAL ART ® Cellulose SB 4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| [Time min] | [scCO2] | mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | Y |
| Device | Agilent 1260 Infinity II SFC with DAD and MS |
| description: | |
| Column: | CHIRAL ART ® Cellulose SC_4.6 × 250 mm_5 μm |
| Column | YMC |
| producer: | |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 65.0 | 35.0 | 4.0 | 40.0 | 2175.0 |
| 10.0 | 65.0 | 35.0 | 4.0 | 40.0 | 2175.0 |

| | |
|---|---|
| Method Name: | Z |
| Device description: | Agilent 1260 Infinity II SFC with DAD and MS |
| Column: | Chiralpak ® IG_4.6 × 250 mm_5 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 65.0 | 35.0 | 4.0 | 40.0 | 2175.0 |
| 10.0 | 65.0 | 35.0 | 4.0 | 40.0 | 2175.0 |

| | |
|---|---|
| Method Name: | AA |
| Device description: | Agilent 1260 Infinity II SFC with DAD and MS |
| Column: | Chiralpak ® IG_3 × 100 mm_3 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 65.0 | 35.0 | 2.0 | 40.0 | 2175.0 |
| 4.0 | 65.0 | 35.0 | 2.0 | 40.0 | 2175.0 |

| | |
|---|---|
| Method Name: | AB |
| Device description: | Agilent 1260 Infinity II SFC with DAD and MS |
| Column: | CHIRAL ART ® Cellulose SB_4.6 × 250 mm_5 |
| | μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

| | |
|---|---|
| Method Name: | AC |
| Device description: | Agilent 1260 Infinity II SFC with DAD and MS |
| Column: | CHIRAL ART ® Cellulose SZ_4.6 × 250 mm_5 |
| | μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 65.0 | 35.0 | 4.0 | 40.0 | 2175.0 |
| 10.0 | 65.0 | 35.0 | 4.0 | 40.0 | 2175.0 |

| | |
|---|---|
| Method Name: | AD |
| Device description: | Agilent 1260 Infinity II SFC with DAD and MS |
| Column: | CHIRAL ART ® Cellulose-SJ_3 × 100 mm_3 μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 95.0 | 5.0 | 2.0 | 40.0 | 2175.0 |
| 3.6 | 40.0 | 60.0 | 2.0 | 40.0 | 2175.0 |
| 4.0 | 40.0 | 60.0 | 2.0 | 40.0 | 2175.0 |

| | |
|---|---|
| Method Name: | AE |
| Device description: | Agilent 1260 Infinity II SFC with DAD |
| Column: | Chiralpak ® IG_3 × 100 mm_3 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 70.0 | 30.0 | 2.0 | 40.0 | 2175.0 |
| 4.0 | 70.0 | 30.0 | 2.0 | 40.0 | 2175.0 |

| | |
|---|---|
| Method Name: | AF |
| Device description: | Agilent 1260 Infinity II SFC with DAD and MS |
| Column: | Chiralcel ® OD-3_3 × 100 mm_3 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 75.0 | 25.0 | 2.0 | 40.0 | 2175.0 |
| 4.0 | 75.0 | 25.0 | 2.0 | 40.0 | 2175.0 |

| | |
|---|---|
| Method Name: | AG |
| Device description: | Agilent 1260 Infinity II SFC with DAD and MS |
| Column: | Chiralpak ® IH_4.6 × 250 mm_5 μm |
| Column producer: | Daicel |

| Gradient/ | | % Sol | | | Back |
|---|---|---|---|---|---|
| Solvent Time | % Sol | [IPA | Flow | Temp | pressure |
| [min] | [scCO2] | 20 mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | AH |
| Device description: | Agilent 1260 Infinity II SFC with DAD and MS |
| Column: | CHIRAL ART ® Cellulose SZ_4.6 × 250 mm_5 |
| | μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | AI |
| Device description: | Agilent 1260 Infinity II SFC with DAD and MS |
| Column: | Chiralpak ® IH_4.6 × 250 mm_5 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

| | |
|---|---|
| Method Name: | AJ |
| Device description: | Agilent 1260 Infinity II SFC with DAD and MS |
| Column: | CHIRAL ART ® Cellulose SC_4.6 × 250 mm_5 |
| | μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 70.0 | 30.0 | 4.0 | 40.0 | 2175.0 |
| 10.0 | 70.0 | 30.0 | 4.0 | 40.0 | 2175.0 |

| | |
|---|---|
| Method Name: | AK |
| Device description: | Agilent 1260 Infinity II SFC with DAD and MS |
| Column: | Chiralcel ® OD-3_3 × 100 mm_3 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 80.0 | 20.0 | 2.0 | 40.0 | 2175.0 |
| 4.0 | 80.0 | 20.0 | 2.0 | 40.0 | 2175.0 |

| | |
|---|---|
| Method Name: | AL |
| Device description: | Agilent 1260 Infinity II SFC with DAD and MS |
| Column: | Chiralpak ® IH_4.6 × 250 mm_5 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

| | |
|---|---|
| Method Name: | AM |
| Device description: | Agilent 1260 Infinity II SFC with DAD and MS |
| Column: | Chiralpak ® IG_4.6 × 250 mm_5 μm |
| Column producer: | Daicel |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

| | |
|---|---|
| Method Name: | AN |
| Device description: | Agilent 1260 Infinity II SFC with DAD |
| Column: | CHIRAL ART ® Cellulose-SZ_3 × 100 mm_3 μm |
| Column producer: | YMC |

| Gradient/ | | % Sol | | | Back |
|---|---|---|---|---|---|
| Solvent Time | % Sol | [MEOH | Flow | Temp | pressure |
| [min] | [scCO2] | 20 mM NH3] | [ml/min] | [° C.] | [PSI] |
| 0.0 | 65.0 | 35.0 | 2.0 | 40.0 | 2175.0 |
| 4.0 | 65.0 | 35.0 | 2.0 | 40.0 | 2175.0 |

| | |
|---|---|
| Method Name: | AO |
| Device description: | Agilent 1260 Infinity II SFC with DAD and MS |
| Column: | Chiralpak ® IG_4.6 × 250 mm_5 μm |
| Column producer: | Daicel |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 95.0 | 5.0 | 4.0 | 40.0 | 2175.0 |
| 9.0 | 40.0 | 60.0 | 4.0 | 40.0 | 2175.0 |
| 10.0 | 40.0 | 60.0 | 4.0 | 40.0 | 2175.0 |

| | |
|---|---|
| Method Name: | AP |
| Device description: | Agilent 1260 Infinity II SFC with DAD and MS |
| Column: | Chiralpak ® IH_3 × 100 mm_3 μm |
| Column producer: | Daicel |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 75.0 | 25.0 | 2.0 | 40.0 | 2175.0 |
| 4.0 | 75.0 | 25.0 | 2.0 | 40.0 | 2175.0 |

| | |
|---|---|
| Method Name: | AR |
| Device description: | Agilent 1260 Infinity II SFC with DAD and MS |
| Column: | CHIRAL ART ® Cellulose SB_4.6 × |
| | 250 mm_5 μm |
| Column producer: | YMC |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 80.0 | 20.0 | 4.0 | 40.0 | 2175.0 |
| 4.0 | 80.0 | 20.0 | 4.0 | 40.0 | 2175.0 |

| | |
|---|---|
| Method Name: | AT |
| Device description: | Agilent 1260 Infinity II SFC with DAD and MS |
| Column: | Lux(R) Cellulose-3_3 × 100 mm_3 μm |
| Column producer: | Phenomenex |

| | | % Sol | | | Back |
|---|---|---|---|---|---|
| Gradient/Solvent | % Sol | [MEOH 20 | Flow | Temp | pressure |
| Time [min] | [scCO2] | mM NH3] | [ml/min] | [° C.] | [PSI] |
| 0.0 | 85.0 | 15.0 | 2.0 | 40.0 | 2175.0 |
| 4.0 | 85.0 | 15.0 | 2.0 | 40.0 | 2175.0 |

| | |
|---|---|
| Method Name: | AU |
| Device description: | Agilent 1260 Infinity II SFC with DAD and MS |
| Column: | Chiralpak ® IG_3 × 100 mm_3 μm |
| Column producer: | Daicel |

| | | % Sol | | | Back |
|---|---|---|---|---|---|
| Gradient/Solvent | % Sol | [IPA 20 | Flow | Temp | pressure |
| Time [min] | [scCO2] | mM NH3] | [ml/min] | [° C.] | [PSI] |
| 0.0 | 65.0 | 35.0 | 2.0 | 40.0 | 2175.0 |
| 4.0 | 65.0 | 35.0 | 2.0 | 40.0 | 2175.0 |

### Mobile Phase Preparations

The mobile phase “MEOH 20 mM NH3” is prepared by adding 3 ml of a commercially available solution of ammonia (7 M in methanol) to 997 ml methanol.

The mobile phase “IPA 20 mM NH3” is prepared by adding 3 ml of a commercially available solution of ammonia (7 M in methanol) to 997 ml isopropyl alcohol.

The mobile phase “ETOH 20 mM NH3” is prepared by adding 3 ml of a commercially available solution of ammonia (7 M in methanol) to 997 ml ethanol.

### Preparations

### Synthesis Scheme of Intermediate 9

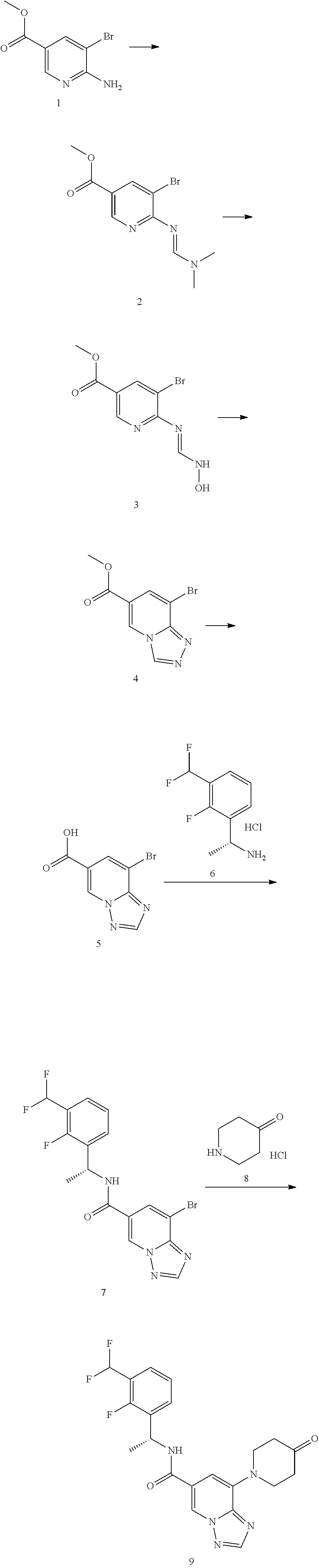

### Synthesis of Intermediate 2

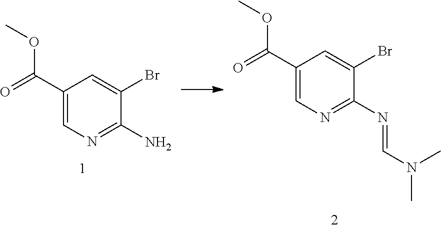

Methyl 6-amino-5-bromonicotinate 1 (14.8 g, 61 mmol) and N,N-dimethylformamide dimethyl acetal (17.2 mL, 122 mmol) in DMF (141 mL) is stirred for 4 h at 100° C. The solvents are evaporated under reduced pressure to obtain intermediate 2. The intermediate is used without further purification.

### Synthesis of Intermediate 3

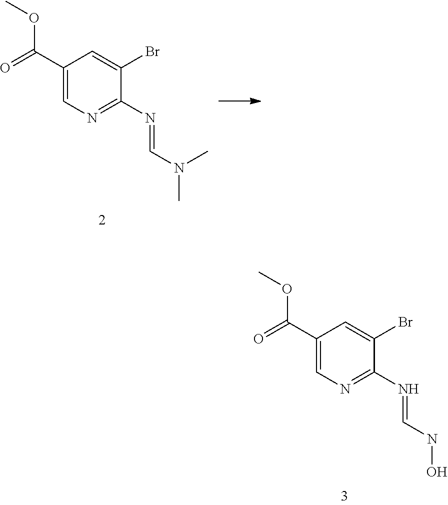

Intermediate 2 (17.5 g, 61 mmol), sodium acetate (11 g, 134 mmol) and hydroxylamine hydrochloride (6.4 g, 91 mmol) in ethanol (249 mL) are stirred for 3 h at 50° C. The mixture is poured into water and is further stirred for 1 h at rt. The solids are filtered off, washed with water and dried in vacuo at 55° C. to obtain intermediate 3. The intermediate 3 is used without further purification.

Intermediate 3
Analytical HPLC-MS Method: D

| | |
|---|---|
| Rₜ [min]: 0.83 | MS [m/z]: 274 [M + H]⁺ |

### Synthesis of Intermediate 4

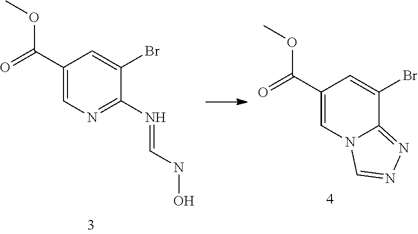

Intermediate 3 (16.7 g, 61 mmol) in THF (201 mL) is cooled to 0° C. Trifluoroacetic anhydride (11 mL, 79 μmol) is added dropwise. After complete addition, the mixture is allowed to reach rt and stirring is continued for 16 h. The mixture is basified with NaHCO₃ solution under ice cooling, diluted with water and stirred for 2 h. The solvent is reduced under reduced pressure and the precipitate is filtered off, washed with water and dried in vacuo at 55° C. to obtain intermediate 4. The intermediate 4 is used without further purification.

Intermediate 4
Analytical HPLC-MS Method: D

| | |
|---|---|
| Rₜ [min]: 0.75 | MS [m/z]: 256 [M + H]⁺ |

### Synthesis of Intermediate 5

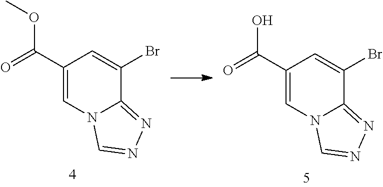

An aqueous NaOH solution (4 M; 14.5 mL, 68 mmol) is added to intermediate 4 (13.5 g, 53 mmol) in MeOH (149 mL) and stirred for 1 h at 50° C. The mixture is further stirred at rt for 16 h. MeOH is evaporated under reduced pressure before the mixture is diluted with water and filtered. The aqueous mixture is acidified with aqueous 4 M HCl solution. The mixture is stirred for 1 h. The precipitate is filtered off, washed with water and dried in vacuo at 55° C. to obtain intermediate 5. The intermediate 5 is used without further purification.

Intermediate 5
Analytical HPLC-MS Method: E

| | |
|---|---|
| Rₜ [min]: 0.66 | MS [m/z]: 242 [M + H]⁺ |

### Synthesis of Intermediate 7

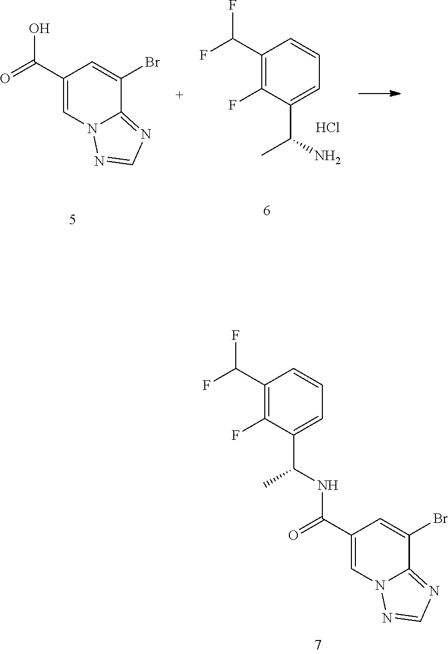 [SMILES: O=C(O)c1cc(Br)c2ncnn2c1 ; C[C@@H](N)c1cccc(C(F)F)c1F ; Cl ; C[C@@H](NC(=O)c1cc(Br)c2ncnn2c1)c1cccc(C(F)F)c1F]

Intermediate 5 (140 mg, 578 μmol) in DMF (9.4 mL) is treated with HATU (330 mg, 868 μmol) and triethylamine (325 μL, 2.3 mmol) and stirred for 15 h at rt. Benzylamine 6 (CAS: 1389852-29-2; 131 mg, 578 μmol) is added and the mixture is stirred for 16 h. MeOH/THF is added, solids are filtered off and the mixture is purified by prep. RP-HPLC (acidic conditions) to obtain intermediate 7.

Intermediate 7
Analytical HPLC-MS Method: D

| | |
|---|---|
| Rₜ [min]: 0.94 | MS [m/z]: 413 [M + H]⁺ |

### Synthesis of Intermediate 9

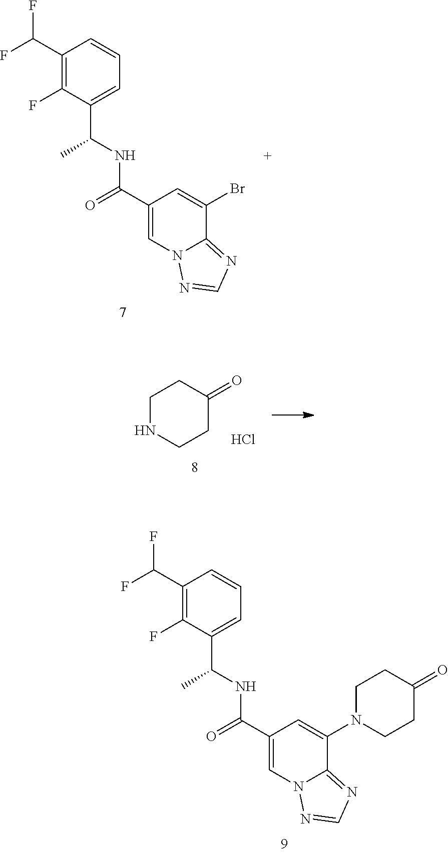 [SMILES: C[C@@H](NC(=O)c1cc(Br)c2ncnn2c1)c1cccc(C(F)F)c1F ; O=C1CCNCC1 ; Cl ; C[C@@H](NC(=O)c1cc(N2CCC(=O)CC2)c2ncnn2c1)c1cccc(C(F)F)c1F]

A mixture of intermediate 7 (7.0 g, 16 mmol), piperidin-4-one hydrochloride 8 (2.5 g, 18 mmol), K₃PO₄ (10.4 g, 48 mmol), and catalyst I (406 mg, 483 μmol) in 1,4-dioxane (70 mL) is stirred under argon for 16 h at 110° C. The mixture is diluted with EtOAc and filtered. The solvent is removed under reduced pressure. The material is taken up in EtOAc and extracted with water and saturated aqueous NaCl solution. The separated organic layer is dried over Na₂SO₄, filtered and the solvent is evaporated under reduced pressure. The material is purified by column chromatography (SiO₂; EtOAc/petroleum ether: 1:3→1:0) to obtain intermediate 9.

Intermediate 9
Analytical HPLC-MS Method: D

| | |
|---|---|
| Rₜ [min]: 0.91 | MS [m/z]: 432 [M + H]⁺ |

¹H NMR (400 MHz, DMSO-d₆) δ (ppm): 1.48-1.57 (3 H), 2.51-2.57
(4 H), 3.97-4.07 (4 H), 5.37-5.47 (1 H), 7.07-7.38 (3 H), 7.49-7.57
(1 H), 7.65-7.74 (1 H), 8.50-8.59 (1 H), 9.05-9.14 (2 H).

### Scheme of Alternative Synthesis of Intermediate 7

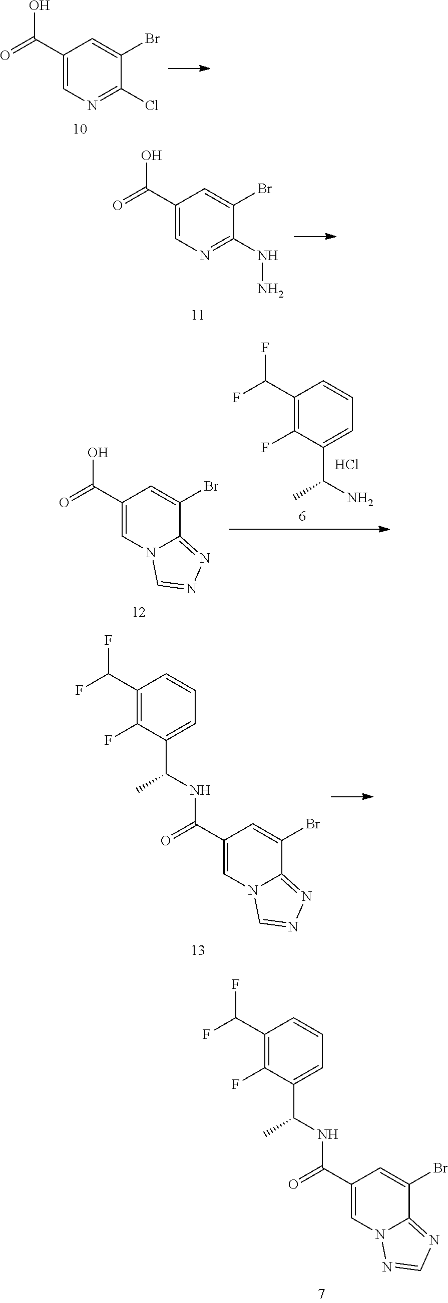

### Synthesis of Intermediate 11

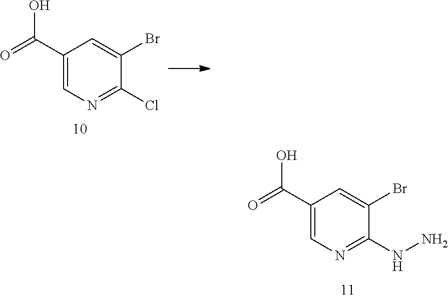 [SMILES: O=C(O)c1cnc(Cl)c(Br)c1 ; NNc1ncc(C(=O)O)cc1Br]

Hydrazine hydrate (45 ml, 603 mmol, 64-65%) is added to a mixture of 5-bromo-6-chloronicotinic acid 10 (50 g, 201 mmol) in ethanol (410 mL). The mixture is refluxed for 16 h. After cooling to rt, the solids are filtered off, washed with ethanol, and dried in vacuo to obtain intermediate 11. The material is used without further purification.

Intermediate 11
Analytical HPLC-MS Method: E

| | |
|---|---|
| Rₜ [min]: 0.16 | MS [m/z]: 232 [M + H]⁺ |

### Synthesis of Intermediate 12

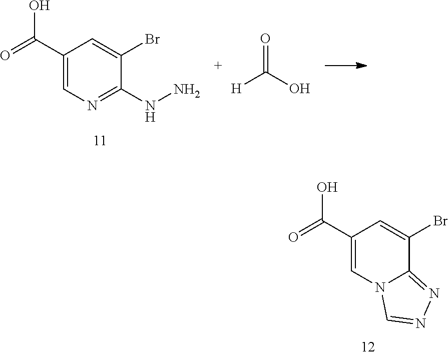 [SMILES: NNc1ncc(C(=O)O)cc1Br ; O=CO ; O=C(O)c1cc(Br)c2nncn2c1]

Intermediate 11 (16.9 g, 73 mmol) is treated with formic acid (40 mL, 1.1 mmol) and stirred at 100° C. for 2 h. The mixture is cooled to 0° C. The solid material is filtered off and dried under reduced pressure to obtain the intermediate 12. The material is used without further purification.

Intermediate 12
Analytical HPLC-MS Method: E

| | |
|---|---|
| Rₜ [min]: 0.56 | MS [m/z]: 242 [M + H]⁺ |

### Synthesis of Intermediate 13

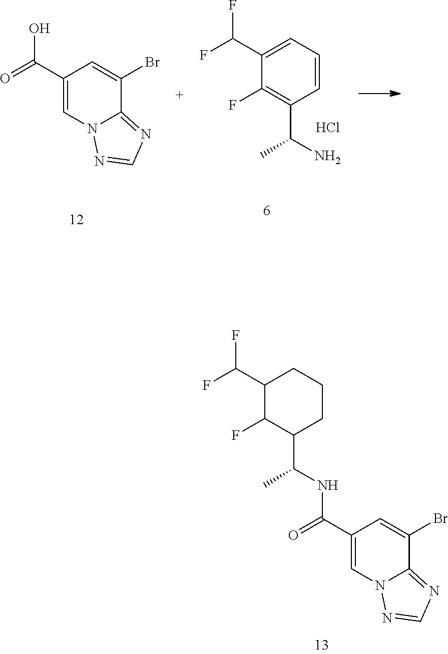 [SMILES: O=C(O)c1cc(Br)c2ncnn2c1 ; C[C@@H](N)c1cccc(C(F)F)c1F ; Cl ; C[C@@H](NC(=O)c1cc(Br)c2ncnn2c1)C1CCCC(C(F)F)C1F]

A mixture of intermediate 12 (6.3 g, 25 mmol), benzylamine 6 (CAS: 1389852-29-2; 6.0 g, 26 mmol) and 4-methylmorpholine (11 mL, 99 mmol) in ACN (60 mL) is cooled down to 0° C. 1-Propanephosphonic acid anhydride (37 mL, 62 mmol) is added dropwise. The mixture is allowed to reach rt before it is poured into water and stirred for 20 min. The solids are filtered and washed with ACN/water (1:1). The material is dried in vacuo (55° C.) to obtain intermediate 13. The material is used without further purification.

Intermediate 13
Analytical HPLC-MS Method: E

| | |
|---|---|
| Rₜ [min]: 0.92 | MS [m/z]: 413 [M + H]⁺ |

¹H NMR (400 MHz, DMSO-d₆) δ (ppm): 1.48-1.72 (9 H), 1.85-1.94
(2 H), 2.43-2.48 (1 H), 2.62-2.72 (1 H), 2.80-2.92 (4 H), 3.02-3.10
(1 H), 4.28-4.41 (3 H), 5.36-5.46 (1 H), 7.07-7.37 (3 H), 7.49-7.56
(1 H), 7.65-7.72 (1 H), 8.47-8.53 (1 H), 9.02-9.10 (2 H), missing
proton(s) presumably hidden by/overlapping with solvent signals.

### Synthesis of Intermediate 7

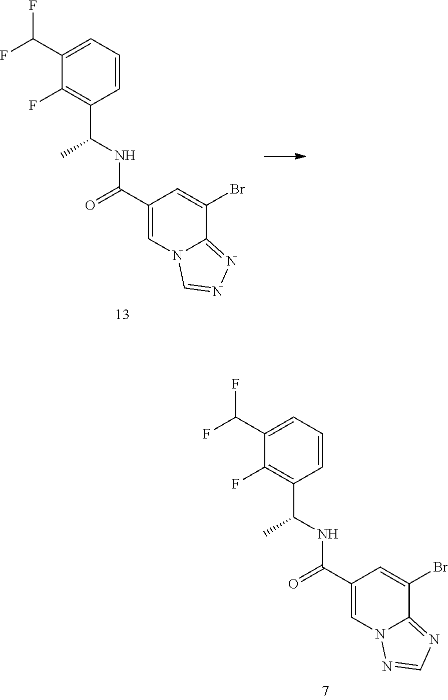 [SMILES: C[C@@H](NC(=O)c1cc(Br)c2nncn2c1)c1cccc(C(F)F)c1F ; C[C@@H](NC(=O)c1cc(Br)c2ncnn2c1)c1cccc(C(F)F)c1F]

A mixture of intermediate 13 (710 mg, 1.7 mmol), Cs₂CO₃ (280 mg, 859 μmol), piperidine (85 μL, 859 μmol) in 1,4-dioxane (10 mL) is stirred 6 h at 100° C. EtOAc and water are added. The aqueous layer is separated and extracted with EtOAc. The combined organic layers are dried over Na₂SO₄. The solids are filtered, and the solvent is evaporated under reduced pressure. The material is purified by column chromatography (SiO₂; EtOAc/cyclohexane: 50:50→100:0) to obtain intermediate 7.

Intermediate 7
Analytical HPLC-MS Method: D

| | |
|---|---|
| Rₜ [min]: 0.94 | MS [m/z]: 413 [M + H]⁺ |

### Synthesis of Example E1

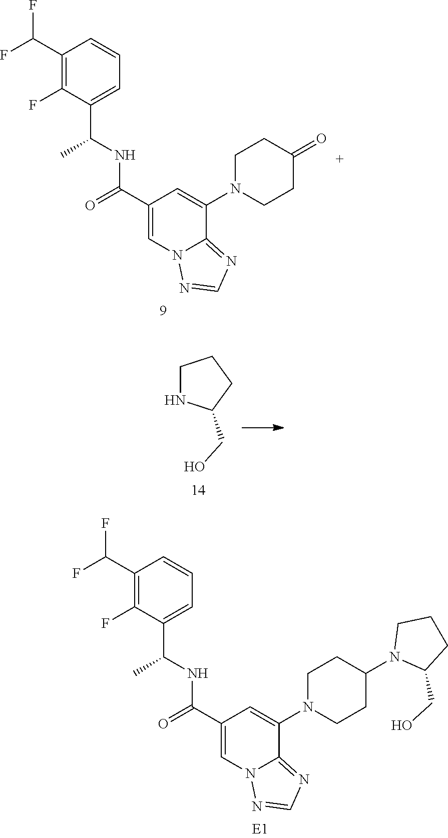 [SMILES: C[C@@H](NC(=O)c1cc(N2CCC(=O)CC2)c2ncnn2c1)c1cccc(C(F)F)c1F ; OC[C@H]1CCCN1 ; C[C@@H](NC(=O)c1cc(N2CCC(N3CCC[C@@H]3CO)CC2)c2ncnn2c1)c1cccc(C(F)F)c1F]

A mixture of Intermediate 9 (34.5 mg, 80 μmol), D-prolinol 14 (12.1 mg, 120 μmol), AcOH (30.0 μL, 51 μmol), and 2-picoline-borane complex (8.6 mg, 80 μmol) in MeOH (2.0 mL) is stirred for 12 h at rt. The mixture was diluted with DMF und purified by prep. RP-HPLC (basic conditions) to obtain the example E1.

Example E1

| | |
|---|---|
| HPLC-MS Method: A | |
| Rₜ [min]: 0.73 | MS [m/z]: 517 [M + H]⁺ |
| Analytical SFC method: AU | |
| Rₜ [min]: 1.66 | d.e. > 95% |

¹H NMR (400 MHz, DMSO-d₆) δ (ppm): 1.48-1.72 (9 H), 1.85-1.94
(2 H), 2.43-2.48 (1 H), 2.62-2.72 (1 H), 2.80-2.92 (4 H), 3.02-3.10
(1 H), 4.28-4.41 (3 H), 5.36-5.46 (1 H), 7.07-7.37 (3 H), 7.49-7.56
(1 H), 7.65-7.72 (1 H), 8.47-8.53 (1 H), 9.02-9.10 (2 H), missing
proton(s) presumably hidden by/overlapping with solvent signals.

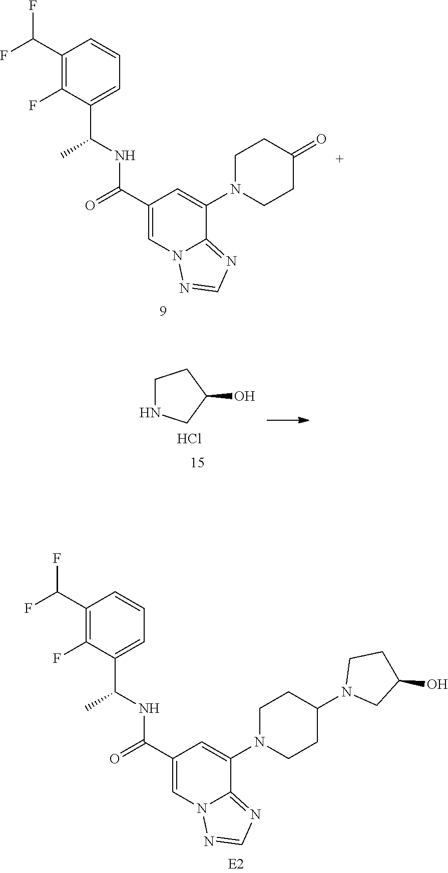 [SMILES: C[C@@H](NC(=O)c1cc(N2CCC(=O)CC2)c2ncnn2c1)c1cccc(C(F)F)c1F ; O[C@@H]1CCNC1 ; Cl ; C[C@@H](NC(=O)c1cc(N2CCC(N3CC[C@@H](O)C3)CC2)c2ncnn2c1)c1cccc(C(F)F)c1F]

Example E2 is prepared in analogy to example E1: Intermediate 9 (80 μmol), (R)-3-hydroxypyrrolidine hydrochloride 15 (0.12 mmol), AcOH (0.51 mmol), and 2-picoline-borane complex (80 μmol), 2 mL MeOH.

Example E2
Analytical HPLC-MS Method: D

| | |
|---|---|
| Rₜ [min]: 0.91 | MS [m/z]: 503 [M + H]⁺ |

Analytical SFC method: L

| | |
|---|---|
| Rₜ [min]: 6.60 | d.e. > 98% |

¹H NMR (400 MHz, DMSO-d₆) δ (ppm): 1.47-1.61 (6 H), 1.88-2.02 (3 H), 2.17-2.27
(1 H), 2.35-2.42 (1 H), 2.60-2.69 (1 H), 2.74-2.83 (1 H), 2.92-3.03 (2 H), 4.13-4.25
(3 H), 4.58-4.70 (1 H), 5.36-5.46 (1 H), 7.06-7.38 (3 H), 7.49-7.56 (1 H), 7.65-7.73
(1 H), 8.50 (1 H), 9.01-9.11 (2 H), missing proton(s) presumably hidden by/overlapping
with solvent signals

### Synthesis of Example E3

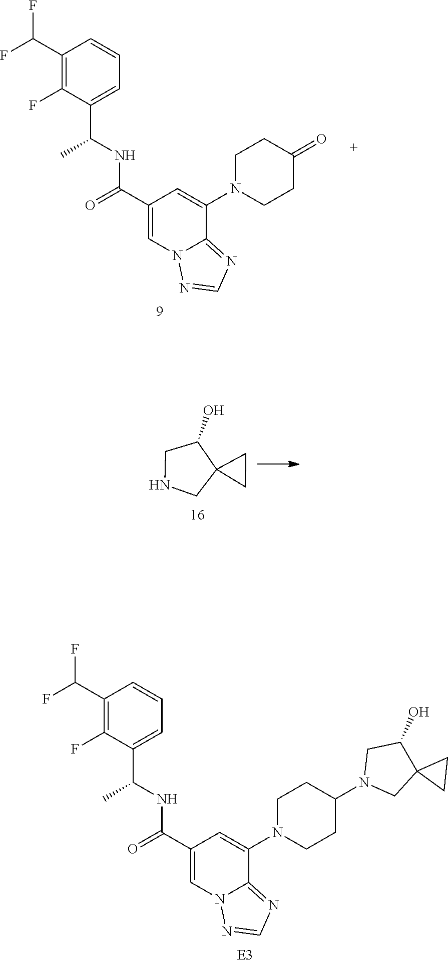 [SMILES: C[C@@H](NC(=O)c1cc(N2CCC(=O)CC2)c2ncnn2c1)c1cccc(C(F)F)c1F ; O[C@H]1CNCC12CC2 ; C[C@@H](NC(=O)c1cc(N2CCC(N3C[C@H](O)C4(CC4)C3)CC2)c2ncnn2c1)c1cccc(C(F)F)c1F]

Example E3 is prepared in analogy to example E1: Intermediate 9 (80 μmol), (7R)-5-azaspiro[2.4]heptan-7-ol 16 (0.12 mmol), AcOH (0.51 mmol), and 2-picoline-borane complex (80 μmol), 2 mL MeOH.

Example E3
Analytical HPLC-MS Method: A

| | |
|---|---|
| Rₜ [min]: 0.76 | MS [m/z]: 529 [M + H]⁺ |

Analytical SFC method: L

| | |
|---|---|
| Rₜ [min]: 2.26 | d.e. > 98% |

¹H NMR (400 MHz, DMSO-d₆) δ (ppm): 0.30-0.40 (1 H), 0.45-0.58 (2 H), 0.77-0.86
(1 H), 1.45-1.61 (5 H), 1.87-1.98 (2 H), 2.18-2.27 (1 H), 2.41-2.48 (1 H), 2.60-2.66
(1 H), 2.93-3.04 (2 H), 3.04-3.12 (1 H), 3.68-3.77 (1 H), 4.16-4.26 (2 H), 4.47-4.56
(1 H), 5.36-5.46 (1 H), 7.07-7.38 (3 H), 7.49-7.56 (1 H), 7.64-7.72 (1 H), 8.47-8.52
(1 H), 9.02-9.11 (2 H), missing proton(s) presumably hidden by/overlapping with solvent
signals

### Synthesis of Example E4

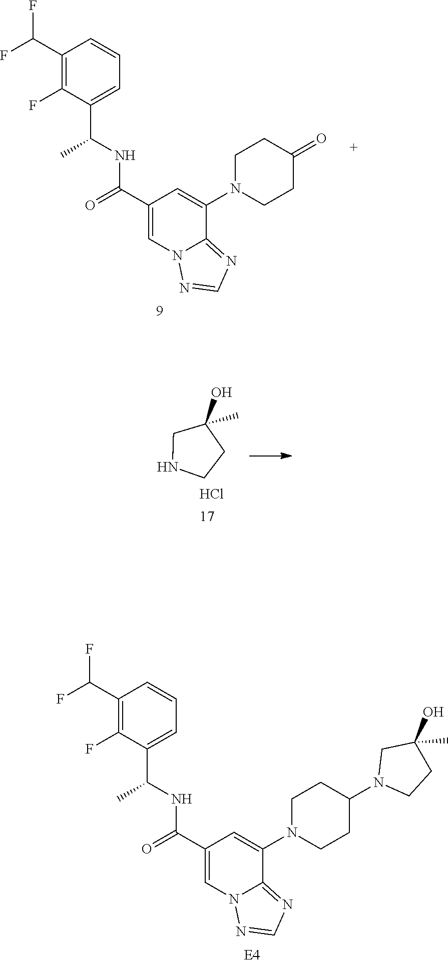

Example E4 is prepared in analogy to example E1: Intermediate 9 (80 μmol), (3S)-3-methylpyrrolidin-3-ol hydrochloride 17 (0.12 mmol), AcOH (0.51 mmol), and 2-picoline-borane complex (80 μmol), 2 mL MeOH.

Example E4
Analytical HPLC-MS Method: A

| | |
|---|---|
| Rₜ [min]: 0.75 | MS [m/z]: 517 [M + H]⁺ |

Analytical SFC method: F

| | |
|---|---|
| Rₜ [min]: 1.56 | d.e. > 90% |

¹H NMR (400 MHz, DMSO-d₆) δ (ppm): 1.21-1.27 (3 H), 1.48-1.61 (5 H), 1.62-1.77
(2 H), 1.87-1.98 (2 H), 2.18-2.29 (1 H), 2.52-2.61 (3 H), 2.65-2.74 (1 H), 2.94-3.05
(2 H), 4.14-4.25 (2 H), 4.41-4.50 (1 H), 5.36-5.46 (1 H), 7.07-7.38 (3 H), 7.48-7.57
(1 H), 7.64-7.72 (1 H), 8.48-8.53 (1 H), 9.02-9.10 (2 H).

### Synthesis of Example E5

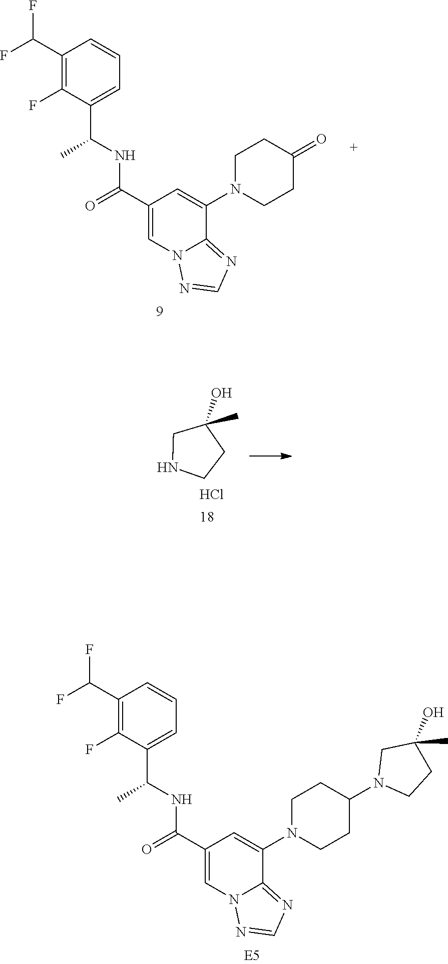

Example E5 is prepared in analogy to example E1: Intermediate 9 (80 μmol), (3R)-3-methylpyrrolidin-3-ol hydrochloride 18 (0.12 mmol), AcOH (0.51 mmol), and 2-picoline-borane complex (80 μmol), 2 mL MeOH.

Example E5
Analytical HPLC-MS Method: A

| | |
|---|---|
| Rₜ [min]: 0.75 | MS [m/z]: 517 [M + H]⁺ |

Analytical SFC method: L

| | |
|---|---|
| Rₜ [min]: 2.02 | d.e. > 98% |

¹H NMR (400 MHz, DMSO-d₆) δ (ppm): 1.19-1.28 (3 H), 1.46-1.60 (5 H), 1.62-1.77
(2 H), 1.87-1.97 (2 H), 2.19-2.29 (1 H), 2.52-2.62 (3 H), 2.65-2.74 (1 H), 2.95-3.05
(2 H), 4.14-4.26 (2 H), 4.41-4.49 (1 H), 5.36-5.46 (1 H), 7.06-7.38 (3 H), 7.48-7.56
(1 H), 7.64-7.72 (1 H), 8.47-8.52 (1 H), 9.02-9.12 (2 H).

### Synthesis of Example E6

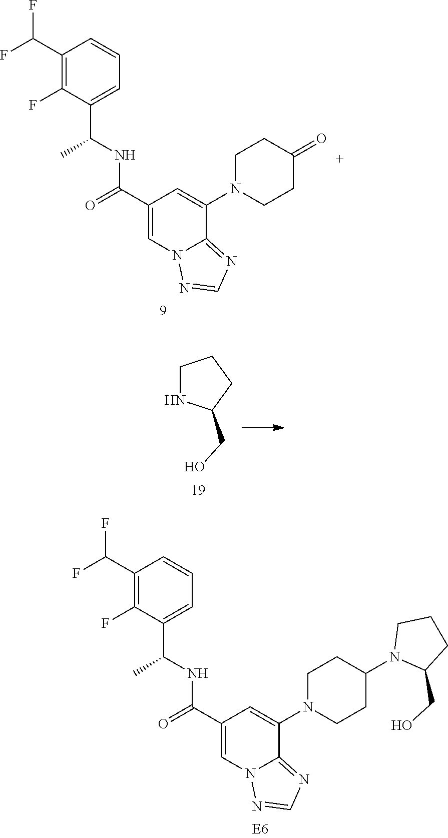 [SMILES: C[C@@H](NC(=O)c1cc(N2CCC(=O)CC2)c2ncnn2c1)c1cccc(C(F)F)c1F ; OC[C@@H]1CCCN1 ; C[C@@H](NC(=O)c1cc(N2CCC(N3CCC[C@H]3CO)CC2)c2ncnn2c1)c1cccc(C(F)F)c1F]

Example E6 is prepared in analogy to example E1: Intermediate 9 (80 μmol), L-prolinol 19 (0.12 mmol), AcOH (0.51 mmol), and 2-picoline-borane complex (80 μmol), 2 mL MeOH.

Example E6
Analytical HPLC-MS Method: A

| | |
|---|---|
| Rₜ [min]: 0.76 | MS [m/z]: 517 [M + H]⁺ |

Analytical SFC method: AP

| | |
|---|---|
| Rₜ [min]: 1.91 | d.e. > 98% |

¹H NMR (400 MHz, DMSO-d₆) δ (ppm): 1.48-1.72 (9 H), 1.85-1.94 (2 H), 2.62-2.71
(1 H), 2.79-2.93 (4 H), 3.01-3.11 (1 H), 4.27-4.41 (3 H), 5.36-5.47 (1 H), 7.07-7.37
(3 H), 7.49-7.56 (1 H), 7.65-7.72 (1 H), 8.48-8.53 (1 H), 9.02-9.11 (2 H), missing
proton(s) presumably hidden by/overlapping with solvent signals

### Synthesis of Example E7

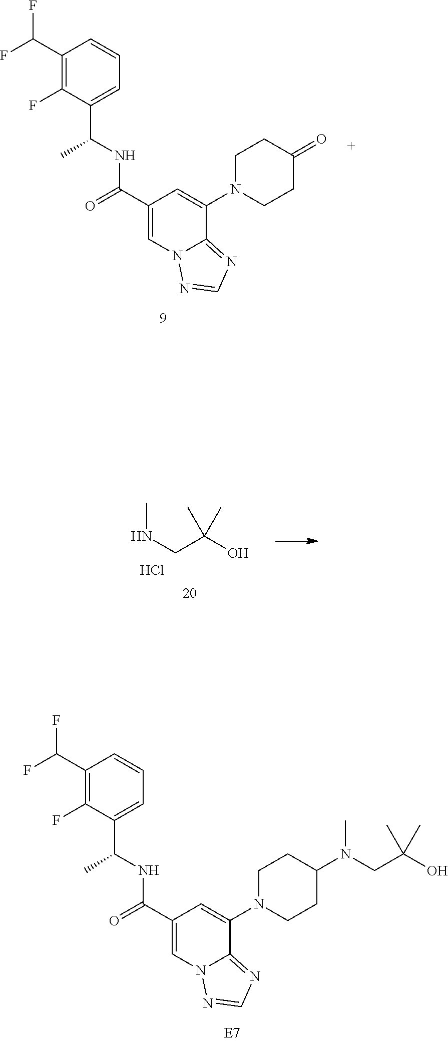

Example E7 is prepared in analogy to example E1: Intermediate 9 (80 μmol), 2-methyl-1-(methylamino)propan-2-ol 20 (0.12 mmol), AcOH (0.51 mmol), and 2-picoline-borane complex (80 μmol), 2 mL MeOH.

Example E7
Analytical HPLC-MS Method: A

| | |
|---|---|
| Rₜ [min]: 0.84 | MS [m/z]: 519 [M + H]⁺ |

Analytical SFC method: AR

| | |
|---|---|
| Rₜ [min]: 5.33 | e.e. > 98% |

¹H NMR (400 MHz, DMSO-d₆) δ (ppm): 1.04-1.11 (6 H), 1.49-1.54 (3 H), 1.55-1.65
(2 H), 1.77-1.86 (2 H), 2.26-2.31 (2 H), 2.31-2.35 (3 H), 2.53-2.62 (1 H), 2.73-2.83
(2 H), 3.92-3.99 (1 H), 4.36-4.46 (2 H), 5.36-5.46 (1 H), 7.08-7.38 (3 H), 7.49-7.56
(1 H), 7.64-7.72 (1 H), 8.49-8.53 (1 H), 9.02-9.11 (2 H).

### Synthesis of Example E8

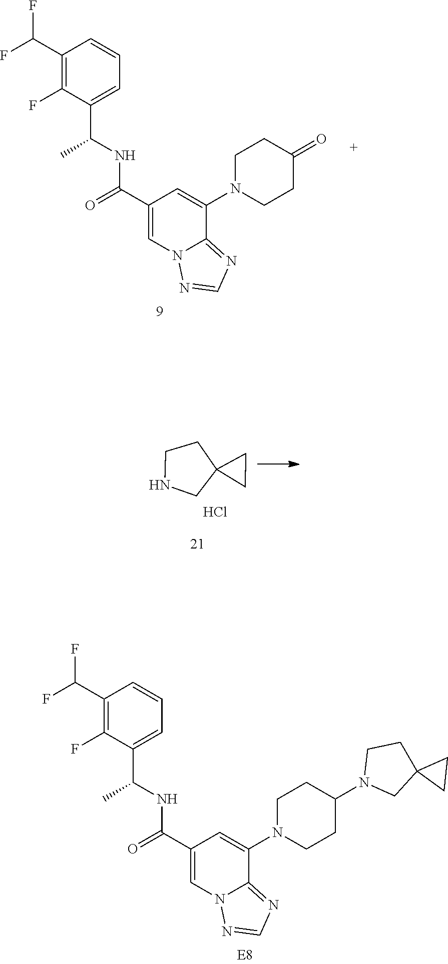 [SMILES: C[C@@H](NC(=O)c1cc(N2CCC(=O)CC2)c2ncnn2c1)c1cccc(C(F)F)c1F ; C1CC2(CC2)CN1 ; Cl ; C[C@@H](NC(=O)c1cc(N2CCC(N3CCC4(CC4)C3)CC2)c2ncnn2c1)c1cccc(C(F)F)c1F]

Example E8 is prepared in analogy to example E1: Intermediate 9 (80 μmol), 5-azaspiro[2.4]heptane hydrochloride 21 (0.12 mmol), AcOH (0.51 mmol), and 2-picoline-borane complex (80 μmol), 2 mL MeOH.

Example E8
Analytical HPLC-MS Method: A

| | |
|---|---|
| Rₜ [min]: 0.94 | MS [m/z]: 513 [M + H]⁺ |

Analytical SFC method: AF

| | |
|---|---|
| Rₜ [min]: 0.98 | e.e. > 98% |

¹H NMR (400 MHz, DMSO-d₆) δ (ppm): 0.44-0.55 (4 H), 1.49-1.62 (5 H), 1.68-1.75
(2 H), 1.89-1.98 (2 H), 2.17-2.26 (1 H), 2.68-2.75 (2 H), 2.93-3.03 (2 H), 4.17-4.26
(2 H), 5.36-5.45 (1 H), 7.08-7.38 (3 H), 7.50-7.56 (1 H), 7.65-7.72 (1 H), 8.49-8.52
(1 H), 9.04-9.09 (2 H), missing proton(s) presumably hidden by/overlapping with solvent
signals

### Synthesis of Example E10

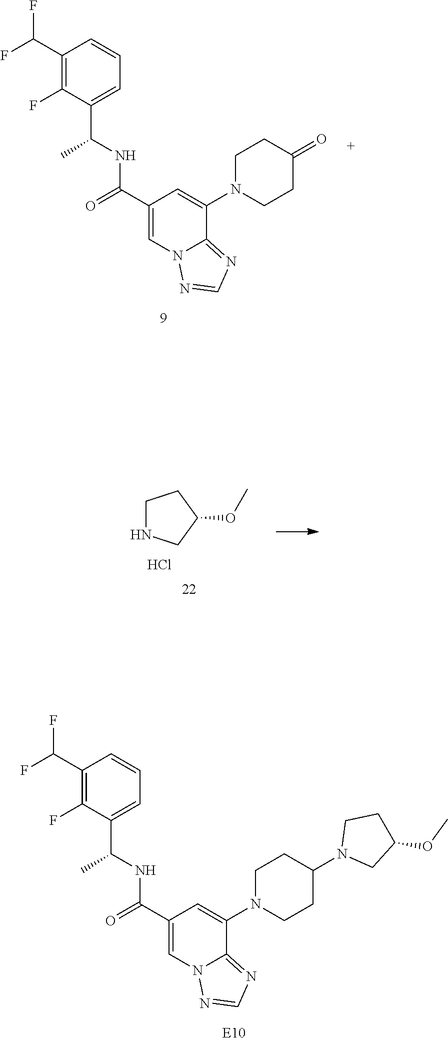

Example E10 is prepared in analogy to example E1: Intermediate 9 (80 μmol), (S)-3-methoxypyrrolidine hydrochloride 22 (0.12 mmol), AcOH (0.51 mmol), and 2-picoline-borane complex (80 μmol), 2 mL MeOH.

Example E10
Analytical HPLC-MS Method: A

| | |
|---|---|
| Rₜ [min]: 0.81 | MS [m/z]: 531 [M + H]⁺ |

Analytical SFC method: AP

| | |
|---|---|
| Rₜ [min]: 1.07 | d.e. > 98% |

¹H NMR (400 MHz, DMSO-d₆) δ (ppm): 1.49-1.69 (6 H), 1.89-2.01 (3 H), 2.17-2.27
(1 H), 2.58-2.66 (1 H), 2.73-2.80 (1 H), 2.92-3.03 (2 H), 3.15-3.19 (3 H), 3.81-3.89
(1 H), 4.15-4.26 (2 H), 5.36-5.46 (1 H), 7.07-7.38 (3 H), 7.49-7.56 (1 H), 7.65-7.73
(1 H), 8.47-8.54 (1 H), 9.02-9.10 (2 H), missing proton(s) presumably hidden by/
overlapping with solvent signals

### Synthesis of Examples Cis-E11a and Cis-E11b

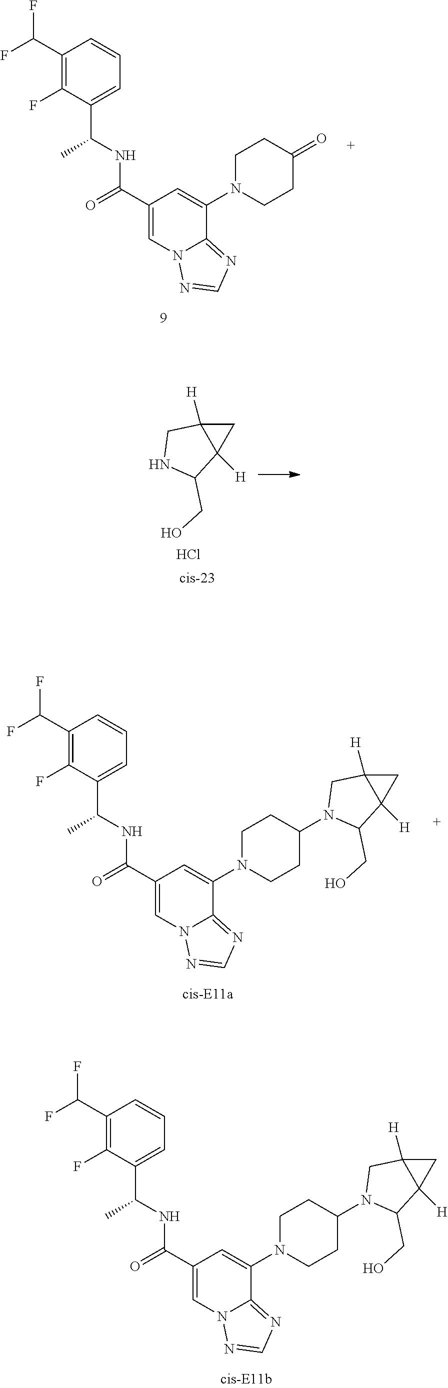 [SMILES: C[C@@H](NC(=O)c1cc(N2CCC(=O)CC2)c2ncnn2c1)c1cccc(C(F)F)c1F ; OCC1NCC2CC21 ; Cl ; C[C@@H](NC(=O)c1cc(N2CCC(N3CC4CC4C3CO)CC2)c2ncnn2c1)c1cccc(C(F)F)c1F ; C[C@@H](NC(=O)c1cc(N2CCC(N3CC4CC4C3CO)CC2)c2ncnn2c1)c1cccc(C(F)F)c1F]

A mixture of Intermediate 9 (250 mg, 579 μmol), (1 r,2s,5s)-rel-3-azabicyclo[3.1.0]hexan-2-ylmethanol hydrochloride cis-23 (134 mg, 869 μmol), AcOH (68 μL, 1.16 mmol), triethylamine (117 μL, 1.16 mmol) in DMF (2.9 mL) is stirred for 4 h at rt, before sodium triacetoxyborohydride (246 mg, 1.16 mmol) is added at rt. The mixture is stirred for 2 h at rt. Water, MeOH, THF are added, the mixture filtered, and directly purified by prep. RP-HPLC (basic conditions) and chiral SFC to obtain examples cis-Ella and cis-E11b as single stereoisomers. The absolute configuration of the hydroxymethyl substituent and [3.1.0] ring system is not known; their relative configuration is cis.

Example cis-E11a
Analytical HPLC-MS Method: D

| | |
|---|---|
| Rₜ [min]: 0.96 | MS [m/z]: 529 [M + H]⁺ |

Analytical SFC method: N

| | |
|---|---|
| Rₜ [min]: 4.47 | d.e. > 96% |

¹H NMR (400 MHz, DMSO-d₆) δ (ppm): 0.14-0.25 (1 H), 0.48-0.56 (1 H), 1.29-1.37
(1 H), 1.47-1.73 (7 H), 1.78-1.86 (1 H), 2.69-2.88 (5 H), 2.98-3.06 (1 H), 3.18-3.26
(1 H), 3.54-3.62 (1 H), 4.34-4.48 (3 H), 5.36-5.46 (1 H), 7.07-7.38 (3 H), 7.49-7.57
(1 H), 7.64-7.73 (1 H), 8.46-8.54 (1 H), 9.02-9.10 (2 H).

Example cis-E11b
Analytical HPLC-MS Method: D

| | |
|---|---|
| Rₜ [min]: 0.96 | MS [m/z]: 529 [M + H]⁺ |

Analytical SFC method: N

| | |
|---|---|
| Rₜ [min]: 3.53 | d.e. > 98% |

¹H NMR (400 MHz, DMSO-d₆) δ (ppm): 0.15-0.24 (1 H), 0.48-0.56 (1 H), 1.28-1.38
(1 H), 1.48-1.72 (7 H), 1.77-1.86 (1 H), 2.69-2.89 (5 H), 2.99-3.07 (1 H), 3.19-3.27
(1 H), 3.54-3.62 (1 H), 4.34-4.49 (3 H), 5.36-5.46 (1 H), 7.06-7.38 (3 H), 7.49-7.57
(1 H), 7.64-7.72 (1 H), 8.46-8.54 (1 H), 9.00-9.10 (2 H)

### Synthesis of Example E12

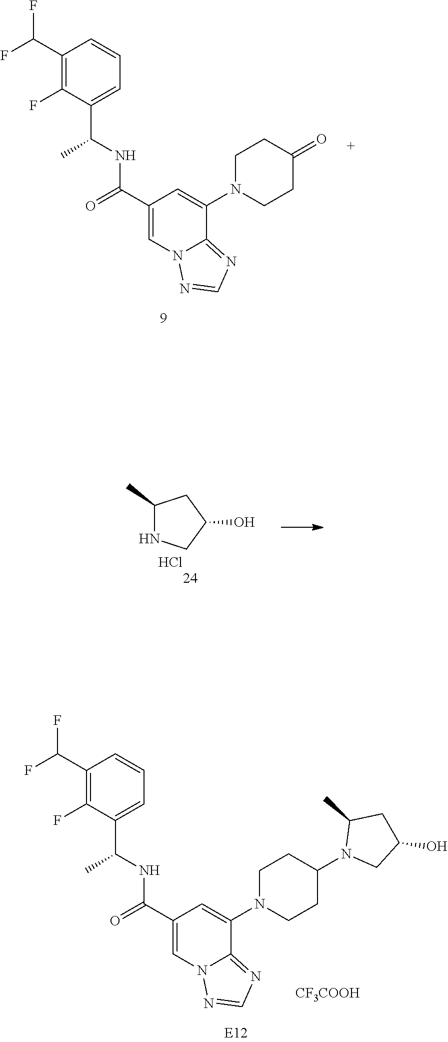

Example E12 is prepared in analogy to example E1: Intermediate 9 (80 μmol), (3S,5S)-5-methylpyrrolidin-3-ol hydrochloride 24 (0.12 mmol), AcOH (0.51 mmol), and 2-picoline-borane complex (80 μmol), 2 mL MeOH.

Example E12
Analytical HPLC-MS Method: A

| | |
|---|---|
| Rₜ [min]: 0.72 | MS [m/z]: 517 [M + H]⁺ |

Analytical SFC method: AL

| | |
|---|---|
| Rₜ [min]: 2.30 | d.e. > 98% |

¹H NMR (400 MHz, DMSO-d₆) δ (ppm): 1.48-1.69 (6 H), 1.89-2.00 (3 H), 2.17-2.26
(1 H), 2.58-2.66 (1 H), 2.73-2.80 (1 H), 2.92-3.03 (2 H), 3.15-3.19 (3 H), 3.81-3.90
(1 H), 4.15-4.26 (2 H), 5.36-5.46 (1 H), 7.07-7.38 (3 H), 7.48-7.56 (1 H), 7.64-7.72
(1 H), 8.47-8.53 (1 H), 9.00-9.12 (2 H), missing proton(s) presumably hidden by/
overlapping with solvent signals

### Synthesis of Example E13

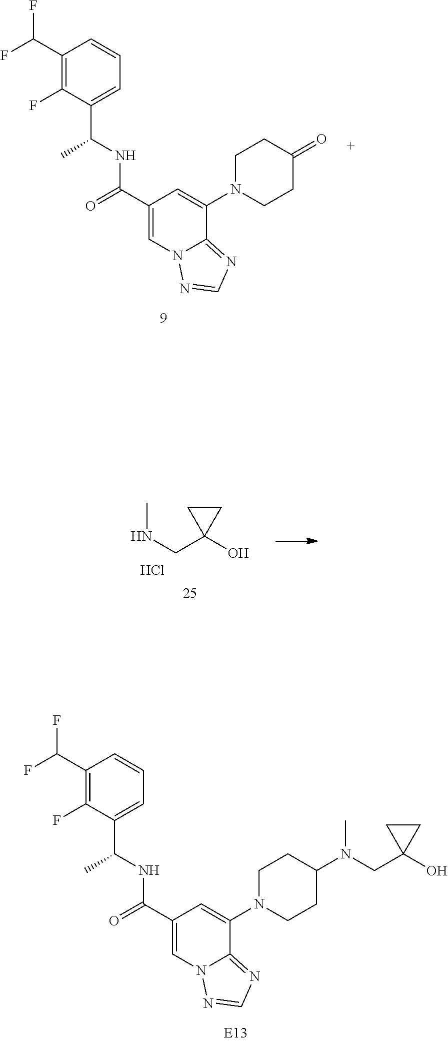

A mixture of Intermediate 9 (200 mg, 464 μmol), 1-[(methylamino)methyl]cyclopropan-1-ol hydrochloride 25 (101 mg, 695 μmol), AcOH (54 μL, 927 μmol), triethylamine (94 mg, 927 μmol) in DMF (2.9 mL) is stirred for 4 h at rt, before sodium triacetoxyborohydride (197 mg, 927 μmol) is added at rt. The mixture is stirred for 2 h at rt. Water, MeOH, THF are added, the mixture is filtered, and directly purified by prep. RP-HPLC (basic conditions) to obtain example E13.

Example E13
Analytical HPLC-MS Method: D

| | |
|---|---|
| Rₜ [min]: 0.97 | MS [m/z]: 517 [M + H]⁺ |

Analytical SFC method: T

| | |
|---|---|
| Rₜ [min]: 4.34 | e.e. = 90% |

¹H NMR (400 MHz, DMSO-d₆) δ (ppm): 0.35-0.42 (2 H), 0.52-0.58 (2 H), 1.48-1.66
(5 H), 1.78-1.89 (2 H), 2.25-2.35 (3 H), 2.52-2.55 (2 H), 2.60-2.70 (1 H), 2.77-2.86
(2 H), 4.33-4.46 (2 H), 4.78-4.81 (1 H), 5.37-5.46 (1 H), 7.07-7.38 (3 H), 7.49-7.56
(1 H), 7.66-7.72 (1 H), 8.48-8.55 (1 H), 9.03-9.10 (2 H).

### Synthesis of Example E14

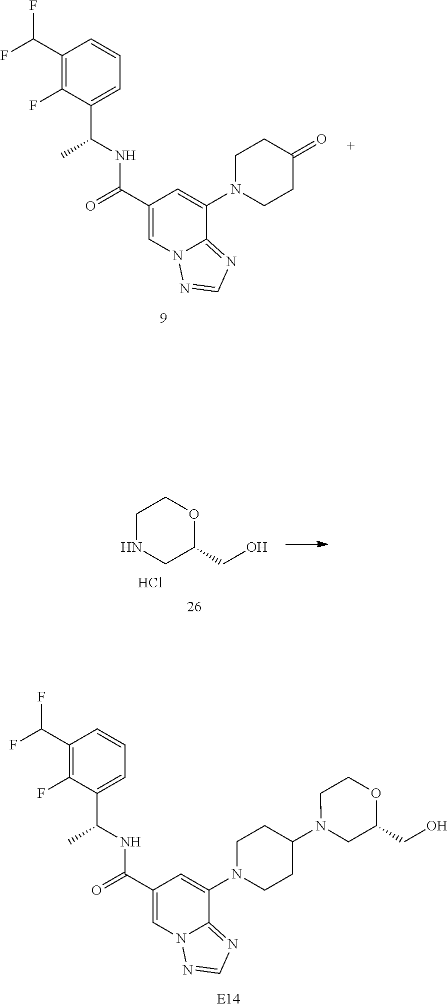 [SMILES: C[C@@H](NC(=O)c1cc(N2CCC(=O)CC2)c2ncnn2c1)c1cccc(C(F)F)c1F ; OC[C@@H]1CNCCO1 ; Cl ; C[C@@H](NC(=O)c1cc(N2CCC(N3CCO[C@H](CO)C3)CC2)c2ncnn2c1)c1cccc(C(F)F)c1F]

A mixture of Intermediate 9 (90 mg, 209 μmol), (S)-morpholin-2-ylmethanol hydrochloride 26 (67 mg, 417 μmol) is stirred in MeOH (2 mL) for 2 h at 35° C. AcOH (18 μL, 313 μmol) and sodium cyanoborohydride (26 mg, 417 μmol) are added and stirred for 16 h at rt. The mixture is diluted with water and MeOH, filtered and basified with triethylamine. The mixture is purified by prep. RP-HPLC (acidic conditions) to obtain the desired compound. The compound is dissolved in MeOH und filtered through a carbonate cartridge, the solvent is evaporated, dissolved in ACN/H₂O and lyophilized to obtain example E14.

Example E14
Analytical HPLC-MS Method: D

| | |
|---|---|
| Rₜ [min]: 0.73 | MS [m/z]: 533 [M + H]⁺ |

Analytical SFC method: Y

| | |
|---|---|
| Rₜ [min]: 5.10 | d.e. > 98% |

¹H NMR (400 MHz, DMSO-d₆) δ (ppm): 1.48-1.64 (5 H), 1.87-1.97 (3 H), 2.16-2.25
(1 H), 2.34-2.46 (1 H), 2.70-2.77 (1 H), 2.81-2.92 (3 H), 3.35-3.52 (3 H), 3.74-3.81
(1 H), 4.31-4.43 (2 H), 4.56-4.63 (1 H), 5.36-5.46 (1 H), 7.08-7.38 (3 H), 7.49-7.56
(1 H), 7.63-7.73 (1 H), 8.48-8.53 (1 H), 9.03-9.10 (2 H), missing proton(s) presumably
hidden by/overlapping with solvent signals

### Synthesis of Example E15

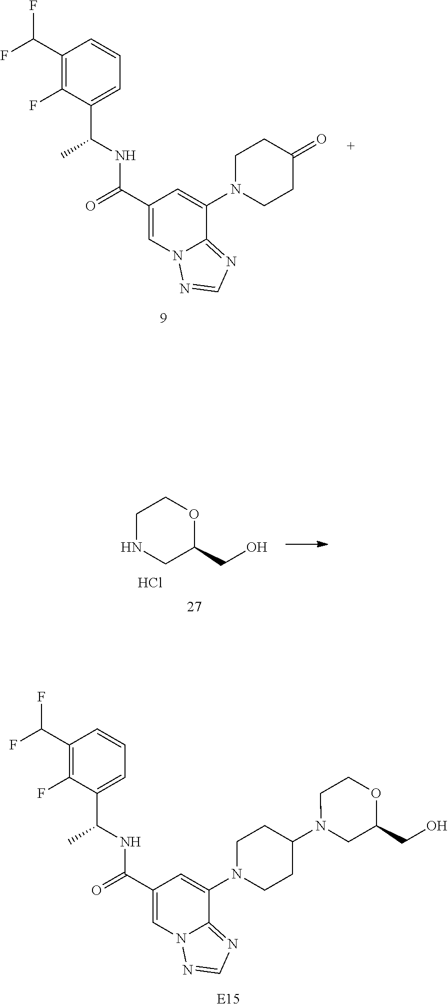 [SMILES: C[C@@H](NC(=O)c1cc(N2CCC(=O)CC2)c2ncnn2c1)c1cccc(C(F)F)c1F ; OC[C@H]1CNCCO1 ; Cl ; C[C@@H](NC(=O)c1cc(N2CCC(N3CCO[C@@H](CO)C3)CC2)c2ncnn2c1)c1cccc(C(F)F)c1F]

Example E15 is prepared in analogy to example E14: Intermediate 9 (209 μmol), (R)-morpholin-2-ylmethanol hydrochloride 27 (417 μmol), AcOH (313 μmol), and sodium cyanoborohydride (417 μmol), 2 mL MeOH.

Example E15
Analytical HPLC-MS Method: D

| | |
|---|---|
| Rₜ [min]: 0.79 | MS [m/z]: 533 [M + H]⁺ |

Analytical SFC method: Y

| | |
|---|---|
| Rₜ [min]: 4.57 | d.e. > 98% |

¹H NMR (400 MHz, DMSO-d₆) δ (ppm): 1.47-1.65 (5 H), 1.87-2.00 (3 H), 2.14-2.26
(1 H), 2.34-2.46 (1 H), 2.69-2.77 (1 H), 2.82-2.93 (3 H), 3.35-3.52 (3 H), 3.72-3.82
(1 H), 4.31-4.44 (2 H), 4.54-4.64 (1 H), 5.35-5.47 (1 H), 7.06-7.38 (3 H), 7.47-7.57
(1 H), 7.64-7.72 (1 H), 8.47-8.54 (1 H), 9.00-9.12 (2 H), missing proton(s) presumably
hidden by/overlapping with solvent signals

### Synthesis of Examples Cis-E16a and Cis-E16b

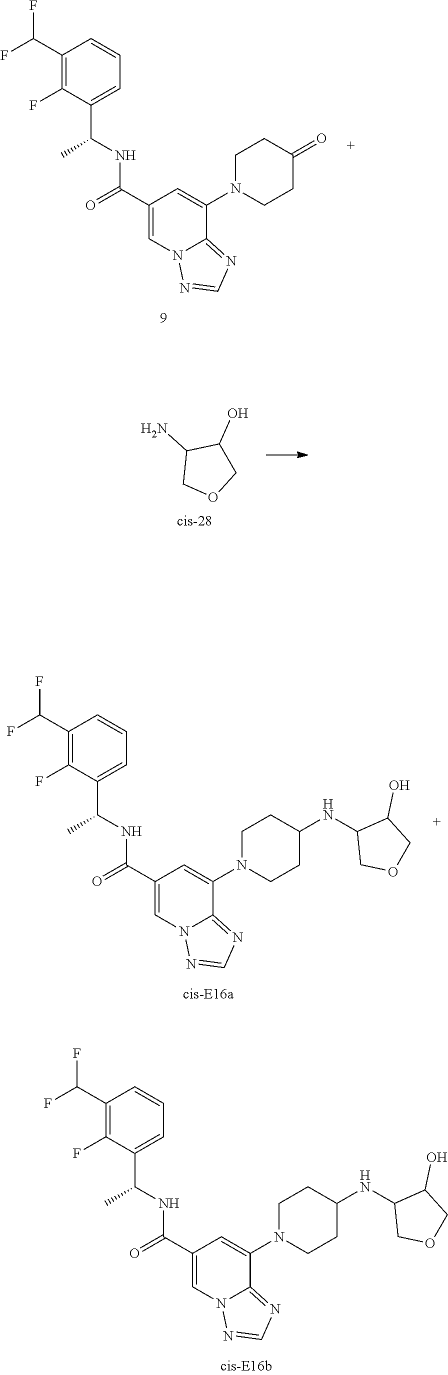 [SMILES: C[C@@H](NC(=O)c1cc(N2CCC(=O)CC2)c2ncnn2c1)c1cccc(C(F)F)c1F ; NC1COCC1O ; C[C@@H](NC(=O)c1cc(N2CCC(NC3COCC3O)CC2)c2ncnn2c1)c1cccc(C(F)F)c1F ; C[C@@H](NC(=O)c1cc(N2CCC(NC3COCC3O)CC2)c2ncnn2c1)c1cccc(C(F)F)c1F]

A mixture of intermediate 9 (100 mg, 232 μmol), cis-4-aminotetrahydrofuran-3-ol cis-28 (27 mg, 255 μmol) and AcOH (15 μL, 255 μmol) in DCM (1 mL) is stirred for 15 min at rt before sodium triacetoxyborohydride (76 mg, 348 μmol) is added. The mixture is stirred for 3 h at rt. Saturated aqueous sodium bicarbonate solution and DCM are added. The separated aqueous layer is extracted with DCM and the combined organic layers are concentrated in vacuo. The material is purified by prep. RP-HPLC (acidic conditions) and by chiral SFC to obtain examples cis-E16a and cis-E16b as single stereoisomers. The absolute configuration of the amino and hydroxy substituents at the THF ring is not known; their relative configuration is cis.

Example cis-E16a
HPLC-MS Method: D

| | |
|---|---|
| Rₜ [min]: 0.87 | MS [m/z]: 519 [M + H]⁺ |

Analytical SFC method: J

| | |
|---|---|
| Rₜ [min]: 4.38 | d.e. > 98% |

¹H NMR (400 MHz, DMSO-d₆) δ (ppm): 1.34-1.56 (5 H), 1.90-2.03 (2 H), 2.67-2.77
(1 H), 2.91-3.02 (2 H), 3.56-3.63 (1 H), 3.77-3.86 (2 H), 4.02-4.09 (1 H), 4.18-4.30
(2 H), 4.74-5.07 (1 H), 5.36-5.46 (1 H), 7.07-7.38 (3 H), 7.48-7.56 (1 H), 7.64-7.72
(1 H), 8.47-8.54 (1 H), 9.02-9.11 (2 H), missing proton(s) presumably hidden by/
overlapping with solvent signals

Example cis-E16b
HPLC-MS Method: D

| | |
|---|---|
| Rₜ [min]: 0.87 | MS [m/z]: 519 [M + H]⁺ |

Analytical SFC method: J

| | |
|---|---|
| Rₜ [min]: 5.00 | d.e. > 98% |

¹H NMR (400 MHz, DMSO-d₆) δ (ppm): 1.37-1.56 (5 H), 1.90-2.01 (2 H), 2.65-2.77
(1 H), 2.90-3.03 (2 H), 3.54-3.63 (1 H), 3.76-3.87 (2 H), 4.01-4.09 (1 H), 4.17-4.30
(2 H), 4.79-5.00 (1 H), 5.36-5.47 (1 H), 7.07-7.39 (3 H), 7.48-7.57 (1 H), 7.64-7.72
(1 H), 8.45-8.54 (1 H), 9.01-9.11 (2 H), missing proton(s) presumably hidden by/
overlapping with solvent signals

### Synthesis of Examples E17a and E17b

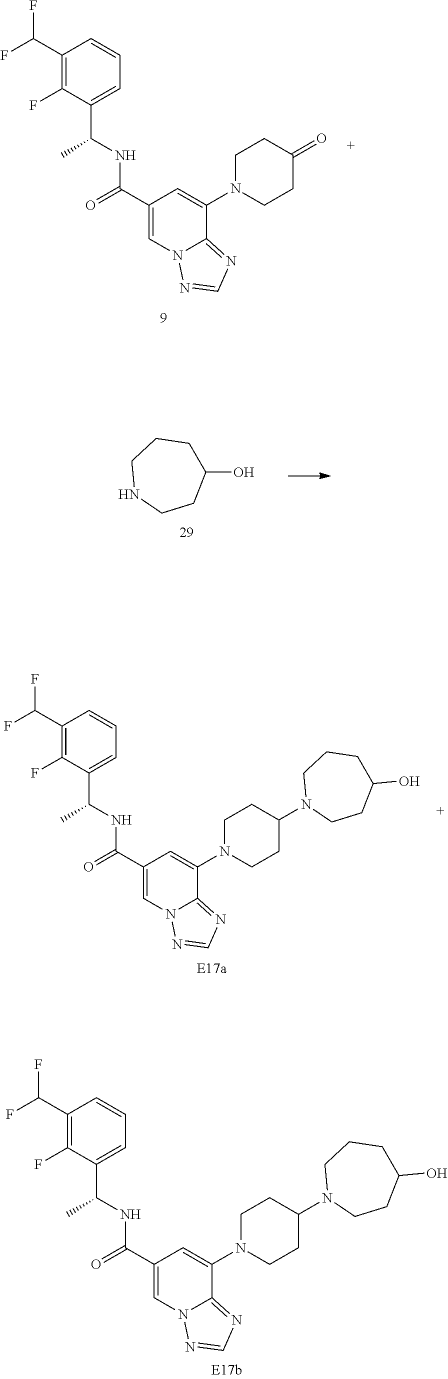 [SMILES: C[C@@H](NC(=O)c1cc(N2CCC(=O)CC2)c2ncnn2c1)c1cccc(C(F)F)c1F ; OC1CCCNCC1 ; C[C@@H](NC(=O)c1cc(N2CCC(N3CCCC(O)CC3)CC2)c2ncnn2c1)c1cccc(C(F)F)c1F ; C[C@@H](NC(=O)c1cc(N2CCC(N3CCCC(O)CC3)CC2)c2ncnn2c1)c1cccc(C(F)F)c1F]

A mixture of Intermediate 9 (200 mg, 464 μmol), azepin-4-ol 29 (111 mg, 695 μmol), AcOH (53 μL, 927 μmol), triethylamine (130 μL, 927 μmol) in DMF (2 mL) is stirred for 4 h at rt, before sodium triacetoxyborohydride (197 mg, 927 μmol) is added at rt. The mixture is stirred for 2 h at rt. Water, MeOH, THF are added, the mixture is filtered and directly purified by prep. RP-HPLC (basic conditions) and chiral SFC to obtain examples E17a and E17b as single stereoisomers. The absolute configuration of the hydroxy substituent is not known.

Example E17a
Analytical HPLC-MS Method: D

| | |
|---|---|
| Rₜ [min]: 0.93 | MS [m/z]: 531 [M + H]⁺ |

Analytical SFC method: AC

| | |
|---|---|
| Rₜ [min]: 5.73 | d.e. > 98% |

¹H NMR (400 MHz, DMSO-d₆) δ (ppm): 1.36-1.85 (13 H), 2.57-2.73 (4 H), 2.77-2.88
(2 H), 3.63-3.74 (1 H), 4.25-4.43 (3 H), 5.36-5.47 (1 H), 7.07-7.38 (3 H), 7.49-7.56
(1 H), 7.65-7.72 (1 H), 8.47-8.53 (1 H), 9.01-9.10 (2 H), missing proton(s) presumably
hidden by/overlapping with solvent signals

Example E17b
Analytical HPLC-MS Method: D

| | |
|---|---|
| Rₜ [min]: 0.93 | MS [m/z]: 531 [M + H]⁺ |

Analytical SFC method: AC

| | |
|---|---|
| Rₜ [min]: 4.48 | d.e. > 98% |

¹H NMR (400 MHz, DMSO-d₆) δ (ppm): 1.37-1.86 (13 H), 2.58-2.73 (4 H), 2.77-2.87
(2 H), 3.63-3.74 (1 H), 4.28-4.44 (3 H), 5.36-5.46 (1 H), 7.08-7.40 (3 H), 7.50-7.57
(1 H), 7.65-7.73 (1 H), 8.50-8.53 (1 H), 9.03-9.09 (2 H), missing proton(s) presumably
hidden by/overlapping with solvent signals

### Synthesis of Example E18

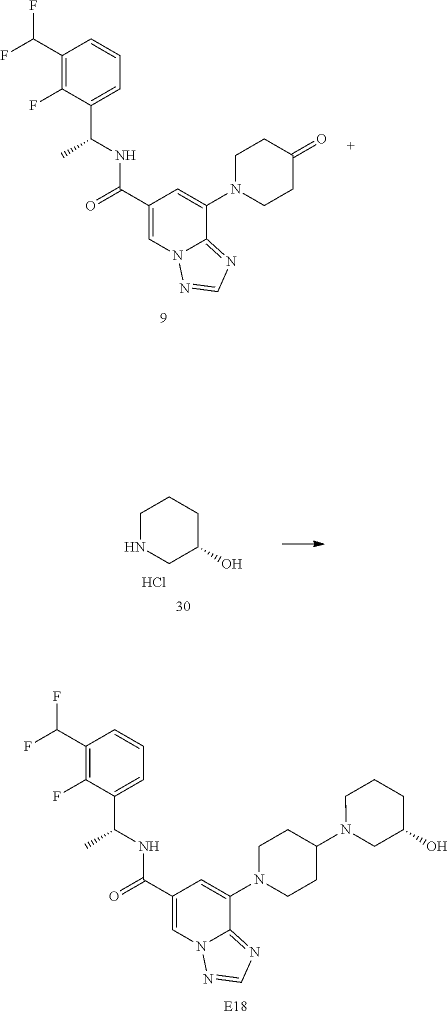 [SMILES: C[C@@H](NC(=O)c1cc(N2CCC(=O)CC2)c2ncnn2c1)c1cccc(C(F)F)c1F ; O[C@H]1CCCNC1 ; Cl ; C[C@@H](NC(=O)c1cc(N2CCC(N3CCC[C@H](O)C3)CC2)c2ncnn2c1)c1cccc(C(F)F)c1F]

A mixture of Intermediate 9 (40 mg, 93 μmol), (S)-3-hydroxypiperidine hydrochloride 30 (20 mg, 139 μmol) and AcOH (5.3 μL, 93 μmol) in DMF (0.8 mL) is stirred at rt for 1 h before sodium triacetoxyborohydride (79 mg, 371 μmol) is added. The mixture is stirred for 4 h at rt. Water is added and the mixture is basified with aqueous NH₃ solution (10%). The mixture is further diluted with THF/MeOH, filtered and directly purified by prep. RP-HPLC (basic conditions) to obtain example E18.

Example E18
Analytical HPLC-MS Method: D

| | |
|---|---|
| Rₜ [min]: 0.93 | MS [m/z]: 517 [M + H]⁺ |

Analytical SFC method: S

| | |
|---|---|
| Rₜ [min]: 7.84 | d.e. > 98% |

¹H NMR (400 MHz, DMSO-d₆) δ (ppm): 0.99-1.15 (1 H), 1.32-1.45 (1 H), 1.47-1.69
(6 H), 1.75-1.90 (3 H), 1.90-2.01 (1 H), 2.01-2.15 (1 H), 2.69-2.77 (1 H), 2.78-2.93
(3 H), 3.38-3.51 (1 H), 4.34-4.47 (2 H), 4.48-4.59 (1 H), 5.36-5.47 (1 H), 7.07-7.39
(3 H), 7.49-7.58 (1 H), 7.65-7.74 (1 H), 8.49-8.55 (1 H), 9.00-9.14 (2 H), missing
proton(s) presumably hidden by/overlapping with solvent signals

### Synthesis of Intermediate 33

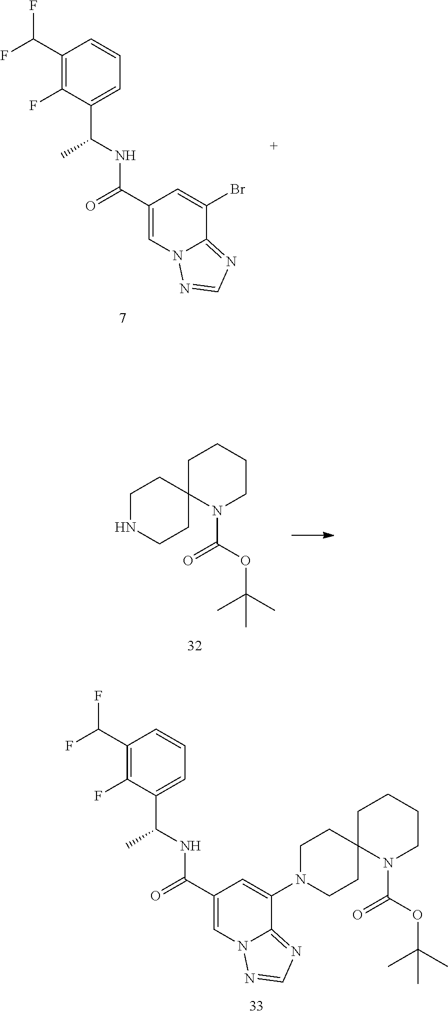 [SMILES: C[C@@H](NC(=O)c1cc(Br)c2ncnn2c1)c1cccc(C(F)F)c1F ; CC(C)(C)OC(=O)N1CCCCC12CCNCC2 ; C[C@@H](NC(=O)c1cc(N2CCC3(CCCCN3C(=O)OC(C)(C)C)CC2)c2ncnn2c1)c1cccc(C(F)F)c1F]

A mixture of bromide 7 (80 mg, 194 μmol), tert-butyl 1,9-diazaspiro[5.5]undecane-1-carboxylate 32 (78 mg, 290 μmol) and catalyst I (7 mg, 8 μmol) in 1,4-dioxane (3 mL) is degassed with argon. K₃PO₄ (126 mg, 581 μmol) is added, and the mixture is heated for 3.5 h at 95° C. The mixture is filtered, rinsed with EtOAc, and concentrated in vacuo. Intermediate 33 is used directly for the next step.

Intermediate 33
Analytical HPLC-MS Method: D

| | |
|---|---|
| Rₜ [min]: 1.19 | MS [m/z]: 587 [M + H]⁺ |

### Synthesis of Example E19

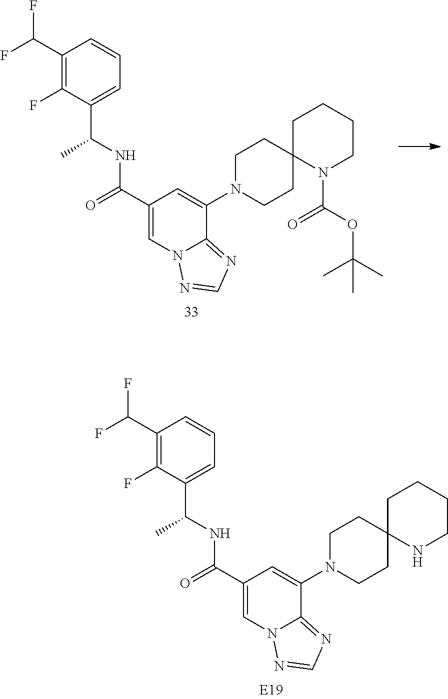 [SMILES: C[C@@H](NC(=O)c1cc(N2CCC3(CCCCN3C(=O)OC(C)(C)C)CC2)c2ncnn2c1)c1cccc(C(F)F)c1F ; C[C@@H](NC(=O)c1cc(N2CCC3(CCCCN3)CC2)c2ncnn2c1)c1cccc(C(F)F)c1F]

A mixture of intermediate 33 (140 mg, 167 μmol) and TFA (0.4 mL) in DCM (2 mL) is stirred for 1.5 h at rt. The mixture is concentrated in vacuo and the residue is dissolved in ACN. Cs₂CO₃ is added, and the resulting suspension is filtered. The filtrate is purified by prep. RP-HPLC (basic conditions) to obtain example E19.

Example E19
Analytical HPLC-MS Method: D

| | |
|---|---|
| Rₜ [min]: 1.01 | MS [m/z]: 487 [M + H]⁺ |

Chiral SFC Method: H

| | |
|---|---|
| Rₜ [min]: 3.67 | e.e. > 98% |

¹H NMR (400 MHz, DMSO-d₆) δ (ppm): 1.30-1.79 (13 H), 2.52-2.80 (3 H), 3.40-3.48
(2 H), 3.64-3.80 (2 H), 5.42 (1 H), 7.08-7.38 (3 H), 7.52 (1 H), 7.69 (1 H), 8.49 (1 H),
9.03 (1 H), 9.07 (1 H).

### Synthesis of Example E20

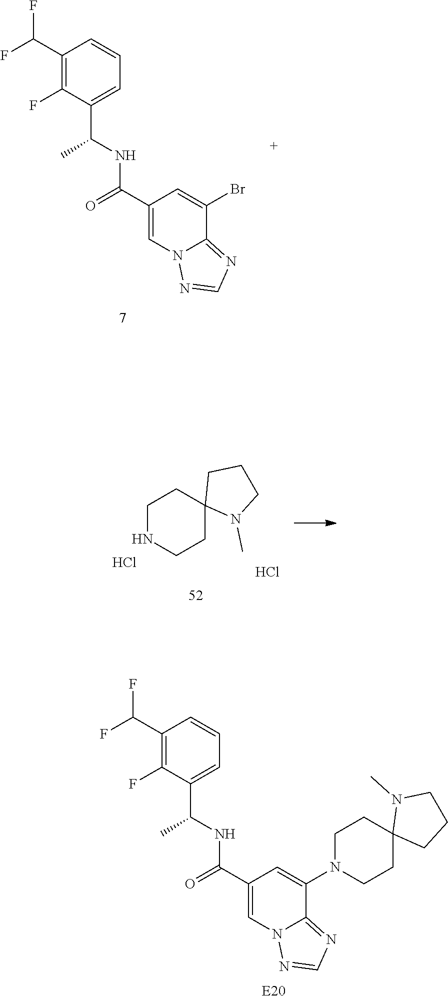 [SMILES: C[C@@H](NC(=O)c1cc(Br)c2ncnn2c1)c1cccc(C(F)F)c1F ; CN1CCCC12CCNCC2 ; Cl ; Cl ; C[C@@H](NC(=O)c1cc(N2CCC3(CCCN3C)CC2)c2ncnn2c1)c1cccc(C(F)F)c1F]

A mixture of intermediate 7 (100 mg, 230 μmol), 1-methyl-1,8-diazaspiro[4.5]decane dihydrochloride 52 (60 mg, 253 μmol) and Cs₂CO₃ (300 mg, 920 μmol) in 1,4-dioxane (2 mL) is degassed with argon. Then catalyst I (19 mg, 23 μmol) is added, and the mixture is heated under argon for 16 h at 120° C. Aqueous NaHCO₃ solution is added, the aqueous layer is extracted with EtOAc and the combined organic layers are dried with MgSO₄. After filtration and evaporation of the solvent the material is purified by prep. RP-HPLC (basic conditions) to obtain example E20.

Example E20
Analytical HPLC-MS Method: D

| | |
|---|---|
| Rₜ [min]: 1.02 | MS [m/z]: 487 [M + H]⁺ |

Analytical SFC method: AD

| | |
|---|---|
| Rₜ [min]: 1.55 | e.e. > 98% |

¹H NMR (400 MHz, DMSO-d₆) δ (ppm): 1.48-1.57 (3 H), 1.64-1.85 (6 H), 2.04-2.09
(1 H), 2.16-2.24 (3 H), 2.65-2.74 (2 H), 2.78-2.91 (2 H), 4.29-4.41 (2 H), 5.36-5.48
(1 H), 7.05-7.39 (3 H), 7.49-7.57 (1 H), 7.64-7.73 (1 H), 8.45-8.55 (1 H), 8.99-9.11
(2 H), missing proton(s) presumably hidden by/overlapping with solvent signals.

### Synthesis of Examples Cis-E21a and Cis-E21b

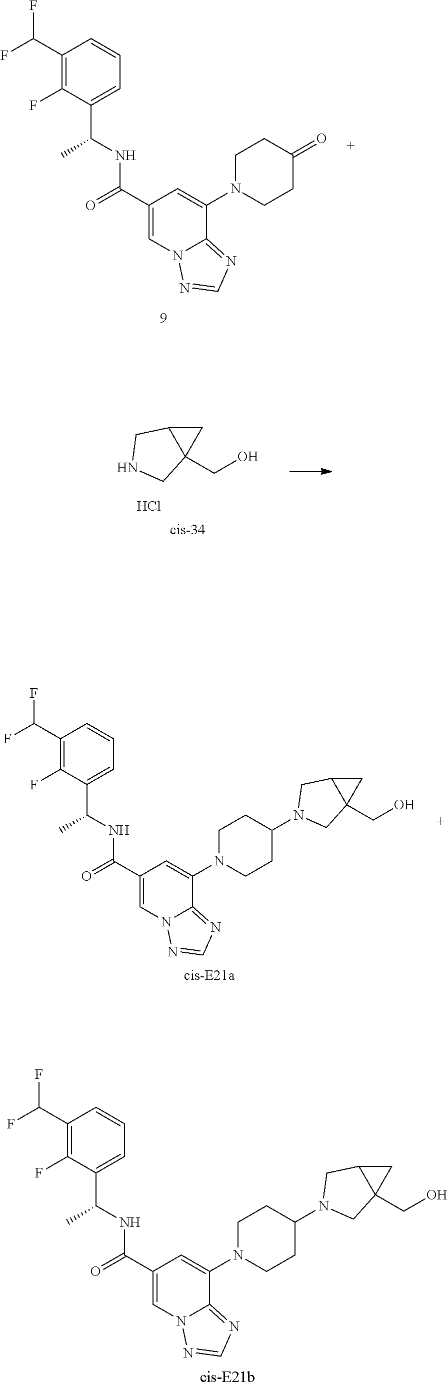 [SMILES: C[C@@H](NC(=O)c1cc(N2CCC(=O)CC2)c2ncnn2c1)c1cccc(C(F)F)c1F ; OCC12CNCC1C2 ; Cl ; C[C@@H](NC(=O)c1cc(N2CCC(N3CC4CC4(CO)C3)CC2)c2ncnn2c1)c1cccc(C(F)F)c1F ; C[C@@H](NC(=O)c1cc(N2CCC(N3CC4CC4(CO)C3)CC2)c2ncnn2c1)c1cccc(C(F)F)c1F]

Intermediate 9 (200 mg, 464 μmol) is dissolved in THF (7.5 mL). Then rac-cis azabicyclo[3.1.0]hexan-1-ylmethanole hydrochloride cis-34 (347 mg, 2.32 mmol), and molecular sieves 4 Å are added and stirring is continued for 1 h at 60° C. After cooling to rt, sodium triacetoxyborohydride (202 mg, 927 μmol) is added and the reaction mixture stirred for 1 h at rt. The crude mixture is filtered through Celite and directly purified by prep. RP-HPLC (basic conditions) and by chiral SFC to obtain example cis-E21a and example cis-E21b as single stereoisomers. The absolute configuration of the bridged carbon atoms is not known; the relative configuration of the [3.1.0] ring system is cis.

Example cis-E21a
Analytical HPLC-MS Method: D

| | |
|---|---|
| Rₜ [min]: 0.95 | MS [m/z]: 529 [M + H]⁺ |

Analytical SFC method: T

| | |
|---|---|
| Rₜ [min]: 4.58 | d.e. = 98% |

¹H NMR (400 MHz, DMSO-d₆) δ (ppm): 0.33-0.41 (1 H), 0.69-0.76 (1 H), 1.17-1.25
(1 H), 1.44-1.57 (5 H), 1.82-1.94 (2 H), 2.24-2.38 (3 H), 2.93-3.08 (4 H), 3.39-3.53
(2 H), 4.09-4.20 (2 H), 4.44-4.50 (1 H), 5.35-5.45 (1 H), 7.08-7.38 (3 H), 7.49-7.56
(1 H), 7.65-7.72 (1 H), 8.46-8.53 (1 H), 9.01-9.10 (2 H).

Example cis-E21b
Analytical HPLC-MS Method: D

| | |
|---|---|
| Rₜ [min]: 0.95 | MS [m/z]: 529 [M + H]⁺ |

Analytical SFC method: T

| | |
|---|---|
| Rₜ [min]: 4.95 | d.e. = 94% |

¹H NMR (400 MHz, DMSO-d₆) δ (ppm): 0.34-0.40 (1 H), 0.70-0.77 (1 H), 1.17-1.26
(1 H), 1.42-1.58 (5 H), 1.82-1.94 (2 H), 2.25-2.39 (3 H), 2.94-3.08 (4 H), 3.40-3.54
(2 H), 4.09-4.20 (2 H), 4.38-4.54 (1 H), 5.35-5.46 (1 H), 7.07-7.39 (3 H), 7.49-7.57
(1 H), 7.64-7.74 (1 H), 8.47-8.54 (1 H), 9.00-9.13 (2 H).

### Synthesis of Example E22

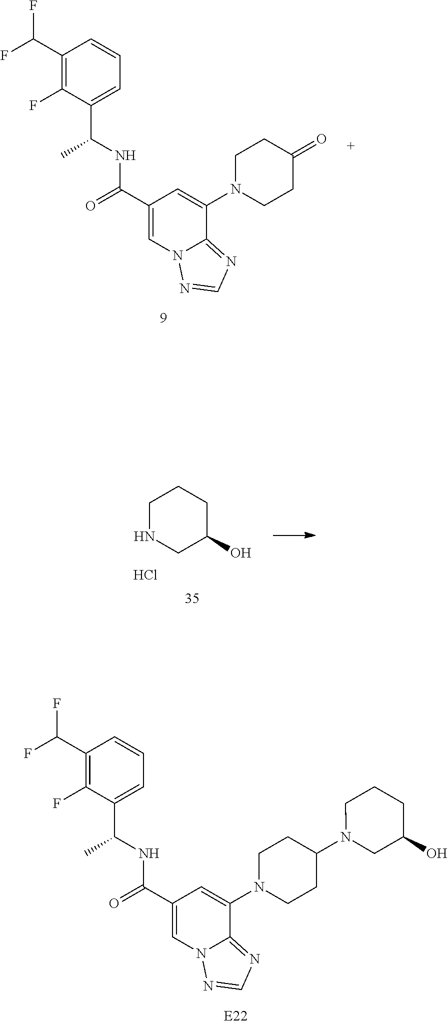 [SMILES: C[C@@H](NC(=O)c1cc(N2CCC(=O)CC2)c2ncnn2c1)c1cccc(C(F)F)c1F ; O[C@@H]1CCCNC1 ; Cl ; C[C@@H](NC(=O)c1cc(N2CCC(N3CCC[C@@H](O)C3)CC2)c2ncnn2c1)c1cccc(C(F)F)c1F]

(R)-3-Hydroxypiperidine hydrochloride 35 (130 mg, 927 μmol) is dissolved in MeOH (5 mL) and filtered through a carbonate cartridge. Intermediate 9 (200 mg, 464 μmol) is added and the mixture is stirred for 2 h at 35° C. AcOH (41 μl, 695 μmol) and sodium cyanoborohydride (58 mg, 927 μmol) are added and the mixture stirred for 16 h at rt. Water is added, filtered and the filtrate is directly purified by prep. RP-HPLC (acidic conditions). After lyophilization, the material is redissolved in MeOH and filtered through a carbonate cartridge to obtain example E22.

Example E22
Analytical HPLC-MS Method: D

| | |
|---|---|
| Rₜ [min]: 0.80 | MS [m/z]: 517 [M + H]⁺ |

Analytical SFC method: Z

| | |
|---|---|
| Rₜ [min]: 4.85 | d.e. > 98% |

¹H NMR (400 MHz, DMSO-d₆) δ (ppm): 1.00-1.14 (1 H), 1.30-1.47 (1 H), 1.49-1.67
(6 H), 1.74-1.89 (3 H), 1.89-1.98 (1 H), 2.03-2.13 (1 H), 2.65-2.77 (1 H), 2.77-2.92
(3 H), 3.37-3.49 (1 H), 4.32-4.46 (2 H), 4.49-4.56 (1 H), 5.36-5.47 (1 H), 7.07-7.40
(3 H), 7.48-7.57 (1 H), 7.66-7.73 (1 H), 8.46-8.53 (1 H), 9.02-9.09 (2 H), missing
proton(s) presumably hidden by/overlapping with solvent signals

### Synthesis Scheme of Intermediate 38

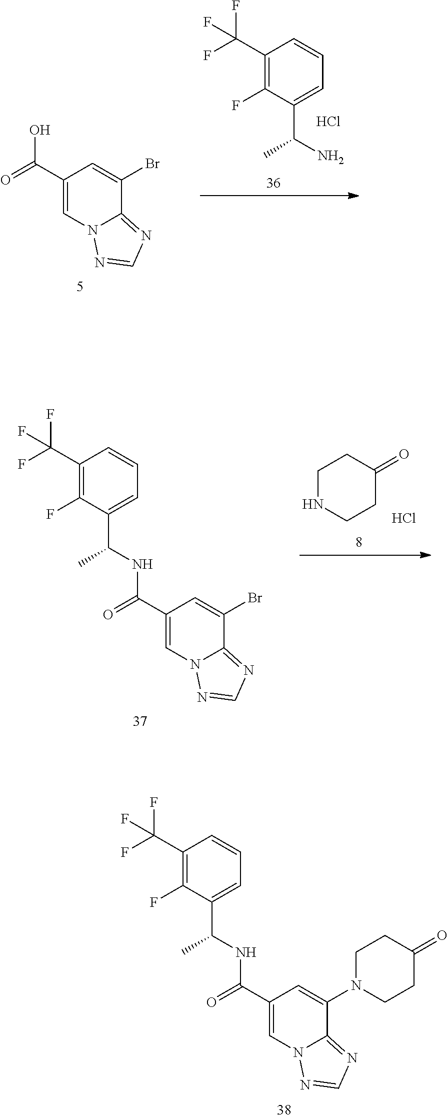

### Synthesis of Intermediate 37

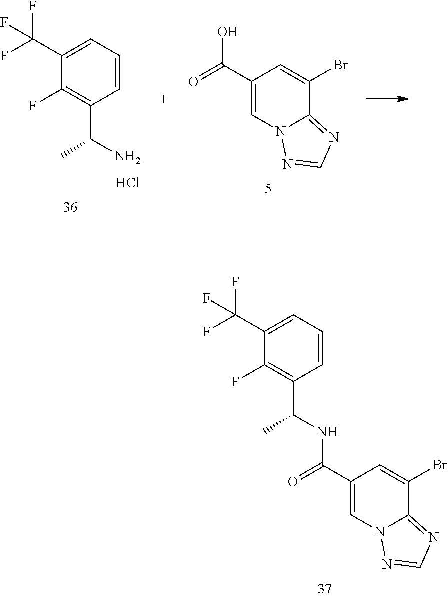 [SMILES: C[C@@H](N)c1cccc(C(F)(F)F)c1F ; O=C(O)c1cc(Br)c2ncnn2c1 ; Cl ; C[C@@H](NC(=O)c1cc(Br)c2ncnn2c1)c1cccc(C(F)(F)F)c1F]

To a mixture of intermediate 5 (12.5 g, 51.6 mmol), (1R)-1-[2-fluoro-3-(trifluoromethyl)phenyl]ethan-1-amine hydrochloride 36 (CAS: 2230840-52-3; 13.8 g, 51.6 mmol) and N-methyl morpholine (20.1 mL, 181 mmol) in ACN (152 mL) is added 1-propanephosphonic anhydride (50%; 36.9 mL, 62.0 mmol) at 0° C. dropwise. The mixture is allowed to reach rt and is stirred for 3 h. ACN is evaporated under reduced pressure. Water and EtOAc are added to the remaining material. The organic layer is separated and extracted with 0.5 M KHSO₄ solution and water sequentially. The organic layer is dried with MgSO₄, filtered, and the solvent is evaporated. The remaining material is dissolved in warm EtOAc and is allowed to cool down over night. The solids are filtered to obtain intermediate 37. The liquid layer is separated, and the solvent evaporated. The remaining solid is stirred in MTBE. The solids are filtered to obtain intermediate 37, and the two batches are combined.

Intermediate 37
HPLC-MS Method: E

| | |
|---|---|
| Rₜ [min]: 1.04 | MS [m/z]: 431 [M + H]⁺ |

### Synthesis of Intermediate 38

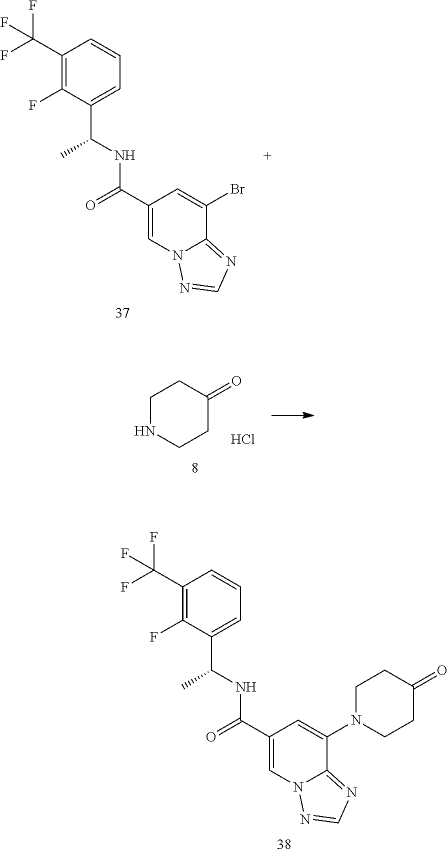 [SMILES: C[C@@H](NC(=O)c1cc(Br)c2ncnn2c1)c1cccc(C(F)(F)F)c1F ; O=C1CCNCC1 ; Cl ; C[C@@H](NC(=O)c1cc(N2CCC(=O)CC2)c2ncnn2c1)c1cccc(C(F)(F)F)c1F]

A mixture of intermediate 37 (1.3 g, 3.1 mmol), piperidin-4-one hydrochloride 8 (881 mg, 6.2 mmol) and Cs₂CO₃ (3.5 g, 10.8 mmol) in 1,4-dioxane (18 mL) is degassed with argon. Catalyst I (78 mg, 93 μmol) is added, and the mixture is heated under argon for 3.5 h at 95° C. The mixture is filtered, the solids are washed with EtOAc, and the combined solvents are evaporated under reduced pressure. The material is purified by column chromatography (SiO₂; EtOAc/cyclohexane: 80:20→100:0) to obtain intermediate 38.

Intermediate 38
Analytical HPLC-MS Method: D

| | |
|---|---|
| Rₜ [min]: 1.00 | MS [m/z]: 450 [M + H]⁺ |

### Scheme of Alternative Synthesis of Intermediate 37

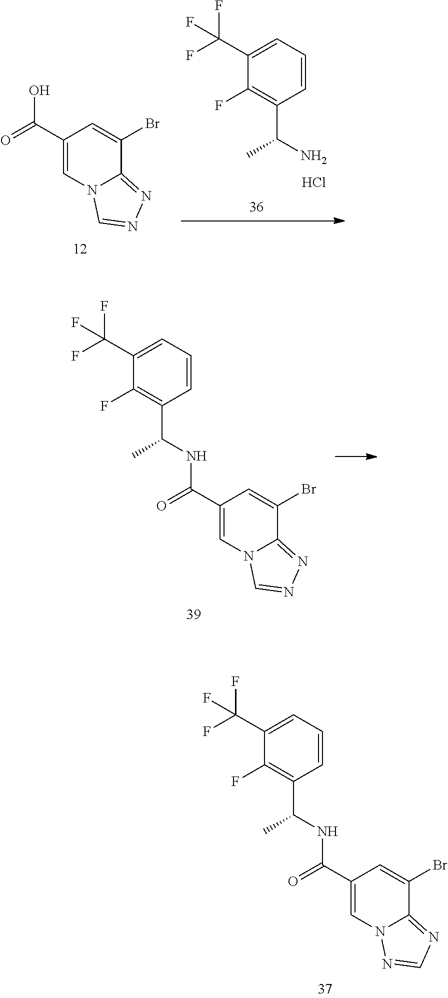 [SMILES: O=C(O)c1cc(Br)c2nncn2c1 ; C[C@@H](N)c1cccc(C(F)(F)F)c1F ; Cl ; C[C@@H](NC(=O)c1cc(Br)c2nncn2c1)c1cccc(C(F)(F)F)c1F ; C[C@@H](NC(=O)c1cc(Br)c2ncnn2c1)c1cccc(C(F)(F)F)c1F]

### Synthesis of Intermediate 39

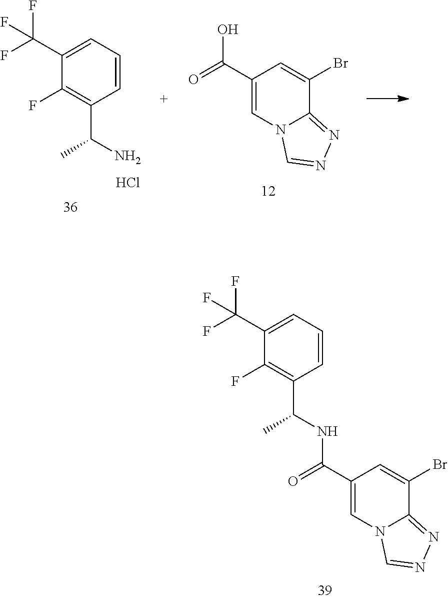 [SMILES: C[C@@H](N)c1cccc(C(F)(F)F)c1F ; O=C(O)c1cc(Br)c2nncn2c1 ; Cl ; C[C@@H](NC(=O)c1cc(Br)c2nncn2c1)c1cccc(C(F)(F)F)c1F]

HATU (189 mg, 496 μmol) and triethylamine (232 μL, 1.7 μmol) are added to intermediate 12 (100 mg, 413 μmol) in DMF (1.6 mL). The mixture is stirred for 15 min at rt before (1R)-1-[2-fluoro-3-(trifluoromethyl)phenyl]ethan-1-amine hydrochloride 36 (CAS: 2230840-52-3; 112 mg, 454 μmol) is added. The mixture is stirred for 16 h at rt before it is diluted with MeOH, filtered, and purified by prep. RP-HPLC (acidic conditions) to obtain intermediate 39.

Intermediate 39
HPLC-MS Method: E

| | |
|---|---|
| Rₜ [min]: 0.98 | MS [m/z]: 431 [M + H]⁺ |

### Synthesis of Intermediate 37

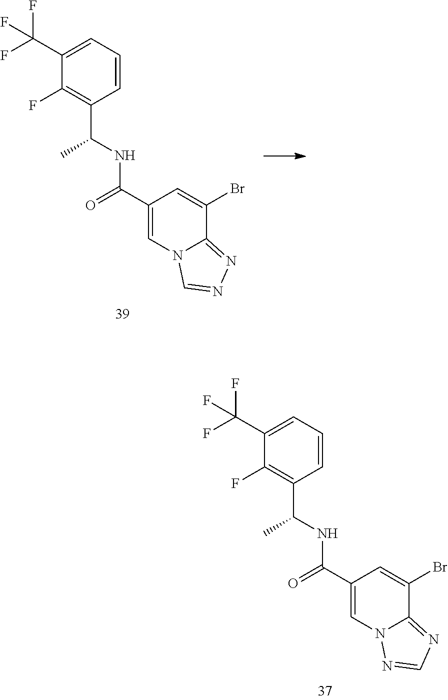 [SMILES: C[C@@H](NC(=O)c1cc(Br)c2nncn2c1)c1cccc(C(F)(F)F)c1F ; C[C@@H](NC(=O)c1cc(Br)c2ncnn2c1)c1cccc(C(F)(F)F)c1F]

A mixture of intermediate 39 (12.0 g, 19.5 mmol), Cs₂CO₃ (6.3 g, 19.5 mmol), piperidine (1.9 mL, 19.5 mmol) in 1,4-dioxane (90 mL) is stirred at 100° C. for 16 h. EtOAc and water are added. The aqueous layer is separated and extracted with EtOAc. The combined organic layers are dried over Na₂SO₄. The solids are filtered, and the solvent is evaporated under reduced pressure. The material is purified by column chromatography (SiO₂; EtOAc/petroleum ether: 10:90→30:70) to obtain intermediate 37.

Intermediate 37
Analytical HPLC-MS Method: D

| | |
|---|---|
| Rₜ [min]: 1.00 | MS [m/z]: 431 [M + H]⁺ |

### Synthesis of Example E24

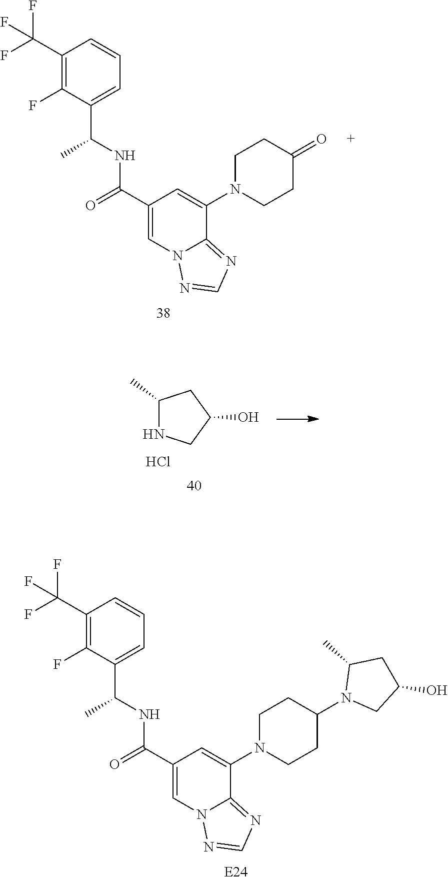

Example E24 is prepared in analogy to example E1: Intermediate 38 (80 μmol), (3R,5S)-5-methylpyrrolidin-3-ol hydrochloride 40 (0.12 mmol), AcOH (0.51 mmol), and 2-picoline-borane complex (80 μmol), 2 mL MeOH.

Example E24
HPLC-MS Method: A

| | |
|---|---|
| Rₜ [min]: 0.80 | MS [m/z]: 535 [M + H]⁺ |

Analytical SFC method: L

| | |
|---|---|
| Rₜ [min]: 2.25 | d.e. > 98% |

¹H NMR (400 MHz, DMSO-d₆) δ (ppm): 1.42-1.49 (3 H), 1.50-1.56 (3 H), 1.62-1.70
(1 H), 1.77-1.92 (2 H), 2.04-2.12 (1 H), 2.18-2.27 (1 H), 2.37-2.45 (1 H), 2.80-2.90
(2 H), 3.82-3.93 (1 H), 4.36-4.43 (1 H), 4.43-4.57 (2 H), 5.38-5.47 (1 H), 7.14-7.20
(1 H), 7.36-7.45 (1 H), 7.63-7.73 (1 H), 7.80-7.86 (1 H), 8.52-8.59 (1 H), 9.09-9.19
(2 H), 9.45-9.58 (1 H), missing proton(s) presumably hidden by/overlapping with solvent
signals.

### Synthesis of Example E25

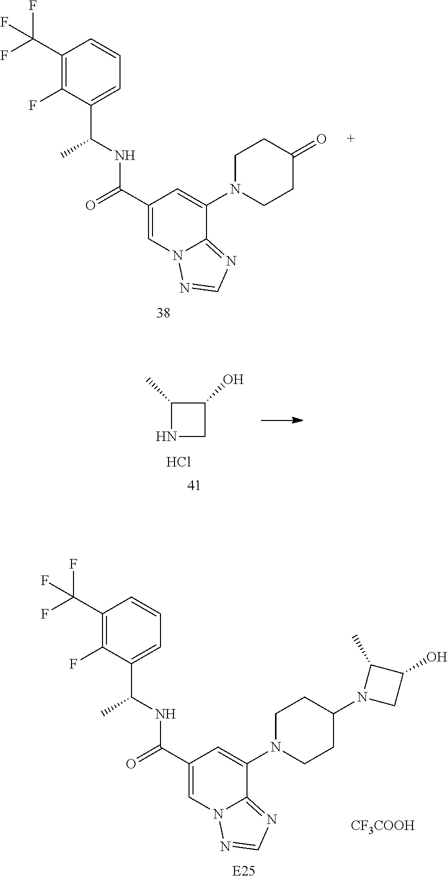

Example E25 is prepared in analogy to example E1: Intermediate 38 (80 μmol), (2R,3R)-2-methylazetidin-3-ol hydrochloride 41 (0.12 mmol), AcOH (0.51 mmol), and 2-picoline-borane complex (80 μmol), 2 mL MeOH.

Example E25
HPLC-MS Method: A

| | |
|---|---|
| Rₜ [min]: 0.77 | MS [m/z]: 521 [M + H]⁺ |

Analytical SFC method: Q

| | |
|---|---|
| Rₜ [min]: 1.76 | d.e. >97% |

¹H NMR (400 MHz, DMSO-d₆) δ (ppm):
1.02-1.09 (3 H), 1.24-1.43 (2 H), 1.49-1.55 (3 H), 1.65-1.83
(2 H), 2.27-2.37 (1 H), 2.93-3.04 (3 H), 4.05-4.19 (3 H), 5.35-5.43
(1 H), 7.08-7.14 (1 H), 7.35-7.44 (1 H), 7.63-7.70 (1 H), 7.77-7.84
(1 H), 8.45-8.52 (1 H), 9.00-9.05 (1 H), missing proton(s) presumably
hidden by/overlapping with solvent signals.

### Synthesis of Example E26

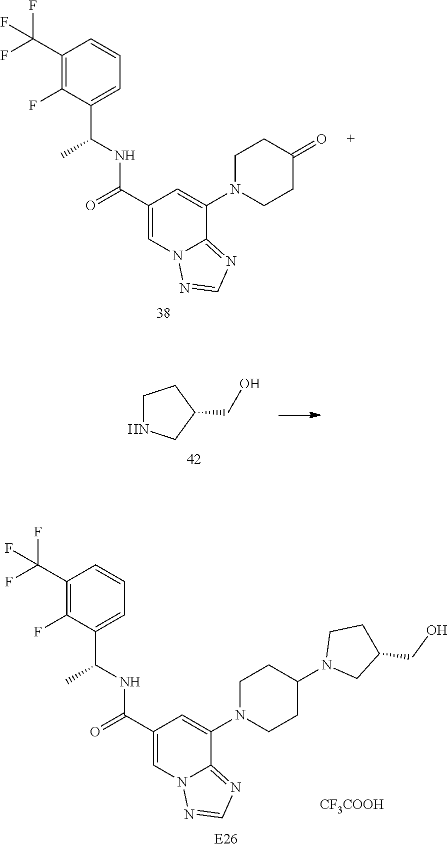 [SMILES: C[C@@H](NC(=O)c1cc(N2CCC(=O)CC2)c2ncnn2c1)c1cccc(C(F)(F)F)c1F ; OC[C@H]1CCNC1 ; C[C@@H](NC(=O)c1cc(N2CCC(N3CC[C@H](CO)C3)CC2)c2ncnn2c1)c1cccc(C(F)(F)F)c1F ; "CF3COOH"]

Example E26 is prepared in analogy to example E1: Intermediate 38 (80 μmol), (S)-pyrrolidin-3-ylmethanol 42 (0.12 mmol), AcOH (0.51 mmol), and 2-picoline-borane complex (80 μmol), 2 mL MeOH; purification by prep. RP-HPLC (acidic conditions).

Example E26
HPLC-MS Method: A

| | |
|---|---|
| Rₜ [min]: 0.78 | MS [m/z]: 535 [M + H]⁺ |

Analytical SFC method: AP

| | |
|---|---|
| Rₜ [min]: 1.67 | e.e./d.e. >98% |

¹H NMR (400 MHz, DMSO-d₆) δ (ppm):
1.49-1.88 (6 H), 1.90-2.24 (3 H), 2.35-2.46 (0.5 H), 2.82-2.93 (2.5 H),
3.07-3.23 (1 H), 4.42-4.55 (2 H), 5.36-5.47 (1 H), 7.15-7.21 (1 H),
7.38-7.45 (1 H), 7.64-7.72 (1 H), 7.80-7.88 (1 H), 8.52-8.58 (1 H),
9.09-9.20 (2 H), 9.50-9.76 (1 H), missing proton(s) presumably
hidden by/overlapping with solvent signals

### Synthesis of Example E27

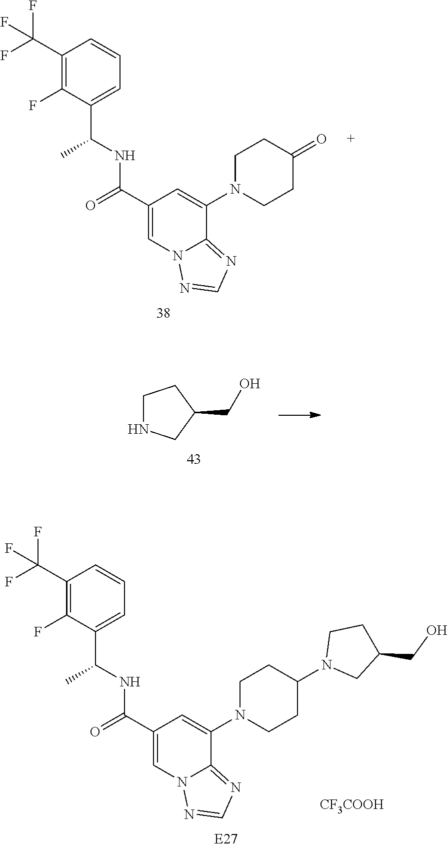 [SMILES: C[C@@H](NC(=O)c1cc(N2CCC(=O)CC2)c2ncnn2c1)c1cccc(C(F)(F)F)c1F ; OC[C@@H]1CCNC1 ; C[C@@H](NC(=O)c1cc(N2CCC(N3CC[C@@H](CO)C3)CC2)c2ncnn2c1)c1cccc(C(F)(F)F)c1F ; "CF3COOH"]

Example E27 is prepared in analogy to example E1: Intermediate 38 (80 μmol), (R)-pyrrolidin-3-ylmethanol 43 (0.12 mmol), AcOH (0.51 mmol), and 2-picoline-borane complex (80 μmol), 2 mL MeOH; purification by prep. RP-HPLC (acidic conditions).

Example E27
HPLC-MS Method: A

| | |
|---|---|
| Rₜ [min]: 0.78 | MS [m/z]: 535 [M + H]⁺ |

Analytical SFC method: L

| | |
|---|---|
| Rₜ [min]: 2.11 | d.e. >98% |

¹H NMR (400 MHz, DMSO-d₆) δ (ppm):
1.48-1.89 (6 H), 1.89-2.24 (3 H), 2.35-2.46 (0.5 H), 2.80-2.95 (2.5 H),
3.07-3.22 (1 H), 4.42-4.55 (2 H), 5.37-5.47 (1 H), 7.15-7.22 (1 H),
7.37-7.45 (1 H), 7.64-7.72 (1 H), 7.79-7.88 (1 H), 8.51-8.59 (1 H),
9.08-9.22 (2 H), 9.47-9.76 (1 H), missing proton(s) presumably
hidden by/overlapping with solvent signals

### Synthesis of Example E28

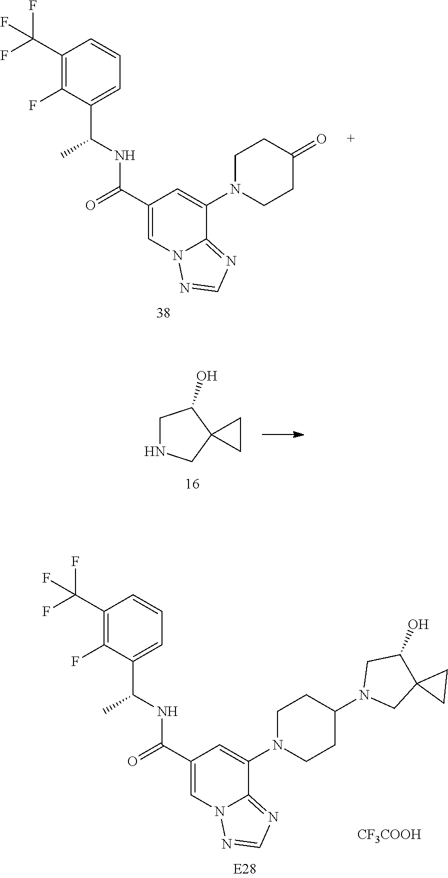 [SMILES: C[C@@H](NC(=O)c1cc(N2CCC(=O)CC2)c2ncnn2c1)c1cccc(C(F)(F)F)c1F ; O[C@H]1CNCC12CC2 ; C[C@@H](NC(=O)c1cc(N2CCC(N3C[C@H](O)C4(CC4)C3)CC2)c2ncnn2c1)c1cccc(C(F)(F)F)c1F ; "CF3COOH"]

Example E28 is prepared in analogy to example E1: Intermediate 38 (80 μmol), (7R)-5-azaspiro[2.4]heptan-7-ol 16 (0.12 mmol), AcOH (0.51 mmol), and 2-picoline-borane complex (80 μmol), 2 mL MeOH; purification by prep. RP-HPLC (acidic conditions).

Example E28
HPLC-MS Method: A

| | |
|---|---|
| Rₜ [min]: 0.80 | MS [m/z]: 547 [M + H]⁺ |

Analytical SFC method: L

| | |
|---|---|
| Rₜ [min]: 2.16 | d.e. >98% |

¹H NMR (400 MHz, DMSO-d₆) δ (ppm):
0.63-0.82 (3 H), 0.89-0.98 (1 H), 1.48-1.59 (3 H), 1.65-1.89 (2 H),
2.07-2.29 (2 H), 2.78-2.95 (2 H), 3.18-3.31 (1 H), 3.86-3.94 (1 H),
4.44-4.55 (2 H), 5.37-5.47 (1 H), 7.15-7.20 (1 H), 7.37-7.45 (1 H),
7.65-7.72 (1 H), 7.79-7.87 (1 H), 8.55 (1 H), 9.09-9.20 (2 H),
9.97-10.29 (1 H), missing proton(s) presumably
hidden by/overlapping with solvent signals

### Synthesis of Example Cis-E29

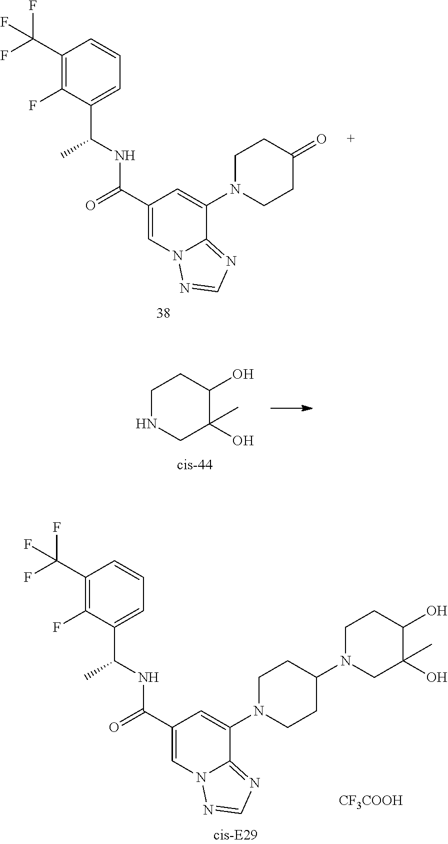

Example cis-E29 is prepared in analogy to example E1: Intermediate 38 (80 μmol), diol cis-44 (0.12 mmol), AcOH (0.51 mmol), and 2-picoline-borane complex (80 μmol), 2 mL MeOH; purification by prep. RP-HPLC (acidic conditions) and is obtained as a mixture of cis-diastereomers.

Example cis-E29
HPLC-MS Method: A

| | |
|---|---|
| Rₜ [min]: 0.76 | MS [m/z]: 565 [M + H]⁺ |

¹H NMR (400 MHz, DMSO-d₆) δ (ppm):
1.15-1.21 (3 H), 1.49-1.58 (3 H), 1.68-2.03 (4 H), 2.04-2.13 (1 H),
2.16-2.25 (1 H), 2.81-2.99 (3 H), 3.01-3.12 (1 H), 4.44-4.61
(2 H), 5.12-5.31 (1 H), 5.37-5.47 (1 H), 7.14-7.20 (1 H),
7.38-7.45 (1 H), 7.64-7.73 (1 H), 7.79-7.87 (1 H), 8.53-8.58
(1 H), 8.74-8.87 (1 H), 9.08-9.18 (2 H), missing proton(s)
presumably hidden by/overlapping with solvent signals

### Synthesis of Example E30

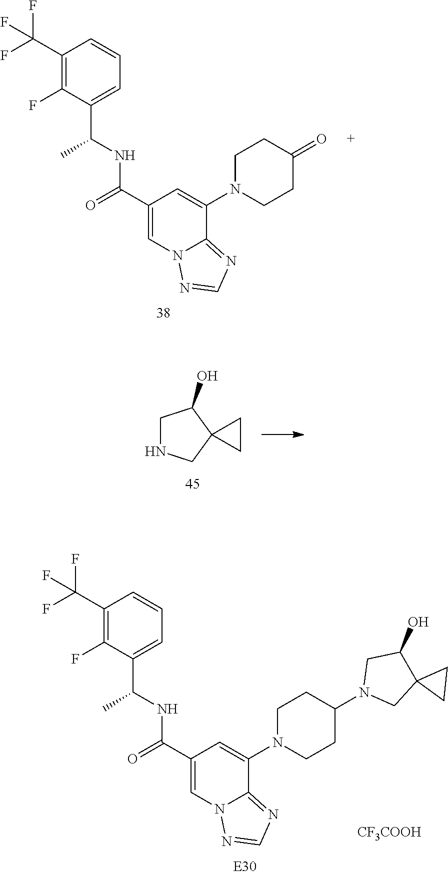 [SMILES: C[C@@H](NC(=O)c1cc(N2CCC(=O)CC2)c2ncnn2c1)c1cccc(C(F)(F)F)c1F ; O[C@@H]1CNCC12CC2 ; C[C@@H](NC(=O)c1cc(N2CCC(N3C[C@@H](O)C4(CC4)C3)CC2)c2ncnn2c1)c1cccc(C(F)(F)F)c1F ; "CF3COOH"]

Example E30 is prepared in analogy to example E1: Intermediate 38 (80 μmol), (7S)-5-azaspiro[2.4]heptan-7-ol 45 (0.12 mmol), AcOH (0.51 mmol), and 2-picoline-borane complex (80 μmol), 2 mL MeOH; purification by prep. RP-HPLC (acidic conditions).

Example E30
HPLC-MS Method: A

| | |
|---|---|
| Rₜ [min]: 0.80 | MS [m/z]: 547 [M + H]⁺ |

Analytical SFC method: F

| | |
|---|---|
| Rₜ [min]: 1.49 | d.e. >98% |

¹H NMR (400 MHz, DMSO-d₆) δ (ppm):
0.61-0.82 (3 H), 0.88-0.98 (1 H), 1.48-1.59 (3 H), 1.66-1.90 (2 H),
2.06-2.28 (2 H), 2.78-2.94 (2 H), 3.17-3.31 (1 H), 3.86-3.95
(1 H), 4.40-4.55 (2 H), 5.38-5.47 (1 H), 7.14-7.21 (1 H),
7.37-7.46 (1 H), 7.64-7.72 (1 H), 7.78-7.87 (1 H), 8.51-8.58
(1 H), 9.09-9.20 (2 H), 9.94-10.28 (1 H), missing proton(s)
presumably hidden by/overlapping with solvent signals.

### Synthesis of Example E31

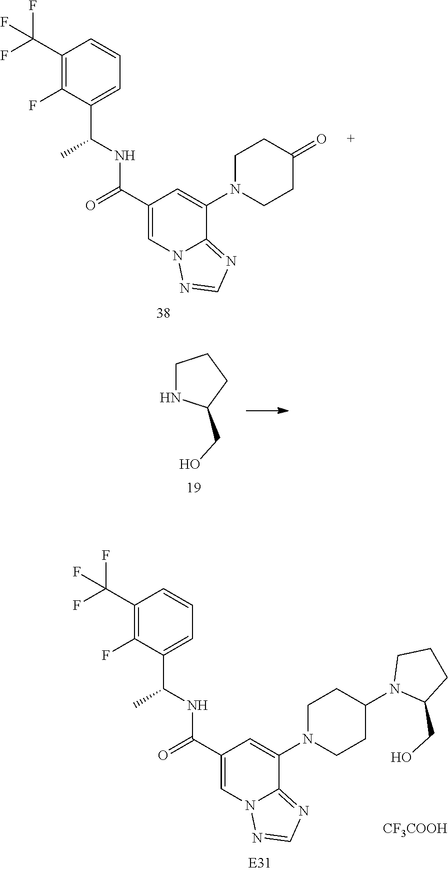 [SMILES: C[C@@H](NC(=O)c1cc(N2CCC(=O)CC2)c2ncnn2c1)c1cccc(C(F)(F)F)c1F ; OC[C@@H]1CCCN1 ; C[C@@H](NC(=O)c1cc(N2CCC(N3CCC[C@H]3CO)CC2)c2ncnn2c1)c1cccc(C(F)(F)F)c1F ; "CF3COOH"]

Example E31 is prepared in analogy to example E1: Intermediate 38 (80 μmol), L-prolinol 19 (0.12 mmol), AcOH (0.51 mmol), and 2-picoline-borane complex (80 μmol), 2 mL MeOH; purification by prep. RP-HPLC (acidic conditions).

Example E31
HPLC-MS Method: A

| | |
|---|---|
| Rₜ [min]: 0.80 | MS [m/z]: 535 [M + H]⁺ |

Analytical SFC method: L

| | |
|---|---|
| Rₜ [min]: 2.06 | d.e. >98% |

¹H NMR (400 MHz, DMSO-d₆) δ (ppm):
1.49-1.57 (3 H), 1.73-2.00 (5 H), 2.00-2.10 (1 H), 2.10-2.26
(2 H), 2.82-2.93 (2 H), 3.23-3.34 (1 H), 3.38-3.47 (1 H), 4.48-
4.57 (2 H), 5.38-5.47 (1 H), 7.14-7.20 (1 H), 7.37-7.45 (1 H),
7.65-7.72 (1 H), 7.81-7.87 (1 H), 8.52-8.57 (1 H), 9.10-9.17
(2 H), 9.17-9.24 (1 H), missing proton(s) presumably
hidden by/overlapping with solvent signals

### Synthesis of Example E32

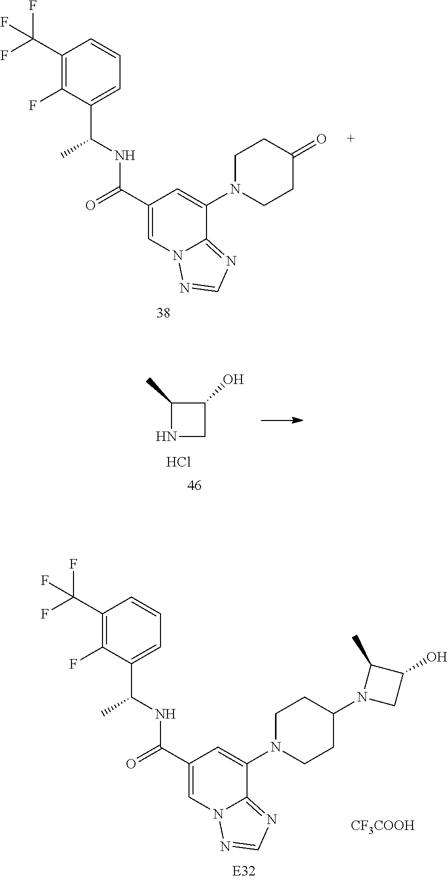

Example E32 is prepared in analogy to example E1: Intermediate 38 (80 μmol), (2S,3R)-2-methylazetidin-3-ol hydrochloride 46 (0.12 mmol), AcOH (0.51 mmol), and 2-picoline-borane complex (80 μmol), 2 mL MeOH; purification by prep. RP-HPLC (acidic conditions).

Example E32
HPLC-MS Method: A

| | |
|---|---|
| Rₜ [min]: 0.73 | MS [m/z]: 521 [M + H]⁺ |

Analytical SFC method: L

| | |
|---|---|
| Rₜ [min]: 1.83 | d.e. >98% |

¹H NMR (400 MHz, DMSO-d₆) δ (ppm):
1.12-1.20 (3 H), 1.26-1.45 (2 H), 1.48-1.55 (3 H), 1.69-1.78 (1 H),
1.80-1.89 (1 H), 2.24-2.31 (1 H), 2.86-2.93 (1 H), 2.93-3.02
(2 H), 4.07-4.21 (2 H), 5.35-5.43 (1 H), 7.07-7.13 (1 H),
7.36-7.43 (1 H), 7.63-7.69 (1 H), 7.77-7.83 (1 H), 8.45-8.50
(1 H), 9.00-9.03 (1 H), missing proton(s) presumably
hidden by/overlapping with solvent signals

### Synthesis of example E33

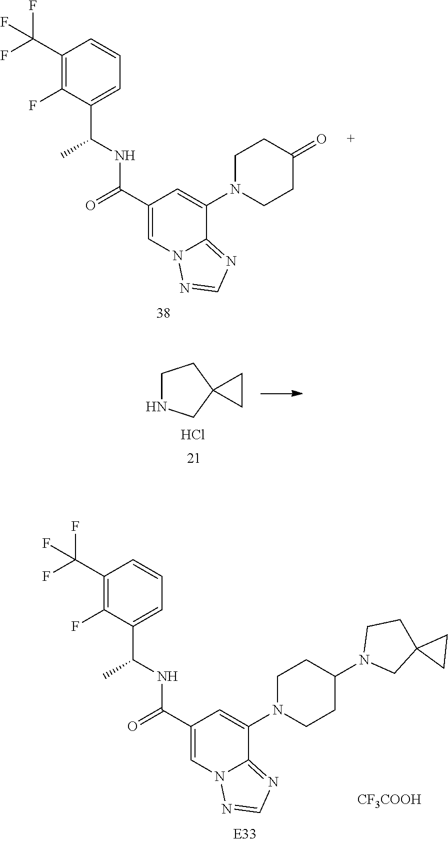 [SMILES: C[C@@H](NC(=O)c1cc(N2CCC(=O)CC2)c2ncnn2c1)c1cccc(C(F)(F)F)c1F ; C1CC2(CC2)CN1 ; Cl ; C[C@@H](NC(=O)c1cc(N2CCC(N3CCC4(CC4)C3)CC2)c2ncnn2c1)c1cccc(C(F)(F)F)c1F ; "CF3COOH"]

Example E33 is prepared in analogy to example E1: Intermediate 38 (80 μmol), 5-azaspiro[2.4]heptane hydrochloride 21 (0.12 mmol), AcOH (0.51 mmol), and 2-picoline-borane complex (80 μmol), 2 mL MeOH; purification by prep. RP-HPLC (acidic conditions).

Example E33
HPLC-MS Method: A

| | |
|---|---|
| Rₜ [min]: 0.98 | MS [m/z]: 531 [M + H]⁺ |

Analytical SFC method: L

| | |
|---|---|
| Rₜ [min]: 2.02 | d.e. >98% |

¹H NMR (400 MHz, DMSO-d₆) δ (ppm):
0.58-0.79 (4 H), 1.50-1.60 (3 H), 1.68-1.90 (3 H), 2.01-2.18 (2 H),
2.18-2.26 (1 H), 2.81-2.93 (2 H), 3.25-3.33 (2 H), 3.34-3.47
(2 H), 4.44-4.54 (2 H), 5.37-5.47 (1 H), 7.16-7.21 (1 H),
7.37-7.45 (1 H), 7.65-7.72 (1 H), 7.80-7.87 (1 H), 8.52-8.59
(1 H), 9.09-9.20 (2 H), 9.85-10.00 (1 H), missing proton(s)
presumably hidden by/overlapping with solvent signals

### Synthesis of Intermediate 48

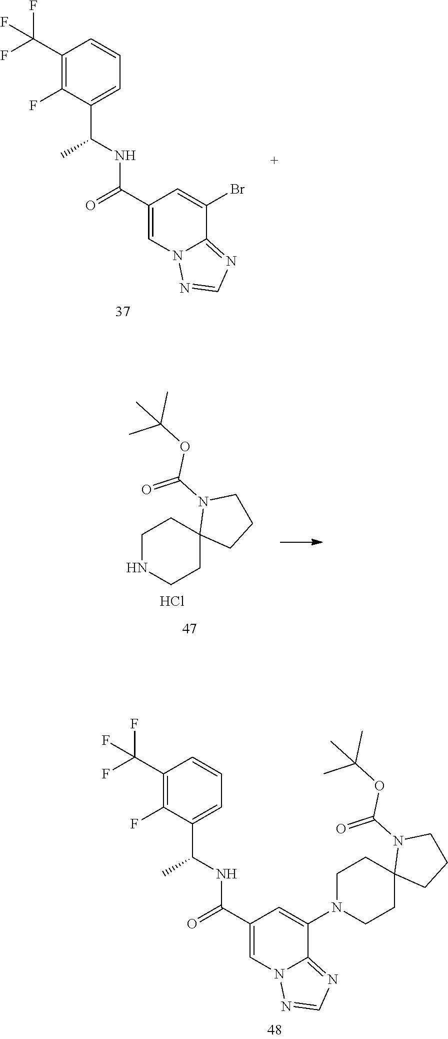 [SMILES: C[C@@H](NC(=O)c1cc(Br)c2ncnn2c1)c1cccc(C(F)(F)F)c1F ; CC(C)(C)OC(=O)N1CCCC12CCNCC2 ; Cl ; C[C@@H](NC(=O)c1cc(N2CCC3(CCCN3C(=O)OC(C)(C)C)CC2)c2ncnn2c1)c1cccc(C(F)(F)F)c1F]

A mixture of bromide 37 (200 mg, 464 μmol), tert-butyl 1,8-diazaspiro[4.5]decane-1-carboxylate hydrochloride 47 (149 mg, 510 μmol) and Cs₂CO₃ (382 mg, 1.16 mmol) and catalyst I (19 mg, 23 μmol) in degassed 1,4-dioxane (2 mL) is stirred under argon for 16 h at 95° C. The mixture is diluted with 1,4-dioxane, filtered through a thiol resin, and washed with DMF/MeOH. The material is purified by prep. RP-HPLC (acidic conditions) to obtain intermediate 48.

Intermediate 48
Analytical HPLC-MS Method: D

| | |
|---|---|
| Rₜ [min]: 1.17 | MS [m/z]: 591 [M + H]⁺ |

### Synthesis of Example E34

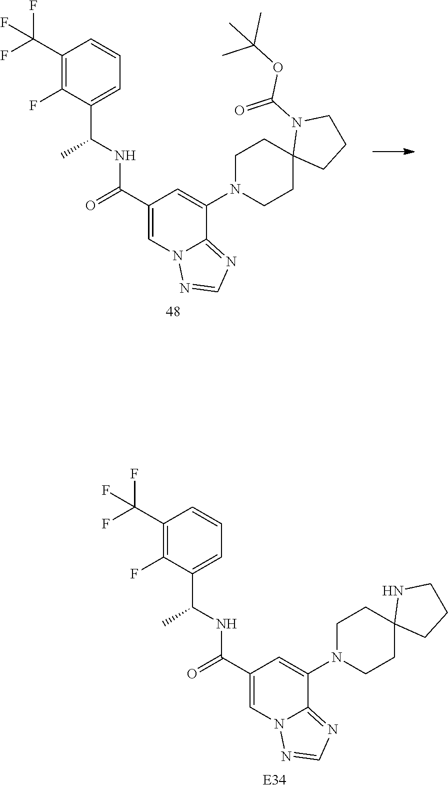 [SMILES: C[C@@H](NC(=O)c1cc(N2CCC3(CCCN3C(=O)OC(C)(C)C)CC2)c2ncnn2c1)c1cccc(C(F)(F)F)c1F ; C[C@@H](NC(=O)c1cc(N2CCC3(CCCN3)CC2)c2ncnn2c1)c1cccc(C(F)(F)F)c1F]

Intermediate 48 (134 mg, 227 μmol) in DCM (3 mL) is treated with 4 N HCl in 1,4-dioxane (142 μL) at 0° C. and stirred for 3 h at 0° C. The solvent is evaporated under reduced pressure and the material is purified by prep. RP-HPLC (basic conditions) to obtain example E34.

Example E34
Analytical HPLC-MS Method: D

| | |
|---|---|
| Rₜ [min]: 1.06 | MS [m/z]: 491 [M + H]⁺ |

Analytical SFC method: X

| | |
|---|---|
| Rₜ [min]: 4.10 | e.e. >98% |

¹H NMR (400 MHz, DMSO-d₆) δ (ppm):
1.48-1.76 (11 H), 1.90-2.16 (1 H), 2.78-2.88 (2 H), 3.55-3.67
(4 H), 5.36-5.47 (1 H), 7.11-7.16 (1 H), 7.38-7.45 (1 H), 7.64-7.71
(1 H), 7.79-7.86 (1 H), 8.47-8.53 (1 H), 9.02-9.06 (1 H), 9.06-9.13 (1 H).

### Synthesis of Example E35

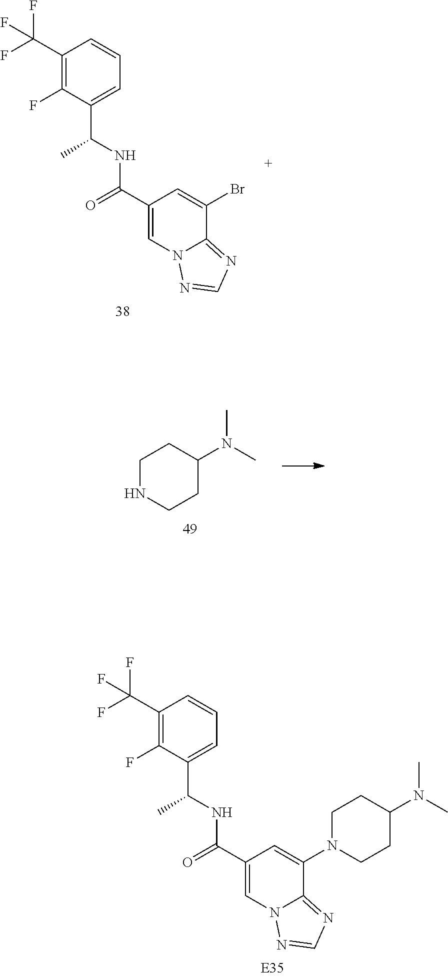

A mixture of bromide 38 (80 mg, 186 μmol), N,N-dimethylpiperidine-4-amine 49 (29 mg, 223 μmol) and Cs₂CO₃ (121 mg, 371 μmol) and catalyst I (16 mg, 19 μmol) in degassed 1,4-dioxane is stirred under argon for 18 h at 105° C. The mixture is diluted with THF and ACN/water and filtered. The material is purified by prep. RP-HPLC (basic conditions) to obtain example E35.

Example E35
Analytical HPLC-MS Method: A

| | |
|---|---|
| Rₜ [min]: 0.83 | MS [m/z]: 479 [M + H]⁺ |

Analytical SFC method: X

| | |
|---|---|
| Rₜ [min]: 4.14 | e.e. >98% |

¹H NMR (400 MHz, DMSO-d₆) δ (ppm):
1.46-1.61 (5 H), 1.84-1.93 (2 H), 2.17-2.22 (6 H), 2.23-2.35 (1 H),
2.80-2.91 (2 H), 4.30-4.37 (2 H), 5.36-5.47 (1 H), 7.14-7.19
(1 H), 7.36-7.44 (1 H), 7.63-7.70 (1 H), 7.82-7.90
(1 H), 8.47-8.52 (1 H), 9.04-9.39 (2 H), missing proton(s)
presumably hidden by/overlapping with solvent signals

### Synthesis of Intermediate 51

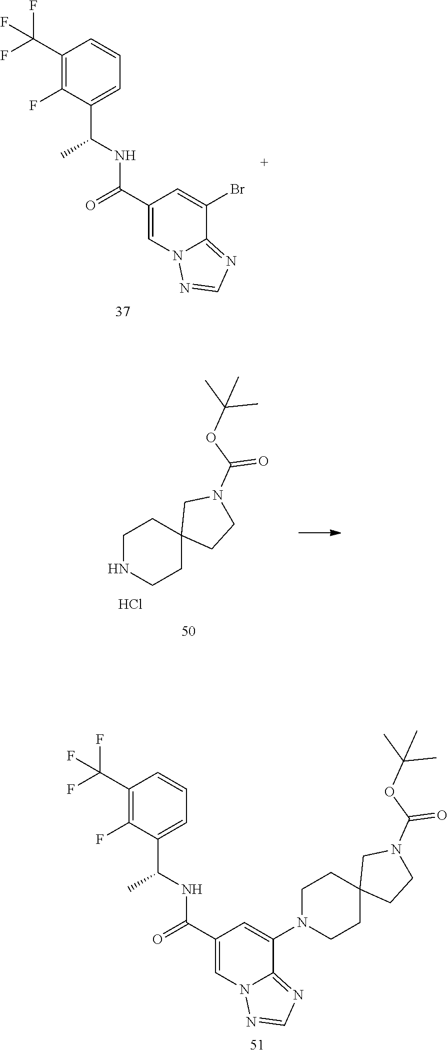 [SMILES: C[C@@H](NC(=O)c1cc(Br)c2ncnn2c1)c1cccc(C(F)(F)F)c1F ; CC(C)(C)OC(=O)N1CCC2(CCNCC2)C1 ; Cl ; C[C@@H](NC(=O)c1cc(N2CCC3(CCN(C(=O)OC(C)(C)C)C3)CC2)c2ncnn2c1)c1cccc(C(F)(F)F)c1F]

A mixture of bromide 37 (300 mg, 647 μmol), tert-butyl 2,8-diazaspiro[4.5]decane-2-carboxylate hydrochloride 50 (246 mg, 971 μmol) and Cs₂CO₃ (532 mg, 1.62 mmol) and catalyst I (27 mg, 32 μmol) in degassed 1,4-dioxane (2 mL) is stirred under argon for 18 h at 105° C. The mixture is diluted with 1,4-dioxane, filtered through a thiol resin and washed with MeOH. The material is purified by prep. RP-HPLC (basic conditions) to obtain intermediate 51.

Intermediate 51
Analytical HPLC-MS Method: D

| | |
|---|---|
| Rₜ [min]: 1.15 | MS [m/z]: 591 [M + H]⁺ |

### Synthesis of Example E36

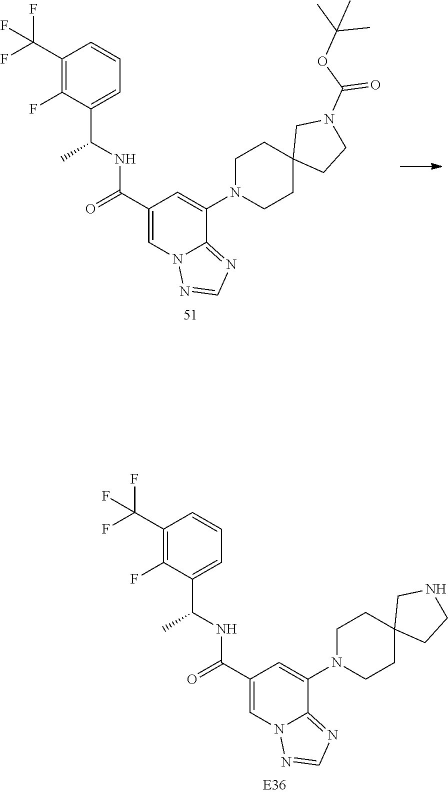 [SMILES: C[C@@H](NC(=O)c1cc(N2CCC3(CCN(C(=O)OC(C)(C)C)C3)CC2)c2ncnn2c1)c1cccc(C(F)(F)F)c1F ; C[C@@H](NC(=O)c1cc(N2CCC3(CCNC3)CC2)c2ncnn2c1)c1cccc(C(F)(F)F)c1F]

Intermediate 51 (370 mg, 626 μmol) in 1,4-dioxane (1 mL) is treated with 4 N HCl in 1,4-dioxane (2 mL) and stirred for 16 h at rt. The mixture is filtered through a carbonate cartridge, the solids are washed with MeOH and the combined solvents are evaporated under reduced pressure. The material is purified by prep. RP-HPLC (basic conditions) to obtain example E36.

Example E36
Analytical HPLC-MS Method: D

| | |
|---|---|
| Rₜ [min]: 1.06 | MS [m/z]: 491 [M + H]⁺ |

Analytical SFC method: H

| | |
|---|---|
| Rₜ [min]: 4.02 | e.e. = 98% |

¹H NMR (400 MHz, DMSO-d₆) δ (ppm):
1.49-1.58 (5 H), 1.60-1.71 (4 H), 2.59-2.64 (2 H), 2.78-2.85 (2 H),
3.48-3.63 (4 H), 5.37-5.47 (1 H), 7.11-7.20 (1 H), 7.38-7.44
(1 H), 7.64-7.71 (1 H), 7.80-7.86 (1 H), 8.48-8.53 (1 H),
9.04-9.07 (1 H), 9.07-9.14 (1 H), missing proton(s)
presumably hidden by/overlapping with solvent signals

### Synthesis of Example E37

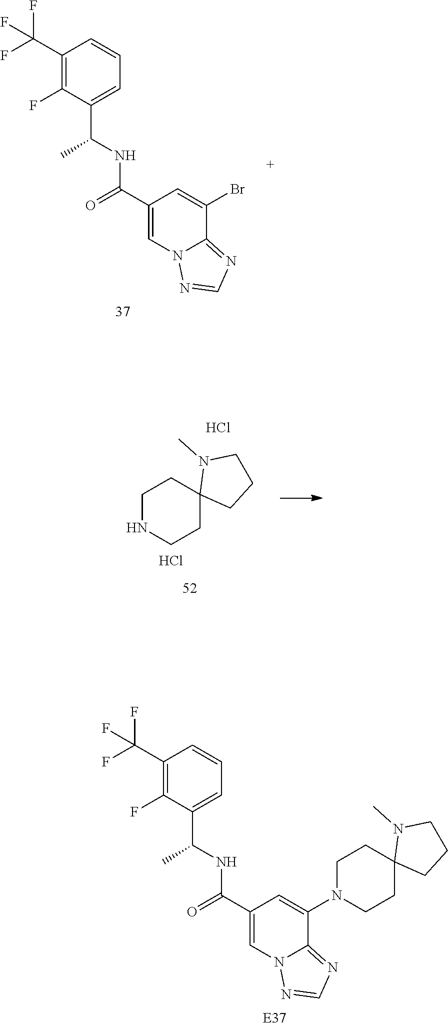 [SMILES: C[C@@H](NC(=O)c1cc(Br)c2ncnn2c1)c1cccc(C(F)(F)F)c1F ; Cl ; CN1CCCC12CCNCC2 ; Cl ; C[C@@H](NC(=O)c1cc(N2CCC3(CCCN3C)CC2)c2ncnn2c1)c1cccc(C(F)(F)F)c1F]

A mixture of bromide 37 (200 mg, 464 μmol), 1-methyl-1,8-diazaspiro[4.5]decane dihydrochloride 52 (122 mg, 510 μmol) and Cs₂CO₃ (534 mg, 1.62 mmol) and catalyst I (19 mg, 23 μmol) in degassed 1,4-dioxane (2 mL) is stirred under argon for 16 h at 90° C. The mixture is diluted with 1,4-dioxane, filtered through thiol resin, and washed with MeOH. The material is purified by prep. RP-HPLC (basic conditions) to obtain example E37.

Example E37
Analytical HPLC-MS Method: D

| | |
|---|---|
| Rₜ [min]: 1.06 | MS [m/z]: 505 [M + H]⁺ |

Analytical SFC method: AB

| | |
|---|---|
| Rₜ [min]: 3.63 | e.e. >98% |

¹H NMR (400 MHz, DMSO-d₆) δ (ppm):
1.28-1.37 (2 H), 1.49-1.57 (3 H), 1.65-1.84 (6 H), 2.17-2.23 (3 H),
2.65-2.74 (2 H), 2.80-2.91 (2 H), 4.30-4.40 (2 H), 5.37-5.48
(1 H), 7.13-7.21 (1 H), 7.37-7.45 (1 H), 7.64-7.71 (1 H),
7.79-7.87 (1 H), 8.47-8.56 (1 H), 9.04-9.13 (2 H).

### Synthesis of Intermediate 54

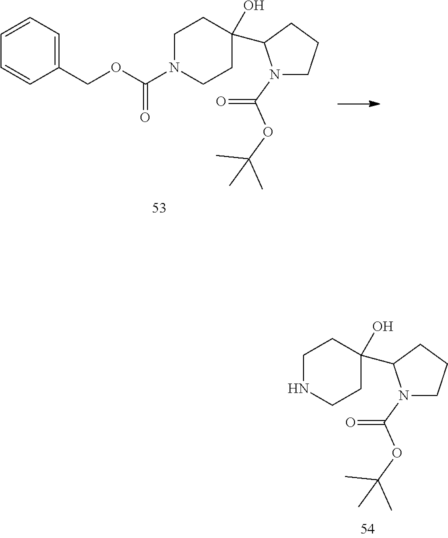 [SMILES: CC(C)(C)OC(=O)N1CCCC1C1(O)CCN(C(=O)OCc2ccccc2)CC1 ; CC(C)(C)OC(=O)N1CCCC1C1(O)CCNCC1]

A mixture of intermediate 53 (WO2016147011; 188 mg, 465 μmol), Pd/C (5 wt. %, 50 mg) and 2 drops aqueous NH₃ solution (7 N) in MeOH (10 mL) is stirred under 1 bar hydrogen for 16 h at rt. The mixture is filtered and the solids are washed with MeOH. The solvent is removed under reduced pressure and intermediate 54 is used in the next reaction step without further purification.

Intermediate 54
Analytical HPLC-MS Method: D

| | |
|---|---|
| Rₜ [min]: 0.98 | MS [m/z]: 271 [M + H]⁺ |

### Synthesis of Intermediate 55

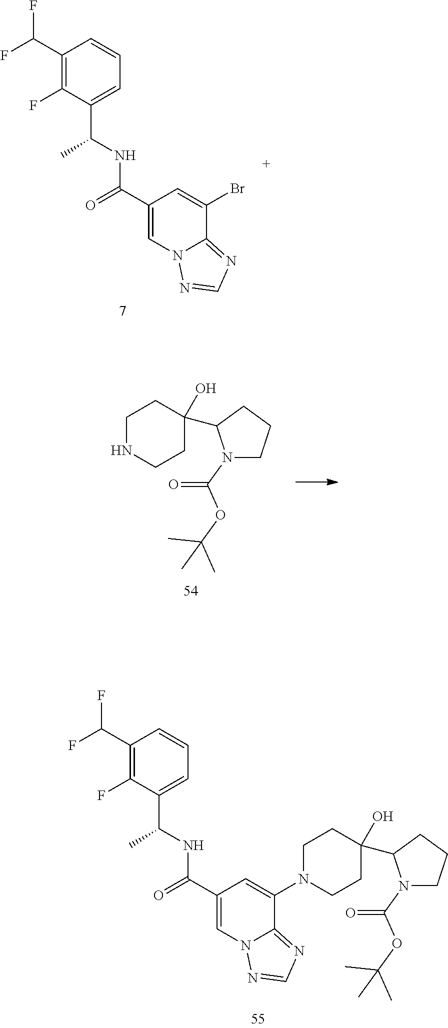 [SMILES: C[C@@H](NC(=O)c1cc(Br)c2ncnn2c1)c1cccc(C(F)F)c1F ; CC(C)(C)OC(=O)N1CCCC1C1(O)CCNCC1 ; C[C@@H](NC(=O)c1cc(N2CCC(O)(C3CCCN3C(=O)OC(C)(C)C)CC2)c2ncnn2c1)c1cccc(C(F)F)c1F]

A mixture of bromide 7 (80 mg, 194 μmol), intermediate 54 (60 mg, 222 μmol) and potassium phosphate (84 mg, 387 μmol) and catalyst I (8 mg, 10 μmol) in degassed 1,4-dioxane (3 mL) is stirred under argon for 18 h at 105° C. The mixture is diluted with DCM, filtered through Celite, and washed with MeOH. The solvent is removed under reduced pressure and the material is used in the next reaction step without further purification.

Intermediate 55
Analytical HPLC-MS Method: D

| | |
|---|---|
| Rₜ [min]: 1.10 | MS [m/z]: 603 [M + H]⁺ |

### Synthesis of Examples E38a and E38b

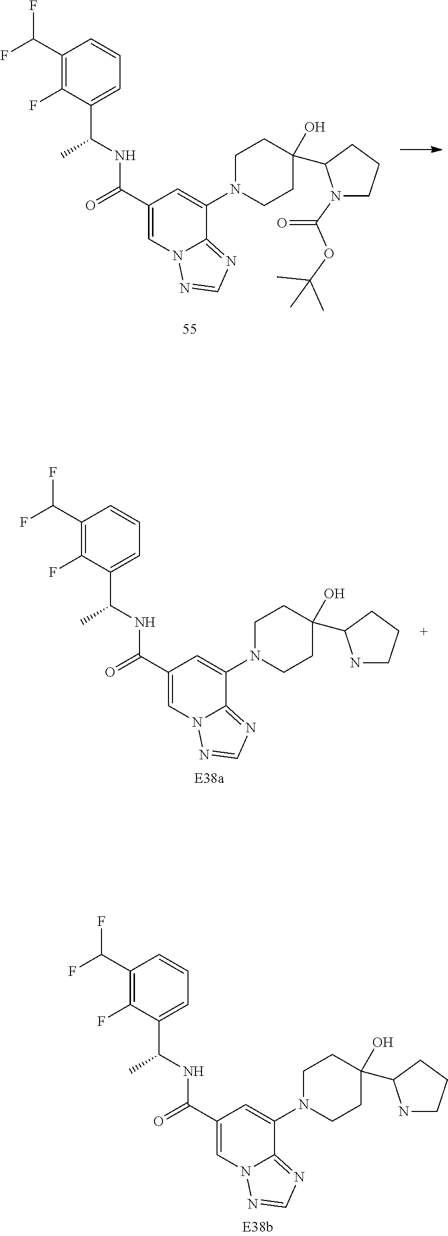 [SMILES: C[C@@H](NC(=O)c1cc(N2CCC(O)(C3CCCN3C(=O)OC(C)(C)C)CC2)c2ncnn2c1)c1cccc(C(F)F)c1F ; C[C@@H](NC(=O)c1cc(N2CCC(O)(C3CCCN3)CC2)c2ncnn2c1)c1cccc(C(F)F)c1F ; C[C@@H](NC(=O)c1cc(N2CCC(O)(C3CCCN3)CC2)c2ncnn2c1)c1cccc(C(F)F)c1F]

Intermediate 55 (117 mg, 194 μmol) in 1,4-dioxane (2 mL) is treated with HCl in 1,4-dioxane (4 M; 242 μL) and stirred for 6 h at rt, then aqueous HCl solution (4 M; 300 μL) is added, and the mixture stirred for 18 h before aqueous HCl solution (6 M; 500 μL) is added and the mixture is stirred for 16 h at rt. The mixture is neutralized with NaOH solution (4 M), basified with aqueous NH₃ solution and extracted with DCM. The organic solvent is evaporated under reduced pressure and the material is purified by prep. RP-HPLC (basic conditions) and chiral SFC to obtain examples E38a and E38b as single stereoisomers. The absolute configuration at the pyrrolidine is not known.

Example E38a
Analytical HPLC-MS Method: D

| | |
|---|---|
| Rₜ [min]: 0.98 | MS [m/z]: 503 [M + H]⁺ |

Analytical SFC method: Al

| | |
|---|---|
| Rₜ [min]: 2.02 | d.e.: >98% |

¹H NMR (400 MHz, DMSO-d₆) δ (ppm):
1.48-1.74 (10 H), 2.74-2.88 (2 H), 2.89-2.95 (1 H), 3.13-3.24
(2 H), 3.54-3.59 (1 H), 4.01-4.29 (3 H), 5.35-5.47 (1 H),
7.06-7.38 (3 H), 7.48-7.56 (1 H), 7.64-7.74 (1 H), 8.47-
8.53 (1 H), 9.01-9.11 (2 H), missing proton(s)
presumably hidden by/overlapping with solvent signals

Example E38b
Analytical HPLC-MS Method: D

| | |
|---|---|
| Rₜ [min]: 0.98 | MS [m/z]: 503 [M + H]⁺ |

Analytical SFC method: Al

| | |
|---|---|
| Rₜ [min]: 1.38 | d.e. = 96% |

¹H NMR (400 MHz, DMSO-d₆) δ (ppm):
1.48-1.74 (11 H), 2.72-2.86 (2 H), 2.86-2.92 (1 H), 3.12-3.22
(2 H), 3.83-4.34 (3 H), 5.36-5.47 (1 H), 7.06-7.38 (3 H), 7.49-7.57
(1 H), 7.64-7.74 (1 H), 8.46-8.53 (1 H), 9.01-9.10 (2 H), missing
proton(s) presumably hidden by/overlapping with solvent signals.

### Synthesis of Example E39

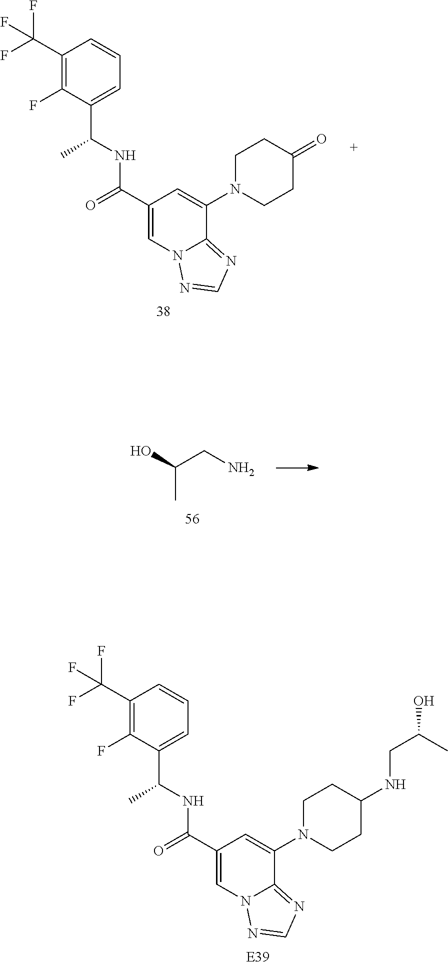

A mixture of intermediate 38 (50 mg, 111 μmol), (2R)-1-aminopropan-2-ol 56 (13 mg, 167 μmol) and AcOH (9.6 μL, 167 μmol) in DCM (0.5 mL) is stirred for 15 min at rt before sodium triacetoxyborohydride (36 mg, 167 μmmol) is added. The mixture is stirred for 3 h at rt, diluted with DCM, and extracted with aqueous saturated NaHCO₃ solution. The volume of the separated organic layer is decreased under reduced pressure. The material is purified by prep. RP-HPLC (acidic conditions). After lyophilization, the material is redissolved in MeOH and filtered through a carbonate cartridge to obtain example E39.

Example E39
Analytical HPLC-MS Method: D

| | |
|---|---|
| Rₜ [min]: 0.97 | MS [m/z]: 509 [M + H]⁺ |

Analytical SFC method: AO

| | |
|---|---|
| Rₜ [min]: 5.90 | d.e. >98% |

¹H NMR (400 MHz, DMSO-d₆) δ (ppm):
1.02-1.09 (3 H), 1.36-1.48 (2 H), 1.51-1.57 (4 H), 1.89-2.00
(2 H), 2.41-2.49 (1 H), 2.58-2.70 (1 H), 2.92-3.04 (2 H), 3.60-3.70
(1 H), 4.16-4.29 (2 H), 4.37-4.57 (1 H), 5.36-5.47 (1 H), 7.10-7.18
(1 H), 7.38-7.46 (1 H), 7.64-7.72 (1 H), 7.80-7.87 (1 H),
8.49-8.54 (1 H), 9.05-9.16 (2 H), missing proton(s)
presumably hidden by/overlapping with solvent signals.

### Synthesis of Example E40

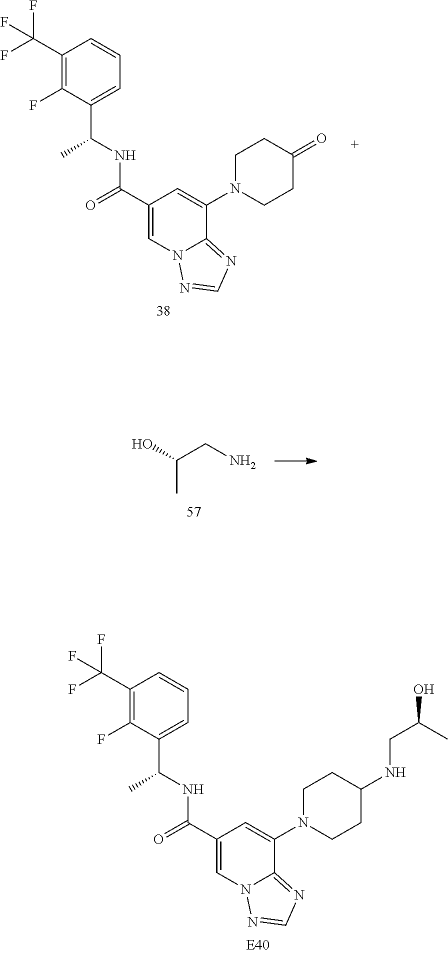

Example E40 is prepared in analogy to example E39: Intermediate 38 (111 μmol), (2S)-1-aminopropan-2-ol 57 (167 μmol), AcOH (167 μmol), and sodium triacetoxyborohydride (167 μmol), 0.5 mL DCM.

Example E40
Analytical HPLC-MS Method: D

| | |
|---|---|
| Rₜ [min]: 0.97 | MS [m/z]: 509 [M + H]⁺ |

Analytical SFC method: AG

| | |
|---|---|
| Rₜ [min]: 5.00 | d.e. >98% |

¹H NMR (400 MHz, DMSO-d₆) δ (ppm):
1.00-1.09 (3 H), 1.31-1.48 (2 H), 1.48-1.67 (4 H), 1.88-1.99
(2 H), 2.42-2.48 (1 H), 2.56-2.65 (1 H), 2.93-3.04 (2 H),
3.58-3.69 (1 H), 4.14-4.27 (2 H), 4.39-4.44 (1 H), 5.36-5.47
(1 H), 7.10-7.16 (1 H), 7.37-7.45 (1 H), 7.64-7.71 (1 H),
7.79-7.87 (1 H), 8.48-8.53 (1 H), 9.04-9.13 (2 H), missing
proton(s) presumably hidden by/overlapping with solvent signals.

### Synthesis of Examples Cis-E41a and Cis-E41b

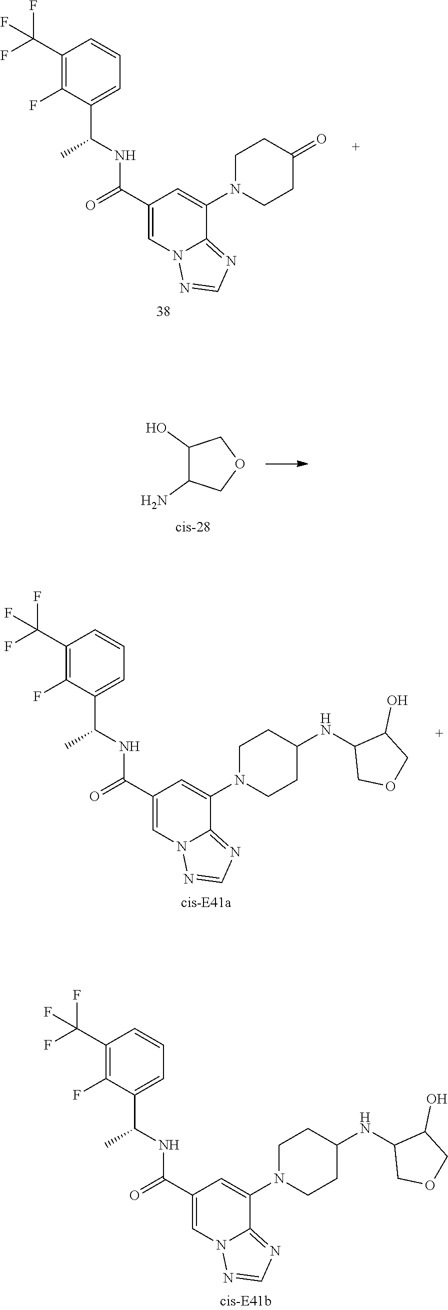 [SMILES: C[C@@H](NC(=O)c1cc(N2CCC(=O)CC2)c2ncnn2c1)c1cccc(C(F)(F)F)c1F ; NC1COCC1O ; C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F]

Examples cis-E41 and cis-E41b are prepared in analogy to example E39: Intermediate 38 (223 μmol), cis-4-aminotetrahydrofuran-3-ol cis-28 (245 μmol), AcOH (245 μmol), and sodium triacetoxyborohydride (334 μmol), 1 mL DCM; in addition: chiral SFC separation (basic conditions). Cis-E41a and cis-E41b are isolated as single stereoisomers. The absolute configuration of the amino and hydroxy substituents at the THF ring is not known; their relative configuration is cis.

Example cis-E41a
Analytical HPLC-MS Method: D

| | |
|---|---|
| Rₜ [min]: 0.83 | MS [m/z]: 537 [M + H]⁺ |

Analytical SFC method: J

| | |
|---|---|
| Rₜ [min]: 3.22 | d.e. = 97% |

¹H NMR (400 MHz, DMSO-d₆) δ (ppm):
1.36-1.58 (5 H), 1.89-2.03 (2 H), 2.67-2.77 (1 H), 2.92-3.03 (2 H),
3.57-3.64 (1 H), 3.76-3.87 (2 H), 4.03-4.09 (1 H), 4.20-4.30 (2 H),
4.68-5.11 (1 H), 5.37-5.48 (1 H), 7.12-7.17 (1 H), 7.38-7.45 (1 H),
7.64-7.72 (1 H), 7.80-7.87 (1 H), 8.48-8.55 (1 H), 9.04-9.16 (2 H),
missing proton(s) presumably hidden by/overlapping with solvent signals.

Example cis-E41b
Analytical HPLC-MS Method: D

| | |
|---|---|
| Rₜ [min]: 0.83 | MS [m/z]: 537 [M + H]⁺ |

Analytical SFC method: J

| | |
|---|---|
| Rₜ [min]: 3.61 | d.e. = 84% |

¹H NMR (400 MHz, DMSO-d₆) δ (ppm):
1.38-1.58 (5 H), 1.90-2.03 (2 H), 2.69-2.79 (1 H), 2.92-3.03
(2 H), 3.56-3.63 (1 H), 3.76-3.87 (2 H), 4.04-4.10 (1 H), 4.20-4.31
(2 H), 5.37-5.47 (1 H), 7.12-7.16 (1 H), 7.39-7.45 (1 H), 7.64-7.72 (1 H),
7.80-7.87 (1 H), 8.47-8.54 (1 H), 9.05-9.15 (2 H), missing proton(s)
presumably hidden by/overlapping with solvent signals.

### Synthesis of Examples Trans-E42a and Trans-E42b

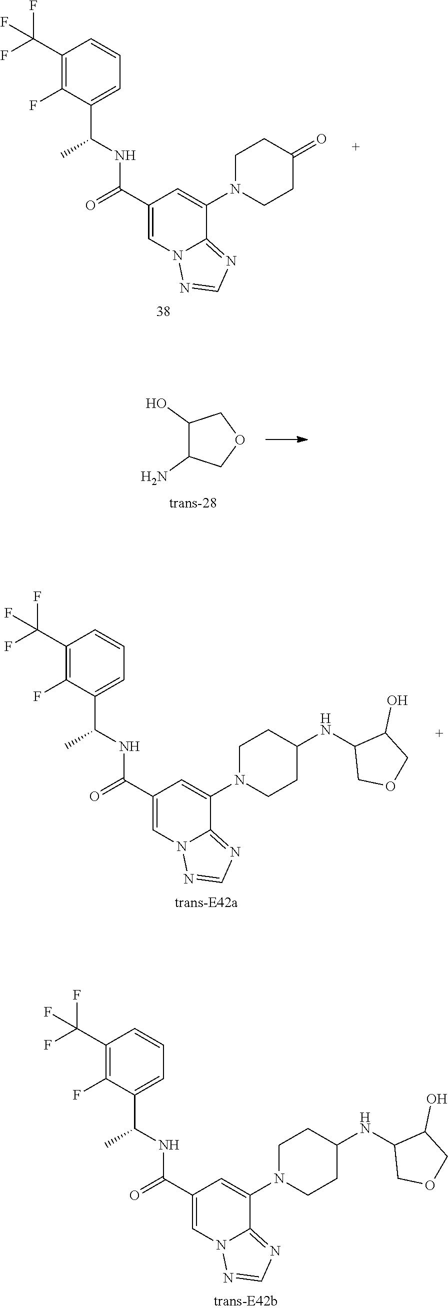 [SMILES: C[C@@H](NC(=O)c1cc(N2CCC(=O)CC2)c2ncnn2c1)c1cccc(C(F)(F)F)c1F ; NC1COCC1O ; C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F]

Examples trans-E42 and trans-E42b are prepared in analogy to example E39: Intermediate 38 (445 μmol), trans-4-aminotetrahydrofuran-3-ol trans-28 (668 μmol), AcOH (668 μmol), and sodium triacetoxyborohydride (668 μmol), 2 mL DCM; in addition: chiral SFC separation (basic conditions). Trans-E42a and trans-E42b are isolated as single stereoisomers. The absolute configuration of the amino and hydroxy substituents at the THF ring is not known; their relative configuration is trans.

Example trans-E42a
HPLC-MS Method: E

| | |
|---|---|
| Rₜ [min]: 0.83 | MS [m/z]: 537 [M + H]⁺ |

Analytical SFC method: G

| | |
|---|---|
| Rₜ [min]: 3.64 | d.e. >98% |

¹H NMR (400 MHz, DMSO-d₆) δ (ppm):
1.33-1.48 (2 H), 1.49-1.57 (3 H), 1.91-2.03 (2 H), 2.69-2.79
(1 H), 2.90-3.01 (2 H), 3.15-3.22 (1 H), 3.37-3.43 (1 H), 3.43-3.50
(1 H), 3.77-3.84 (1 H), 3.86-3.92 (1 H), 3.94-3.99 (1 H), 4.20-4.30 (2 H),
4.87-4.96 (1 H), 5.37-5.47 (1 H), 7.10-7.15 (1 H), 7.37-7.44 (1 H), 7.64-
7.71 (1 H), 7.79-7.87 (1 H), 8.49-8.53 (1 H), 9.03-9.14 (2 H), missing
proton(s) presumably hidden by/overlapping with solvent signals.

Example trans-E42b
Analytical HPLC-MS Method: C

| | |
|---|---|
| Rₜ [min]: 0.83 | MS [m/z]: 537 [M + H]⁺ |

Analytical SFC method: G

| | |
|---|---|
| Rₜ [min]: 5.52 | d.e. >98% |

¹H NMR (400 MHz, DMSO-d₆) δ (ppm):
1.33-1.48 (2 H), 1.49-1.58 (3 H), 1.92-2.03 (2 H), 2.68-2.79 (1 H),
2.90-3.02 (2 H), 3.15-3.22 (1 H), 3.37-3.44 (1 H), 3.44-3.50
(1 H), 3.77-3.84 (1 H), 3.86-3.93 (1 H), 3.93-4.01 (1 H),
4.20-4.31 (2 H), 4.85-4.96 (1 H), 5.36-5.47 (1 H), 7.10-
7.17 (1 H), 7.38-7.45 (1 H), 7.64-7.71 (1 H), 7.79-7.86
(1 H), 8.48-8.54 (1 H), 9.03-9.14 (2 H), missing proton(s)
presumably hidden by/overlapping with solvent signals.

### Synthesis of Examples E43a and E43b

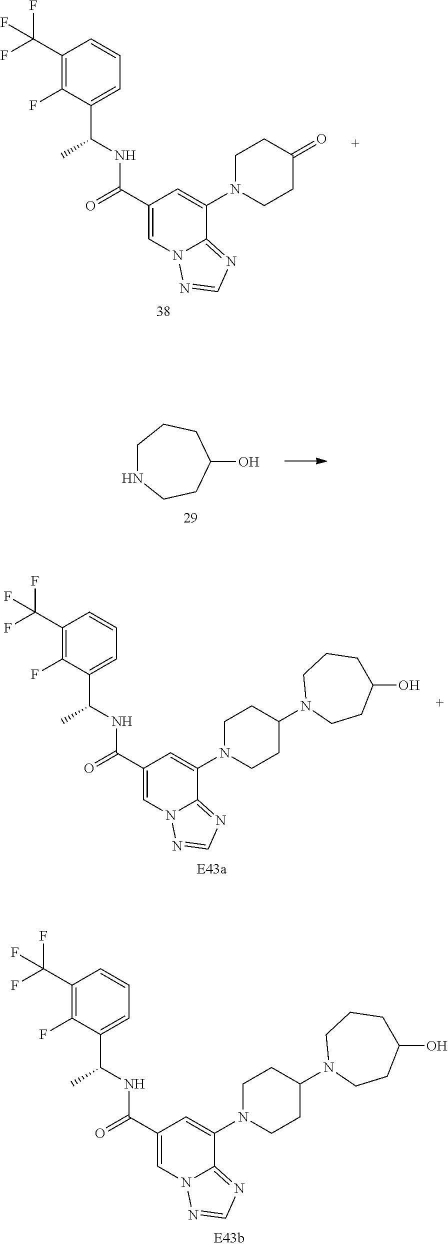 [SMILES: C[C@@H](NC(=O)c1cc(N2CCC(=O)CC2)c2ncnn2c1)c1cccc(C(F)(F)F)c1F ; OC1CCCNCC1 ; C[C@@H](NC(=O)c1cc(N2CCC(N3CCCC(O)CC3)CC2)c2ncnn2c1)c1cccc(C(F)(F)F)c1F ; C[C@@H](NC(=O)c1cc(N2CCC(N3CCCC(O)CC3)CC2)c2ncnn2c1)c1cccc(C(F)(F)F)c1F]

Examples E43a and E43b are prepared in analogy to example E39: Intermediate 38 (556 μmol), azepin-4-ol 29 (834 μmol), AcOH (834 μmol), and sodium triacetoxyborohydride (1.11 mmol), 2 mL DCM; in addition: chiral SFC separation (basic conditions). E43a and E43b are isolated as single stereoisomers. The absolute configuration of the hydroxy substituent is not known.

Example E43a
HPLC-MS Method: E

| | |
|---|---|
| Rₜ [min]: 0.83 | MS [m/z]: 549 [M + H]⁺ |

Analytical SFC method: AC

| | |
|---|---|
| Rₜ [min]: 4.46 | d.e. >98% |

¹H NMR (400 MHz, DMSO-d₆) δ (ppm):
1.36-1.85 (13 H), 2.58-2.73 (4 H), 2.77-2.88 (2 H), 3.63-3.74 (1 H),
4.17-4.47 (3 H), 5.35-5.46 (1 H), 7.10-7.15 (1 H), 7.38-7.44
(1 H), 7.64-7.71 (1 H), 7.79-7.87 (1 H), 8.48-8.52 (1 H),
9.03-9.14 (2 H), missing proton(s) presumably
hidden by/overlapping with solvent signals.

Example E43b
HPLC-MS Method: E

| | |
|---|---|
| Rₜ [min]: 0.83 | MS [m/z]: 549 [M + H]⁺ |

Analytical SFC method: AC

| | |
|---|---|
| Rₜ [min]: 3.51 | d.e. >98% |

¹H NMR (400 MHz, DMSO-d₆) δ (ppm):
1.35-1.90 (13 H), 2.56-2.75 (4 H), 2.75-2.89 (2 H), 3.61-3.76 (1 H),
4.16-4.47 (3 H), 5.37-5.48 (1 H), 7.08-7.16 (1 H), 7.36-7.45
(1 H), 7.60-7.73 (1 H), 7.78-7.88 (1 H), 8.44-8.59 (1 H),
9.00-9.18 (2 H), missing proton(s) presumably hidden
by/overlapping with solvent signals.

### Synthesis of Example E44

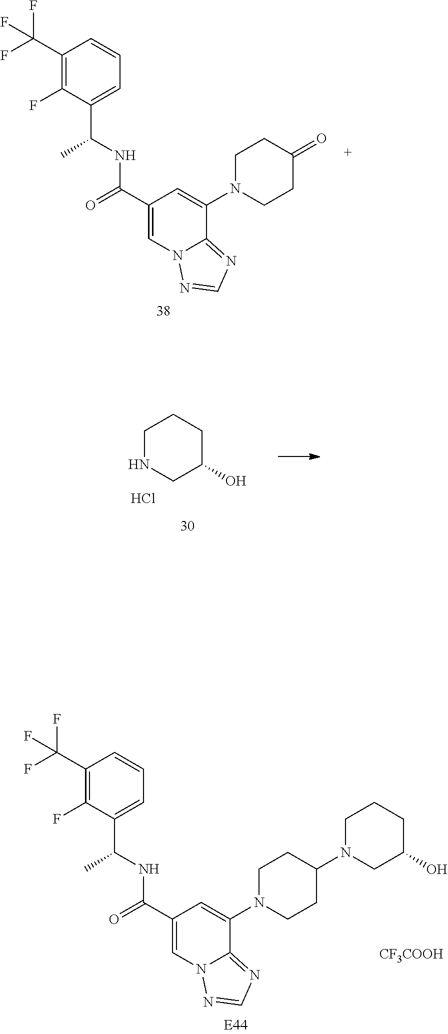 [SMILES: C[C@@H](NC(=O)c1cc(N2CCC(=O)CC2)c2ncnn2c1)c1cccc(C(F)(F)F)c1F ; O[C@H]1CCCNC1 ; Cl ; C[C@@H](NC(=O)c1cc(N2CCC(N3CCC[C@H](O)C3)CC2)c2ncnn2c1)c1cccc(C(F)(F)F)c1F ; "CF3COOH"]

2-Picoline-borane complex (6.2 mg, 60 μmol) is added to a mixture of intermediate 38 (26 mg, 158 μmol), (S)-3-hydroxypiperidine hydrochloride 30 (17 mg, 116 μmol) and AcOH (10 μL, 514 μM) in MeOH (0.5 mL). The mixture is stirred for 16 h at rt. The mixture is neutralized with AcOH, diluted with MeOH and purified by prep. RP-HPLC (acidic conditions) to obtain example E44.

Example E44
Analytical HPLC-MS Method: E

| | |
|---|---|
| Rₜ [min]: 0.79 | MS [m/z]: 535 [M + H]⁺ |

Analytical SFC method: AE

| | |
|---|---|
| Rₜ [min]: 1.48 | d.e. >98% |

¹H NMR (400 MHz, DMSO-d₆) δ (ppm):
1.50-2.25 (11 H), 2.80-2.94 (3 H), 2.98-3.14 (2 H), 4.53
(2 H), 5.42 (1 H), 7.18 (1 H), 7.41 (1 H), 7.64-7.72 (1 H),
7.83 (1 H), 8.55 (1 H), 8.90-9.34 (3 H), missing proton(s)
presumably hidden by/overlapping with solvent signals.

### Synthesis of Intermediate 58

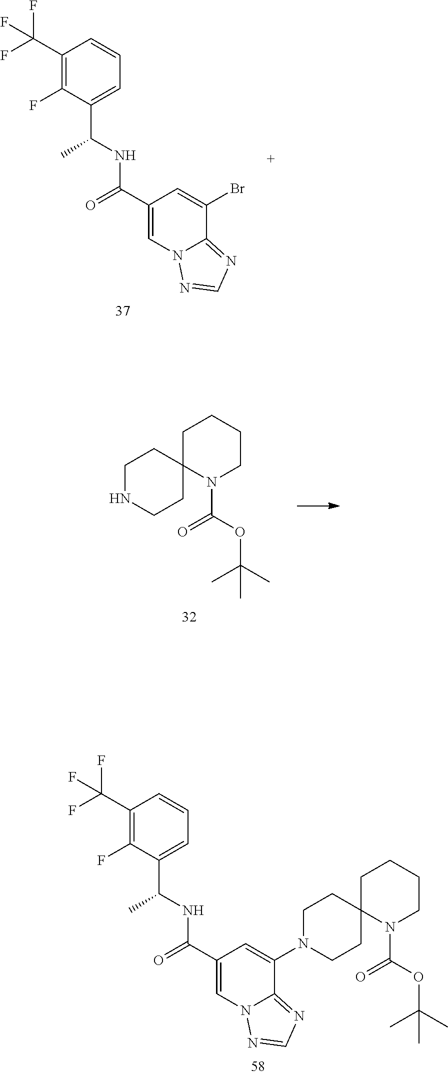 [SMILES: C[C@@H](NC(=O)c1cc(Br)c2ncnn2c1)c1cccc(C(F)(F)F)c1F ; CC(C)(C)OC(=O)N1CCCCC12CCNCC2 ; C[C@@H](NC(=O)c1cc(N2CCC3(CCCCN3C(=O)OC(C)(C)C)CC2)c2ncnn2c1)c1cccc(C(F)(F)F)c1F]

A mixture of bromide 37 (120 mg, 278 μmol), tert-butyl 1,9-diazaspiro[5.5]undecane-1-carboxylate 32 (112 mg, 418 μmol) and Cs₂CO₃ (272 mg, 835 μmol) in 1,4-dioxane (4 mL) is degassed with argon. Catalyst I (14 mg, 17 μmol) is added, and the mixture is heated under argon for 3 h at 90° C. The mixture is diluted with ACN, filtered, and the solvent is evaporated under reduced pressure. The desired material is used directly for the next step without further purification.

Intermediate 58
Analytical HPLC-MS Method: D

| | |
|---|---|
| Rₜ [min]: 1.23 | MS [m/z]: 605 [M + H]⁺ |

### Synthesis of Example E45

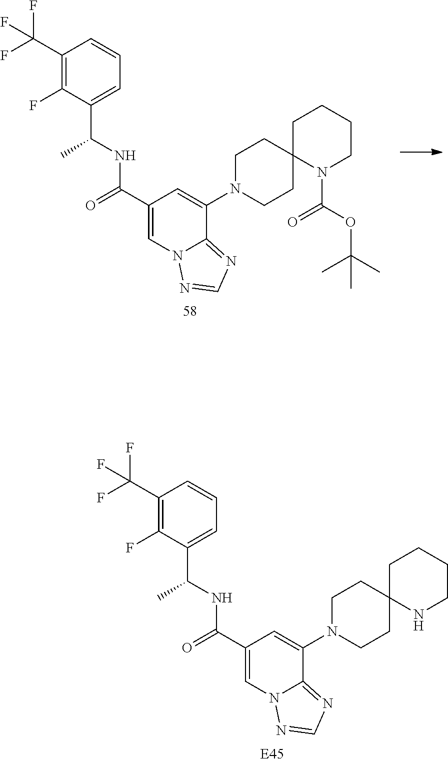 [SMILES: C[C@@H](NC(=O)c1cc(N2CCC3(CCCCN3C(=O)OC(C)(C)C)CC2)c2ncnn2c1)c1cccc(C(F)(F)F)c1F ; C[C@@H](NC(=O)c1cc(N2CCC3(CCCCN3)CC2)c2ncnn2c1)c1cccc(C(F)(F)F)c1F]

A mixture of intermediate 58 (168 mg, 278 μmol) and TFA (0.4 mL) in DCM (1.5 mL) is stirred for 1.5 h at rt. The mixture is concentrated under reduced pressure, saturated aqueous Na₂CO₃ solution is added, and the mixture is extracted with EtOAc. The combined organic layers are washed with saturated NaCl solution, dried over MgSO₄, and concentrated under reduced pressure. The residue is purified by prep. RP-HPLC (basic conditions) to give example E45.

Example E45
HPLC-MS Method: E

| | |
|---|---|
| Rₜ [min]: 0.89 | MS [m/z]: 505 [M + H]⁺ |

Analytical SFC method: AL

| | |
|---|---|
| Rₜ [min]: 2.75 | e.e. >98% |

¹H NMR (400 MHz, DMSO-d₆) δ (ppm):
1.38 (4 H), 1.51-1.78 (10 H), 2.64-2.70 (2 H), 3.40-3.48 (2 H), 3.70-3.81
(2 H), 5.39-5.46 (1 H), 7.12-7.16 (1 H), 7.39-7.43 (1 H), 7.66-7.69
(1 H), 7.81-7.84 (1 H), 8.49 (1 H), 9.02-9.04 (1 H), 9.08-9.10 (1 H).

### Synthesis of Example E46

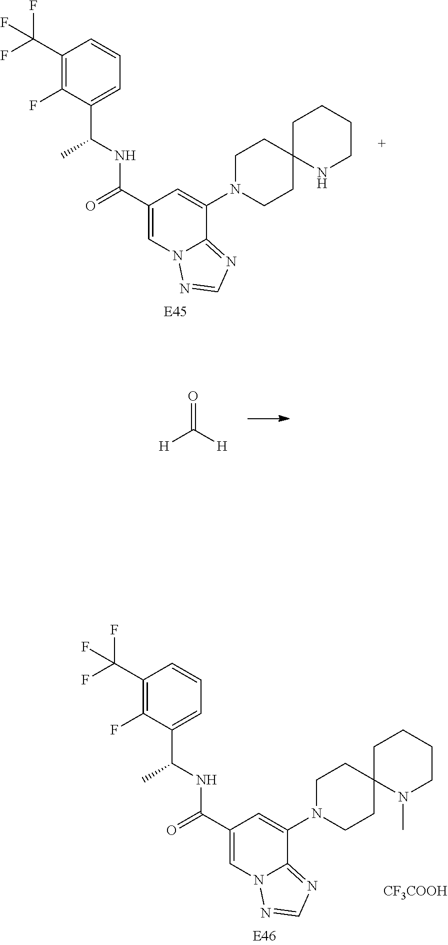 [SMILES: C[C@@H](NC(=O)c1cc(N2CCC3(CCCCN3)CC2)c2ncnn2c1)c1cccc(C(F)(F)F)c1F ; C=O ; C[C@@H](NC(=O)c1cc(N2CCC3(CCCCN3C)CC2)c2ncnn2c1)c1cccc(C(F)(F)F)c1F ; "CF3COOH"]

Example E46 is prepared in analogy to example E39: E45 (142 μmol), aqueous formaldehyde solution (37%, 213 μmol), AcOH (142 μmol), and sodium triacetoxyborohydride (213 μmol), 3 mL DCE; purification by prep. RP-HPLC (acidic conditions).

Example E46
Analytical HPLC-MS Method: D

| | |
|---|---|
| Rₜ [min]: 1.09 | MS [m/z]: 519 [M + H]⁺ |

Analytical SFC method: AL

| | |
|---|---|
| Rₜ [min]: 2.57 | e.e. >98% |

¹H NMR (400 MHz, DMSO-d₆) δ (ppm):
1.53-1.55 (3 H), 1.58-1.71 (3 H), 1.74-1.97 (3 H),
1.99-2.21 (4 H), 2.80-2.82 (3 H), 2.95-3.24 (3 H),
3.32-3.48 (1 H), 5.39-5.46 (1 H), 7.17-7.20 (1 H),
7.39-7.43 (1 H), 7.66-7.70 (1 H), 7.82-7.85 (1 H),
8.55 (1 H), 9.13-9.15 (2 H), 9.41 (1 H), missing proton(s)
presumably hidden by/overlapping with solvent signals.

### Synthesis of Examples E47a and E47b

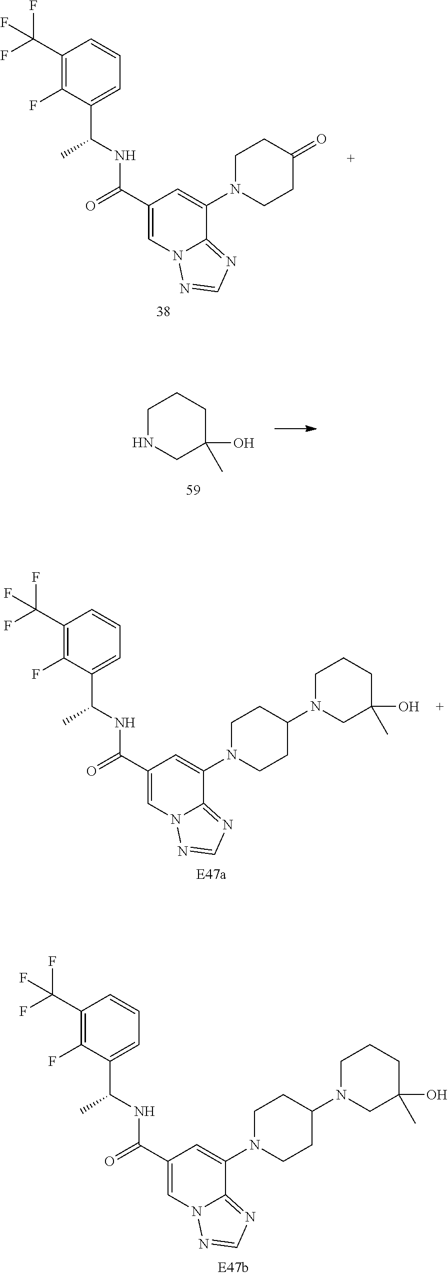

Examples E47a and E47b are prepared in analogy to example E1: Intermediate 38 (223 μmol), 3-methylpiperidin-3-ol 59 (334 μmol), AcOH (445 μmol), and 2-picoline-borane complex (445 μmol), 2 mL MeOH; reaction time: 16 h and in addition: purification by chiral SFC (basic conditions). E47a and E47b are isolated as single stereoisomers. The absolute configuration of the hydroxy substituent is not known.

Example E47a
Analytical HPLC-MS Method: D

| | |
|---|---|
| Rₜ [min]: 1.08 | MS [m/z]: 549 [M + H]⁺ |

Analytical SFC method: P

| | |
|---|---|
| Rₜ [min]: 4.80 | d.e. >96% |

¹H NMR (400 MHz, DMSO-d₆) δ (ppm):
1.08-1.12 (3 H), 1.28-1.48 (3 H), 1.50-1.68 (6 H),
1.78-1.88 (2 H), 2.21-2.34 (2 H), 2.37-2.46 (2 H),
2.79-2.91 (2 H), 3.97-4.06 (1 H), 4.31-4.44 (2 H),
5.36-5.48 (1 H), 7.10-7.15 (1 H), 7.37-7.45 (1 H), 7.64-7.71 (1 H),
7.80-7.86 (1 H), 8.48-8.54 (1 H), 9.05-9.14 (2 H), missing proton(s)
presumably hidden by/overlapping with solvent signals.

Example E47b
Analytical HPLC-MS Method: D

| | |
|---|---|
| Rₜ [min]: 1.08 | MS [m/z]: 549 [M + H]⁺ |

Analytical SFC method: P

| | |
|---|---|
| Rₜ [min]: 5.40 | d.e. >93% |

¹H NMR (400 MHz, DMSO-d₆) δ (ppm):
1.08-1.12 (3 H), 1.29-1.49 (3 H), 1.50-1.66 (6 H), 1.76-1.88
(2 H), 2.21-2.35 (2 H), 2.37-2.46 (2 H), 2.79-2.90
(2 H), 3.98-4.05 (1 H), 4.31-4.44 (2 H), 5.37-5.47
(1 H), 7.10-7.16 (1 H), 7.37-7.45 (1 H), 7.64-7.71 (1 H), 7.79-7.87
(1 H), 8.48-8.53 (1 H), 9.05-9.13 (2 H), missing proton(s) presumably
hidden by/overlapping with solvent signals.

### Synthesis of Example E48

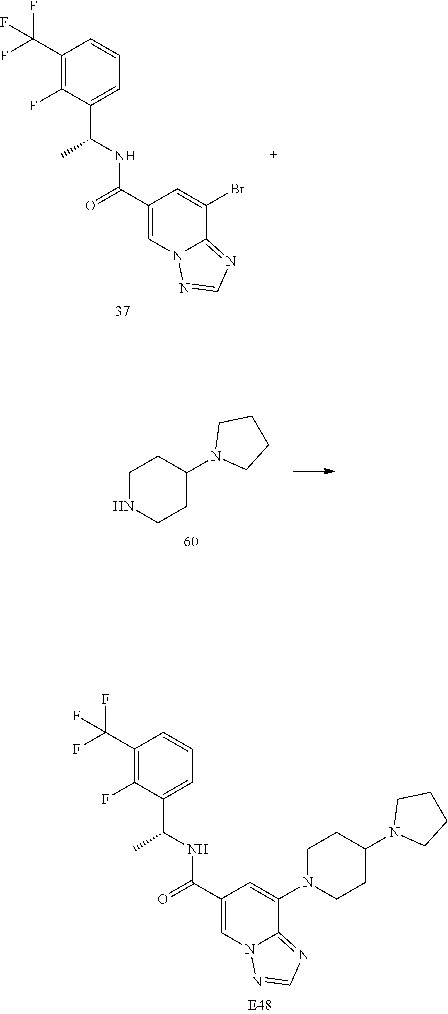 [SMILES: C[C@@H](NC(=O)c1cc(Br)c2ncnn2c1)c1cccc(C(F)(F)F)c1F ; C1CCN(C2CCNCC2)C1 ; C[C@@H](NC(=O)c1cc(N2CCC(N3CCCC3)CC2)c2ncnn2c1)c1cccc(C(F)(F)F)c1F]

A mixture of intermediate 37 (50 mg, 116 μmol), 4-(1-pyrrolidinyl)piperidine 60 (36 mg, 232 μmol), Cs₂CO₃ (94 mg, 290 μmol) in 1,4-dioxane (4 mL) is degassed with argon. Catalyst I (4 mg, 5 μmol) is added, and the mixture is stirred under argon at 90° C. for 16 h. The mixture is filtered and purified by prep. RP-HPLC (basic conditions) to obtain example E48.

Example E48
HPLC-MS Method: E

| | |
|---|---|
| Rₜ [min]: 0.90 | MS [m/z]: 505 [M + H]⁺ |

Analytical SFC method: S

| | |
|---|---|
| Rₜ [min]: 7.15 | e.e. >98% |

1H NMR (400 MHz, DMSO-d₆) δ (ppm):
1.49-1.76 (9 H), 1.92-2.02 (2 H), 2.15-2.25 (1 H), 2.93-3.04
(2 H), 4.14-4.27 (2 H), 5.35-5.47 (1 H), 7.11-7.17
(1 H), 7.37-7.45 (1 H), 7.63-7.71 (1 H), 7.79-7.87
(1 H), 8.48-8.52 (1 H), 9.04-9.08 (1 H), 9.08-9.16 (1 H),
missing proton(s) presumably hidden by/overlapping with solvent signals.

### Synthesis of Example E49

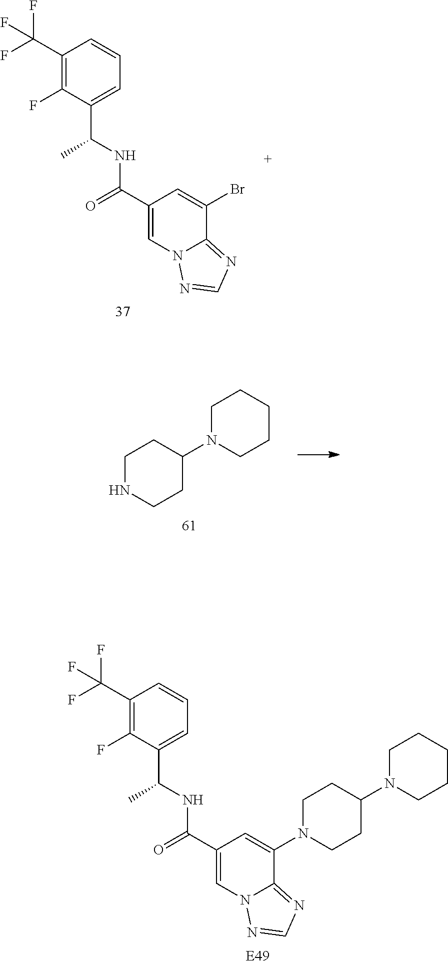 [SMILES: C[C@@H](NC(=O)c1cc(Br)c2ncnn2c1)c1cccc(C(F)(F)F)c1F ; C1CCN(C2CCNCC2)CC1 ; C[C@@H](NC(=O)c1cc(N2CCC(N3CCCCC3)CC2)c2ncnn2c1)c1cccc(C(F)(F)F)c1F]

Example E49 is prepared in analogy to example E48: Intermediate 37 (116 μmol), 4-piperidinopiperidine 61 (232 μmol), Cs₂CO₃ (290 μmol), and catalyst I (5 μmol), 2 mL 1,4-dioxane.

Example E49
HPLC-MS Method: E

| | |
|---|---|
| Rₜ [min]: 0.92 | MS [m/z]: 519 [M + H]⁺ |

Analytical SFC method: S

| | |
|---|---|
| Rₜ [min]: 7.55 | e.e. >98% |

¹H NMR (400 MHz, DMSO-d₆) δ (ppm):
1.32-1.71 (11 H), 1.81-1.89 (2 H), 2.38-2.49 (4 H), 2.75-2.87
(2 H), 4.33-4.45 (2 H), 5.37-5.47 (1 H), 7.10-7.14 (1 H),
7.37-7.44 (1 H), 7.64-7.71 (1 H), 7.79-7.87 (1 H), 8.47-
8.55 (1 H), 9.04-9.08 (1 H), 9.08-9.16 (1 H), missing proton(s)
presumably hidden by/overlapping with solvent signals.

### Synthesis of Example E50

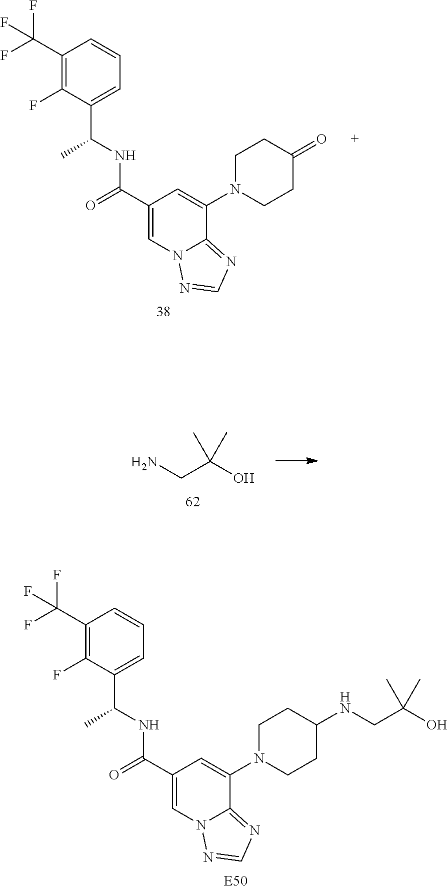

2-Picoline borane complex (12 mg, 111 μmol) is added to a mixture of intermediate 38 (50 mg, 111 μmol), 1-amino-2-methyl-propan-2-ol 62 (21 mg, 223 μmol) and AcOH (60 μL, 1.03 mmol) in MeOH (1 mL) and the resulting mixture is stirred for 16 h at rt. The mixture is neutralized with aqueous NaOH solution (4 N), diluted with MeOH, and purified by prep. RP-HPLC (basic conditions) to obtain example E50.

Example E50
HPLC-MS Method: E

| | |
|---|---|
| Rₜ [min]: 0.89 | MS [m/z]: 523 [M + H]⁺ |

Analytical SFC method: V

| | |
|---|---|
| Rₜ [min]: 6.91 | e.e. >98% |

¹H NMR (400 MHz, DMSO-d₆) δ (ppm):
1.02-1.15 (6 H), 1.35-1.57 (6 H), 1.89-2.00 (2 H), 2.42-
2.48 (2 H), 2.55-2.64 (1 H), 2.94-3.05 (2 H), 4.09-4.17 (1 H),
4.17-4.29 (2 H), 5.37-5.48 (1 H), 7.07-7.17 (1 H), 7.38-
7.44 (1 H), 7.64-7.72 (1 H), 7.79-7.87 (1 H), 8.49-
8.57 (1 H), 9.03-9.08 (1 H), 9.08-9.16 (1 H).

### Synthesis of Intermediate Cis-64

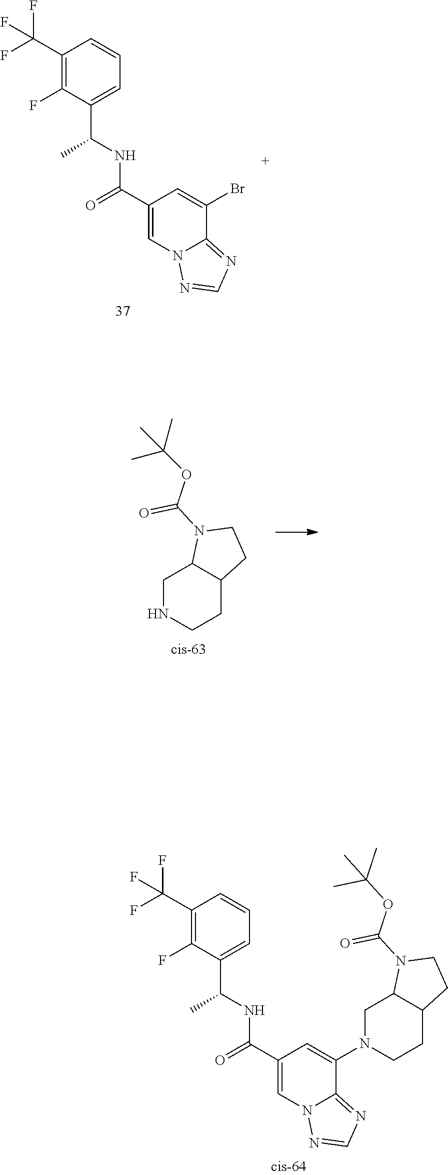 [SMILES: C[C@@H](NC(=O)c1cc(Br)c2ncnn2c1)c1cccc(C(F)(F)F)c1F ; CC(C)(C)OC(=O)N1CCC2CCNCC21 ; C[C@@H](NC(=O)c1cc(N2CCC3CCN(C(=O)OC(C)(C)C)C3C2)c2ncnn2c1)c1cccc(C(F)(F)F)c1F]

A mixture of intermediate 37 (130 mg, 271 μmol), tert-butyl octahydro-1H-pyrrolo[2,3-c]pyridine-1-carboxylate cis-63 (84 mg, 353 μmol) is degassed with argon. Cs₂CO₃ (354 mg, 1.1 mmol) in 1,4-dioxane (2.3 mL) and catalyst I (22.8 mg, 27 μmol) are added, and the mixture is heated under argon for 16 h at 120° C. The mixture is diluted with MeOH, filtered, and purified by prep. RP-HPLC (basic conditions) to give intermediate cis-64 as mixture of stereoisomers.

Intermediate cis-64
Analytical HPLC-MS Method: D

| | |
|---|---|
| Rₜ [min]: 1.15 | MS [m/z]: 577 [M + H]⁺ |

### Synthesis of Examples Cis-E52a and Cis-E52b

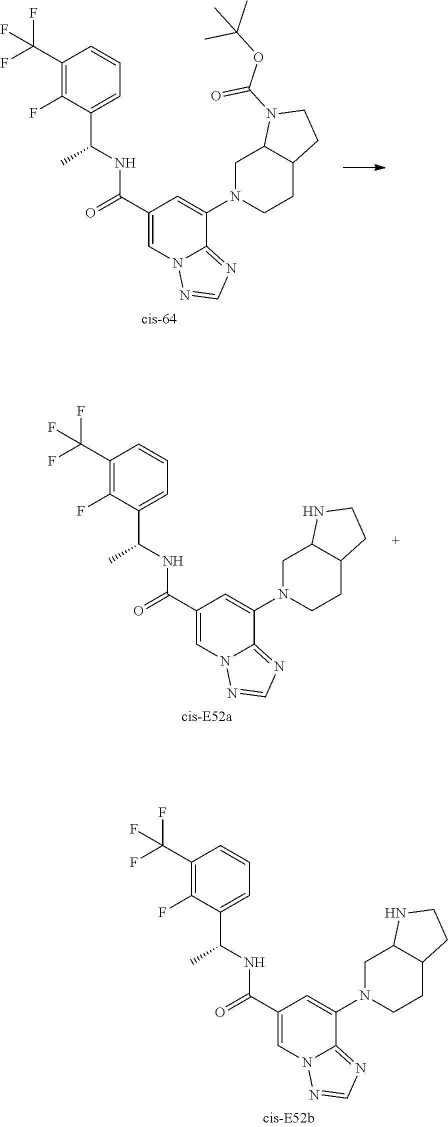 [SMILES: C[C@@H](NC(=O)c1cc(N2CCC3CCN(C(=O)OC(C)(C)C)C3C2)c2ncnn2c1)c1cccc(C(F)(F)F)c1F ; C[C@@H](NC(=O)c1cc(N2CCC3CCNC3C2)c2ncnn2c1)c1cccc(C(F)(F)F)c1F ; C[C@@H](NC(=O)c1cc(N2CCC3CCNC3C2)c2ncnn2c1)c1cccc(C(F)(F)F)c1F]

A mixture of intermediate cis-64 (100 mg, 173 μmol) in 1,4-dioxane (0.3 mL) is treated with HCl in 1,4-dioxane (4 M; 434 μL) and stirring is continued for 2 h at rt. The mixture is diluted with MeOH, neutralized with aqueous NH₃ solution, and purified by prep. RP-HPLC (basic conditions) and chiral SFC to obtain example cis-E52a and example cis-E52b as single stereoisomers. The absolute configuration of the bridgehead carbon atoms is not known; their relative configuration is cis.

Example cis-E52a
Analytical HPLC-MS Method: D

| | |
|---|---|
| Rₜ [min]: 1.00 | MS [m/z]: 477 [M + H]⁺ |

Analytical SFC method: AJ

| | |
|---|---|
| Rₜ [min]: 2.93 | d.e. >97% |

¹H NMR (400 MHz, DMSO-d₆) δ (ppm):
1.40-1.57 (4 H), 1.68-1.94 (3 H), 2.19-2.30 (1 H), 2.75-2.84 (1 H),
2.91-3.02 (1 H), 3.10-3.16 (1 H), 3.38-3.47 (1 H), 3.67-3.77
(1 H), 3.93-4.03 (1 H), 5.36-5.47 (1 H), 7.05-7.09 (1 H),
7.37-7.45 (1 H), 7.64-7.72 (1 H), 7.79-7.86 (1 H),
8.47-8.51 (1 H), 8.98-9.03 (1 H), 9.04-9.12 (1 H), missing
proton(s) presumably hidden by/overlapping with solvent signals.

Example cis-E52b
Analytical HPLC-MS Method: D

| | |
|---|---|
| Rₜ [min]: 1.00 | MS [m/z]: 477 [M + H]⁺ |

Analytical SFC method: AJ

| | |
|---|---|
| Rₜ [min]: 3.30 | d.e. >89% |

¹H NMR (400 MHz, DMSO-d₆) δ (ppm):
1.38-1.58 (4 H), 1.66-1.95 (3 H), 2.19-2.30 (1 H), 2.73-2.84
(1 H), 2.91-3.00 (1 H), 3.09-3.15 (1 H), 3.37-3.47 (1 H), 3.70-
3.79 (1 H), 3.93-4.02 (1 H), 5.34-5.47 (1 H), 7.05-7.08
(1 H), 7.37-7.46 (1 H), 7.63-7.71 (1 H), 7.77-7.87 (1 H),
8.47-8.51 (1 H), 8.98-9.01 (1 H), 9.05-9.10 (1 H), missing
proton(s) presumably hidden by/overlapping with solvent signals.

### Synthesis of Examples Trans-E53a and Trans-E53b

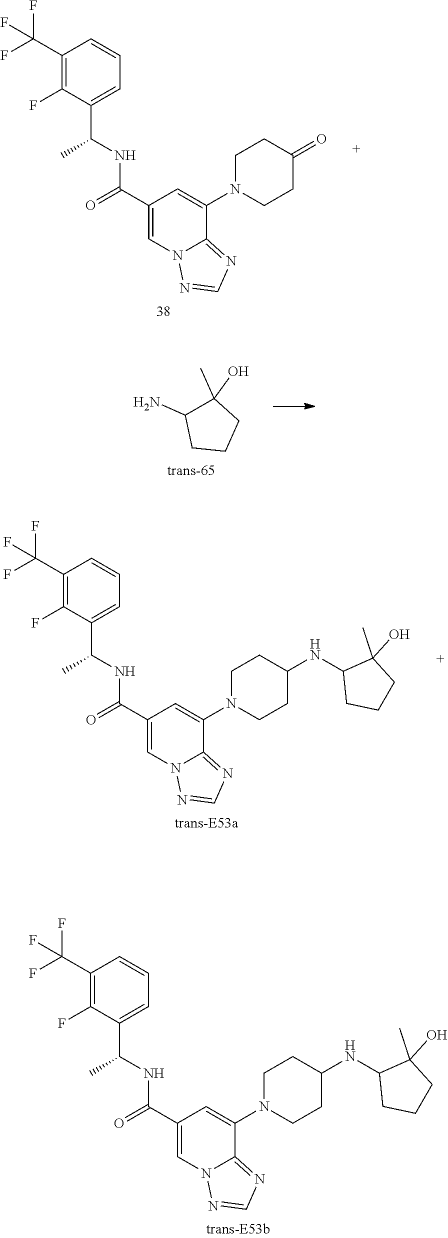

Intermediate 38 (200 mg, 445 μmol) is dissolved in THF (7.2 mL) and 2-amino-1-methylcyclopentan-1-ol trans-65 (103 mg, 890 μmol), AcOH (51 μL, 890 μmol) and molecular sieves 4 Å are added and the mixture is stirred for 1 h at 60° C. After cooling to rt, sodium triacetoxyborohydride (194 mg, 890 μmol) is added and the reaction mixture is stirred 1 h at rt. The reaction mixture is filtered through Celite, the solvent is evaporated, and the mixture is purified by prep. RP-HPLC (basic conditions) and by chiral SFC to obtain example trans-E53a and example trans-E53b as single stereoisomers. The absolute configuration of the amino and hydroxy substituents is not known; their relative configuration is trans.

Example trans-E53a
Analytical HPLC-MS Method: D

| | |
|---|---|
| Rₜ [min]: 1.01 | MS [m/z]: 549 [M + H]⁺ |

Analytical SFC method: AE

| | |
|---|---|
| Rt [min]: 0.85 | d.e. >98% |

¹H NMR (400 MHz, DMSO-d₆) δ (ppm):
1.03-1.13 (3 H), 1.17-1.64 (11 H), 1.86-2.02 (3 H), 2.68-2.79
(1 H), 2.84-2.92 (1 H), 2.92-3.02 (2 H), 4.13-4.32 (3 H), 5.35-5.47
(1 H), 7.10-7.16 (1 H), 7.37-7.44 (1 H), 7.64-7.72 (1 H), 7.79-7.88
(1 H), 8.46-8.55 (1 H), 9.03-9.07 (1 H), 9.07-9.15 (1 H).

Example trans-E53b
Analytical HPLC-MS Method: D

| | |
|---|---|
| Rₜ [min]: 1.01 | MS [m/z]: 549 [M + H]⁺ |

Analytical SFC method: AE

| | |
|---|---|
| Rₜ [min]: 1.17 | d.e. >98% |

¹H NMR (400 MHz, DMSO-d₆) δ (ppm):
1.02-1.11 (3 H), 1.17-1.61 (11 H), 1.83-2.02 (3 H), 2.68-2.79
(1 H), 2.84-2.91 (1 H), 2.91-3.02 (2 H), 4.17-4.29 (3 H), 5.37-
5.47 (1 H), 7.08-7.17 (1 H), 7.37-7.45 (1 H), 7.64-7.72
(1 H), 7.79-7.87 (1 H), 8.48-8.53 (1 H), 9.03-9.07 (1 H),
9.07-9.13 (1 H), missing proton(s) presumably hidden
by/overlapping with solvent signals.

### Synthesis of Example E54

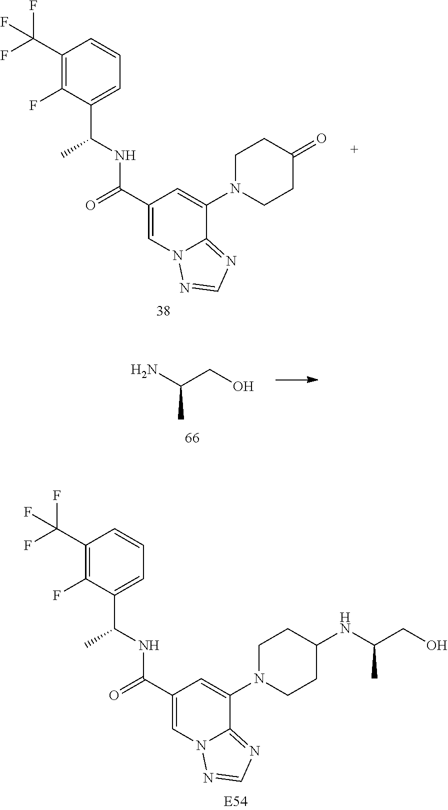

Example E54 is prepared in analogy to example E1: Intermediate 38 (111 μmol), D-alaninol 66 (223 μmol), AcOH (1.03 mmol), and 2-picoline-borane complex (200 μmol), 2 mL MeOH; purification prep. RP-HPLC (basic conditions).

Example E54
Analytical HPLC-MS Method: D

| | |
|---|---|
| Rₜ [min]: 0.96 | MS [m/z]: 509 [M + H]⁺ |

Analytical SFC method: AH

| | |
|---|---|
| Rₜ [min]: 5.51 | d.e. >98% |

¹H NMR (400 MHz, DMSO-d₆) δ (ppm):
0.90-0.98 (3 H), 1.28-1.48 (2 H), 1.50-1.57 (3 H), 1.87-2.01 (2 H),
2.71-2.85 (2 H), 2.92-3.03 (2 H), 3.22-3.26 (2 H), 4.16-4.30
(2 H), 4.33-4.57 (1 H), 5.37-5.48 (1 H), 7.11-7.17 (1 H),
7.39-7.45 (1 H), 7.64-7.73 (1 H), 7.80-7.87 (1 H), 8.48-
8.55 (1 H), 9.03-9.08 (1 H), 9.09-9.13 (1 H), missing
proton(s) presumably hidden by/overlapping with solvent signals.

### Synthesis of Example E55

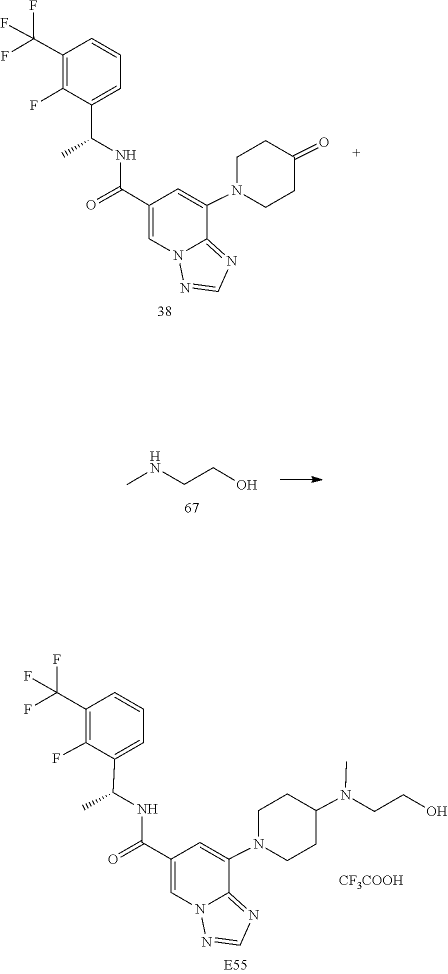

Example E55 is prepared in analogy to example E1: Intermediate 38 (49 μmol), 2-(methylamino)ethanol 67 (98 μmol), AcOH (514 μmol), and 2-picoline-borane complex (49 μmol), 0.5 mL MeOH; reaction time: 16 h; purification by prep. RP-HPLC (acidic conditions).

Example E55
HPLC-MS Method: E

| | |
|---|---|
| Rₜ [min]: 0.79 | MS [m/z]: 509 [M + H]⁺ |

Analytical SFC method: X

| | |
|---|---|
| Rₜ [min]: 4.62 | e.e. >98% |

¹H NMR (400 MHz, DMSO-d₆) δ (ppm):
1.46-1.59 (3 H), 1.76-1.90 (2 H), 2.08-2.21 (2 H), 2.76-2.82 (3 H),
2.83-2.95 (2 H), 3.04-3.13 (1 H), 3.32-3.37 (1 H), 4.48-4.59
(2 H), 5.38-5.47 (1 H), 7.15-7.20 (1 H), 7.37-7.46 (1 H),
7.64-7.71 (1 H), 7.79-7.87 (1 H), 8.50-8.58 (1 H), 9.09-
9.17 (2 H), 9.20-9.29 (1 H), missing proton(s) presumably
hidden by/overlapping with solvent signals.

### Synthesis of Example E57

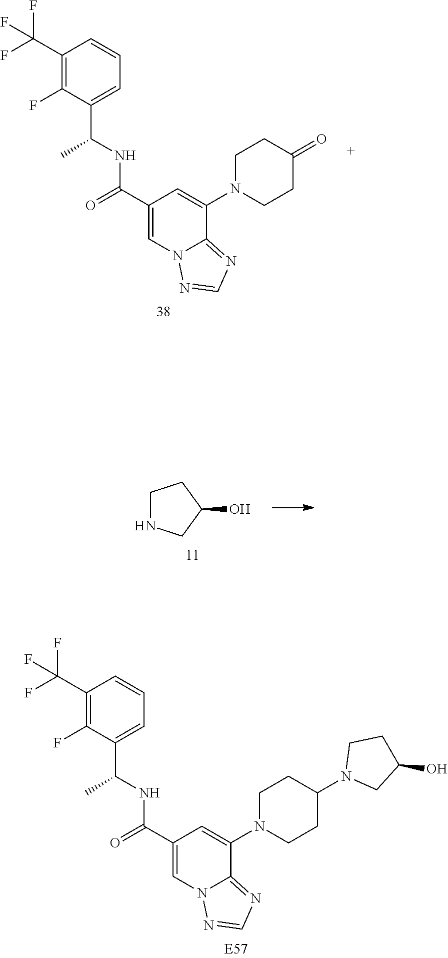 [SMILES: C[C@@H](NC(=O)c1cc(N2CCC(=O)CC2)c2ncnn2c1)c1cccc(C(F)(F)F)c1F ; O[C@@H]1CCNC1 ; C[C@@H](NC(=O)c1cc(N2CCC(N3CC[C@@H](O)C3)CC2)c2ncnn2c1)c1cccc(C(F)(F)F)c1F]

Example E57 is prepared in analogy to example E1: Intermediate 38 (89 μmol), (R)-3-hydroxypyrrolidine 11 (178 μmol), AcOH (1.03 μmol), and 2-picoline-borane complex (89 μmol), 0.6 mL MeOH; purification by prep. RP-HPLC (basic conditions).

Example E57
HPLC-MS Method: E

| | |
|---|---|
| Rₜ [min]: 0.79 | MS [m/z]: 521 [M + H]⁺ |

Analytical SFC method: AN

| | |
|---|---|
| Rₜ [min]: 1.14 | d.e. >98% |

¹H NMR (400 MHz, DMSO-d₆) δ (ppm):
1.47-1.60 (6 H), 1.89-2.01 (3 H), 2.17-2.26 (1 H), 2.35-2.41
(1 H), 2.60-2.69 (1 H), 2.74-2.82 (1 H), 2.93-3.03 (2 H), 4.13-
4.27 (3 H), 4.43-4.86 (1 H), 5.36-5.48 (1 H), 7.10-7.18 (1 H),
7.38-7.45 (1 H), 7.63-7.71 (1 H), 7.79-7.87 (1 H), 8.47-8.54
(1 H), 9.04-9.08 (1 H), 9.08-9.16 (1 H), missing proton(s)
presumably hidden by/overlapping with solvent signals.

### Synthesis Scheme of Intermediate 71

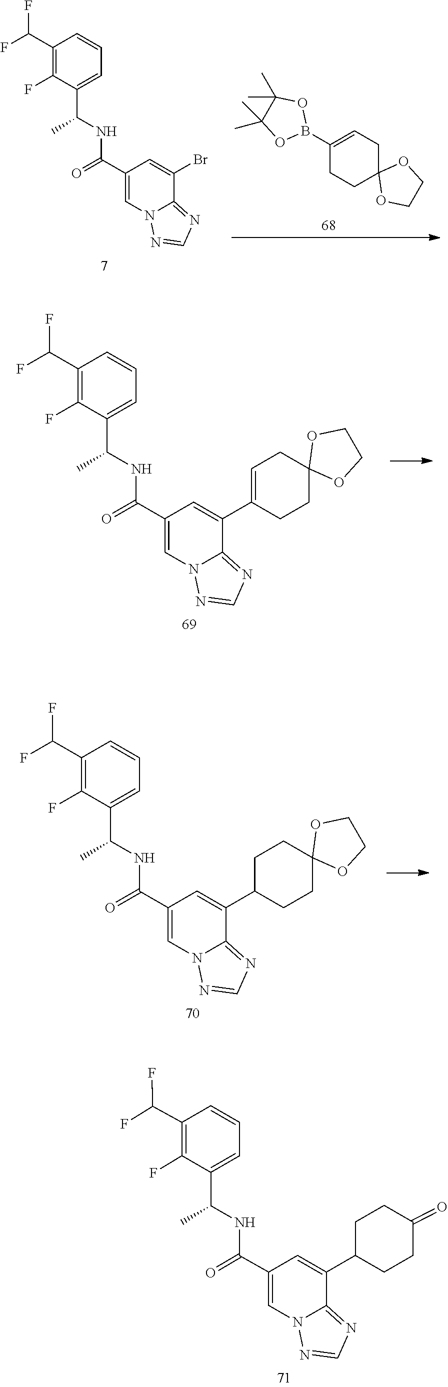 [SMILES: C[C@@H](NC(=O)c1cc(Br)c2ncnn2c1)c1cccc(C(F)F)c1F ; CC1(C)OB(C2=CCC3(CC2)OCCO3)OC1(C)C ; C[C@@H](NC(=O)c1cc(C2=CCC3(CC2)OCCO3)c2ncnn2c1)c1cccc(C(F)F)c1F ; C[C@@H](NC(=O)c1cc(C2CCC3(CC2)OCCO3)c2ncnn2c1)c1cccc(C(F)F)c1F ; C[C@@H](NC(=O)c1cc(C2CCC(=O)CC2)c2ncnn2c1)c1cccc(C(F)F)c1F]

### Synthesis of Intermediate 69

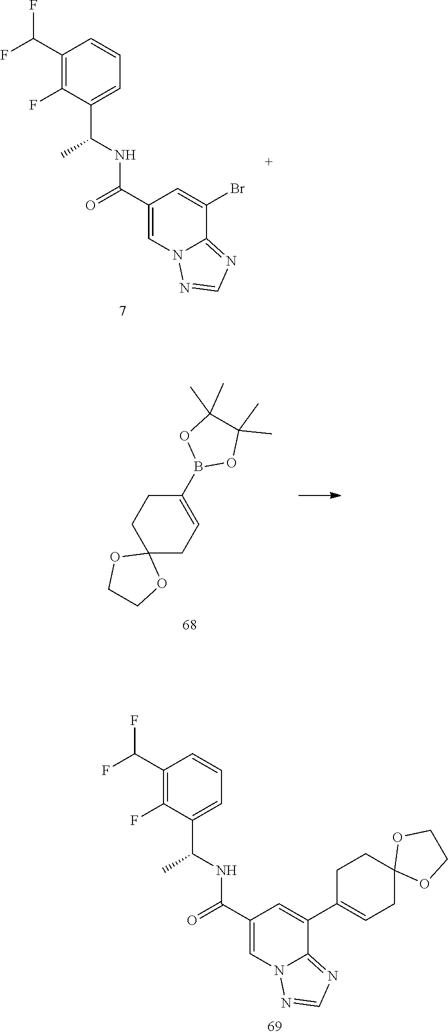 [SMILES: C[C@@H](NC(=O)c1cc(Br)c2ncnn2c1)c1cccc(C(F)F)c1F ; CC1(C)OB(C2=CCC3(CC2)OCCO3)OC1(C)C ; C[C@@H](NC(=O)c1cc(C2=CCC3(CC2)OCCO3)c2ncnn2c1)c1cccc(C(F)F)c1F]

An aqueous Na₂CO₃ solution (2 M; 7.7 mL, 15 mmol), intermediate 7 (2.0 g, 4.8 mmol) and borane 68 (1.6 g, 5.8 mmol) in 1,4-dioxane (24 mL) is stirred under argon atmosphere. Catalyst II (198 mg, 242 μmol) is added, and the mixture is stirred at 95° C. for 1.5 h. Aqueous NaCl solution is added. The layers are separated, and the aqueous layer is extracted with EtOAc. The combined organic layers are washed with saturated NaCl solution, dried over Na₂SO₄, filtered and the solvent is evaporated. The residue is purified by column chromatography (SiO₂; EtOAc/cyclohexane: 60:40→100:0) to obtain intermediate 69.

Intermediate 69
Analytical HPLC-MS Method: D

| | |
|---|---|
| Rₜ [min]: 0.99 | MS [m/z]: 473 [M + H]⁺ |

### Synthesis of Intermediate 70

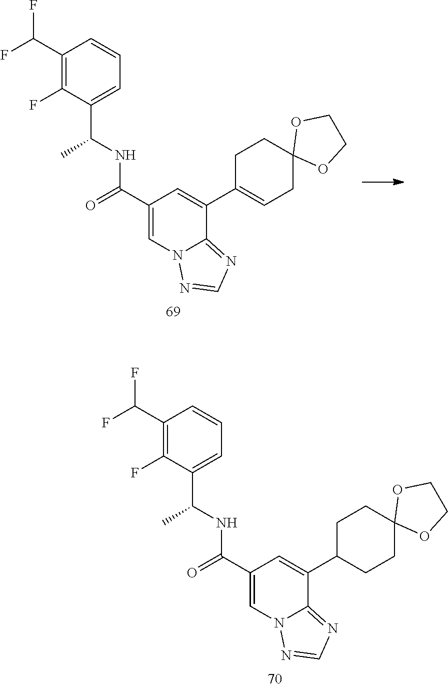 [SMILES: C[C@@H](NC(=O)c1cc(C2=CCC3(CC2)OCCO3)c2ncnn2c1)c1cccc(C(F)F)c1F ; C[C@@H](NC(=O)c1cc(C2CCC3(CC2)OCCO3)c2ncnn2c1)c1cccc(C(F)F)c1F]

A mixture of intermediate 69 (1.6 g, 3.4 mmol) and Pd/C (10% w/w, 200 mg) in MeOH (30 mL) is stirred under H₂ atmosphere (50 psi) for 18 h at rt. The mixture is filtered, and the solvent evaporated to obtain intermediate 70. The material is used in the next reaction step without further purification.

Intermediate 70
HPLC-MS Method: E

| | |
|---|---|
| Rₜ [min]: 1.02 | MS [m/z]: 475 [M + H]⁺ |

### Synthesis of Intermediate 71

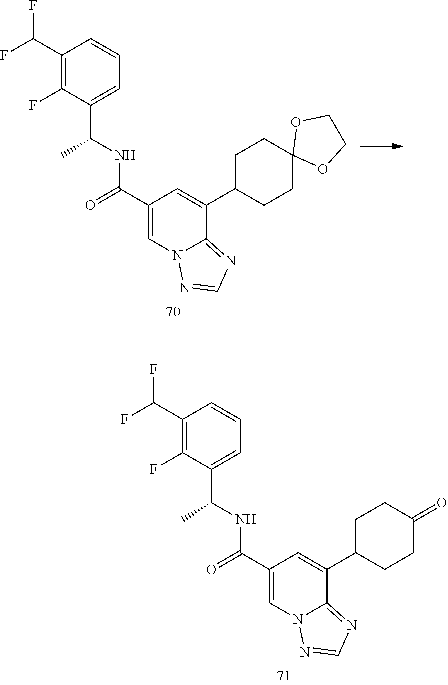 [SMILES: C[C@@H](NC(=O)c1cc(C2CCC3(CC2)OCCO3)c2ncnn2c1)c1cccc(C(F)F)c1F ; C[C@@H](NC(=O)c1cc(C2CCC(=O)CC2)c2ncnn2c1)c1cccc(C(F)F)c1F]

Intermediate 70 (1.5 g, 3.2 mmol) in THF (20 mL) is treated with aqueous HCl solution (4 M; 5 mL, 20 mmol). The mixture is stirred for 24 h at rt. An aqueous NaOH solution (4 M; 5 mL) and saturated NaHCO₃ solution are added, and the mixture is extracted with EtOAc. The separated organic layers are extracted with aqueous saturated NaCl solution. The solvents are evaporated under reduced pressure. The desired material is treated with MTBE and the solvent evaporated to obtain intermediate 71. The material is used in the next reaction step without further purification.

Intermediate 71
Analytical HPLC-MS Method: D

| | |
|---|---|
| Rₜ [min]: 0.93 | MS [m/z]: 431 [M + H]⁺ |

### Synthesis of Example E58

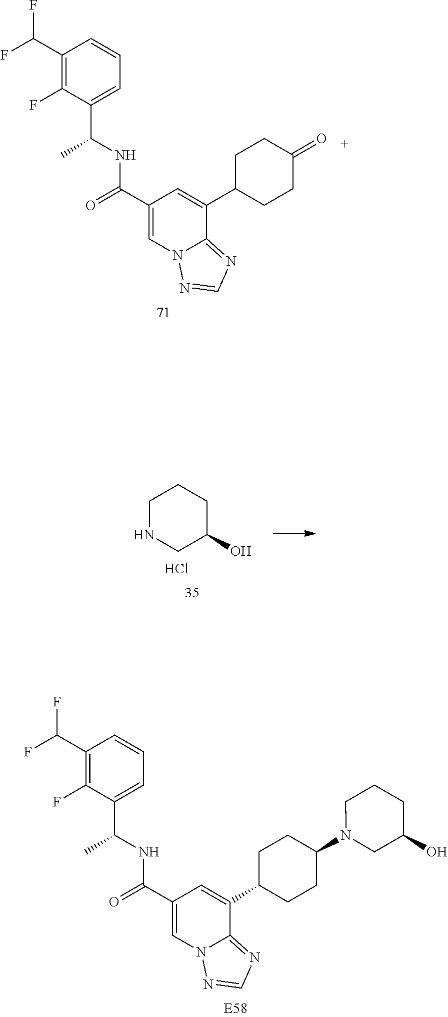 [SMILES: C[C@@H](NC(=O)c1cc(C2CCC(=O)CC2)c2ncnn2c1)c1cccc(C(F)F)c1F ; O[C@@H]1CCCNC1 ; Cl ; C[C@@H](NC(=O)c1cc([C@H]2CC[C@H](N3CCC[C@@H](O)C3)CC2)c2ncnn2c1)c1cccc(C(F)F)c1F]

A mixture of intermediate 71 (160 mg, 372 μmol) and (R)-3-hydroxypiperidine hydrochloride 35 (104 mg, 743 μmol) in MeOH (3 mL) is prepared and AcOH (150 μL, 2.6 mmol) added followed by 2-picoline-borane complex (49 mg, 446 μmol). The mixture is stirred for 1.5 h at rt. An aqueous NaOH solution (4 M) and MeOH are added. The mixture is filtered and directly purified by prep. RP-HPLC (basic conditions) to obtain example E58.

Example E58
Analytical HPLC-MS Method: D

| | |
|---|---|
| Rₜ [min]: 0.95 | MS [m/z]: 516 [M + H]⁺ |

Chiral SFC Method: Y

| | |
|---|---|
| Rt [min]: 5.21 | d.e. > 98% |

¹H NMR (400 MHz, DMSO-d₆) δ (ppm): 0.99-1.11 (1 H), 1.32-1.51 (3 H), 1.51-1.56
(3 H), 1.56-1.84 (4 H), 1.84-1.93 (2 H), 1.94-2.06 (3 H), 2.08-2.18 (1 H), 2.38-2.48
(1 H), 2.65-2.73 (1 H), 2.83-2.92 (1 H), 3.02-3.14 (1 H), 3.38-3.48 (1 H), 4.47-4.55
(1 H), 5.37-5.47 (1 H), 7.07-7.39 (2 H), 7.49-7.57 (1 H), 7.66-7.74 (1 H), 7.80-7.86
(1 H), 8.55-8.62 (1 H), 9.06-9.15 (1 H), 9.37-9.43 (1 H).

### Synthesis of Example E59

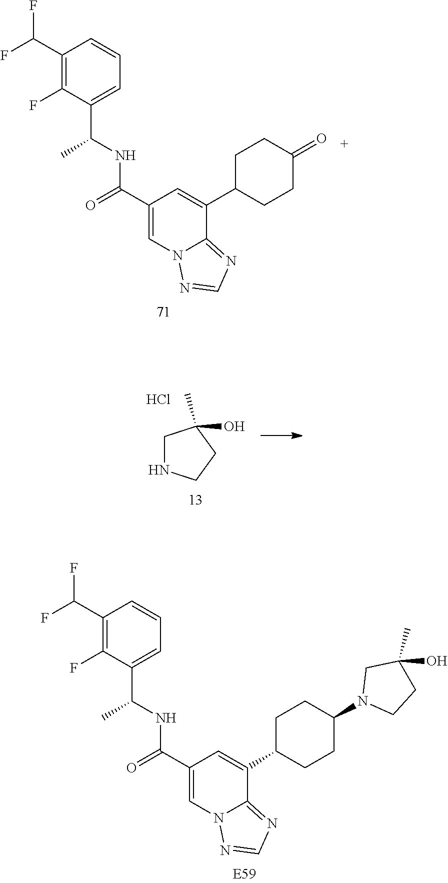

AcOH (11 μL, 186 μmol) is added to a mixture of intermediate 71 (80 mg, 186 μmol) and (3S)-3-methylpyrrolidin-3-ol 13 (40 mg, 279 μmol) in THF (1.5 mL) and the mixture is stirred for 16 h at rt. Sodium triacetoxyborohydride (158 mg, 744 μmol) is added and stirring is continued for 2 h at rt. The crude mixture is directly purified by prep. RP-HPLC (basic conditions) to give example E59.

Example E59
Analytical HPLC-MS Method: D

| | |
|---|---|
| Rₜ [min]: 0.96 | MS [m/z]: 516 [M + H]⁺ |

Chiral SFC Method: AT

| | |
|---|---|
| Rₜ [min]: 0.65 | d.e. > 98% |

¹H NMR (400 MHz, DMSO-d₆) δ (ppm): 1.21-1.41 (5 H), 1.53 (3 H), 1.61-1.79 (4 H),
1.93-2.19 (5 H), 2.67-2.75 (1 H) , 3.05-3.15 (1 H), 4.44 (1 H), 5.42 (1 H), 7.03-7.41
(2 H), 7.53 (1 H), 7.70 (1 H), 7.84 (1 H), 8.58 (1 H), 9.11 (1 H), 9.40 (1 H), missing
proton(s) presumably hidden by/ overlapping with solvent signals.

### Synthesis of Example E60

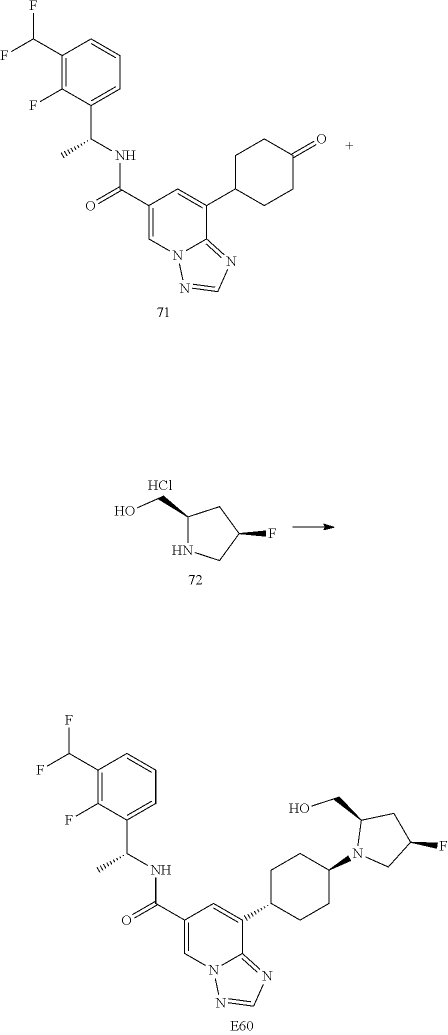 [SMILES: C[C@@H](NC(=O)c1cc(C2CCC(=O)CC2)c2ncnn2c1)c1cccc(C(F)F)c1F ; Cl ; OC[C@H]1C[C@@H](F)CN1 ; C[C@@H](NC(=O)c1cc([C@H]2CC[C@H](N3C[C@H](F)C[C@@H]3CO)CC2)c2ncnn2c1)c1cccc(C(F)F)c1F]

AcOH (13 μL, 232 μmol) is added to a mixture of intermediate 71 (100 mg, 232 μmol) and [(2R,4R)-4-fluoropyrrolidin-2-yl]methanol hydrochloride 72 (56 mg, 349 μmol) in THF (1.9 mL) and the mixture is stirred for 16 h at rt. Sodium triacetoxyborohydride (197 mg, 929 μmol) is added and stirring is continued for 2 h at rt. The crude mixture is directly purified by prep. RP-HPLC to give example E60.

Example E60
Analytical HPLC-MS Method: D

| | |
|---|---|
| Rₜ [min]: 0.94 | MS [m/z]: 534 [M + H]⁺ |

Chiral SFC Method: AK

| | |
|---|---|
| Rₜ [min]: 1.06 | d.e. > 98% |

¹H NMR (400 MHz, DMSO-d₆) δ (ppm): 1.31-1.56 (5 H), 1.64-2.19 (8 H), 2.62-2.88
(2 H), 2.95 (1 H), 3.04-3.23 (3 H), 3.41-3.50 (1 H), 4.41 (1 H), 5.02-5.26 (1 H), 5.42
(1 H), 7.08-7.39 (2 H), 7.53 (1 H), 7.70 (1 H), 7.84 (1 H), 8.58 (1 H), 9.12 (1 H), 9.41 (1
H).

### Synthesis of Examples Trans-E61a and Trans-E61b

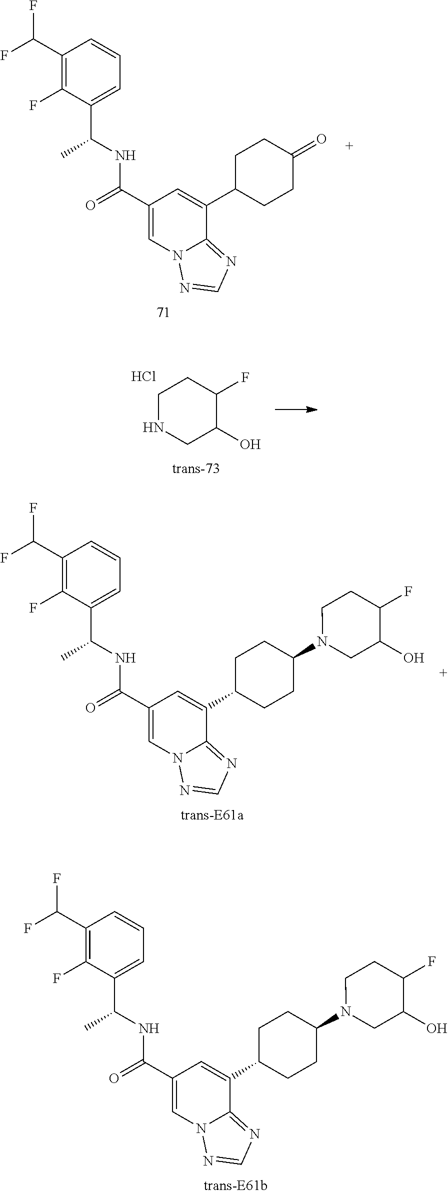 [SMILES: C[C@@H](NC(=O)c1cc(C2CCC(=O)CC2)c2ncnn2c1)c1cccc(C(F)F)c1F ; Cl ; OC1CNCCC1F ; C[C@@H](NC(=O)c1cc([C@H]2CC[C@H](N3CCC(F)C(O)C3)CC2)c2ncnn2c1)c1cccc(C(F)F)c1F ; C[C@@H](NC(=O)c1cc([C@H]2CC[C@H](N3CCC(F)C(O)C3)CC2)c2ncnn2c1)c1cccc(C(F)F)c1F]

A mixture of intermediate 71 (150 mg, 348 μmol) and trans-4-fluoropiperidin-3-ol hydrochloride trans-73 (114 mg, 697 μmol) in AcOH (100 μL, 1.75 mmol) and isopropanol (2 mL) is stirred for 10 min at rt. 2-Picoline-borane complex (58 mg, 523 μmol) is added, and the reaction mixture is stirred for 2 days at rt. An aqueous NaOH solution (4 M) is added, and the mixture is diluted with MeOH, filtered, and directly purified via RP-HPLC (basic conditions) and chiral SFC to obtain examples trans-E61a and trans-61b as single stereoisomers. The absolute configuration of the fluorine and hydroxy substituents is not known; their relative configuration is trans.

Analytical HPLC-MS Method: D

| | |
|---|---|
| Rₜ [min]: 0.95 | MS [m/z]: 534 [M + H]⁺ |

Analytical SFC method: R

| | |
|---|---|
| Rₜ [min]: 3.49 | d.e. > 89% |

¹H NMR (400 MHz, DMSO-d₆) δ (ppm): 1.37 - 1.61 (6 H), 1.65-1.79 (2 H), 1.84-1.93
(2 H), 1.94-2.15 (4 H), 2.26 (1 H), 2.38-2.49 (1 H), 2.74-2.84 (1 H), 2.86-2.95 (1 H),
3.03-3.13 (1 H), 3.41-3.54 (1 H), 4.00-4.32 (1 H), 5.11 (1 H), 5.42 (1 H), 7.07-7.37
(2 H), 7.53 (1 H), 7.70 (1 H), 7.83 (1 H), 8.58 (1 H), 9.11 (1 H), 9.41 (1 H).

Example trans-E61b
Analytical HPLC-MS Method: D

| | |
|---|---|
| Rₜ [min]: 0.95 | MS [m/z]: 534 [M + H]⁺ |

Analytical SFC method: R

| | |
|---|---|
| Rₜ [min]: 4.40 | d.e. > 94% |

¹H NMR (400 MHz, DMSO-d₆) δ (ppm): 1.35-1.62 (6 H), 1.64-1.79 (2 H), 1.83-1.92
(2 H), 1.94-2.14 (4 H), 2.26 (1 H), 2.39-2.49 (1 H), 2.75-2.83 (1 H), 2.85-2.94 (1 H),
3.03-3.14 (1 H), 3.41-3.54 (1 H), 4.07-4.33 (1 H), 5.11 (1 H), 5.42 (1 H), 7.07-7.38
(2 H), 7.55 (1 H), 7.70 (1 H), 7.83 (1 H), 8.58 (1 H), 9.11 (1 H), 9.41 (1 H).

### Synthesis of Example E62

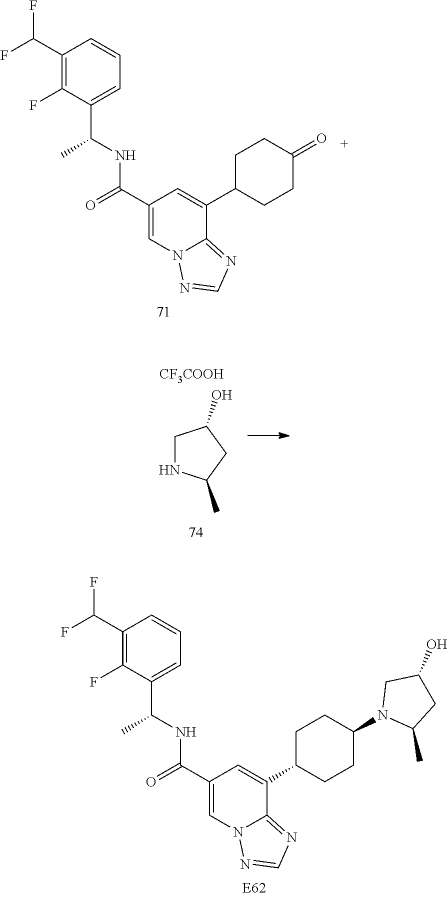

A mixture of intermediate 71 (140 mg, 325 μmol), (3R,5R)-5-methylpyrrolidin-3-ol trifluoroacetate 74 (119 mg, 553 μmol), AcOH (38 μL, 651 μmol), and 2-picoline-borane complex (45 mg, 423 μmol) in MeOH (4 mL) is stirred for 3 days at rt. The mixture is neutralized with aqueous NaOH solution (1 N) and diluted with MeOH. The mixture is separated via prep. RP-HPLC (basic conditions) to give example E62.

Example E62
Analytical HPLC-MS Method: D

| | |
|---|---|
| Rₜ [min]: 0.96 | MS [m/z]: 516 [M + H]⁺ |

Analytical SFC method: AJ

| | |
|---|---|
| Rₜ [min]: 4.83 | d.e. > 98% |

¹H NMR (400 MHz, DMSO-d₆) δ (ppm): 0.98 (3 H), 1.32-1.56 (6 H), 1.64-2.07 (7 H),
2.41-2.47 (1 H), 2.60-2.70 (1 H), 2.98-3.14 (3 H), 4.10-4.18 (1 H), 4.59 (1 H), 5.43
(1 H), 7.09-7.38 (2 H), 7.53 (1 H), 7.70 (1 H), 7.83-7.87 (1 H), 8.58 (1 H), 9.12 (1 H),
9.40 (1 H).

### Synthesis of Intermediate 75

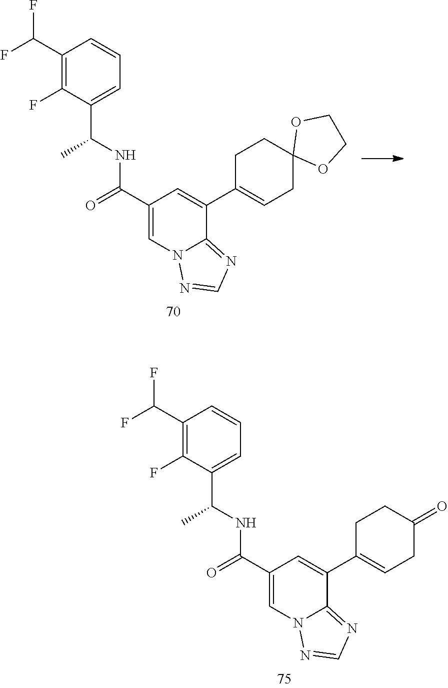 [SMILES: C[C@@H](NC(=O)c1cc(C2=CCC3(CC2)OCCO3)c2ncnn2c1)c1cccc(C(F)F)c1F ; C[C@@H](NC(=O)c1cc(C2=CCC(=O)CC2)c2ncnn2c1)c1cccc(C(F)F)c1F]

An aqueous HCl solution (4 M; 2 mL, 8 mmol) is added to intermediate 70 (690 mg, 1.46 mmol) in THF (10 mL) and the mixture is stirred at rt for 16 h. An aqueous NaOH solution (4 M; 1.8 mL) and saturated NaHCO₃ solution is added, and the mixture is extracted with EtOAc. The combined organic layers are washed with aqueous saturated NaCl solution, and the solvent is evaporated under reduced pressure. The material is purified by column chromatography (SiO₂; EtOAc/cyclohexane: 80:20) to obtain intermediate 75.

Intermediate 75
HPLC-MS Method: E

| | |
|---|---|
| Rₜ [min]: 0.97 | MS [m/z]: 429 [M + H]⁺ |

### Synthesis of Example E63

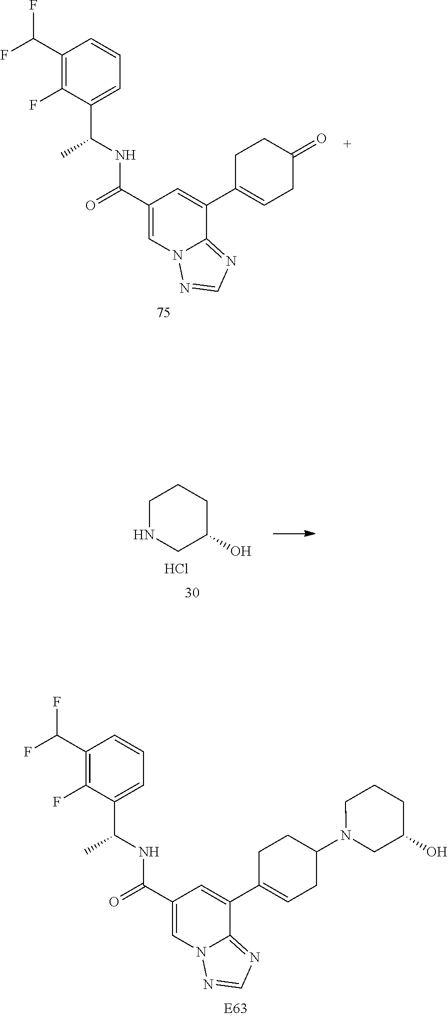 [SMILES: C[C@@H](NC(=O)c1cc(C2=CCC(=O)CC2)c2ncnn2c1)c1cccc(C(F)F)c1F ; O[C@H]1CCCNC1 ; Cl ; C[C@@H](NC(=O)c1cc(C2=CCC(N3CCC[C@H](O)C3)CC2)c2ncnn2c1)c1cccc(C(F)F)c1F]

AcOH (100 μL, 1.7 mmol) and 2-picoline-borane complex (39 mg, 350 μmol) are added to a mixture of intermediate 75 (100 mg, 233 μmol) and (S)-3-hydroxypiperidine hydrochloride 30 (66 mg, 467 μmol) in MeOH (3 mL). The resulting mixture is stirred for 16 h at rt. Additional 2-picoline-borane complex (20 mg) is added, and the reaction mixture is stirred for 2 days at rt. An aqueous NaOH solution (4 M, 400 μL) is added, and the mixture is diluted with MeOH, filtered, and directly purified by prep. RP-HPLC (basic conditions) to obtain example E63. Separation of the diastereomers is possible by standard purification methods.

Example E63
Analytical HPLC-MS Method: D

| | |
|---|---|
| Rₜ [min]: 0.95 | MS [m/z]: 514 [M + H]⁺ |

¹H NMR (400 MHz, DMSO-d₆) δ (ppm): 1.01-1.15 (1 H), 1.32-1.47 (1 H), 1.49-1.68
(5 H), 1.76-1.87 (1 H), 1.90-2.19 (3 H), 2.24-2.46 (2 H), 2.55-2.81 (4 H), 2.85-2.98
(1 H), 3.39-3.52 (1 H), 4.51-4.56 (1 H), 5.38-5.48 (1 H), 7.08-7.40 (3 H), 7.50-7.57
(1 H), 7.67-7.74 (1 H), 7.88-7.93 (1 H), 8.59-8.65 (1 H), 9.12-9.20 (1 H), 9.38-9.44
(1 H).

### Synthesis of Examples E64a and E64b

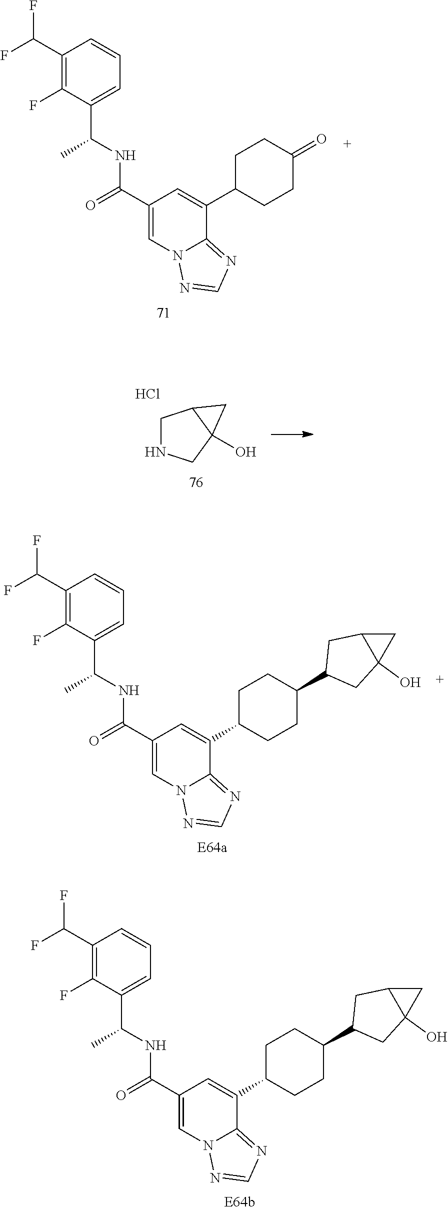 [SMILES: C[C@@H](NC(=O)c1cc(C2CCC(=O)CC2)c2ncnn2c1)c1cccc(C(F)F)c1F ; Cl ; OC12CNCC1C2 ; C[C@@H](NC(=O)c1cc([C@H]2CC[C@H](C3CC4CC4(O)C3)CC2)c2ncnn2c1)c1cccc(C(F)F)c1F ; C[C@@H](NC(=O)c1cc([C@H]2CC[C@H](C3CC4CC4(O)C3)CC2)c2ncnn2c1)c1cccc(C(F)F)c1F]

AcOH (49 μl, 836 μmol) is added to a mixture of intermediate 71 (120 mg, 279 μmol) and 3-azabicyclo[3.1.0]hexan-1-ol hydrochloride 76 (76 mg, 558 μmol) in dimethyl sulfoxide (1 mL) and the mixture is stirred for 10 min at rt. 2-Picoline-borane complex (49 mg, 446 μmol) is added, and the reaction mixture is stirred at rt for 16 h. The mixture is neutralized with aqueous NaOH solution (1 N), diluted with ACN, filtered, and directly purified by prep. RP-HPLC (basic conditions) and chiral SFC to obtain example E64a and E64b as single stereoisomers. The absolute configuration of bridged carbon atoms is not known; the relative configuration of the [3.1.0] ring system is cis.

Example E64a
Analytical HPLC-MS Method: D

| | |
|---|---|
| Rₜ [min]: 0.94 | MS [m/z]: 514 [M + H]⁺ |

Analytical SFC method: AE

| | |
|---|---|
| Rₜ [min]: 1.11 | d.e. > 98% |

¹H NMR (400 MHz, DMSO-d₆) δ (ppm): 0.66 (1 H), 0.79 (1 H), 1.17-1.35 (3 H), 1.53 (3
H), 1.63-1.79 (2 H), 1.92-2.06 (4 H), 2.15-2.25 (1 H), 2.82 (1 H), 3.01-3.13 (2 H),
5.42 (1 H), 5.55 (1 H), 7.09-7.38 (2 H), 7.53 (1 H), 7.70 (1 H), 7.83 (1 H), 8.58 (1 H),
9.12 (1 H), 9.40 (1 H), missing proton(s) presumably hidden by/ overlapping with solvent
signals.

Example E64b
Analytical HPLC-MS Method: D

| | |
|---|---|
| Rₜ [min]: 0.94 | MS [m/z]: 514 [M + H]⁺ |

Analytical SFC method: AE

| | |
|---|---|
| Rₜ [min]: 1.37 | d.e. > 96% |

¹H NMR (400 MHz, DMSO-d₆) δ (ppm): 0.66 (1 H), 0.79 (1 H), 1.17-1.34 (3 H), 1.53 (3
H), 1.64-1.79 (2 H), 1.91-2.05 (4 H), 2.14-2.24 (1 H), 2.45-2.49 (2 H), 2.82 (1 H),
3.01-3.14 (2 H), 5.42 (1 H), 5.54 (1 H), 7.08-7.38 (2 H), 7.53 (1 H), 7.70 (1 H), 7.83 (1
H), 8.58 (1 H), 9.12 (1 H), 9.40 (1 H).

### Synthesis Scheme of Intermediate 79

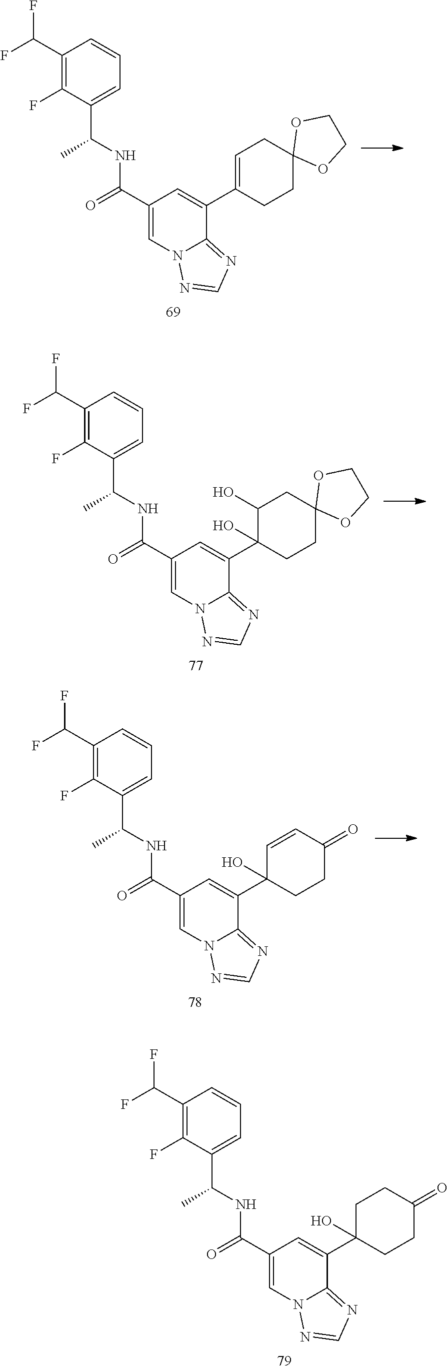 [SMILES: C[C@@H](NC(=O)c1cc(C2=CCC3(CC2)OCCO3)c2ncnn2c1)c1cccc(C(F)F)c1F ; C[C@@H](NC(=O)c1cc(C2(O)CCC3(CC2O)OCCO3)c2ncnn2c1)c1cccc(C(F)F)c1F ; C[C@@H](NC(=O)c1cc(C2(O)C=CC(=O)CC2)c2ncnn2c1)c1cccc(C(F)F)c1F ; C[C@@H](NC(=O)c1cc(C2(O)CCC(=O)CC2)c2ncnn2c1)c1cccc(C(F)F)c1F]

### Synthesis of Intermediate 77

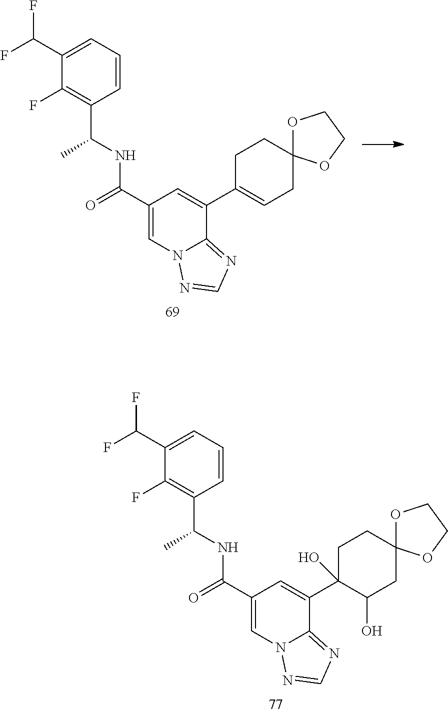 [SMILES: C[C@@H](NC(=O)c1cc(C2=CCC3(CC2)OCCO3)c2ncnn2c1)c1cccc(C(F)F)c1F ; C[C@@H](NC(=O)c1cc(C2(O)CCC3(CC2O)OCCO3)c2ncnn2c1)c1cccc(C(F)F)c1F]

Tert-butanole (103 mL) is added to AD-Mix beta (43 g, 55 mmol) in water (80 mL) and the mixture is stirred 20 min at rt. The mixture is cooled to 0° C. Methanesulfonamide (1.05 g, 11.1 mmol) and a solution of intermediate 69 (5.2 g, 11.1 mmol) in THF (45 mL) are added and stirring is continued for 3 days at rt. An aqueous Na₂S2O₃ solution (10%; 100 mL) is added and stirring continued for 30 min at rt. EtOAc and saturated aqueous NaHCO₃ solution are added, the organic layer is washed with saturated aqueous NaHCO₃ solution and dried over MgSO₄. After filtering and evaporation of the solvents, intermediate 77 is used for the next step without further purification.

Intermediate 77
Analytical HPLC-MS Method: D

| | |
|---|---|
| Rₜ [min]: 0.89 | MS [m/z]: 507 [M + H]⁺ |

### Synthesis of Intermediate 78

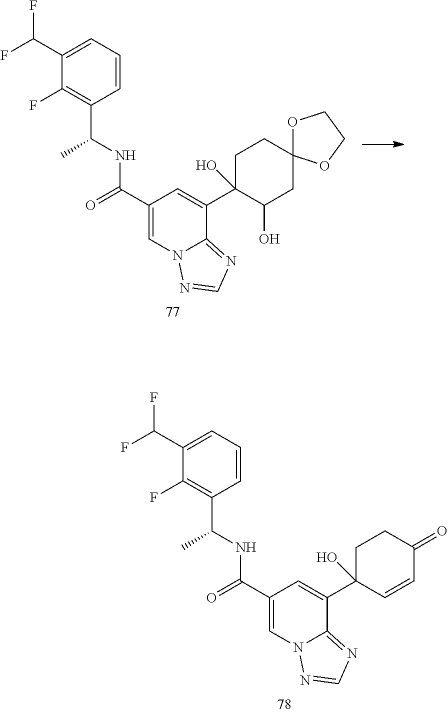 [SMILES: C[C@@H](NC(=O)c1cc(C2(O)CCC3(CC2O)OCCO3)c2ncnn2c1)c1cccc(C(F)F)c1F ; C[C@@H](NC(=O)c1cc(C2(O)C=CC(=O)CC2)c2ncnn2c1)c1cccc(C(F)F)c1F]

Aqueous HCl (4 M; 19 mL) is added to a solution of intermediate 77 (5.6 g, 11.1 mmol) in THF (27 mL) and stirring continued for 48 h at rt. THF is evaporated and the solution is basified with aqueous NH₃ solution. EtOAc and water are added, the organic layer is separated and dried over MgSO₄. After filtration and evaporation, the material is purified by prep. RP-HPLC (acidic conditions) to obtain intermediate 78.

Intermediate 78
HPLC-MS Method: E

| | |
|---|---|
| Rₜ [min]: 0.92 | MS [m/z]: 445 [M + H]⁺ |

### Synthesis of Intermediate 79

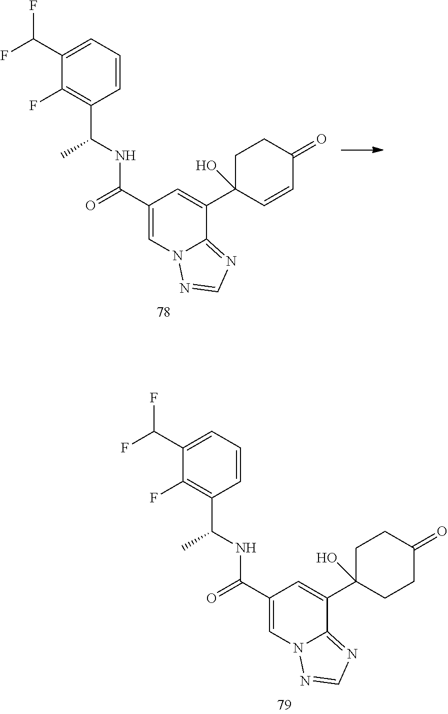 [SMILES: C[C@@H](NC(=O)c1cc(C2(O)C=CC(=O)CC2)c2ncnn2c1)c1cccc(C(F)F)c1F ; C[C@@H](NC(=O)c1cc(C2(O)CCC(=O)CC2)c2ncnn2c1)c1cccc(C(F)F)c1F]

A mixture of intermediate 78 (2.6 g, 5.74 mmol) and Pd/C (10% w/w, 200 mg) in EtOAc (168 mL) is stirred under H₂ atmosphere (30 psi) for 2 h at rt. The mixture is filtered, the solvent evaporated, and the residue purified by prep. RP-HPLC (acidic conditions) to obtain intermediate 79.

Intermediate 79
HPLC-MS Method: E

| | |
|---|---|
| Rₜ [min]: 0.93 | MS [m/z]: 447 [M + H]⁺ |

### Synthesis of Example E65

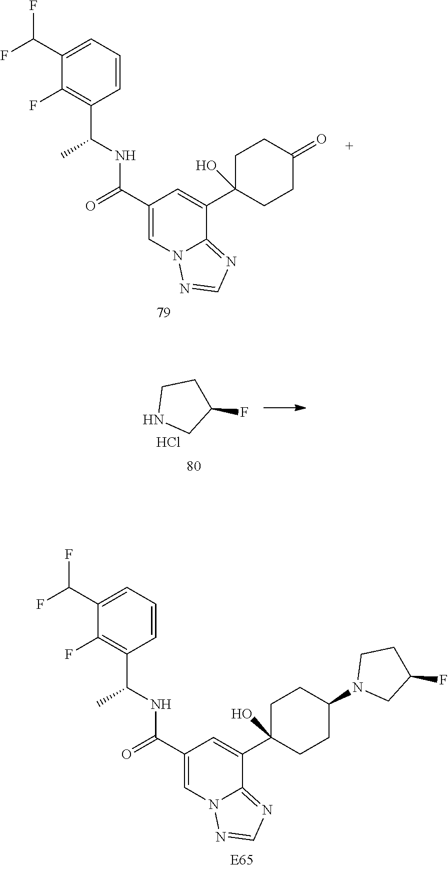 [SMILES: C[C@@H](NC(=O)c1cc(C2(O)CCC(=O)CC2)c2ncnn2c1)c1cccc(C(F)F)c1F ; F[C@@H]1CCNC1 ; Cl ; C[C@@H](NC(=O)c1cc([C@]2(O)CC[C@@H](N3CC[C@@H](F)C3)CC2)c2ncnn2c1)c1cccc(C(F)F)c1F]

Intermediate 79 (100 mg, 224 μmol) is dissolved in 1.8 mL THF. Then (R)-3-fluoropyrrolidine hydrochloride 80 (42 mg, 336 μmol) and AcOH (13 μL, 224 μmol) are added and stirring is continued for 2 h at rt. Then sodium triacetoxyborohydride (189 mg, 896 μmol) is added and the reaction mixture stirred for additional 2 h at rt. Water is added, filtered and the filtrate is directly purified by prep. RP-HPLC (basic conditions) to give example E65.

Example E65
Analytical HPLC-MS Method: D

| | |
|---|---|
| Rₜ [min]: 0.96 | MS [m/z]: 520 [M + H]⁺ |

Analytical SFC method: K

| | |
|---|---|
| Rₜ [min]: 5.78 | d.e. > 98% |

¹H NMR (400 MHz, DMSO-d₆) δ (ppm): 1.44-1.59 (5 H), 1.64-1.95 (5 H), 1.99-2.17
(1 H), 2.19-2.30 (1 H), 2.35-2.46 (1 H), 2.61-2.99 (5 H), 5.06-5.33 (2 H), 5.35-5.55
(1 H), 7.06-7.41 (2 H), 7.46-7.60 (1 H), 7.64-7.82 (1 H), 8.13-8.29 (1 H), 8.60 (1 H),
9.06-9.26 (1 H), 9.39-9.54 (1 H).

### Synthesis of Example E66

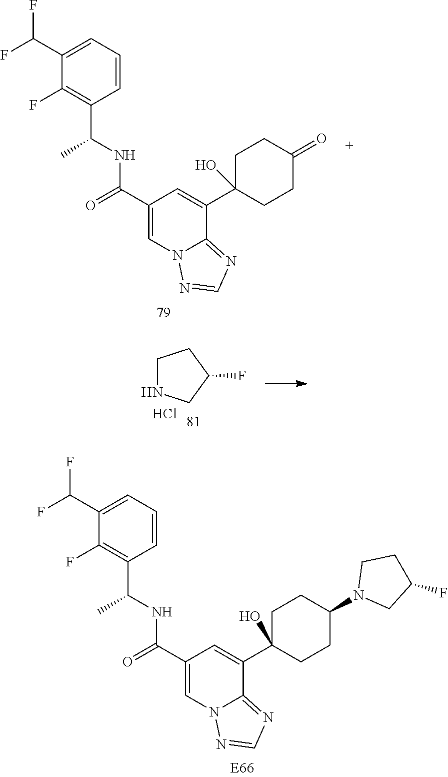 [SMILES: C[C@@H](NC(=O)c1cc(C2(O)CCC(=O)CC2)c2ncnn2c1)c1cccc(C(F)F)c1F ; F[C@H]1CCNC1 ; Cl ; C[C@@H](NC(=O)c1cc([C@]2(O)CC[C@@H](N3CC[C@H](F)C3)CC2)c2ncnn2c1)c1cccc(C(F)F)c1F]

Intermediate 79 (100 mg, 224 μmol) is dissolved in THF (1.8 mL). Then (S)-3-fluoropyrrolidine hydrochloride 81 (42.2 mg, 336 μmol) and AcOH (12.8 μL, 224 μmol) are added and stirring is continued for 2 h at rt. Then sodium triacetoxyborohydride (190 mg, 896 μmol) is added and the reaction mixture stirred for additional 2 h at rt. The mixture is filtered and directly purified by prep. RP-HPLC (basic conditions) to give example E66.

Example E66
Analytical HPLC-MS Method: D

| | |
|---|---|
| Rₜ [min]: 0.96 | MS [m/z]: 520 [M + H]⁺ |

Analytical SFC method: K

| | |
|---|---|
| Rₜ [min]: 6.06 | d.e. > 98% |

1H NMR (400 MHz, DMSO-d₆) δ (ppm): 1.42-1.63 (5 H), 1.64-2.34 (7 H), 2.59-3.03
(5 H), 5.01-5.54 (3 H), 7.03-7.39 (2 H), 7.45-7.81 (2 H), 8.18-8.29 (1 H), 8.51-8.71
(1 H), 9.07-9.24 (1 H), 9.42-9.56 (1 H), missing proton(s) presumably hidden by/
overlapping with solvent signals.

### Synthesis Scheme of Intermediate 84

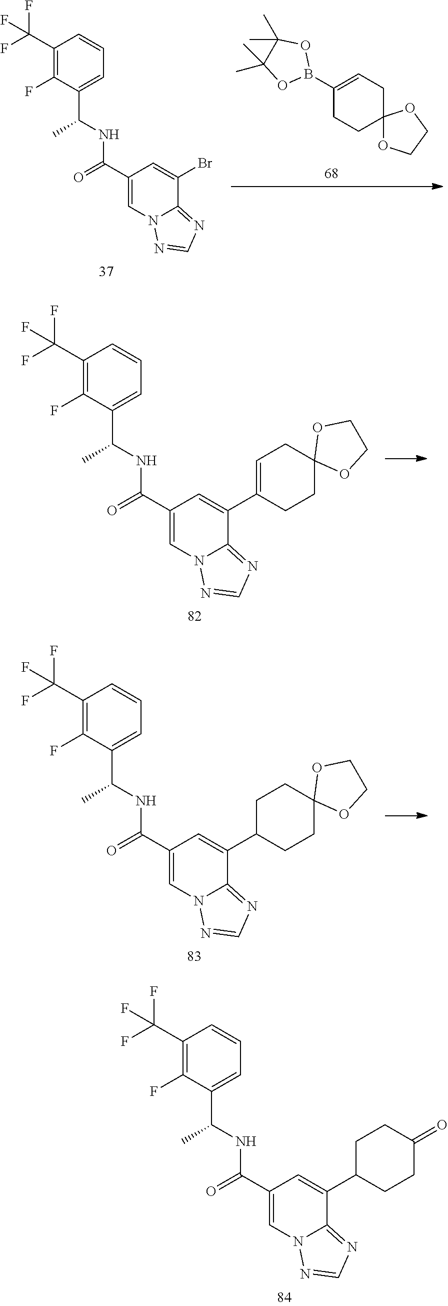 [SMILES: C[C@@H](NC(=O)c1cc(Br)c2ncnn2c1)c1cccc(C(F)(F)F)c1F ; CC1(C)OB(C2=CCC3(CC2)OCCO3)OC1(C)C ; C[C@@H](NC(=O)c1cc(C2=CCC3(CC2)OCCO3)c2ncnn2c1)c1cccc(C(F)(F)F)c1F ; C[C@@H](NC(=O)c1cc(C2CCC3(CC2)OCCO3)c2ncnn2c1)c1cccc(C(F)(F)F)c1F ; C[C@@H](NC(=O)c1cc(C2CCC(=O)CC2)c2ncnn2c1)c1cccc(C(F)(F)F)c1F]

### Synthesis of Intermediate 82

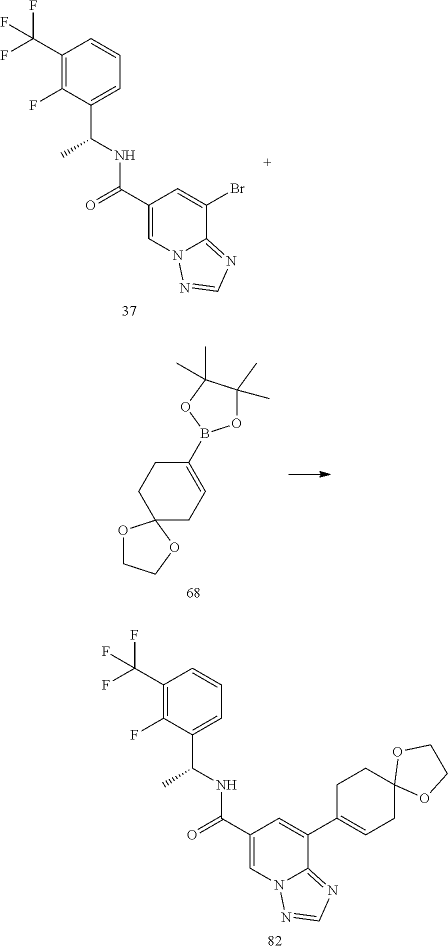 [SMILES: C[C@@H](NC(=O)c1cc(Br)c2ncnn2c1)c1cccc(C(F)(F)F)c1F ; CC1(C)OB(C2=CCC3(CC2)OCCO3)OC1(C)C ; C[C@@H](NC(=O)c1cc(C2=CCC3(CC2)OCCO3)c2ncnn2c1)c1cccc(C(F)(F)F)c1F]

A mixture of an aqueous Na₂CO₃ solution (2 M, 7.0 mL, 14 mmol), intermediate 37 (2.0 g, 4.6 mmol) and borane 68 (1.4 g, 5.1 mmol) in 1,4-dioxane (15 mL) is stirred under argon atmosphere. Catalyst II (227 mg, 0.28 mmol) is added, and the mixture is stirred under argon for 1.5 h at 95° C. Aqueous saturated NaCl solution is added, and the mixture is extracted with EtOAc. The combined organic layers are washed with saturated NaCl solution, dried over Na₂SO₄, filtered and the solvent is evaporated. The remaining material is purified by column chromatography (SiO₂; EtOAc/cyclohexane: 50:50→100:0) to obtain intermediate 82.

Intermediate 82
Analytical HPLC-MS Method: D

| | |
|---|---|
| Rₜ [min]: 1.04 | MS [m/z]: 491 [M + H]⁺ |

### Synthesis of Intermediate 83

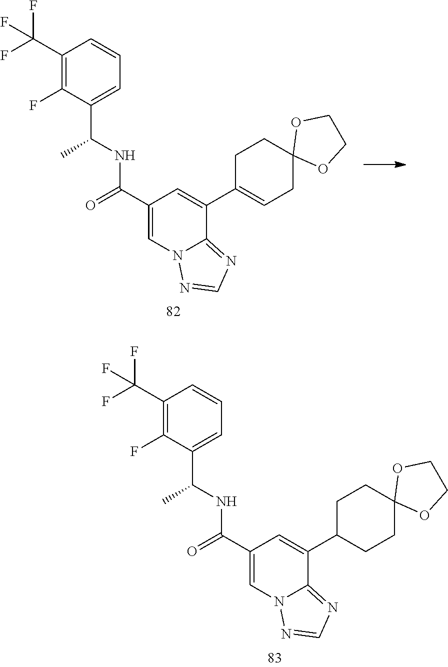 [SMILES: C[C@@H](NC(=O)c1cc(C2=CCC3(CC2)OCCO3)c2ncnn2c1)c1cccc(C(F)(F)F)c1F ; C[C@@H](NC(=O)c1cc(C2CCC3(CC2)OCCO3)c2ncnn2c1)c1cccc(C(F)(F)F)c1F]

Intermediate 82 (2.1 g, 4.3 mmol) in MeOH (50 mL) and Pd/C (10% w/w, 200 mg) is stirred under H₂ atmosphere (50 psi) for 18 h at rt. The mixture is filtered, and the solvent evaporated to obtain intermediate 83. The material is used in the next reaction step without further purification.

Intermediate 83
Analytical HPLC-MS Method: D

| | |
|---|---|
| Rₜ [min]: 1.04 | MS [m/z]: 493 [M + H]⁺ |

### Synthesis of Intermediate 84

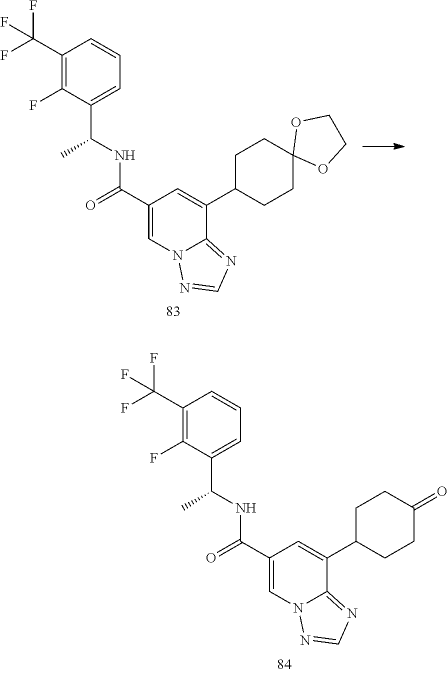 [SMILES: C[C@@H](NC(=O)c1cc(C2CCC3(CC2)OCCO3)c2ncnn2c1)c1cccc(C(F)(F)F)c1F ; C[C@@H](NC(=O)c1cc(C2CCC(=O)CC2)c2ncnn2c1)c1cccc(C(F)(F)F)c1F]

Intermediate 83 (2.1 g, 4.3 mmol) in THF (20 mL) is treated with aqueous HCl solution (4 M; 5.3 mL). The mixture is stirred for 16 h at rt. Aqueous NaOH solution and aqueous saturated NaHCO₃ solution are added, and the mixture is extracted with EtOAc. The separated organic layer is washed with aqueous saturated NaCl solution. The organic layer is evaporated under reduced pressure. The remaining material is purified by column chromatography (SiO₂; EtOAc/MeOH: 100:0→80:20) to obtain intermediate 84.

Intermediate 84
HPLC-MS Method: E

| | |
|---|---|
| Rₜ [min]: 1.01 | MS [m/z]: 449 [M + H]⁺ |

### Synthesis of Example E67

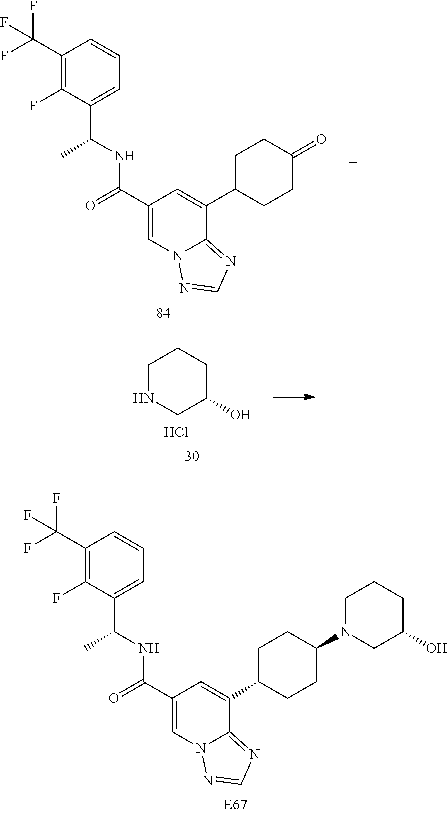 [SMILES: C[C@@H](NC(=O)c1cc(C2CCC(=O)CC2)c2ncnn2c1)c1cccc(C(F)(F)F)c1F ; O[C@H]1CCCNC1 ; Cl ; C[C@@H](NC(=O)c1cc([C@H]2CC[C@H](N3CCC[C@H](O)C3)CC2)c2ncnn2c1)c1cccc(C(F)(F)F)c1F]

A mixture of intermediate 84 (60 mg, 134 μmol) and (S)-3-hydroxypiperidine hydrochloride 30 (50 mg, 352 μmol) in MeOH (2 mL) is prepared. AcOH (16 μL, 268 μmol) and 2-picoline-borane complex (15 mg, 134 μmol) are added. The mixture is stirred for 16 h at rt. Additional AcOH (30 μL) and 2-picoline-borane complex (15 mg, 134 μmol) are added and the mixture is stirred for 24 h at rt. (S)-3-Hydroxypiperidine hydrochloride 30 (20 mg, 141 μmol) and 2-picoline-borane complex (20 mg, 179 μmol) are added, and the mixture is stirred for 24 h at rt. An aqueous NaOH solution (4 M; 230 μL) and MeOH are added. The mixture is filtered and directly purified by reversed phase (basic conditions) to obtain example E67.

Example E67
Analytical HPLC-MS Method: D

| | |
|---|---|
| Rₜ [min]: 1.00 | MS [m/z]: 534 [M + H]⁺ |

Analytical SFC method: Y

| | |
|---|---|
| Rₜ [min]: 3.28 | d.e. > 98% |

¹H NMR (400 MHz, DMSO-d₆) δ (ppm): 0.99-1.12 (1 H), 1.31-1.52 (3 H), 1.52-1.58
(3 H), 1.58-1.85 (4 H), 1.85-1.94 (2 H), 1.94-2.07 (3 H), 2.09-2.19 (1 H), 2.39-2.47
(1 H), 2.64-2.73 (1 H), 2.84-2.91 (1 H), 3.03-3.13 (1 H), 3.40-3.48 (1 H), 4.33-4.71
(1 H), 5.39-5.48 (1 H), 7.37-7.45 (1 H), 7.65-7.72 (1 H), 7.80-7.88 (2 H), 8.56-8.61
(1 H), 9.13-9.22 (1 H), 9.38-9.44 (1 H).

### Synthesis of Example E68

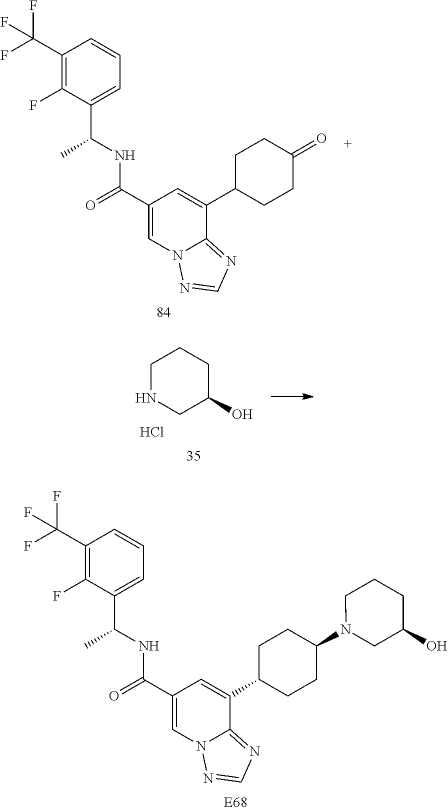 [SMILES: C[C@@H](NC(=O)c1cc(C2CCC(=O)CC2)c2ncnn2c1)c1cccc(C(F)(F)F)c1F ; O[C@@H]1CCCNC1 ; Cl ; C[C@@H](NC(=O)c1cc([C@H]2CC[C@H](N3CCC[C@@H](O)C3)CC2)c2ncnn2c1)c1cccc(C(F)(F)F)c1F]

To a mixture of intermediate 84 (95 mg, 212 μmol) and (R)-3-hydroxypiperidine hydrochloride 35 (59 mg, 424 μmol) in MeOH (2 mL), AcOH (100 μL, 1.72 mmol) and 2-picoline-borane complex (25 mg, 222 μmol) are added. The mixture is stirred for 16 h at rt. (R)-3-Hydroxypiperidine hydrochloride 35 (20 mg, 141 μmol) and 2-picoline-borane complex (20 mg, 179 μmol) are added, and the mixture is stirred for 24 h at rt. The mixture is neutralized with an aqueous NaOH solution (4 M; 400 μL) and MeOH is added. The solids are filtered off and the filtrate is purified by prep. RP-HPLC (basic conditions) to obtain example E68.

Example E68
Analytical HPLC-MS Method: D

| | |
|---|---|
| Rₜ [min]: 1.00 | MS [m/z]: 534 [M + H]⁺ |

Analytical SFC method: S

| | |
|---|---|
| Rₜ [min]: 7.04 | d.e. > 98% |

¹H NMR (400 MHz, DMSO-d₆) δ (ppm): 0.98-1.13 (1 H), 1.30-1.51 (3 H), 1.51-1.58
(3 H), 1.58-1.84 (4 H), 1.84-1.93 (2 H), 1.93-2.06 (3 H), 2.09-2.18 (1 H), 2.39-2.48
(1 H), 2.64-2.74 (1 H), 2.82-2.90 (1 H), 3.03-3.14 (1 H), 3.41-3.48 (1 H), 4.40-4.61
(1 H), 5.37-5.49 (1 H), 7.36-7.47 (1 H), 7.63-7.74 (1 H), 7.81-7.88 (2 H), 8.56-8.61
(1 H), 9.14-9.21 (1 H), 9.37-9.44 (1 H).

### Synthesis of Example E69

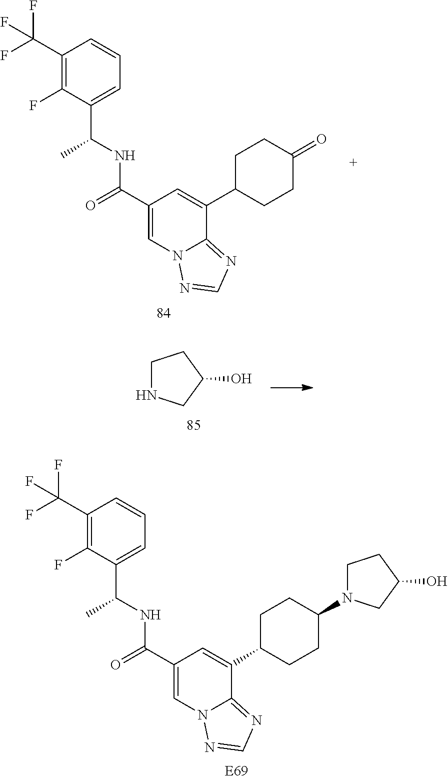 [SMILES: C[C@@H](NC(=O)c1cc(C2CCC(=O)CC2)c2ncnn2c1)c1cccc(C(F)(F)F)c1F ; O[C@H]1CCNC1 ; C[C@@H](NC(=O)c1cc([C@H]2CC[C@H](N3CC[C@H](O)C3)CC2)c2ncnn2c1)c1cccc(C(F)(F)F)c1F]

To a mixture of intermediate 84 (95 mg, 212 μmol) and (S)-3-hydroxypyrrolidine 85 (37 mg, 424 μmol) in MeOH (2 mL), AcOH (100 μL, 1.7 mmol) and 2-picoline-borane complex (25 mg, 222 μmol) are added. The mixture is stirred for 16 h at rt. The mixture is neutralized with an aqueous NaOH solution (4 M) and MeOH is added. The solids are filtered off and the filtrate is purified by prep. RP-HPLC (basic conditions) to obtain example E69.

Example E69
Analytical HPLC-MS Method: D

| | |
|---|---|
| Rₜ [min]: 0.98 | MS [m/z]: 520 [M + H]⁺ |

Analytical SFC method: T

| | |
|---|---|
| Rₜ [min]: 3.74 | d.e. > 98% |

¹H NMR (400 MHz, DMSO-d₆) δ (ppm): 1.24-1.40 (2 H), 1.47-1.58 (4 H), 1.65-1.79
(2 H), 1.90-2.02 (3 H), 2.02-2.15 (3 H), 2.36-2.42 (1 H), 2.61-2.69 (1 H), 2.75-2.82
(1 H), 3.03-3.15 (1 H), 4.12-4.22 (1 H), 4.45-4.82 (1 H), 5.37-5.48 (1 H), 7.37-7.45
(1 H), 7.63-7.72 (1 H), 7.82-7.88 (2 H), 8.56-8.60 (1 H), 9.13-9.22 (1 H), 9.40-9.44
(1 H), missing proton(s) presumably hidden by/ overlapping with solvent signals.

### Synthesis of Example Trans-E70a and Trans-E70b

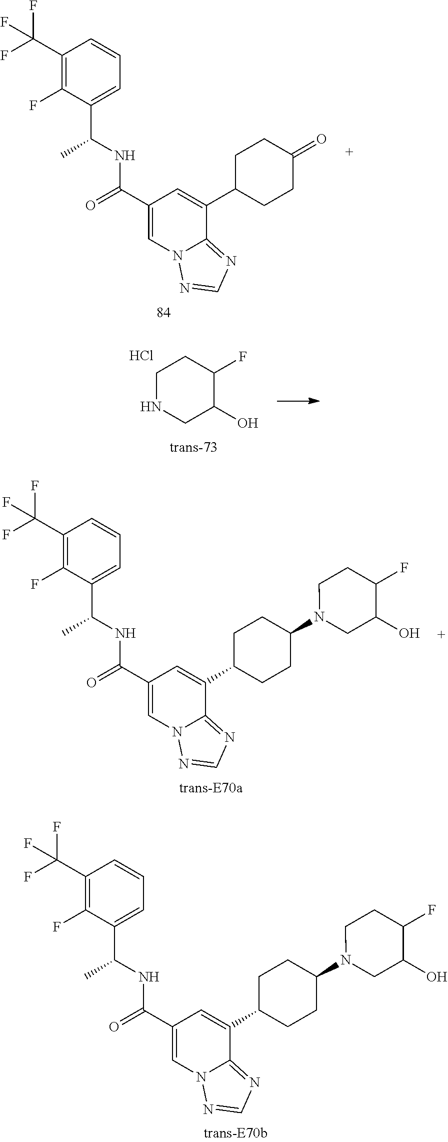 [SMILES: C[C@@H](NC(=O)c1cc(C2CCC(=O)CC2)c2ncnn2c1)c1cccc(C(F)(F)F)c1F ; Cl ; OC1CNCCC1F ; C[C@@H](NC(=O)c1cc([C@H]2CC[C@H](N3CCC(F)C(O)C3)CC2)c2ncnn2c1)c1cccc(C(F)(F)F)c1F ; C[C@@H](NC(=O)c1cc([C@H]2CC[C@H](N3CCC(F)C(O)C3)CC2)c2ncnn2c1)c1cccc(C(F)(F)F)c1F]

Sodium triacetoxyborohydride (180 mg, 849 μmol) is added to a mixture of intermediate 84 (200 mg, 446 μmol) and trans-4-fluoropiperidin-3-ol hydrochloride trans-73 (88 mg, 535 μmol) in DCM. The reaction mixture is stirred for 18 h at rt. The solvent is evaporated, aqueous NaHCO₃ solution is added, and the mixture is extracted with DCM. The combined extracts are purified via prep. RP-HPLC (basic conditions) and by chiral SFC to obtain example trans-E70a and trans-E70b as single stereoisomers. The absolute configuration of the fluorine and hydroxy substituent is not known; their relative configuration is trans.

Example trans-E70a
Analytical HPLC-MS Method: D

| | |
|---|---|
| Rₜ [min]: 1.00 | MS [m/z]: 552 [M + H]⁺ |

Analytical SFC method: AA

| | |
|---|---|
| Rₜ [min]: 0.88 | d.e. > 98% |

¹H NMR (400 MHz, DMSO-d₆) δ (ppm): 1.35- 1.61 (6 H), 1.65-1.78 (2 H), 1.89 (2 H),
1.94-2.12 (4 H), 2.26 (1 H), 2.73-2.83 (1 H), 2.86-2.96 (1 H), 3.01-3.15 (1 H), 3.42-
3.54 (1 H), 4.07-4.29 (1 H), 5.11 (1 H), 5.43 (1 H), 7.41 (1 H), 7.68 (1 H), 7.81-7.87 (2
H), 8.58 (1 H), 9.15 (1 H), 9.42 (1 H), missing proton(s) presumably hidden by/
overlapping with solvent signals.

Example trans-E70b
Analytical HPLC-MS Method: D

| | |
|---|---|
| Rₜ [min]: 1.00 | MS [m/z]: 552 [M + H]⁺ |

Analytical SFC method: AA

| | |
|---|---|
| Rₜ [min]: 1.81 | d.e. > 98% |

¹H NMR (400 MHz, DMSO-d₆) δ (ppm): 1.36-1.62 (6 H), 1.64-1.79 (2 H), 1.88 (2 H),
1.94-2.13 (4 H), 2.26 (1 H), 2.73-2.84 (1 H), 2.86-2.96 (1 H), 3.02-3.14 (1 H), 3.43-
3.54 (1 H), 4.06-4.30 (1 H), 5.11 (1 H), 5.43 (1 H), 7.41 (1 H), 7.68 (1 H), 7.81-7.87 (2
H), 8.58 (1 H), 9.16 (1 H), 9.42 (1 H), missing proton(s) presumably hidden by/
overlapping with solvent signals.

### Synthesis Scheme of Intermediate 88

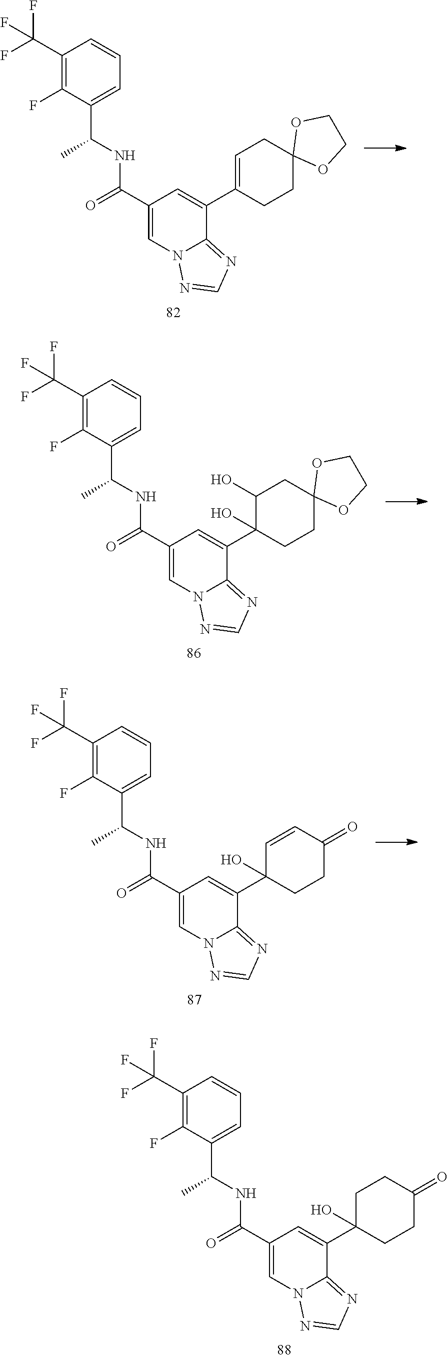 [SMILES: C[C@@H](NC(=O)c1cc(C2=CCC3(CC2)OCCO3)c2ncnn2c1)c1cccc(C(F)(F)F)c1F ; C[C@@H](NC(=O)c1cc(C2(O)CCC3(CC2O)OCCO3)c2ncnn2c1)c1cccc(C(F)(F)F)c1F ; C[C@@H](NC(=O)c1cc(C2(O)C=CC(=O)CC2)c2ncnn2c1)c1cccc(C(F)(F)F)c1F ; C[C@@H](NC(=O)c1cc(C2(O)CCC(=O)CC2)c2ncnn2c1)c1cccc(C(F)(F)F)c1F]

### Synthesis of Intermediate 86

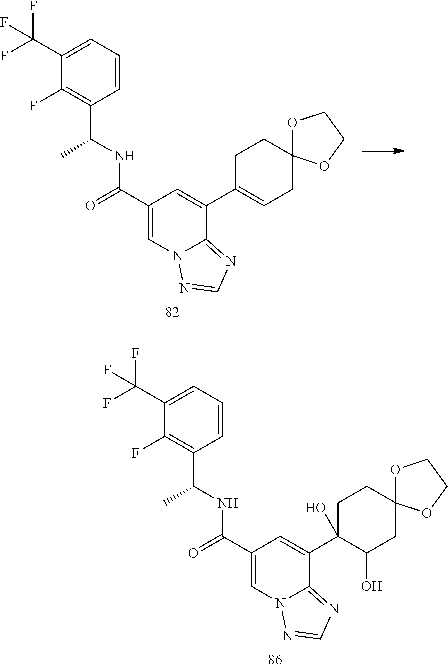 [SMILES: C[C@@H](NC(=O)c1cc(C2=CCC3(CC2)OCCO3)c2ncnn2c1)c1cccc(C(F)(F)F)c1F ; C[C@@H](NC(=O)c1cc(C2(O)CCC3(CC2O)OCCO3)c2ncnn2c1)c1cccc(C(F)(F)F)c1F]

Tert-butanol (95 mL, 1019 mmol) is added to a mixture of AD-Mix beta (39.7 g, 51 mmol) in water (83 mL) and the mixture is stirred for 20 min at rt. The mixture is cooled to 0° C. Methanesulfonamide (970 mg, 10.2 mmol) and a solution of intermediate 82 (5.0 g, 10.2 mol) in THF (83 mL) are added and stirring is continued at rt for 16 h. An aqueous Na₂S₂O₃ solution (10%, 100 mL) is added and stirring continued for 30 min at rt. EtOAc is added, the organic layer washed with saturated aqueous NaHCO₃ solution and dried over MgSO₄. After filtering and evaporation of the solvents, the remaining mixture is purified via column chromatography (SiO₂; EtOAc/petroleum ether: 50/50→0:100) to obtain intermediate 86.

Intermediate 86
Analytical HPLC-MS Method: D

| | |
|---|---|
| Rₜ [min]: 0.94 | MS [m/z]: 525 [M + H]⁺ |

### Synthesis of Intermediate 87

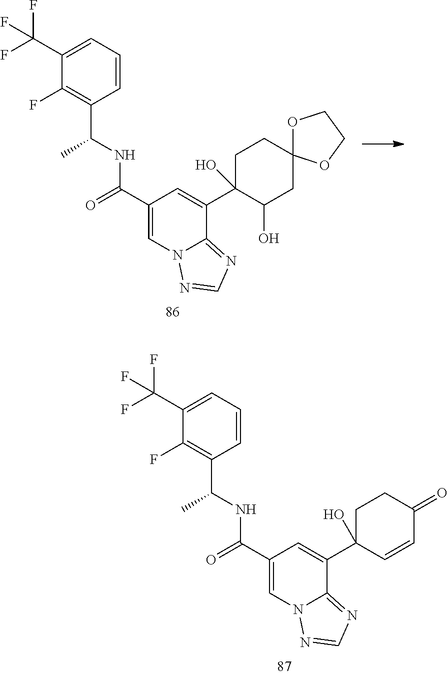 [SMILES: C[C@@H](NC(=O)c1cc(C2(O)CCC3(CC2O)OCCO3)c2ncnn2c1)c1cccc(C(F)(F)F)c1F ; C[C@@H](NC(=O)c1cc(C2(O)C=CC(=O)CC2)c2ncnn2c1)c1cccc(C(F)(F)F)c1F]

A mixture of intermediate 86 (4.7 g, 9.0 mmol) in THF (22 mL) is treated with aqueous HCl solution (4 M; 16 mL) and stirring is continued at rt for 24 h. THF is evaporated, and the aqueous layer is extracted with EtOAc. The combined organic layers are dried with MgSO₄, filtered and concentrated. The intermediate 87 is used for the next reaction step without further purification.

Intermediate 87
HPLC-MS Method: E

| | |
|---|---|
| Rₜ [min]: 0.97 | MS [m/z]: 463 [M + H]⁺ |

### Synthesis of Intermediate 88

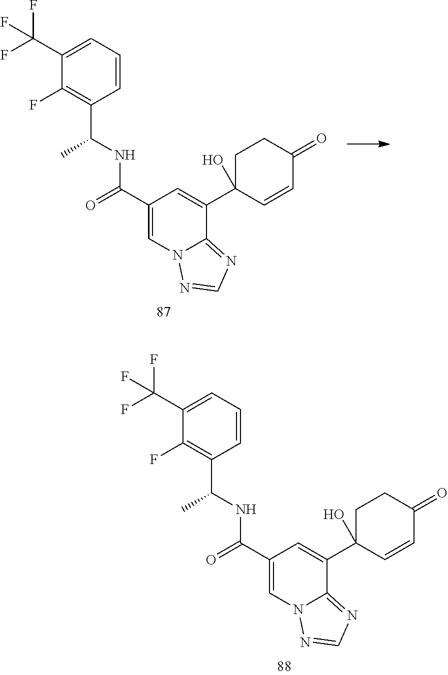 [SMILES: C[C@@H](NC(=O)c1cc(C2(O)C=CC(=O)CC2)c2ncnn2c1)c1cccc(C(F)(F)F)c1F ; C[C@@H](NC(=O)c1cc(C2(O)C=CC(=O)CC2)c2ncnn2c1)c1cccc(C(F)(F)F)c1F]

A mixture of intermediate 87 (4.8 g, 10 mmol) in 1,4-dioxane (85 mL) is treated with HCl in 1,4-dioxane (4 M; 25 mL) and Pd/C (10% wt/wt 500 mg). The mixture is stirred under H₂ atmosphere (50 psi) for 24 h at rt. The mixture is filtered, the solvents are evaporated, and the residue purified by prep. RP-HPLC (acidic conditions) to obtain intermediate 88.

Intermediate 88
HPLC-MS Method: E

| | |
|---|---|
| Rₜ [min]: 0.99 | MS [m/z]: 465 [M + H]⁺ |

### Synthesis of Example E71

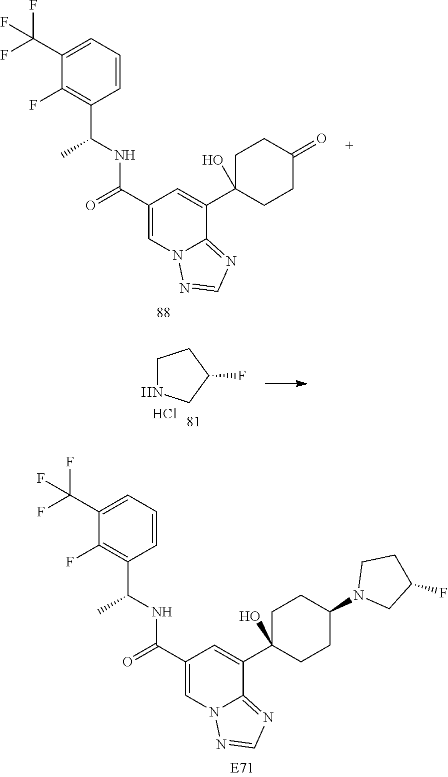 [SMILES: C[C@@H](NC(=O)c1cc(C2(O)CCC(=O)CC2)c2ncnn2c1)c1cccc(C(F)(F)F)c1F ; F[C@H]1CCNC1 ; Cl ; C[C@@H](NC(=O)c1cc([C@]2(O)CC[C@@H](N3CC[C@H](F)C3)CC2)c2ncnn2c1)c1cccc(C(F)(F)F)c1F]

Intermediate 88 (123 mg, 265 μmol) in THF (2.1 mL) is treated with (S)-3-fluoropyrrolidine hydrochloride 81 (50 mg, 397 μmol), AcOH (30 μL, 530 μmol) and molecular sieves 4 A and is stirred at 55° C. for 2 h. After cooling to rt, sodium triacetoxyborohydride (225 mg, 1.06 mmol) is added and the reaction mixture stirred for 1 h at rt. The crude mixture is filtered through Celite and directly purified by prep. RP-HPLC (basic conditions) to give example E71.

Example E71
Analytical HPLC-MS Method: D

| | |
|---|---|
| Rₜ [min]: 1.01 | MS [m/z]: 538 [M + H]⁺ |

Analytical SFC method: I

| | |
|---|---|
| Rₜ [min]: 1.91 | d.e. > 98% |

¹H NMR (400 MHz, DMSO-d₆) δ (ppm): 1.50-1.97 (10 H), 2.02-2.21 (1 H), 2.24-2.37
(1 H), 2.61-3.04 (5 H), 5.08-5.32 (2 H), 5.37-5.50 (1 H), 7.37-7.45 (1 H), 7.65-7.73
(1 H), 7.80-7.90 (1 H), 8.20-8.25 (1 H), 8.58-8.62 (1 H), 9.16-9.24 (1 H), 9.47-9.52
(1 H), missing proton(s) presumably hidden by/ overlapping with solvent signals.

### Synthesis Scheme of Intermediate 92

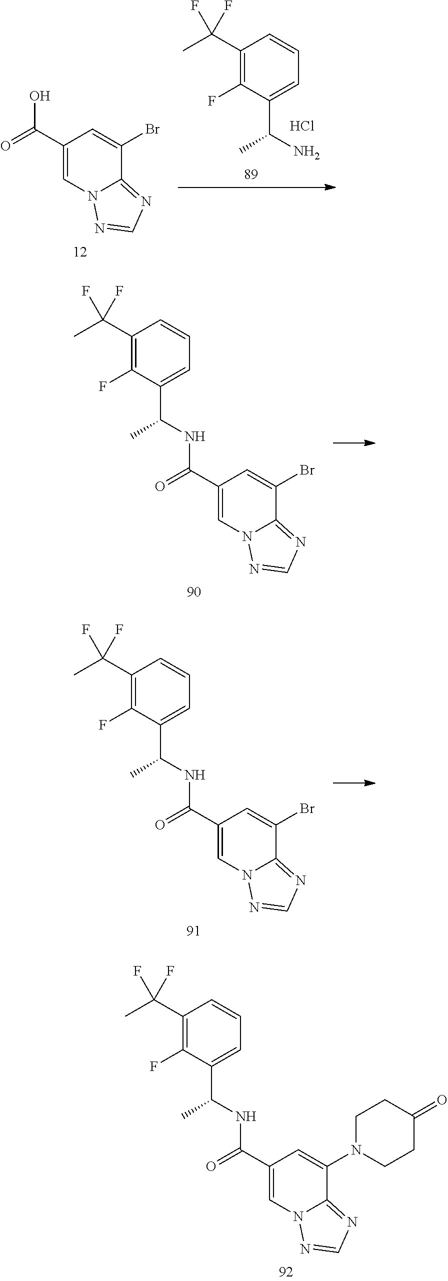 [SMILES: O=C(O)c1cc(Br)c2ncnn2c1 ; C[C@@H](N)c1cccc(C(C)(F)F)c1F ; Cl ; C[C@@H](NC(=O)c1cc(Br)c2ncnn2c1)c1cccc(C(C)(F)F)c1F ; C[C@@H](NC(=O)c1cc(Br)c2ncnn2c1)c1cccc(C(C)(F)F)c1F ; C[C@@H](NC(=O)c1cc(N2CCC(=O)CC2)c2ncnn2c1)c1cccc(C(C)(F)F)c1F]

### Synthesis of Intermediate 90

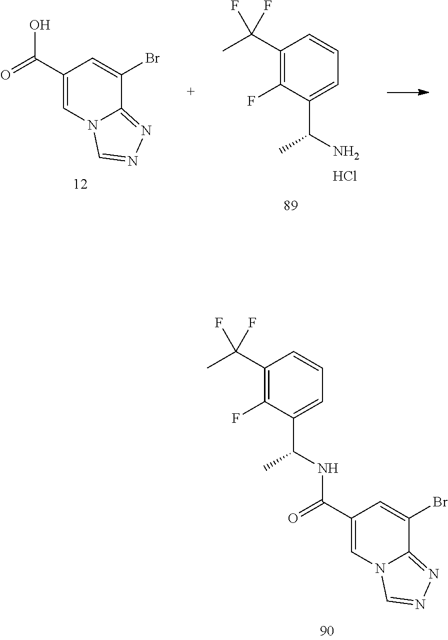 [SMILES: O=C(O)c1cc(Br)c2nncn2c1 ; C[C@@H](N)c1cccc(C(C)(F)F)c1F ; Cl ; C[C@@H](NC(=O)c1cc(Br)c2nncn2c1)c1cccc(C(C)(F)F)c1F]

HATU (1.56 g, 4.09 mmol) and triethylamine (2.09 mL, 14.9 mmol) are added to intermediate 12 (1.00 g, 3.72 mmol) in DMF (5 mL) and the mixture is stirred for 10 min at rt. (R)-1-(3-(1,1-Difluoroethyl)-2-fluorophenyl)ethanamine hydrochloride 89 (CAS: 2569698-48-0; 949 mg, 3.90 mmol) is added and the mixture is stirred for 16 h at rt. The mixture is diluted with water and extracted with EtOAc. The combined organic layers are washed with aqueous saturated NaCl solution, dried over MgSO₄ and concentrated in vacuo. The material is purified by column chromatography (SiO₂; EtOAc/MeOH: 96:4→80:20) to obtain intermediate 90.

Intermediate 90
Analytical HPLC-MS Method: D

| | |
|---|---|
| Rₜ [min]: 0.92 | MS [m/z]: 427 [M + H]⁺ |

### Synthesis of Intermediate 91

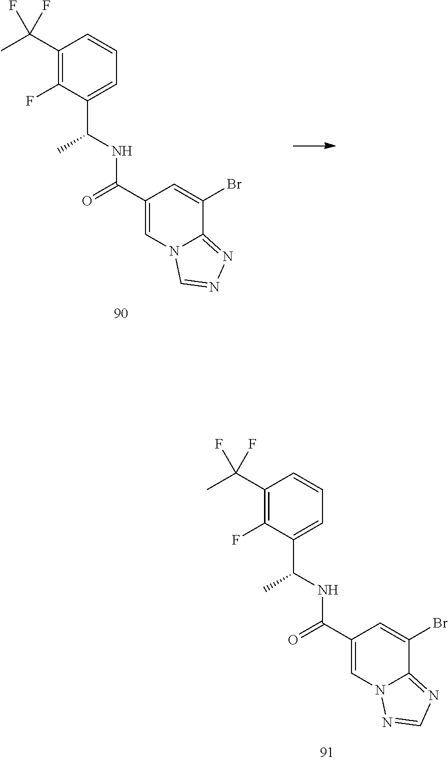 [SMILES: C[C@@H](NC(=O)c1cc(Br)c2nncn2c1)c1cccc(C(C)(F)F)c1F ; C[C@@H](NC(=O)c1cc(Br)c2ncnn2c1)c1cccc(C(C)(F)F)c1F]

A mixture of intermediate 90 (1.00 g, 2.34 mmol), Cs₂CO₃ (385 mg, 1.17 mmol), piperidine (116 μL, 1.17 mmol) in 1,4-dioxane (10 mL) is stirred for 4 h at 80° C. The mixture is diluted with EtOAc, filtered over a pad of silica gel, rinsed with EtOAc, and concentrated in vacuo. The residue is dissolved in EtOAc and washed with water. The organic layer is concentrated under reduced pressure and purified by prep. RP-HPLC (basic conditions) to obtain intermediate 91.

Intermediate 91
HPLC-MS Method: E

| | |
|---|---|
| Rₜ [min]: 1.03 | MS [m/z]: 427 [M + H]⁺ |

### Synthesis of Intermediate 92

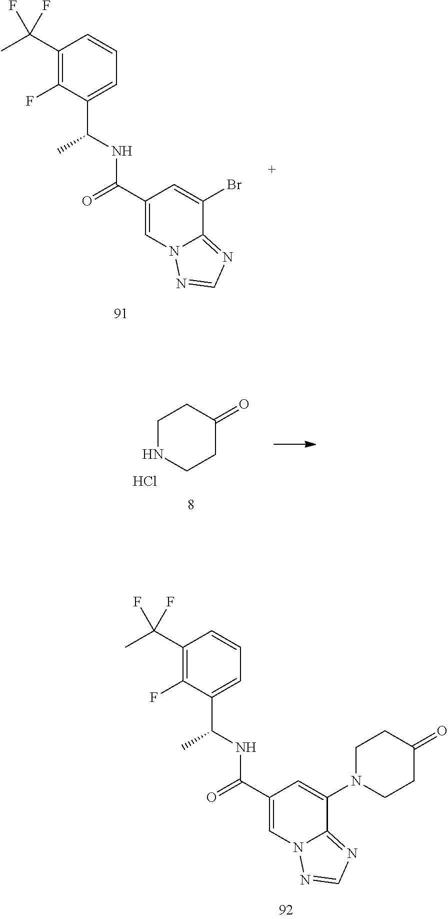 [SMILES: C[C@@H](NC(=O)c1cc(Br)c2ncnn2c1)c1cccc(C(C)(F)F)c1F ; O=C1CCNCC1 ; Cl ; C[C@@H](NC(=O)c1cc(N2CCC(=O)CC2)c2ncnn2c1)c1cccc(C(C)(F)F)c1F]

A mixture of intermediate 91 (330 mg, 772 μmol), piperidin-4-one hydrochloride 8 (220 mg, 1.55 mmol) and Cs₂CO₃ (1.01 g, 3.09 mmol) in 1,4-dioxane (6 mL) is degassed with argon. Catalyst I (26 mg, 31 μmol) is added, and the mixture is stirred under argon for 3 h at 95° C. The mixture is diluted with water and extracted with EtOAc. The combined organic layers are washed with aqueous saturated NaCl solution, dried over MgSO₄ and the solvent is evaporated under reduced pressure. The material is purified by prep. RP-HPLC (basic conditions) to obtain intermediate 92.

Intermediate 92
Analytical HPLC-MS Method: D

| | |
|---|---|
| Rₜ [min]: 0.96 | MS [m/z]: 446 [M + H]⁺ |

### Synthesis of Example E72

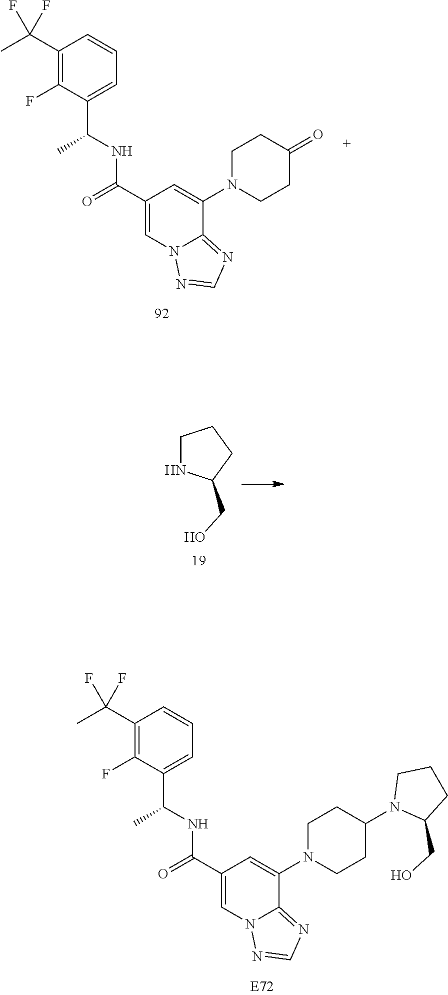 [SMILES: C[C@@H](NC(=O)c1cc(N2CCC(=O)CC2)c2ncnn2c1)c1cccc(C(C)(F)F)c1F ; OC[C@@H]1CCCN1 ; C[C@@H](NC(=O)c1cc(N2CCC(N3CCC[C@H]3CO)CC2)c2ncnn2c1)c1cccc(C(C)(F)F)c1F]

2-Picoline borane complex (16 mg, 146 μmol) is added to a mixture of intermediate 92 (50 mg, 112 μmol), L-prolinol 19 (23 mg, 224 μmol), and AcOH (20 μL, 337 μmol) in MeOH (2 mL) and the resulting mixture is stirred for 16 h at rt. The reaction mixture is neutralized with aqueous NaOH solution (1 N), diluted with MeOH, filtered, and purified by prep. RP-HPLC (basic conditions) to obtain example E72.

Example E72
Analytical HPLC-MS Method: D

| | |
|---|---|
| Rₜ [min]: 0.99 | MS [m/z]: 531 [M + H]⁺ |

Analytical SFC method: Y

| | |
|---|---|
| Rₜ [min]: 5.24 | d.e. > 98% |

¹H NMR (400 MHz, DMSO-d₆) δ (ppm): 1.47-1.72 (9 H), 1.85-1.95 (2 H), 1.95-2.10 (3
H), 2.62-2.73 (1 H), 2.80-2.94 (4 H), 3.02-3.11 (1 H), 4.30-4.40 (3 H), 5.37-5.47 (1
H), 7.10-7.18 (1 H), 7.26-7.33 (1 H), 7.42-7.51 (1 H), 7.60-7.69 (1 H), 8.47-8.53 (1
H), 9.01-9.08 (2 H), missing proton(s) presumably hidden by/ overlapping with solvent
signals.

### Synthesis of Example E73

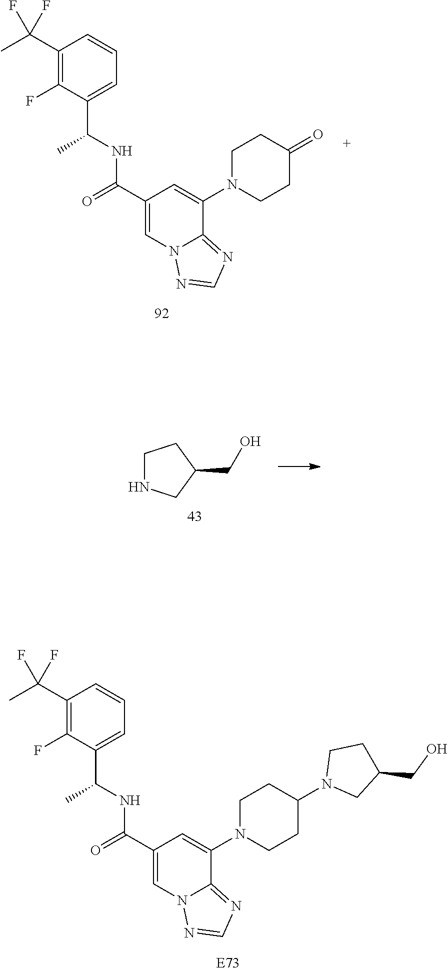 [SMILES: C[C@@H](NC(=O)c1cc(N2CCC(=O)CC2)c2ncnn2c1)c1cccc(C(C)(F)F)c1F ; OC[C@@H]1CCNC1 ; C[C@@H](NC(=O)c1cc(N2CCC(N3CC[C@@H](CO)C3)CC2)c2ncnn2c1)c1cccc(C(C)(F)F)c1F]

2-Picoline borane complex (16 mg, 146 μmol) is added to a mixture of intermediate 92 (50 mg, 112 μmol), (R)-pyrrolidin-3-ylmethanol 43 (23 mg, 224 μmol) and AcOH (20 μL, 337 μmol) in MeOH (2 mL) and the resulting mixture is stirred at rt for 16 h. The reaction mixture is neutralized with aqueous NaOH solution (1 N), diluted with MeOH, and purified by prep. RP-HPLC (basic conditions) to obtain example E73.

Example E73
Analytical HPLC-MS Method: D

| | |
|---|---|
| Rₜ [min]: 0.96 | MS [m/z]: 531 [M + H]⁺ |

Analytical SFC method: Y

| | |
|---|---|
| Rₜ [min]: 3.74 | d.e. > 97% |

¹H NMR (400 MHz, DMSO-d₆) δ (ppm): 1.30-1.42 (1 H), 1.48-1.63 (5 H), 1.73-1.85 (1
H), 1.90-2.11 (5 H), 2.12-2.26 (2 H), 2.30-2.37 (1 H), 2.61-2.69 (1 H), 2.95-3.06 (2
H), 4.14-4.30 (2 H), 4.38-4.61 (1 H), 5.37-5.48 (1 H), 7.13-7.18 (1 H), 7.26-7.33 (1
H), 7.43-7.51 (1 H), 7.61-7.69 (1 H), 8.49-8.53 (1 H), 9.03-9.11 (2 H), missing
proton(s) presumably hidden by/ overlapping with solvent signals.

### Synthesis Scheme of Intermediate 95

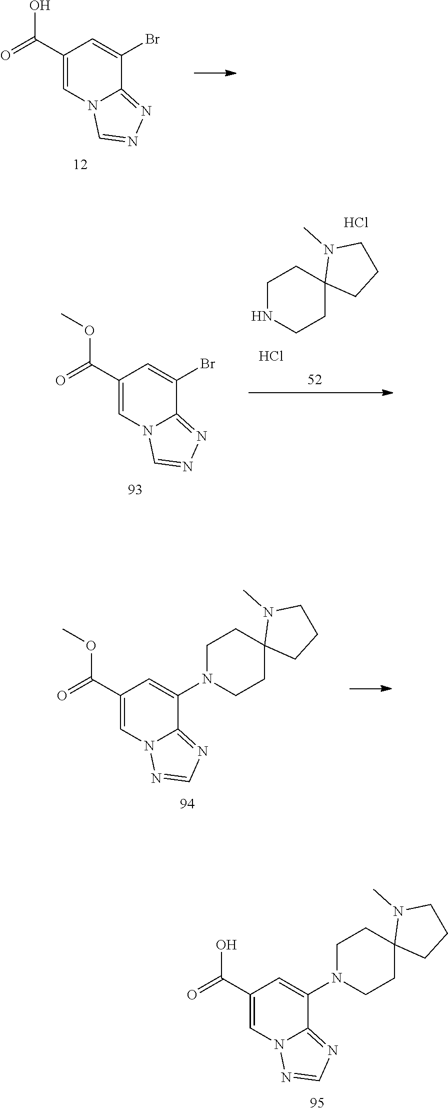

### Synthesis of Intermediate 93

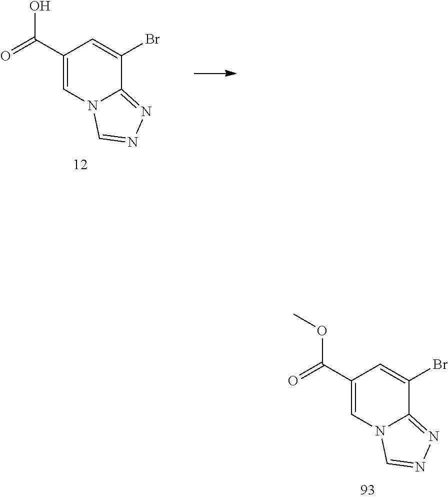

A mixture of intermediate 12 (1.00 g, 4.13 mmol) in DCM (7 mL) and MeOH (1.5 mL) is treated dropwise with trimethysilyldiazomethane (0.6 M in hexane; 8.26 mL, 4.96 mmol) whereby the temperature in the reaction mixture is kept below 25° C. After complete addition, stirring is continued for 1 h at rt. AcOH (0.28 mL, 4.96 mmol) is added and stirring is continued for 15 min at rt. Aqueous 2 M NaHCO₃ solution is added, the aqueous layer extracted with DCM, and the combined organic layers dried with MgSO₄. The mixture is filtered, and the solvent is evaporated to obtain intermediate 93, which is used in the next reaction step without further purification.

Intermediate 93
HPLC-MS Method: E

| | |
|---|---|
| Rₜ [min]: 0.69 | MS [m/z]: 256 [M + H]⁺ |

### Synthesis of Intermediate 94

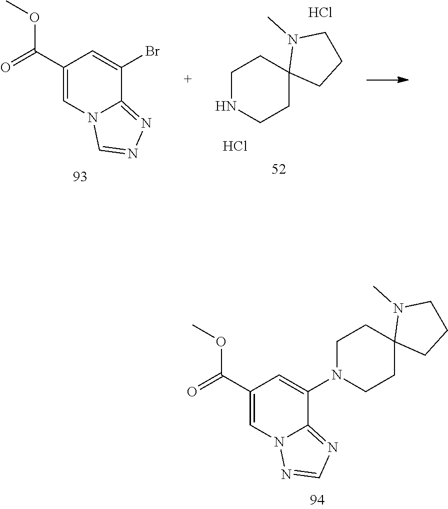 [SMILES: COC(=O)c1cc(Br)c2nncn2c1 ; Cl ; CN1CCCC12CCNCC2 ; Cl ; COC(=O)c1cc(N2CCC3(CCCN3C)CC2)c2ncnn2c1]

A mixture of intermediate 93 (400 mg, 1.48 mmol), 1-methyl-1,8-diazaspiro[4.5]decane dihydrochloride 52 (390 mg, 1.63 mmol) and Cs₂CO₃ (1.93 g, 5.94 mmol) in 1,4-dioxane (13 mL) is degassed with argon. Catalyst I (125 mg, 148 μmol) is added, and the mixture is stirred under argon at 90° C. for 16 h. The solvent is evaporated, water is added and the aqueous layer extracted with EtOAc. The organic layer is dried with MgSO₄. After filtration and evaporation of the solvent, the material is purified by prep. RP-HPLC (basic conditions) to obtain intermediate 94.

Intermediate 94
Analytical HPLC-MS Method: D

| | |
|---|---|
| Rₜ [min]: 0.91 | MS [m/z]: 330 [M + H]⁺ |

### Synthesis of Intermediate 95

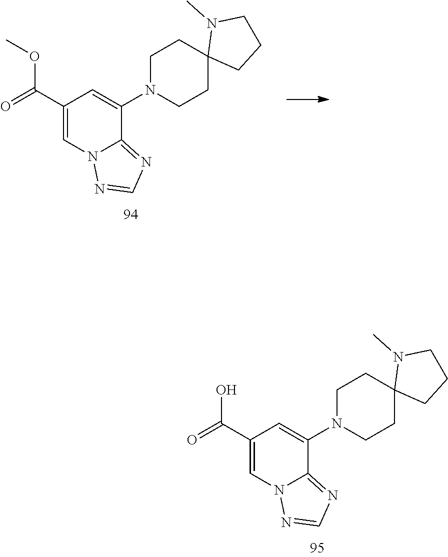 [SMILES: COC(=O)c1cc(N2CCC3(CCCN3C)CC2)c2ncnn2c1 ; CN1CCCC12CCN(c1cc(C(=O)O)cn3ncnc13)CC2]

A mixture of intermediate 94 (107 mg, 325 μmol) in MeOH (1.3 mL) is treated with aqueous NaOH solution (4 M; 0.24 mL, 975 μmol) and stirring is continued for 16 h at rt. After evaporation of the solvents, water and aqueous HCl solution (4 M; 0.24 mL, 975 μmol) are added and stirring is continued for 30 min at 0° C. The reaction mixture is filtered and purified by prep. RP-HPLC (acidic conditions) to obtain the desired intermediate 95.

Intermediate 95
HPLC-MS Method: E

| | |
|---|---|
| Rₜ [min]: 0.61 | MS [m/z]: 314 [M + H]⁺ |

### Synthesis of Example E74

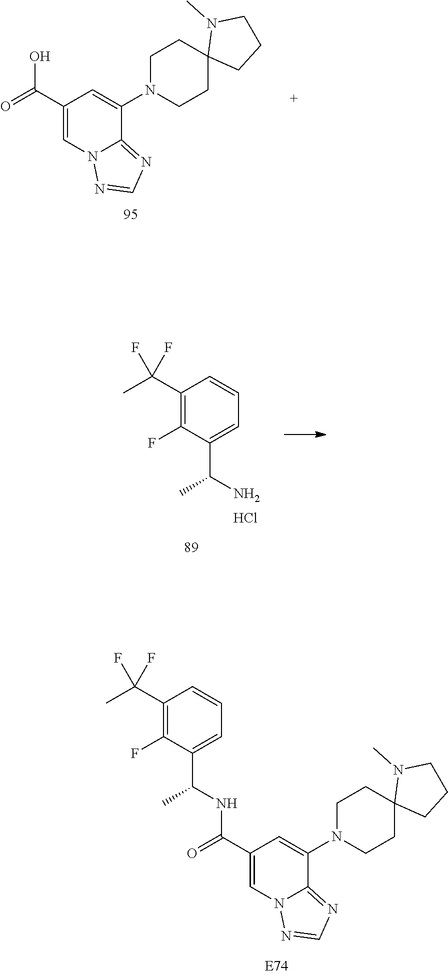 [SMILES: CN1CCCC12CCN(c1cc(C(=O)O)cn3ncnc13)CC2 ; C[C@@H](N)c1cccc(C(C)(F)F)c1F ; Cl ; C[C@@H](NC(=O)c1cc(N2CCC3(CCCN3C)CC2)c2ncnn2c1)c1cccc(C(C)(F)F)c1F]

A mixture of intermediate 95 (65 mg, 206 μmol) in THF (3.3 mL) is treated with HATU (94 mg, 247 μmol) and triethylamine (116 μL, 824 μmol) and the mixture is stirred for 15 min at rt. (R)-1-(3-(1,1-Difluoroethyl)-2-fluorophenyl)ethanamine hydrochloride 89 (54 mg, 227 μmol) is added and stirring is continued for 16 h at rt. The mixture is diluted with MeOH, filtered, and the mixture is purified by prep. RP-HPLC (acidic conditions) and filtered through a carbonate cartridge. The solvent is evaporated to obtain example E74.

Example E74
HPLC-MS Method: E

| | |
|---|---|
| Rₜ [min]: 0.84 | MS [m/z]: 501 [M + H]⁺ |

Analytical SFC method: F

| | |
|---|---|
| Rₜ [min]: 1.74 | e.e. > 98% |

¹H NMR (400 MHz, DMSO-d₆) δ (ppm): 1.28-1.38 (2 H), 1.46-1.56 (3 H), 1.65-1.86
(6 H), 1.96-2.10 (3 H), 2.18-2.24 (3 H), 2.65-2.74 (2 H), 2.80-2.91 (2 H), 4.29-4.40
(2 H), 5.37-5.48 (1 H), 7.13-7.19 (1 H), 7.26-7.34 (1 H), 7.43-7.50 (1 H), 7.60-7.68
(1 H), 8.48-8.53 (1 H), 9.02-9.09 (2 H).

### Synthesis of Intermediate 96

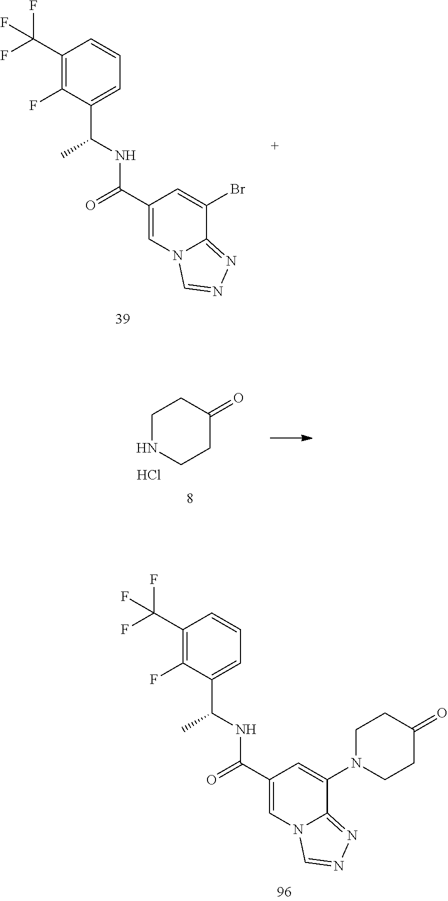 [SMILES: C[C@@H](NC(=O)c1cc(Br)c2nncn2c1)c1cccc(C(F)(F)F)c1F ; O=C1CCNCC1 ; Cl ; C[C@@H](NC(=O)c1cc(N2CCC(=O)CC2)c2nncn2c1)c1cccc(C(F)(F)F)c1F]

A mixture of intermediate 39 (1.5 g, 3.5 mmol), piperidin-4-one hydrochloride 8 (1.0 g, 7.0 mmol) and K₃PO₄ (2.7 g, 12.2 mmol) in 1,4-dioxane (50 mL) is degassed with argon. Catalyst I (88 mg, 104 μmol) is added, and the mixture is stirred for 3 h at 95° C. under an argon atmosphere. The mixture is filtered, and the filter is rinsed with EtOAc. The organic solvents are evaporated under reduced pressure. The material is purified by column chromatography (SiO₂; EtOAc/MeOH: 95:5→80:20) to obtain intermediate 96.

Intermediate 96
Analytical HPLC-MS Method: D

| | |
|---|---|
| Rₜ [min]: 0.94 | MS [m/z]: 450 [M + H]⁺ |

### Synthesis of Example E75

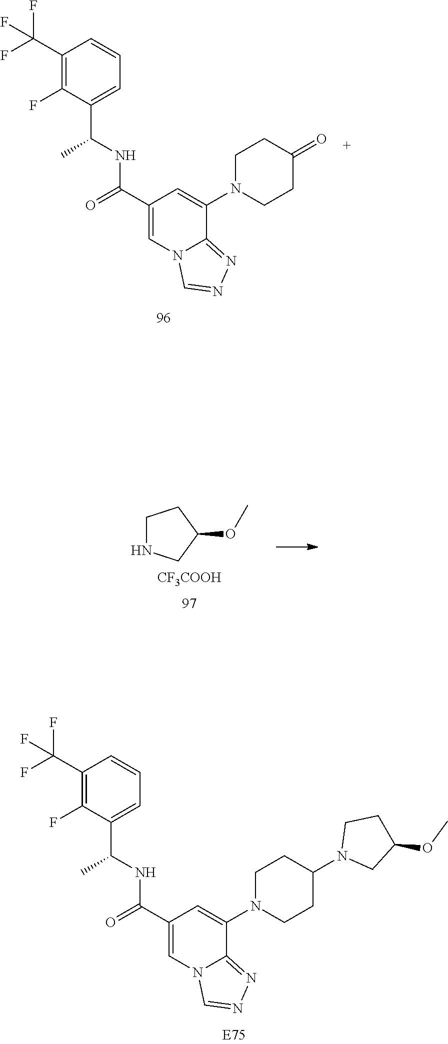

A mixture of intermediate 96 (31 mg, 70 μmol), (3R)-3-methoxypyrrolidine trifluoroacetate 97 (24 mg, 0.11 mmol), AcOH (30.0 μL, 0.51 mmol), and 2-picoline borane complex (7.5 mg, 70 μmol) in MeOH (1.5 mL) is stirred for 12 h at rt. The mixture is diluted with DMF und purified by prep. RP-HPLC (basic conditions) to obtain example E75.

Example E75
HPLC-MS Method: A

| | |
|---|---|
| Rₜ [min]: 0.80 | MS [m/z]: 535 [M + H]⁺ |

Analytical SFC method: AH

| | |
|---|---|
| Rₜ [min]: 7.22 | d.e. > 98% |

¹H NMR (400 MHz, DMSO-d₆ + ND₄OD) δ (ppm): 1.43-1.58 (5 H), 1.58-1.69 (1 H), 1.85-
1.98 (3 H), 2.12-2.29 (1 H), 2.39-2.47 (1 H), 2.52-2.58 (2 H), 2.79-2.90 (3 H), 3.11-
3.17 (3 H), 5.28-5.37 (1 H), 6.66-6.80 (1 H), 7.29-7.39 (1 H), 7.59-7.66 (1 H), 7.68-
7.75 (1 H), 8.46-8.56 (1 H), 9.14-9.25 (1 H), missing proton(s) presumably hidden by/
overlapping with solvent signals.

### Synthesis of Example E76

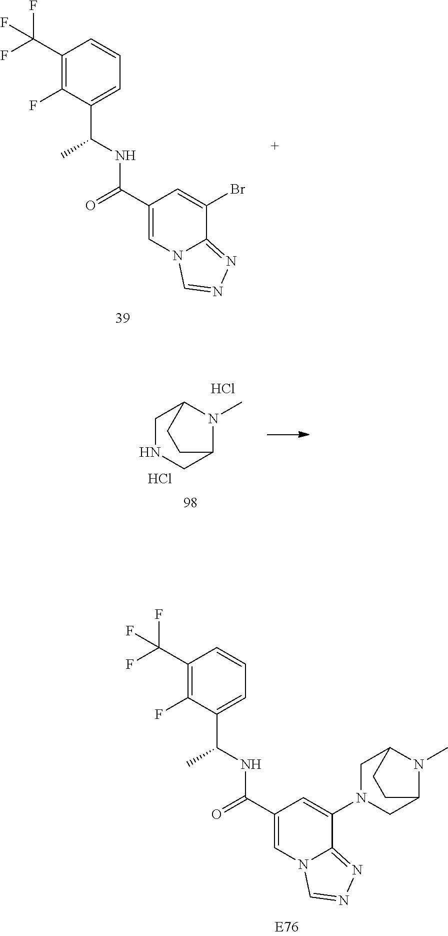

A mixture of bromide 39 (70 mg, 154 μmol), intermediate 98 (36 mg, 170 μmol), Cs₂CO₃ (201 mg, 617 μmol) and catalyst I (13 mg, 15 μmol) in degassed 1,4-dioxane (1.3 mL) is stirred under argon for 16 h at 90° C. Water and MeOH are added, the mixture is filtered and the filtrate is concentrated in vacuo. The residue is purified by prep. RP-HPLC (basic conditions) to obtain example E76.

Example E76
Analytical HPLC-MS Method: D

| | |
|---|---|
| Rₜ [min]: 0.96 | MS [m/z]: 477 [M + H]⁺ |

Analytical SFC method: T

| | |
|---|---|
| Rₜ [min]: 4.92 | e.e. > 94% |

¹H NMR (400 MHz, DMSO-d₆) δ (ppm): 1.47-1.58 (3 H), 1.71-1.83 (2 H), 1.92-2.04 (2
H), 2.19-2.29 (3 H), 2.97-3.07 (2 H), 3.18-3.27 (2 H), 4.11-4.26 (2 H), 5.34-5.49 (1
H), 6.58-6.68 (1 H), 7.35-7.47 (1 H), 7.64-7.73 (1 H), 7.75-7.86 (1 H), 8.58-8.66 (1
H), 8.97-9.10 (1 H), 9.26-9.35 (1 H).

### Synthesis of Example E77

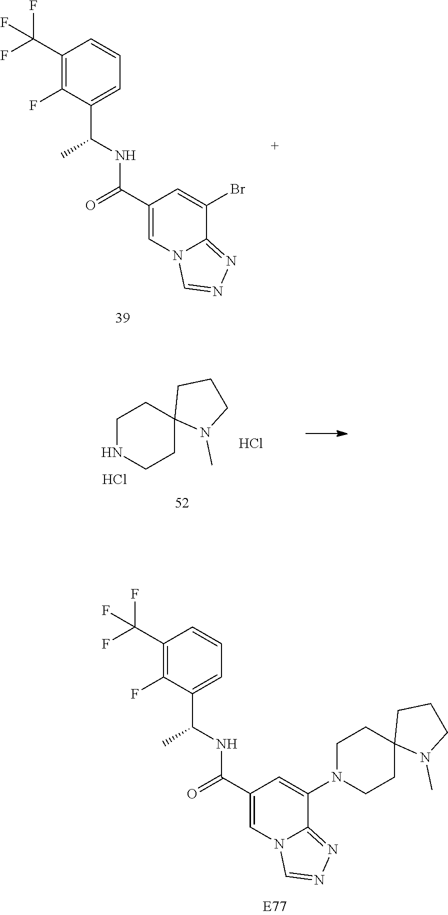 [SMILES: C[C@@H](NC(=O)c1cc(Br)c2nncn2c1)c1cccc(C(F)(F)F)c1F ; CN1CCCC12CCNCC2 ; Cl ; Cl ; C[C@@H](NC(=O)c1cc(N2CCC3(CCCN3C)CC2)c2nncn2c1)c1cccc(C(F)(F)F)c1F]

A mixture of intermediate 39 (125 mg, 0.275 mmol) and 1-methyl-1,8-diazaspiro[4.5]decane dihydrochloride 52 (72 mg, 303 μmol) in 1,4-dioxane (2.4 mL) is degassed with argon. Cs₂CO₃ (359 mg, 1.10 mmol) and catalyst I (23 mg, 28 μmol) is added, and the mixture is heated for 16 h at 120° C. Aqueous NaHCO₃ solution is added and the mixture is extracted with EtOAc. The organic layer is dried with MgSO₄. After filtration and evaporation of the solvent, the mixture is purified by prep. RP-HPLC (basic conditions) to obtain example E77.

Example E77
Analytical HPLC-MS Method: D

| | |
|---|---|
| Rₜ [min]: 1.01 | MS [m/z]: 505 [M + H]⁺ |

Analytical SFC method: AB

| | |
|---|---|
| Rₜ [min]: 6.31 | e.e. > 98% |

¹H NMR (400 MHz, DMSO-d₆) δ (ppm): 1.48-1.58 (3 H), 1.65-1.88 (6 H), 2.18-2.26
(3 H), 2.59-2.77 (2 H), 2.82-2.93 (2 H), 4.39-4.52 (2 H), 5.35-5.47 (1 H), 6.77-6.84
(1 H), 7.37-7.48 (1 H), 7.64-7.73 (1 H), 7.77-7.86 (1 H), 8.63-8.69 (1 H), 9.02-9.11
(1 H), 9.27-9.34 (1 H), missing proton(s) presumably hidden by/ overlapping with solvent
signals.

### Synthesis Scheme of Intermediate 102

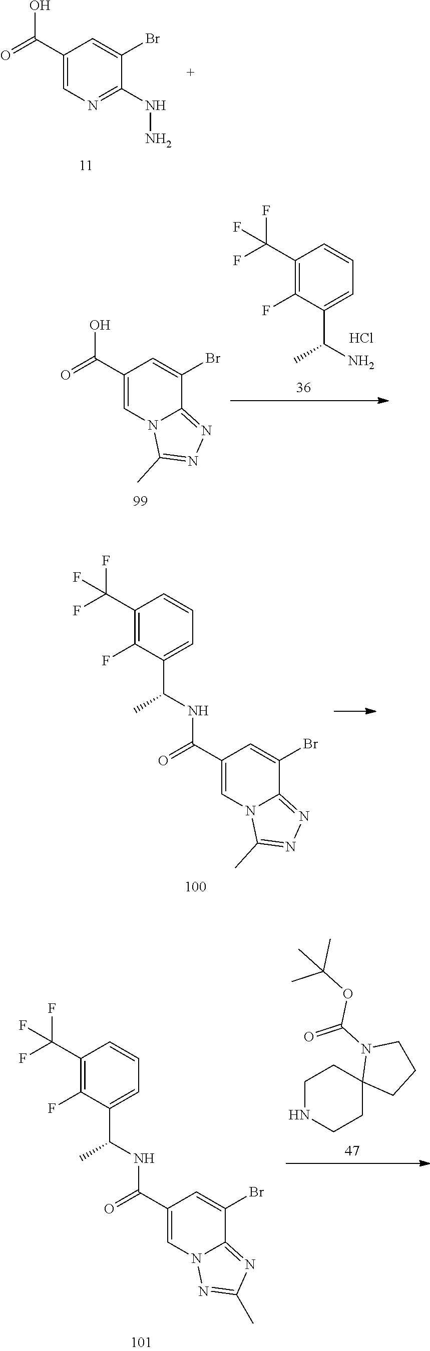

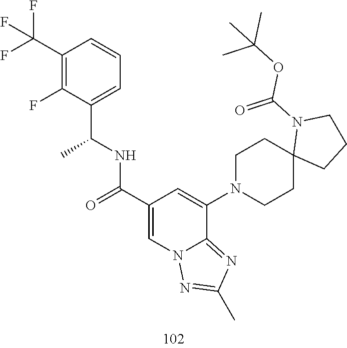 [SMILES: Cc1nc2c(N3CCC4(CCCN4C(=O)OC(C)(C)C)CC3)cc(C(=O)N[C@H](C)c3cccc(C(F)(F)F)c3F)cn2n1]

### Synthesis of Intermediate 99

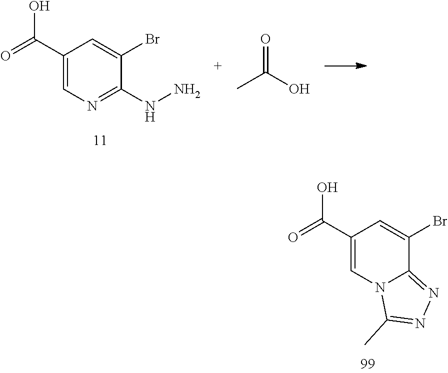

A mixture of intermediate 11 (460 mg, 2.0 mmol) in AcOH (1.7 mL, 30 mmol) is stirred at 140° C. for 5 h. The mixture is cooled to rt and poured into ice-water. The solid material is filtered off, washed with water and dried under reduced pressure at 55° C. to obtain the intermediate 99. The material is used without further purification.

Intermediate 99
HPLC-MS Method: E

| | |
|---|---|
| Rₜ [min]: 0.60 | MS [m/z]: 256 [M + H]⁺ |

### Synthesis of Intermediate 100

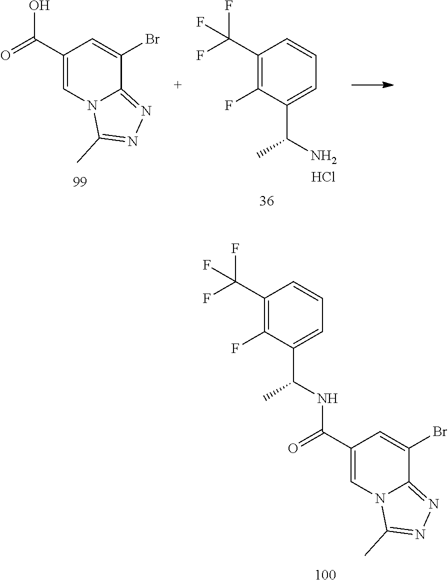 [SMILES: Cc1nnc2c(Br)cc(C(=O)O)cn12 ; C[C@@H](N)c1cccc(C(F)(F)F)c1F ; Cl ; Cc1nnc2c(Br)cc(C(=O)N[C@H](C)c3cccc(C(F)(F)F)c3F)cn12]

Intermediate 99 is synthesized in analogy to intermediate 39: Intermediate 99 (27 mmol), (1R)-1-[2-fluoro-3-(trifluoromethyl)phenyl]ethan-1-amine hydrochloride 36 (27 mmol), 1-propanephosphonic anhydride (53 mmol), N-methylmorpholine (80 mmol), 70 mL ACN.

Intermediate 100
HPLC-MS Method: E

| | |
|---|---|
| Rₜ [min]: 0.99 | MS [m/z]: 445 [M + H]⁺ |

### Synthesis of Intermediate 101

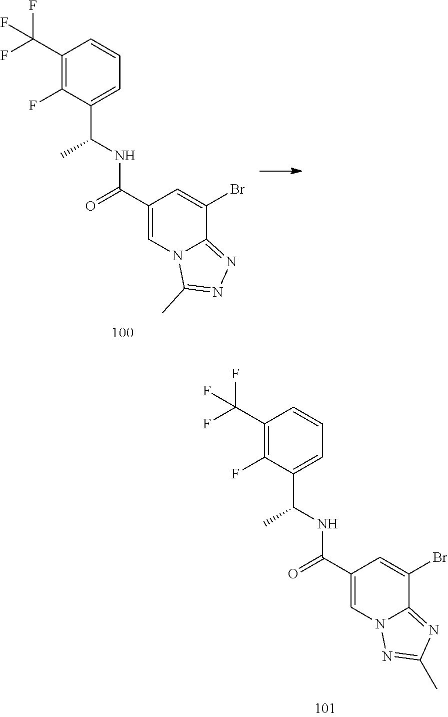 [SMILES: Cc1nnc2c(Br)cc(C(=O)N[C@H](C)c3cccc(C(F)(F)F)c3F)cn12 ; Cc1nc2c(Br)cc(C(=O)N[C@H](C)c3cccc(C(F)(F)F)c3F)cn2n1]

A mixture of intermediate 100 (150 mg, 337 μmol), Cs₂CO₃ (220 mg, 674 μmol), piperidine (57 mg, 674 μmol) in 1,4-dioxane (3 mL) is stirred for 2.5 h at 90° C. The mixture is poured into aqueous 0.5 M KHSO₄ solution and is extracted with EtOAc. The solvent is evaporated under reduced pressure and the material is used in the next reaction step without purification.

Intermediate 101
Analytical HPLC-MS Method: D

| | |
|---|---|
| Rₜ [min]: 1.01 | MS [m/z]: 445 [M + H]⁺ |

### Synthesis of Intermediate 102

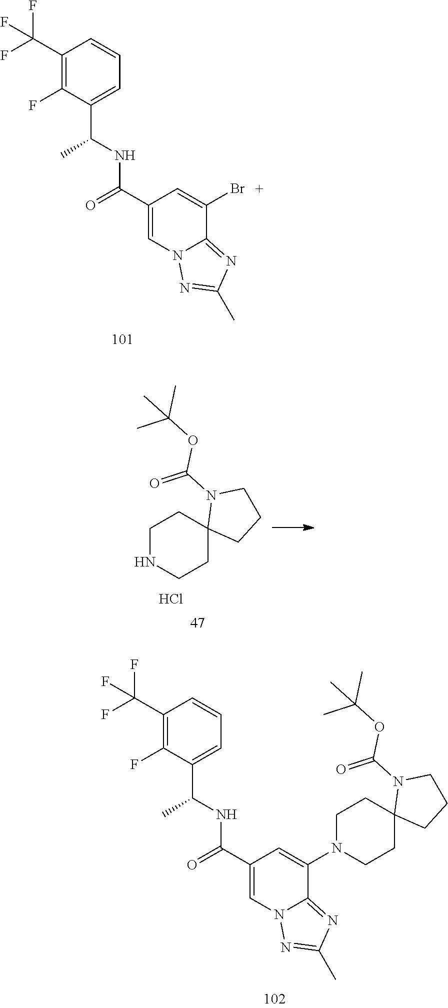 [SMILES: Cc1nc2c(Br)cc(C(=O)N[C@H](C)c3cccc(C(F)(F)F)c3F)cn2n1 ; CC(C)(C)OC(=O)N1CCCC12CCNCC2 ; Cl ; Cc1nc2c(N3CCC4(CCCN4C(=O)OC(C)(C)C)CC3)cc(C(=O)N[C@H](C)c3cccc(C(F)(F)F)c3F)cn2n1]

A mixture of bromide 101 (60 mg, 135 μmol), tert-butyl 1,8-diazaspiro[4.5]decane-1-carboxylate hydrochloride 47 (47 mg, 162 μmol) and Cs₂CO₃ (154 mg, 472 μmol) and catalyst I (11 mg, 13 μmol) in degassed 1,4-dioxane (2 mL) is stirred under argon for 16 h at 90° C. The mixture is diluted with DMF/MeOH, filtered, and washed with DMF/MeOH. The material is purified by prep. RP-HPLC (basic conditions) to obtain intermediate 102.

Intermediate 102
Analytical HPLC-MS Method: D

Rₜ [min]: 1.18

### Synthesis of Example E78

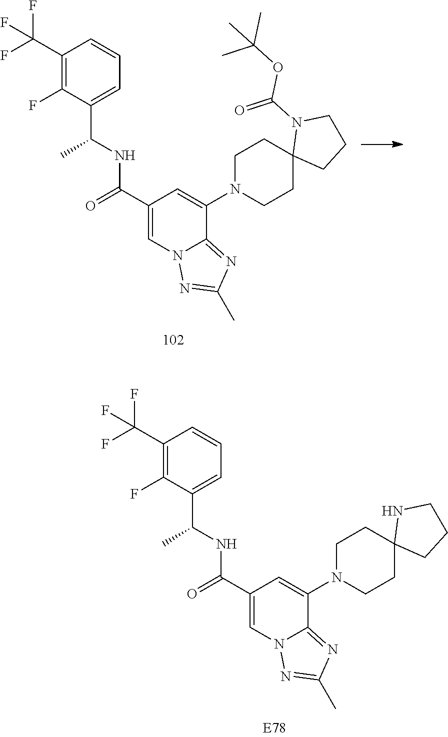 [SMILES: Cc1nc2c(N3CCC4(CCCN4C(=O)OC(C)(C)C)CC3)cc(C(=O)N[C@H](C)c3cccc(C(F)(F)F)c3F)cn2n1 ; Cc1nc2c(N3CCC4(CCCN4)CC3)cc(C(=O)N[C@H](C)c3cccc(C(F)(F)F)c3F)cn2n1]

Intermediate 102 (68 mg, 112 μmol) in 1,4-dioxane (2 mL) is treated with 4 N HCl in 1,4-dioxane (2 mL) and stirred for 3 h at rt. The solvent is evaporated under reduced pressure and the material is purified by prep. RP-HPLC (basic conditions) to obtain example E78.

Example E78
Analytical HPLC-MS Method: D

| | |
|---|---|
| Rₜ [min]: 1.04 | MS [m/z]: 505 [M + H]⁺ |

Analytical SFC method: H

| | |
|---|---|
| Rₜ [min]: 3.16 | e.e. > 98% |

¹H NMR (400 MHz, DMSO-d₆) δ (ppm): 1.48-1.77 (11 H), 2.81-2.89 (2 H), 3.50-3.65
(4 H), 5.34-5.47 (1 H), 7.08-7.15 (1 H), 7.37-7.45 (1 H), 7.63-7.72 (1 H), 7.78-7.86
(1 H), 8.88-8.95 (1 H), 9.03-9.10 (1 H), missing proton(s) presumably hidden by/
overlapping with solvent signals.

### Synthesis of Example E79

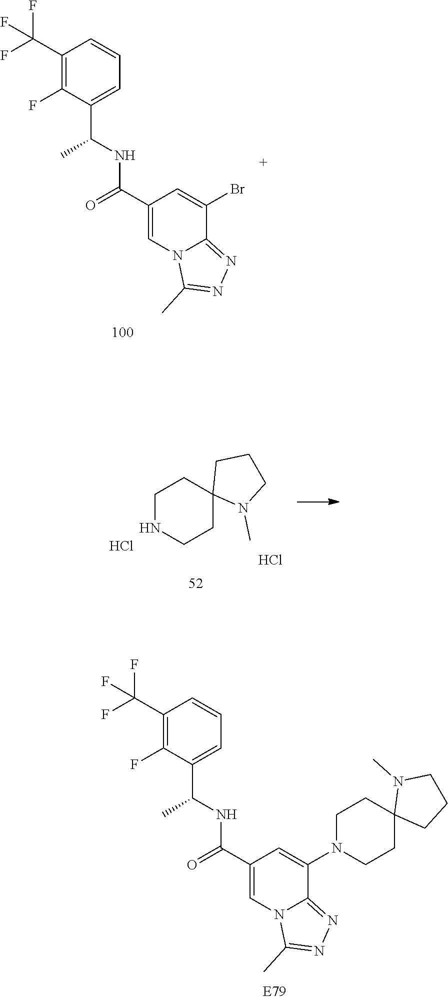 [SMILES: Cc1nnc2c(Br)cc(C(=O)N[C@H](C)c3cccc(C(F)(F)F)c3F)cn12 ; CN1CCCC12CCNCC2 ; Cl ; Cl ; Cc1nnc2c(N3CCC4(CCCN4C)CC3)cc(C(=O)N[C@H](C)c3cccc(C(F)(F)F)c3F)cn12]

A mixture of bromide 100 (200 mg, 449 μmol), 1-methyl-1,8-diazaspiro[4.5]decane dihydrochloride 52 (161 mg, 674 μmol) and Cs₂CO₃ (585 g, 1.8 mmol) in 1,4-dioxane (8 mL) is degassed with argon. Catalyst I (15 mg, 18 μmol) is added, and the mixture is heated under argon for 3 h at 110° C. The mixture is filtered, washed with EtOAc, and the solvent is evaporated under reduced pressure. The material is purified by prep. RP-HPLC (basic conditions) to obtain example E79.

Example E79
Analytical HPLC-MS Method: D

| | |
|---|---|
| Rₜ [min]: 1.00 | MS [m/z]: 519 [M + H]⁺ |

Analytical SFC method: U

| | |
|---|---|
| Rₜ [min]: 3.15 | e.e. > 98% |

¹H NMR (400 MHz, DMSO-d₆) δ (ppm): 1.29-1.37 (2 H), 1.49-1.61 (3 H), 1.66-1.86 (6
H), 2.16-2.26 (3 H), 2.65-2.76 (5 H), 2.79-2.93 (2 H), 4.32-4.53 (2 H), 5.34-5.49 (1
H), 6.74-6.86 (1 H), 7.34-7.48 (1 H), 7.64-7.74 (1 H), 7.77-7.86 (1 H), 8.36-8.49 (1
H), 8.93-9.09 (1 H).

### Synthesis of Intermediate 104

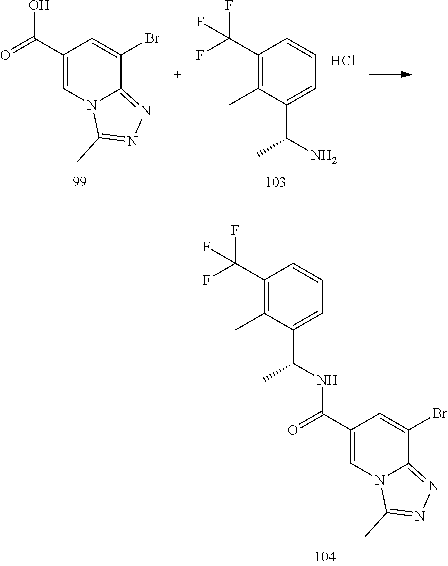 [SMILES: Cc1nnc2c(Br)cc(C(=O)O)cn12 ; Cc1c([C@@H](C)N)cccc1C(F)(F)F ; Cl ; Cc1c([C@@H](C)NC(=O)c2cc(Br)c3nnc(C)n3c2)cccc1C(F)(F)F]

A mixture of intermediate 99 (7.0 g, 27 mmol), (R)-1-(2-methyl-3-(trifluoromethyl)phenyl)ethanamine 103 (CAS: 2230840-58-9; 7.4 g, 27 mmol) and 4-methylmorpholine (8.8 mL, 80 mmol) in ACN (70 mL) is cooled to 0° C. PPA (50%, 32 mL, 53 mmol) is added dropwise and the mixture is allowed to reach rt. The mixture is poured into ice-water, ACN is evaporated under reduced pressure and water is added to the suspension. The solids are filtered, washed with water and tert-butylmethyl ether and dried to obtain intermediate 104. The material is used without further purification.

Intermediate 104
HPLC-MS Method: E

| | |
|---|---|
| Rₜ [min]: 0.99 | MS [m/z]: 445 [M + H]⁺ |

### Synthesis of Example E80

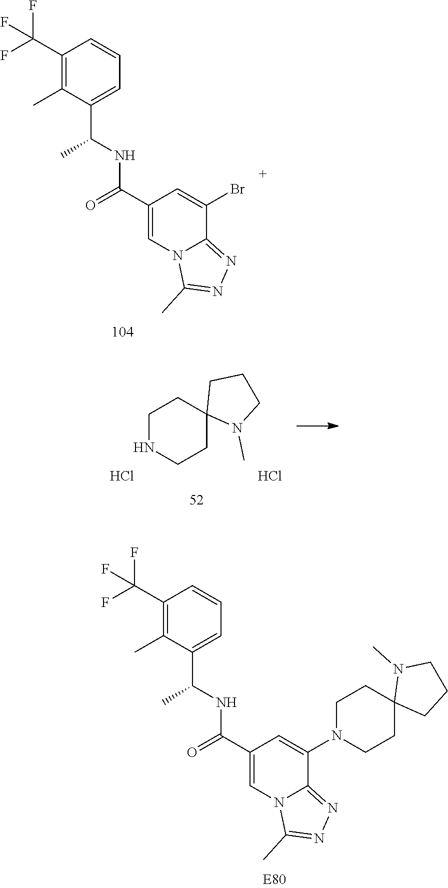 [SMILES: Cc1c([C@@H](C)NC(=O)c2cc(Br)c3nnc(C)n3c2)cccc1C(F)(F)F ; CN1CCCC12CCNCC2 ; Cl ; Cl ; Cc1c([C@@H](C)NC(=O)c2cc(N3CCC4(CCCN4C)CC3)c3nnc(C)n3c2)cccc1C(F)(F)F]

A mixture of bromide 104 (60 mg, 136 μmol), 1-methyl-1,8-diazaspiro[4.5]decane dihydrochloride 52 (49 mg, 204 μmol) and potassium phosphate (133 g, 612 μmol) in 1,4-dioxane (1 mL) is degassed with argon. Catalyst I (6 mg, 7 μmol) is added and the mixture is heated under argon for 16 h at 110° C. The mixture is diluted with 1,4-dioxane, filtered through a thiol resin, and washed with DMF/MeOH (9:1). The mixture is directly purified by prep. RP-HPLC (basic conditions) to obtain example E80.

Example E80
Analytical HPLC-MS Method: D

| | |
|---|---|
| Rₜ [min]: 1.03 | MS [m/z]: 515 [M + H]⁺ |

Analytical SFC method: AL

| | |
|---|---|
| Rₜ [min]: 2.13 | e.e. > 98% |

¹H NMR (400 MHz, DMSO-d₆) δ (ppm): 1.27-1.40 (2 H), 1.45-1.55 (3 H), 1.66-1.87 (6
H), 2.15-2.24 (3 H), 2.62-2.76 (5 H), 2.79-2.92 (2 H), 4.33-4.50 (2 H), 5.35-5.48 (1
H), 6.74-6.84 (1 H), 7.37-7.46 (1 H), 7.53-7.63 (1 H), 7.70-7.79 (1 H), 8.36-8.46 (1
H), 8.92-9.02 (1 H), missing proton(s) presumably hidden by/ overlapping with solvent
signals.

### Synthesis Scheme of Intermediate 111

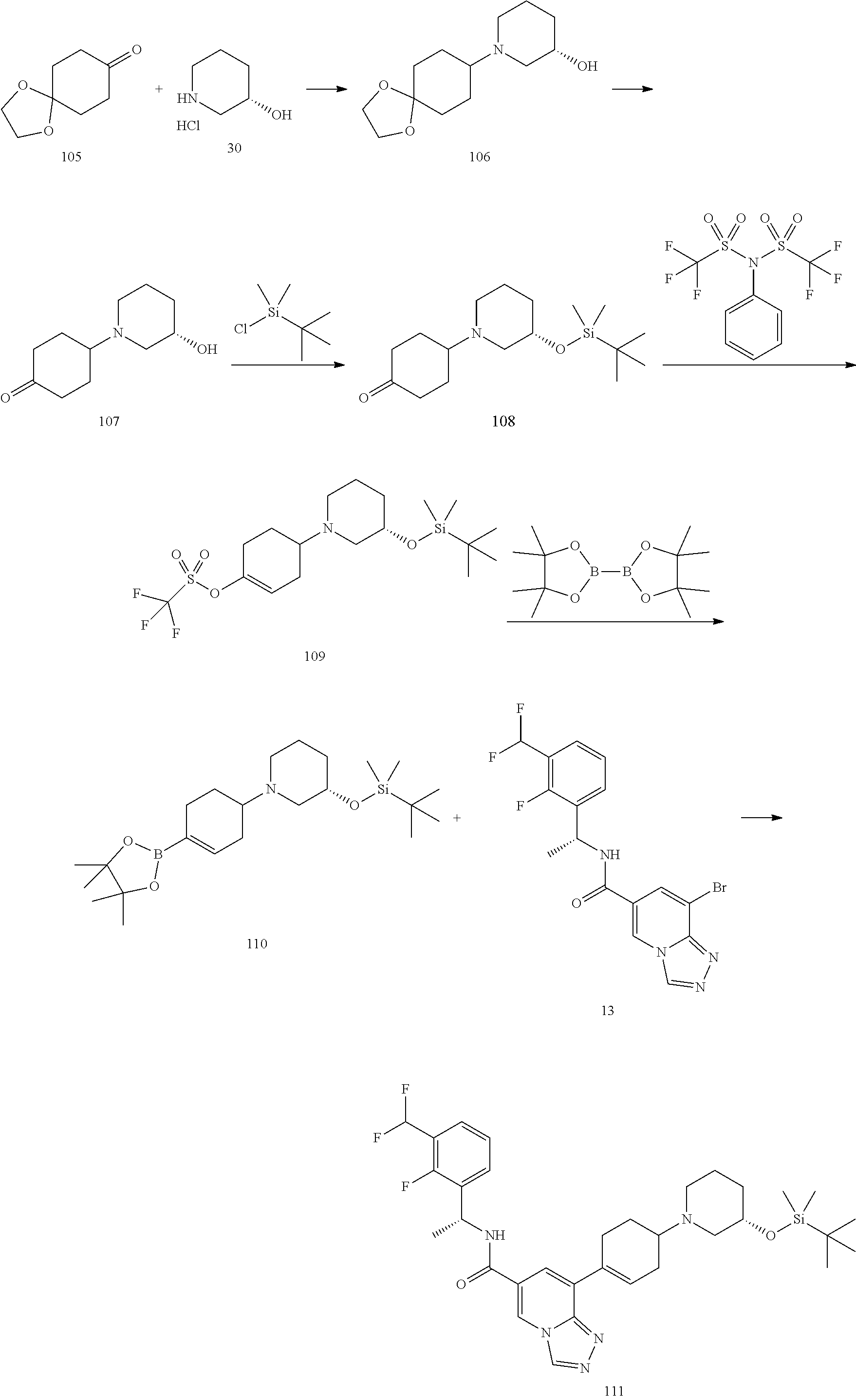

### Synthesis of Intermediate 106

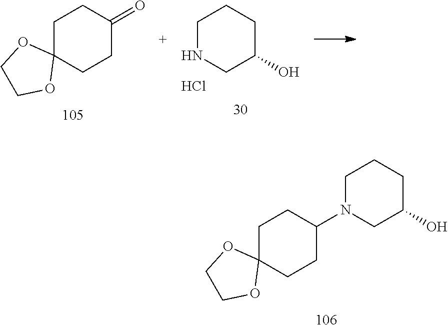 [SMILES: O=C1CCC2(CC1)OCCO2 ; Cl ; O[C@H]1CCCNC1 ; O[C@H]1CCCN(C2CCC3(CC2)OCCO3)C1]

A mixture of 1,4-dioxaspiro[4.5]decan-8-one 105 (3.0 g, 18.8 mmol) and (S)-3-hydroxypiperidine hydrochloride 30 (2.9 g, 20.7 mmol) in THF (30 mL) is stirred at rt for 1 h. Sodium triacetoxyborohydride (5.2 g, 24.5 mmol) is added and the reaction mixture is stirred at rt for 16 h. The mixture is concentrated in vacuo and the residue is submitted to column chromatography (SiO₂; (DCM/MeOH/7 N NH₃ in MeOH=50:48:2)/DCM: 10:90→60:40) to obtain intermediate 106.

Intermediate 106
Analytical HPLC-MS Method: D

| | |
|---|---|
| Rₜ [min]: 0.70 | MS [m/z]: 242 [M + H]⁺ |

### Synthesis of Intermediate 107

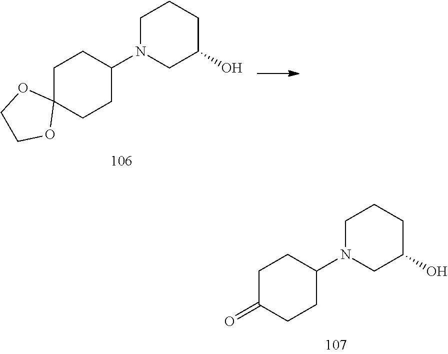 [SMILES: O[C@H]1CCCN(C2CCC3(CC2)OCCO3)C1 ; O=C1CCC(N2CCC[C@H](O)C2)CC1]

A mixture of intermediate 106 (2.66 g, 11.0 mmol) and aqueous HCl (4 N; 10 mL, 40 mmol) in acetone (20 mL) is stirred at rt for 16 h. Aqueous NaOH solution (4 N; 10 mL, 40 mmol) and saturated aqueous NaHCO₃ solution (10 mL) are added, and the mixture is extracted with EtOAc. The organic layers are washed with aqueous saturated NaCl solution, dried over MgSO₄, and concentrated in vacuo. The intermediate 107 is used for the next step without further purification.

Intermediate 107
Analytical HPLC-MS Method: D

| | |
|---|---|
| Rₜ [min]: 0.61 | MS [m/z]: 198 [M + H]⁺ |

### Synthesis of Intermediate 108

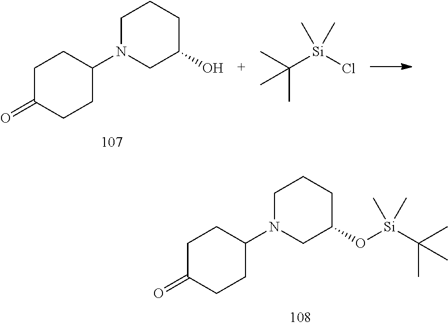

A mixture of intermediate 107 (2.1 g, 10.8 mmol), imidazole (1.8 g, 27.0 mmol) and tert-butyldimethylsilyl chloride (2.4 g, 16.2 mmol) in DMF (20 mL) is stirred for 16 h at rt. The mixture is diluted with water and extracted with EtOAc. The combined organic layers are washed with aqueous saturated NaCl solution, dried over MgSO₄, and concentrated in vacuo. The residue is purified by column chromatography (SiO₂; (DCM/MeOH/7 N NH₃ in MeOH (50:48:2))/DCM: 4:96→30:70) to give intermediate 108.

Intermediate 108
Analytical HPLC-MS Method: D

| | |
|---|---|
| Rₜ [min]: 1.23 | MS [m/z]: 312 [M + H]⁺ |

### Synthesis of Intermediate 109

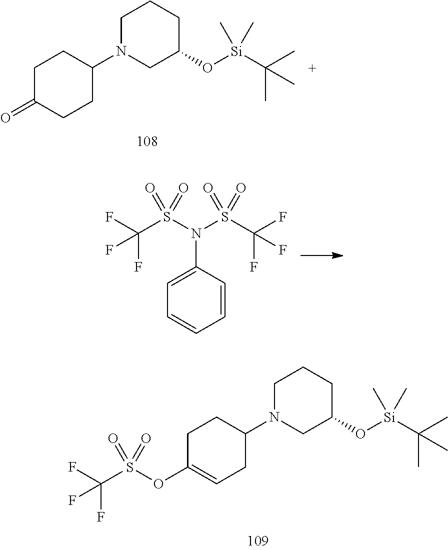 [SMILES: CC(C)(C)[Si](C)(C)O[C@H]1CCCN(C2CCC(=O)CC2)C1 ; O=S(=O)(N(c1ccccc1)S(=O)(=O)C(F)(F)F)C(F)(F)F ; CC(C)(C)[Si](C)(C)O[C@H]1CCCN(C2CC=C(OS(=O)(=O)C(F)(F)F)CC2)C1]

LDA (1 N solution in THF; 16.3 mL, 16.3 mmol) is added to intermediate 108 (2.54 g, 8.15 mmol) in THF (30 mL) at −75° C. under argon atmosphere and the resulting mixture is stirred for 2 h. A solution of N,N-bis(trifluoromethylsulfonyl)aniline (4.08 g, 11.4 mmol) in THF (30 mL) is slowly added at −75° C. and the reaction mixture is stirred for 16 h, being allowed to warm to rt. The reaction mixture is quenched with a saturated aqueous solution of NH₄Cl (50 mL) and extracted with EtOAc. The combined organic layers are washed with aqueous saturated NaCl solution, dried over MgSO₄, and concentrated in vacuo. The residue is purified by column chromatography (SiO₂; cyclohexane/EtOAc: 98:2→70:30) to give intermediate 109.

Intermediate 109
Analytical HPLC-MS Method: D

| | |
|---|---|
| Rₜ [min]: 1.35 | MS [m/z]: 444 [M + H]⁺ |

### Synthesis of Intermediate 110

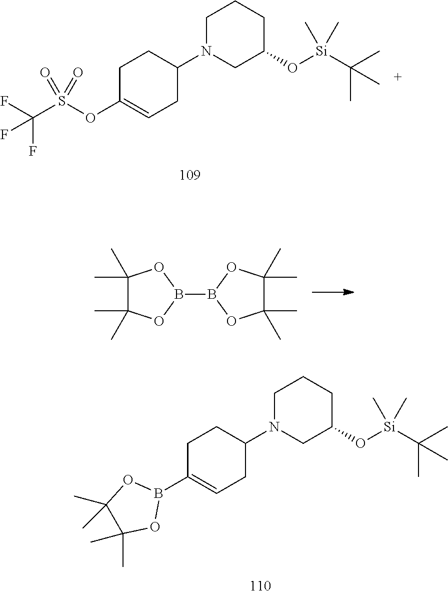

A mixture of intermediate 109 (2.64 g, 5.95 mmol), bis(pinacolato)diboron (1.51 g, 5.95 mmol) and the potassium acetate (1.46 g, 14.9 mmol) in 1,4-dioxane (30 mL) is degassed with argon. Catalyst II (218 mg, 298 μmol) is added, and the mixture is stirred for 1 h at 80° C. under an argon atmosphere. The solvent is evaporated, and EtOAc is added. The organic layer is washed with water and aqueous saturated NaCl solution, dried over MgSO₄, and concentrated in vacuo. The residue is dissolved in DCM and purified by column chromatography (SiO₂; DCM/MeOH: 96:4→85:15). The product is dissolved in ACN/water, filtered, and purified via prep. RP-HPLC (basic conditions) to give intermediate 110.

Intermediate 110
HPLC-MS Method: M

| | |
|---|---|
| Rₜ [min]: 1.25 | MS [m/z]: 422 [M + H]⁺ |

### Synthesis of Intermediate 111

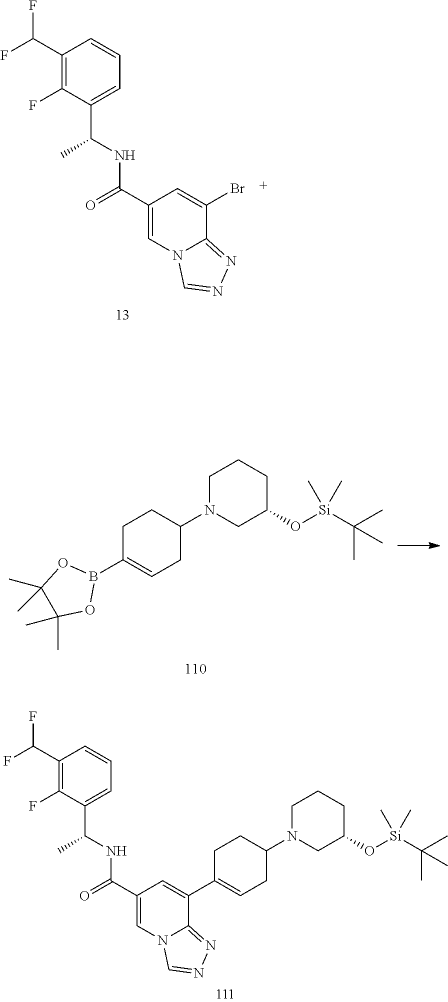 [SMILES: C[C@@H](NC(=O)c1cc(Br)c2nncn2c1)c1cccc(C(F)F)c1F ; CC1(C)OB(C2=CCC(N3CCC[C@H](O[Si](C)(C)C(C)(C)C)C3)CC2)OC1(C)C ; C[C@@H](NC(=O)c1cc(C2=CCC(N3CCC[C@H](O[Si](C)(C)C(C)(C)C)C3)CC2)c2nncn2c1)c1cccc(C(F)F)c1F]

An aqueous Na₂CO₃ solution (2 M; 0.77 mL, 1.55 mmol) is added to a mixture of intermediate 13 (200 mg, 484 μmol) and borane 110 (245 mg, 581 μmol) in 1,4-dioxane (6 mL) and the mixture is degassed with argon. Catalyst II (20 mg, 24 μmol) is added, and the mixture is stirred for 12 h at 95° C. under an argon atmosphere. The mixture is diluted with aqueous saturated NaCl solution and extracted with EtOAc. The combined organic phases are washed with aqueous saturated NaCl solution, dried over MgSO₄, and concentrated in vacuo. The material is dissolved in ACN/MeOH/water, filtered, and purified by prep. RP-HPLC (basic conditions) to give intermediate 111.

Intermediate 111
Analytical HPLC-MS Method: D

| | |
|---|---|
| Rₜ [min]: 1.28 | MS [m/z]: 628 [M + H]⁺ |

### Synthesis of Example E82

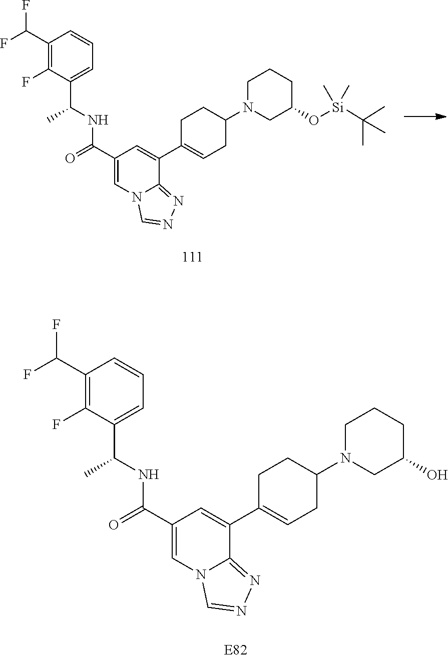 [SMILES: C[C@@H](NC(=O)c1cc(C2=CCC(N3CCC[C@H](O[Si](C)(C)C(C)(C)C)C3)CC2)c2nncn2c1)c1cccc(C(F)F)c1F ; C[C@@H](NC(=O)c1cc(C2=CCC(N3CCC[C@H](O)C3)CC2)c2nncn2c1)c1cccc(C(F)F)c1F]

Tetrabutylammonium fluoride (573 μl, 573 μmol) is added to intermediate 111 (120 mg, 191 μmol) in THF (3 mL) under an argon atmosphere and the resulting mixture is stirred at rt for 20 h. The mixture is diluted with water/ACN and purified via RP-HPLC (basic conditions) to obtain example E82.

Example E82
Analytical HPLC-MS Method: D

| | |
|---|---|
| Rₜ [min]: 0.91 | MS [m/z]: 514 [M + H]⁺ |

¹H NMR (400 MHz, DMSO-d₆) δ (ppm): 1.01-1.21 (1 H), 1.31-1.49 (1 H), 1.49-1.71 (5
H), 1.75-1.88 (1 H), 1.90-2.23 (3 H), 2.25-2.48 (2 H), 2.54-2.86 (4 H), 2.86-3.02 (1
H), 3.39-3.55 (1 H), 4.43-4.64 (1 H), 5.38-5.48 (1 H), 7.07-7.39 (2 H), 7.48-7.58 (2
H), 7.59-7.65 (1 H), 7.65-7.72 (1 H), 8.98-9.07 (1 H), 9.12-9.19 (1 H), 9.34-9.41 (1
H).

### Synthesis of Example E81

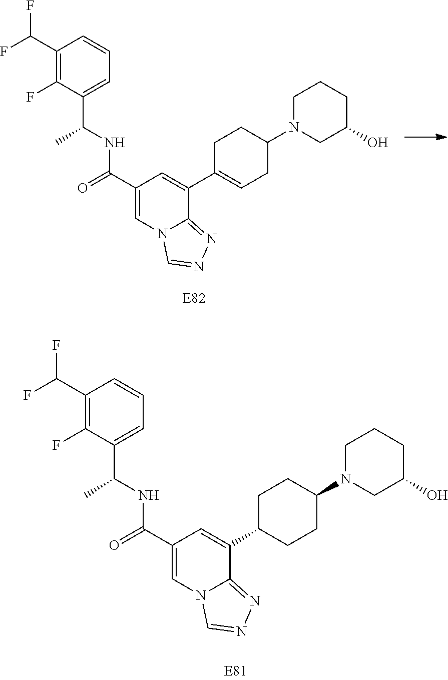 [SMILES: C[C@@H](NC(=O)c1cc(C2=CCC(N3CCC[C@H](O)C3)CC2)c2nncn2c1)c1cccc(C(F)F)c1F ; C[C@@H](NC(=O)c1cc([C@H]2CC[C@H](N3CCC[C@H](O)C3)CC2)c2nncn2c1)c1cccc(C(F)F)c1F]

Example E82 (55 mg, 107 μmol) in MeOH (3 mL) is stirred in the presence of Pd/C (10% w/w, 10 mg) under an H₂ atmosphere (50 psi) for 5 h at rt. The mixture is filtered and concentrated in vacuo. The residue is dissolved in MeOH, filtered, and purified by prep. RP-HPLC (basic conditions) to obtain example E81.

Example E81
Analytical HPLC-MS Method: D:

| | |
|---|---|
| Rₜ [min]: 0.91 | MS [m/z]: 516 [M + H]⁺ |

Analytical SFC method: Z

| | |
|---|---|
| Rₜ [min]: 2.93 | d.e. > 98% |

¹H NMR (400 MHz, DMSO-d₆) δ ppm): 0.99-1.14 (1 H), 1.33-1.55 (6 H), 1.58-1.84 (4
H), 1.86-2.19 (6 H), 2.39-2.48 (1 H), 2.64-2.73 (1 H), 2.84-2.92 (1 H), 3.04-3.16 (1
H), 3.39-3.48 (1 H), 4.51 (1 H), 5.42 (1 H), 7.08-7.38 (2 H), 7.51-7.57 (2 H), 7.68 (1
H), 9.00 (1 H), 9.08 (1 H), 9.34 (1 H).

### Synthesis of Intermediate 113

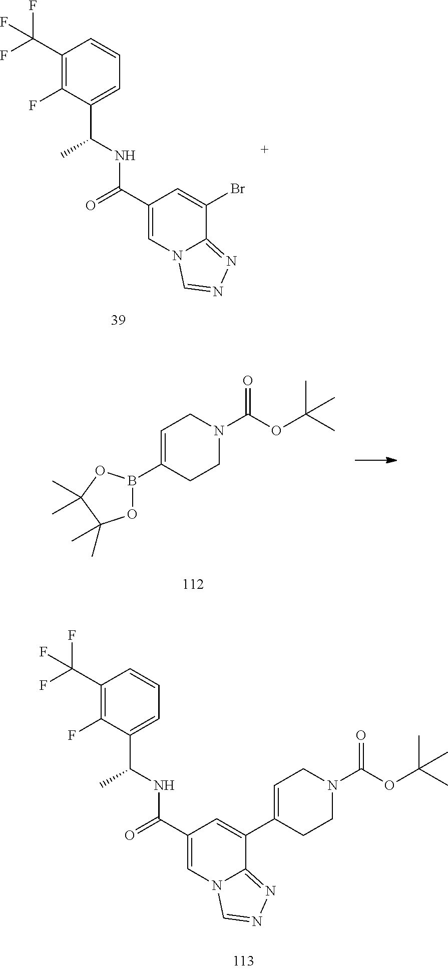 [SMILES: C[C@@H](NC(=O)c1cc(Br)c2nncn2c1)c1cccc(C(F)(F)F)c1F ; CC(C)(C)OC(=O)N1CC=C(B2OC(C)(C)C(C)(C)O2)CC1 ; C[C@@H](NC(=O)c1cc(C2=CCN(C(=O)OC(C)(C)C)CC2)c2nncn2c1)c1cccc(C(F)(F)F)c1F]

A mixture of intermediate 39 (150 mg, 348 μmol) and borane 112 (170 mg, 522 μmol) in 1,4-dioxane (3 mL), MeOH (1.1 mL) and aqueous Na₂CO₃ solution (2 M; 0.55 mL, 1.11 mmol) is degassed with argon. Catalyst II (28 mg, 35 μmol) is added, and the mixture is heated 3 h under argon at 110° C. To the reaction mixture water is added, the aqueous layer is extracted with EtOAc, and the combined organic layers dried with MgSO₄. After filtration and evaporation of the solvent, the mixture is purified by prep. RP-HPLC (basic conditions) to obtain intermediate 113.

Intermediate 113
Analytical HPLC-MS Method: D

| | |
|---|---|
| Rₜ [min]: 1.08 | MS [m/z]: 534 [M + H]⁺ |

### Synthesis of Intermediate 114

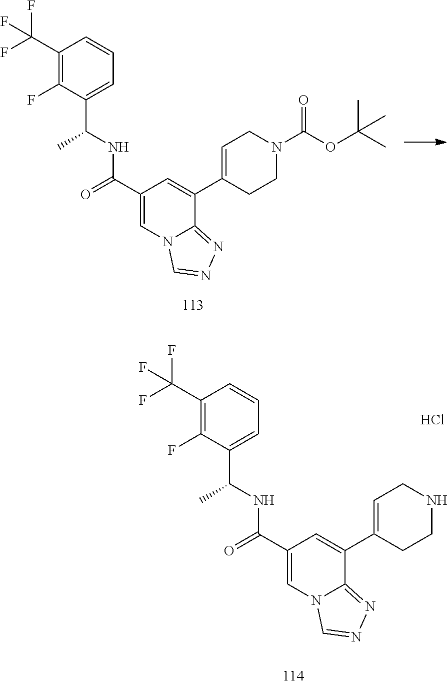 [SMILES: C[C@@H](NC(=O)c1cc(C2=CCN(C(=O)OC(C)(C)C)CC2)c2nncn2c1)c1cccc(C(F)(F)F)c1F ; C[C@@H](NC(=O)c1cc(C2=CCNCC2)c2nncn2c1)c1cccc(C(F)(F)F)c1F ; Cl]

Intermediate 113 (135 mg, 253 μmol) in 1,4-dioxane (2.2 mL) is treated with HCl solution (4 M in 1,4-dioxane; 1.26 mL, 5.06 mmol). The mixture is stirred for 16 h at rt. The solvent is evaporated, the residue co-evaporated with toluene to give intermediate 114, which is used in the next reaction step without further purification.

Intermediate 114
Analytical HPLC-MS Method: D

| | |
|---|---|
| Rₜ [min]: 0.91 | MS [m/z]: 434 [M + H]⁺ |

### Synthesis of Example E83

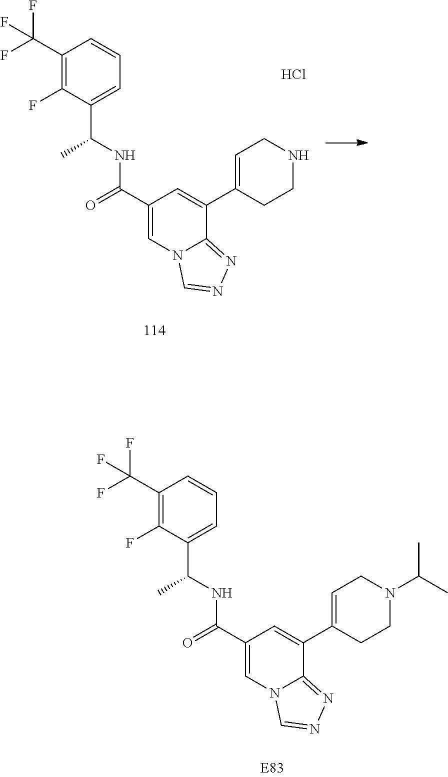 [SMILES: C[C@@H](NC(=O)c1cc(C2=CCNCC2)c2nncn2c1)c1cccc(C(F)(F)F)c1F ; Cl ; CC(C)N1CC=C(c2cc(C(=O)N[C@H](C)c3cccc(C(F)(F)F)c3F)cn3cnnc23)CC1]

A mixture of intermediate 114 (135 mg, 287 μmol) in DMF (1.2 mL) is treated with acetone (73 μL, 1.01 mmol), AcOH (16 μL, 287 μmol), and sodium triacetoxyborohydride (244 mg, 1.15 mmol). The mixture is stirred for 2 h at rt. Additional acetone (73 μL, 1.01 mmol) is added and stirring is continued for 1 h at rt. Water is added, the mixture is neutralized with 10% aqueous NH₃ solution, diluted with THF and MeOH and filtered. The mixture is purified by prep. RP-HPLC (basic conditions) to obtain example E83.

Example E83
Analytical HPLC-MS Method: D

| | |
|---|---|
| Rₜ [min]: 1.02 | MS [m/z]: 476 [M + H]⁺ |

Analytical SFC method: AH

| | |
|---|---|
| Rₜ [min]: 6.68 | e.e. >98% |

¹H NMR (400 MHz, DMSO-d₆) δ (ppm): 0.97-1.12 (6 H), 1.49-1.59
(3 H), 2.57-2.85 (5 H), 5.37-5.49 (1 H), 7.37-7.46 (1 H), 7.59-7.73 (3 H),
7.78-7.87 (1 H), 9.00-9.08 (1 H), 9.13-9.22 (1 H), 9.36-9.42 (1 H),
missing proton(s) presumably hidden by/overlapping with solvent
signals.

### Synthesis Scheme of Intermediate 119

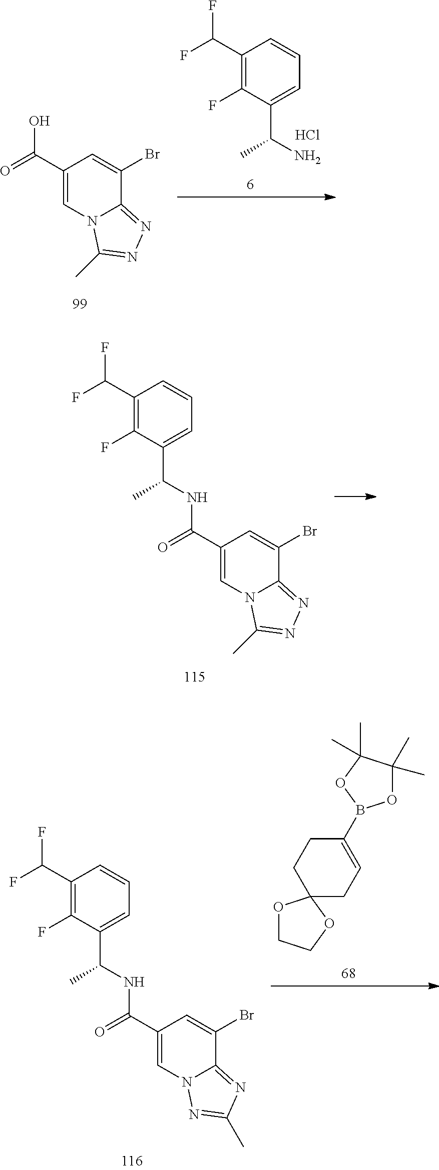 [SMILES: Cc1nnc2c(Br)cc(C(=O)O)cn12 ; C[C@@H](N)c1cccc(C(F)F)c1F ; Cl ; Cc1nnc2c(Br)cc(C(=O)N[C@H](C)c3cccc(C(F)F)c3F)cn12 ; Cc1nc2c(Br)cc(C(=O)N[C@H](C)c3cccc(C(F)F)c3F)cn2n1 ; CC1(C)OB(C2=CCC3(CC2)OCCO3)OC1(C)C]

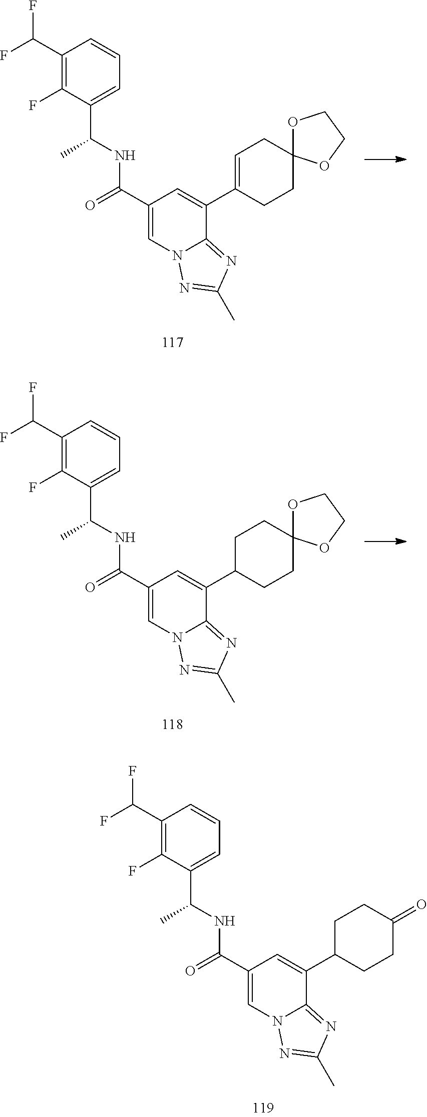 [SMILES: Cc1nc2c(C3=CCC4(CC3)OCCO4)cc(C(=O)N[C@H](C)c3cccc(C(F)F)c3F)cn2n1 ; Cc1nc2c(C3CCC4(CC3)OCCO4)cc(C(=O)N[C@H](C)c3cccc(C(F)F)c3F)cn2n1 ; Cc1nc2c(C3CCC(=O)CC3)cc(C(=O)N[C@H](C)c3cccc(C(F)F)c3F)cn2n1]

### Synthesis of Intermediate 115

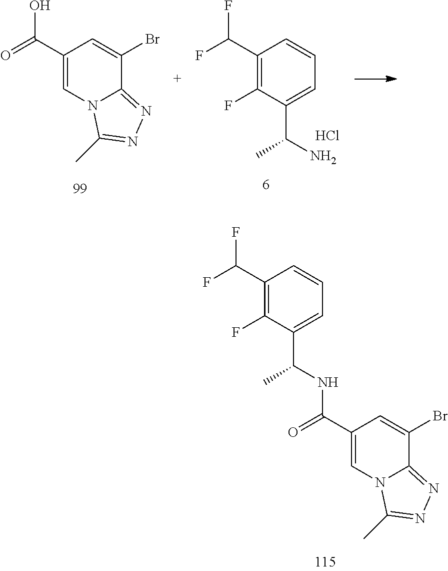 [SMILES: Cc1nnc2c(Br)cc(C(=O)O)cn12 ; C[C@@H](N)c1cccc(C(F)F)c1F ; Cl ; Cc1nnc2c(Br)cc(C(=O)N[C@H](C)c3cccc(C(F)F)c3F)cn12]

A mixture of intermediate 99 (2.0 g, 7.6 mmol), benzylamine 6 (CAS: 1389852-29-2; 1.8 g, 8.0 mmol), 4-methylmorpholine (2.7 g, 27 mmol) in ACN (20 mL) is cooled to 0° C. 1-Propanephosphonic anhydride (50%, 5.0 mL, 8.4 mmol) is added dropwise and the mixture is stirred for 16 h at rt. The mixture is poured into ice-water and treated with aqueous HCl solution. The solid material is filtered off, washed with water and dried in vacuo at 55° C. to obtain intermediate 115.

Intermediate 115
Analytical HPLC-MS Method: D

| | |
|---|---|
| Rₜ [min]: 0.90 | MS [m/z]: 427 [M + H]⁺ |

### Synthesis of Intermediate 116

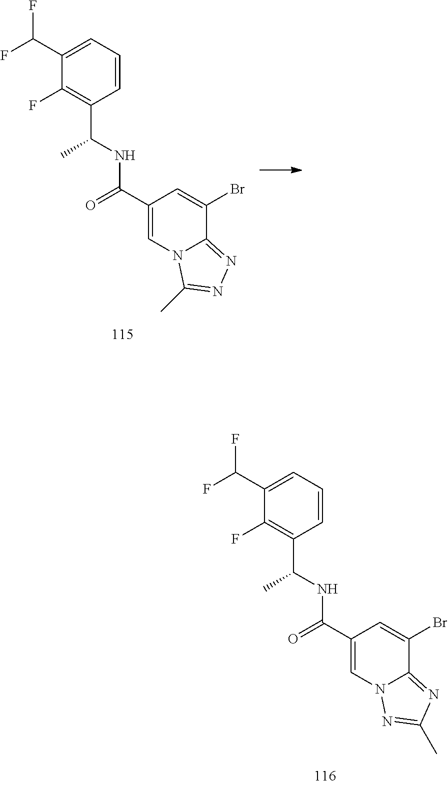 [SMILES: Cc1nnc2c(Br)cc(C(=O)N[C@H](C)c3cccc(C(F)F)c3F)cn12 ; Cc1nc2c(Br)cc(C(=O)N[C@H](C)c3cccc(C(F)F)c3F)cn2n1]

A mixture of intermediate 115 (2.6 g, 6.1 mmol), Cs₂CO₃ (2.0 g, 6.1 mmol), piperidine (603 μL, 6.1 mmol) in 1,4-dioxane (16 mL) is stirred for 7 h at 120° C. and for 48 h at rt. The solvent is evaporated under reduced pressure. EtOAc and water are added. The aqueous layer is separated and extracted with EtOAc. The combined organic layers are dried over MgSO₄. The solids are filtered, and the solvent is evaporated under reduced pressure. The material is purified by prep. RP-HPLC (basic conditions) to obtain intermediate 116.

Intermediate 116
Analytical HPLC-MS Method: D

| | |
|---|---|
| Rₜ [min]: 0.95 | MS [m/z]: 427 [M + H]⁺ |

### Synthesis of Intermediate 117

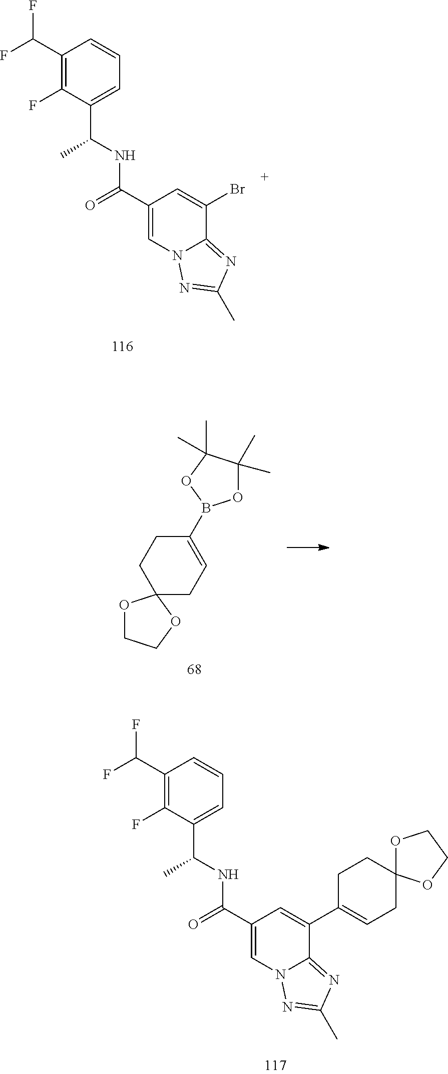 [SMILES: Cc1nc2c(Br)cc(C(=O)N[C@H](C)c3cccc(C(F)F)c3F)cn2n1 ; CC1(C)OB(C2=CCC3(CC2)OCCO3)OC1(C)C ; Cc1nc2c(C3=CCC4(CC3)OCCO4)cc(C(=O)N[C@H](C)c3cccc(C(F)F)c3F)cn2n1]

A mixture of intermediate 116 (800 mg, 1.8 mmol), aqueous Na₂CO₃ solution (2 M; 2.8 mL, 5.7 mmol), and borane 68 (575 mg, 2.1 mmol) in 1,4-dioxane (10 mL) is stirred under argon atmosphere. Catalyst II (87 mg, 107 μmol) is added, and the mixture is stirred at 95° C. for 2 h. The mixture is diluted with water and extracted with EtOAc. The combined organic layers are washed with aqueous saturated NaCl solution, dried over Na₂SO₄, filtered and the solvents are evaporated. The material is purified by column chromatography (SiO₂; EtOAc/cyclohexane: 50:50→100:0) to obtain intermediate 117.

Intermediate 117
HPLC-MS Method: E

| | |
|---|---|
| Rₜ [min]: 1.03 | MS [m/z]: 487 [M + H]⁺ |

### Synthesis of Intermediate 118

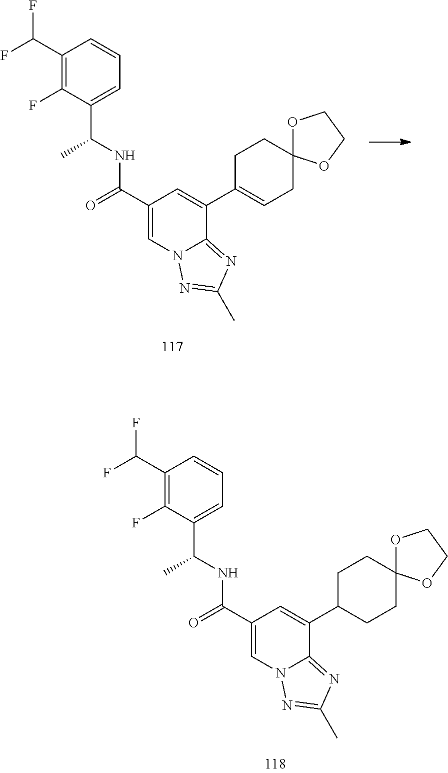 [SMILES: Cc1nc2c(C3=CCC4(CC3)OCCO4)cc(C(=O)N[C@H](C)c3cccc(C(F)F)c3F)cn2n1 ; Cc1nc2c(C3CCC4(CC3)OCCO4)cc(C(=O)N[C@H](C)c3cccc(C(F)F)c3F)cn2n1]

Intermediate 117 (940 mg, 1.9 mmol) in MeOH (10 mL) is stirred under H₂ atmosphere (50 psi) in the presence of Pd/C (10% w/w, 100 mg) for 18 h at rt. The mixture is filtered, and the solvent is evaporated to obtain intermediate 118. The material is used in the next reaction step without further purification.

Intermediate 118
HPLC-MS Method: E

| | |
|---|---|
| Rₜ [min]: 1.02 | MS [m/z]: 489 [M + H]⁺ |

### Synthesis of Intermediate 119

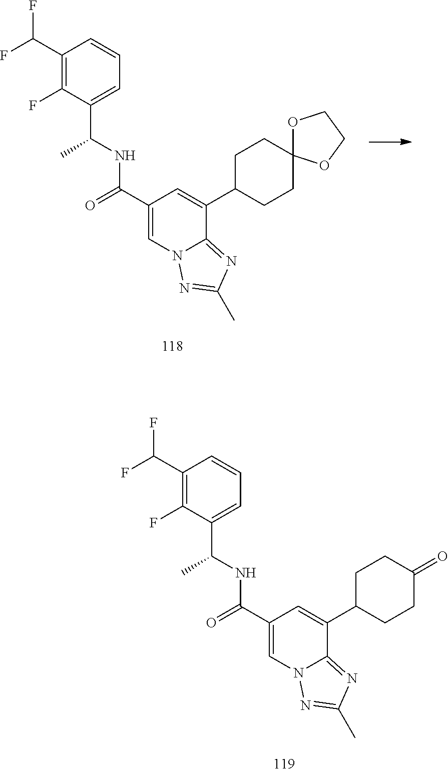 [SMILES: Cc1nc2c(C3CCC4(CC3)OCCO4)cc(C(=O)N[C@H](C)c3cccc(C(F)F)c3F)cn2n1 ; Cc1nc2c(C3CCC(=O)CC3)cc(C(=O)N[C@H](C)c3cccc(C(F)F)c3F)cn2n1]

Intermediate 118 (783 mg, 1.6 mmol) in THF (10 mL) is treated with aqueous HCl solution (4 M; 2.0 mL). The mixture is stirred for 24 h at rt. A saturated NaHCO₃ solution is added, and the mixture is extracted with EtOAc. The separated organic layer is extracted with aqueous saturated NaCl solution. The solvent of the combined organic layers is evaporated under reduced pressure to obtain intermediate 119. The material is used in the next reaction step without further purification.

Intermediate 119
HPLC-MS Method: E

| | |
|---|---|
| Rₜ [min]: 0.97 | MS [m/z]: 445 [M + H]⁺ |

### Synthesis of Example E84

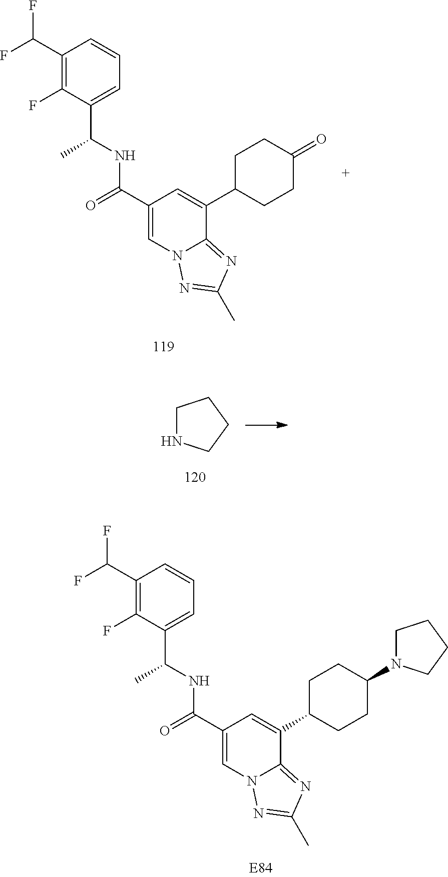 [SMILES: Cc1nc2c(C3CCC(=O)CC3)cc(C(=O)N[C@H](C)c3cccc(C(F)F)c3F)cn2n1 ; C1CCNC1 ; Cc1nc2c([C@H]3CC[C@H](N4CCCC4)CC3)cc(C(=O)N[C@H](C)c3cccc(C(F)F)c3F)cn2n1]

A mixture of intermediate 119 (50 mg, 112 μmol) and pyrrolidine 120 (19 μL, 225 μmol) in MeOH (1 mL) is prepared and AcOH (60 μL, 1.03 mmol) is added followed by 2-picoline-borane complex (12 mg, 112 μmol). The mixture is stirred for 60 h at rt. An aqueous NaOH solution is added and directly purified by prep. RP-HPLC (basic conditions) to obtain example E84.

Example E84
Analytical HPLC-MS Method: D

| | |
|---|---|
| Rₜ [min]: 1.07 | MS [m/z]: 500 [M + H]⁺ |

Analytical SFC method: Y

| | |
|---|---|
| Rₜ [min]: 5.57 | e.e. >98% |

¹H NMR (400 MHz, DMSO-d₆) δ (ppm): 1.27-1.41 (2 H), 1.49-1.56
(3 H), 1.62-1.77 (6 H), 1.92-2.02 (2 H), 2.03-2.14 (3 H), 2.99-3.11 (1 H),
5.37-5.47 (1 H), 7.07-7.37 (2 H), 7.49-7.56 (1 H), 7.65-7.72 (1 H),
7.79-7.83 (1 H), 9.04-9.09 (1 H), 9.22-9.28 (1 H), missing proton(s)
presumably hidden by/overlapping with solvent signals.

### Synthesis of Example E85

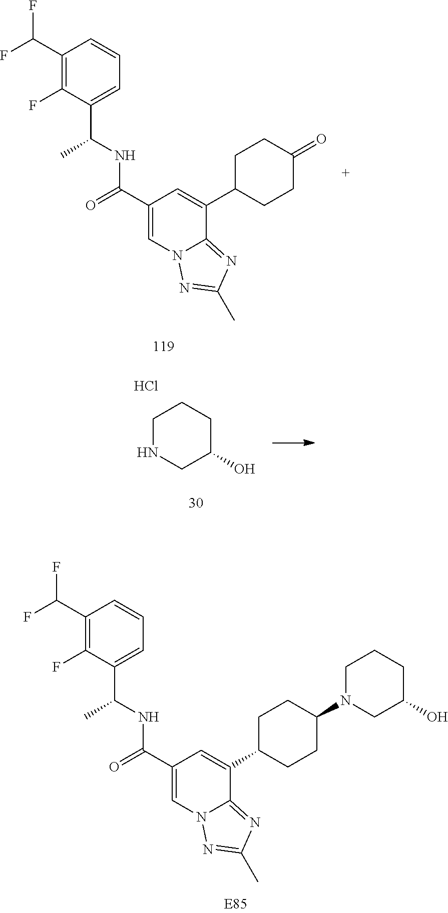 [SMILES: Cc1nc2c(C3CCC(=O)CC3)cc(C(=O)N[C@H](C)c3cccc(C(F)F)c3F)cn2n1 ; Cl ; O[C@H]1CCCNC1 ; Cc1nc2c([C@H]3CC[C@H](N4CCC[C@H](O)C4)CC3)cc(C(=O)N[C@H](C)c3cccc(C(F)F)c3F)cn2n1]

A mixture of intermediate 119 (180 mg, 405 μmol) and (S)-3-hydroxypiperidine hydrochloride 30 (115 mg, 810 μmol) in iPrOH (3 mL) is prepared and AcOH (500 μL, 8.6 mmol) is added followed by 2-picoline-borane complex (45 mg, 405 μmol). The mixture is stirred for 18 h at rt. An aqueous NaOH solution (4 M; 2 mL) is added and directly purified by prep. RP-HPLC (basic conditions) to obtain example E85.

Example E85
Analytical HPLC-MS Method: D

| | |
|---|---|
| Rₜ [min]: 0.97 | MS [m/z]: 530 [M + H]⁺ |

Analytical SFC method: N

| | |
|---|---|
| Rₜ [min]: 3.05 | d.e. >98% |

¹H NMR (400 MHz, DMSO-d₆) δ (ppm): 0.99-1.12 (1 H), 1.30-1.92
(13 H), 1.93-2.05 (3 H), 2.08-2.17 (1 H), 2.38-2.47 (1 H), 2.64-2.73
(1 H), 2.83-2.90 (1 H), 2.98-3.08 (1 H), 3.38-3.48 (1 H), 4.47-4.52
(1 H), 5.37-5.46 (1 H), 7.06-7.37 (2 H), 7.49-7.56 (1 H), 7.65-7.72
(1 H), 7.78-7.82 (1 H), 9.03-9.10 (1 H), 9.24-9.28 (1 H), missing
proton(s) presumably hidden by/overlapping with solvent signals.

### Synthesis of Intermediate 122

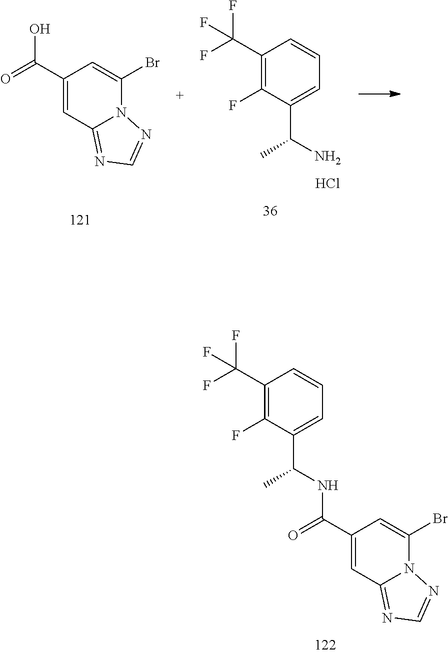 [SMILES: O=C(O)c1cc(Br)n2ncnc2c1 ; C[C@@H](N)c1cccc(C(F)(F)F)c1F ; Cl ; C[C@@H](NC(=O)c1cc(Br)n2ncnc2c1)c1cccc(C(F)(F)F)c1F]

HATU (864 mg, 2.27 mmol) and triethylamine (0.87 mL, 6.20 mmol) are added to intermediate 121 (500 mg, 2.07 mmol) in THF (33 mL). The mixture is stirred for 15 min at rt then (1R)-1-[2-fluoro-3-(trifluoromethyl)phenyl]ethan-1-amine hydrochloride 36 (CAS: 2230840-52-3; 503 mg, 2.07 mmol) is added. Stirring is continued for 16 h at rt. Semi-saturated aqueous NaHCO₃ solution is added, and the aqueous layer extracted with EtOAc. The combined organic layers are dried with MgSO₄. After filtration and evaporation of the solvent, the material is purified by prep. RP-HPLC (basic conditions) to obtain intermediate 122.

Intermediate 122
Analytical HPLC-MS Method: D

| | |
|---|---|
| Rₜ [min]: 1.00 | MS [m/z]: 431 [M + H]⁺ |

### Synthesis of Example E86

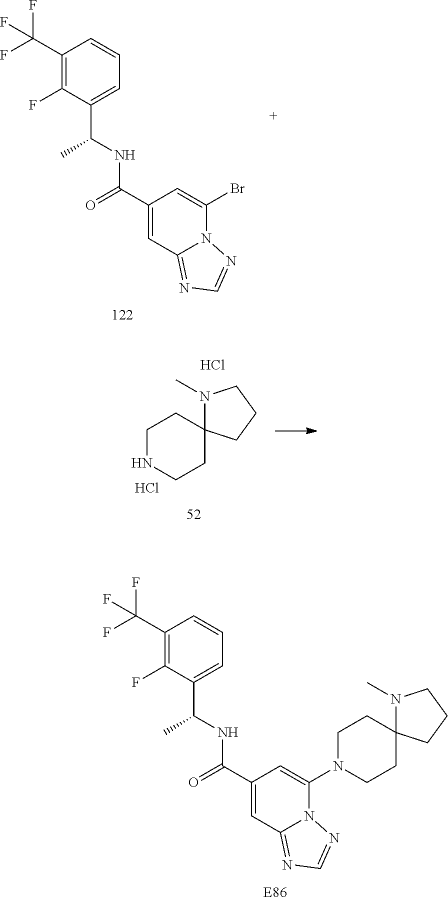 [SMILES: C[C@@H](NC(=O)c1cc(Br)n2ncnc2c1)c1cccc(C(F)(F)F)c1F ; Cl ; CN1CCCC12CCNCC2 ; Cl ; C[C@@H](NC(=O)c1cc(N2CCC3(CCCN3C)CC2)n2ncnc2c1)c1cccc(C(F)(F)F)c1F]

A mixture of intermediate 122 (70 mg, 154 μmol) and 1-methyl-1,8-diazaspiro[4.5]decane dihydrochloride 52 (41 mg, 170 μmol) in 1,4-dioxane (1.3 mL) is degassed with argon. Cs₂CO₃ (201 mg, 617 μmol) and catalyst I (8 mg, 9 μmol) are added, and the mixture is stirred at 120° C. for 16 h. The reaction mixture is diluted with DMF/MeOH/THF, filtered, concentrated, and purified by prep. RP-HPLC (basic conditions) to obtain example E86.

Example E86
Analytical HPLC-MS Method: D

| | |
|---|---|
| Rₜ [min]: 1.05 | MS [m/z]: 505 [M + H]⁺ |

Analytical SFC method: L

| | |
|---|---|
| Rₜ [min]: 1.91 | e.e. >98% |

¹H NMR (400 MHz, DMSO-d₆) δ (ppm): 1.37-1.48 (2 H), 1.50-1.58
(3 H), 1.66-1.94 (6 H), 2.24-2.35 (3 H), 2.69-2.99 (4 H), 4.01-4.15 (2 H),
5.39-5.49 (1 H), 6.86-6.95 (1 H), 7.37-7.47 (1 H), 7.64-7.73 (1 H),
7.80-7.88 (1 H), 7.97-8.04 (1 H), 8.52-8.63 (1 H), 9.20-9.29 (1 H).

### Pharmacological Activity—Biological Assays and Data

### KRAS::SOS1 Alphascreen Binding Assay

This assay can be used to examine the potency with which compounds inhibit the protein-protein interaction between SOS1 and KRAS G12C or G12D. This demonstrates the molecular mode of action of compounds. Low IC₅₀ values are indicative of high potency of the SOS1 inhibitor compound in this assay setting:

### Reagents:

GST-tagged SOS1 (564_1049_GST_TEV_ECO) produced in-house
6xHis-Tev-K-RasG12D/G12C(1-169)Avi produced in-house
GDP (Sigma Cat No G7127)
AlphaLISA Glutathione Acceptor Beads (PerkinElmer, Cat No AL109)
AlphaScreen Streptavidin Donor Beads (PerkinElmer Cat No 6760002)
Assay plates: Proxiplate-384 PLUS, white (PerkinElmer, Cat No 6008289)

### Assay Buffer:

$$1 \times PBS \\ 0.1 \% BSA \\ 0.05 \% Tween 20$$

KRAS::SOS1 GDP mix:
7.5 nM (final assay concentration) K-RasG12C, 10 nM (final assay concentration) K-RasG12D, 10 μM (final assay concentration) GDP and 5 nM (final assay concentration) GST-SOS1 are mixed in assay buffer prior to use and kept at rt. Bead mix:
AlphaLISA Glutathione Acceptor Beads and AlphaScreen Streptavidin Donor Beads are mixed in the dark in assay buffer at a concentration of 10 μg/mL (final assay concentration) each prior to use and kept at rt.

### Assay Protocol:

Compounds are diluted to a final start concentration of 100 μM and are tested in duplicate. Assay-ready plates (ARPs) are generated using an Access Labcyte Workstation with a Labcyte Echo 550 or 555 acoustic dispenser. For compound a start concentration of 100 μM, 150 nL of compound solution is transferred per well in 11 concentrations in duplicate with serial 1:5 dilutions.

The assay is run using a fully automated robotic system in a darkened room below 100 Lux. 10 μL of KRAS::SOS1 GDP mix is added into columns 1-24 to the 150 nL of compound solution (final dilution in the assay 1:100, final DMSO concentration 1%).

After a 30 minute incubation time, 5 μL of bead mix is added into columns 1-23. Plates are kept at rt in a darkened incubator. After a further 60 minutes incubation, the signal is measured using a PerkinElmer Envision HTS Multilabel Reader using the AlphaScreen specifications from PerkinElmer. Each plate contains the following controls:

diluted DMSO+KRAS::SOS1 GDP mix+bead mix

diluted DMSO+KRAS::SOS1 GDP mix

Result calculation: IC₅₀ values are calculated and analyzed using a 4 parametric logistic model.

Table 1: Inhibition values for KRAS G12C as well as for KRAS G12D. Data obtained with the disclosed assay for a selection of compounds (I) according to the invention. The value indicates an average value of at least two measurements.

| Example | IC₅₀ [nM] | IC₅₀ [nM] |
|---|---|---|
| number | G12C | G12D |
| E1 | 33 | 30 |
| E2 | 55 | 46 |
| E3 | 59 | 50 |
| E4 | 82 | 70 |
| E5 | 65 | 57 |
| E6 | 36 | 37 |
| E7 | 50 | 43 |
| E8 | 70 | 69 |
| E10 | 74 | 67 |
| cis-E11a | 22 | 15 |
| cis-E11b | 28 | 19 |
| E12 | 30 | 24 |
| E13 | 52 | 54 |
| E14 | 69 | 51 |
| E15 | 68 | 46 |
| cis-E16a | 45 | 41 |
| cis-E16b | 26 | 19 |
| E17a | 39 | 28 |
| E17b | 38 | 32 |
| E18 | 49 | 37 |
| E19 | 32 | 27 |
| E20 | 44 | 33 |
| cis-E21a | 81 | 61 |
| cis-E21b | 78 | 82 |
| E22 | 48 | 49 |
| E24 | 56 | 46 |
| E25 | 63 | 53 |
| E26 | 86 | 78 |
| E27 | 77 | 73 |
| E28 | 70 | 70 |
| cis-E29 | 56 | 46 |
| E30 | 59 | 51 |
| E31 | 33 | 28 |
| E32 | 44 | 43 |
| E33 | 80 | 73 |
| E34 | 42 | 34 |
| E35 | 57 | 48 |
| E36 | 28 | 22 |
| E37 | 31 | 21 |
| E38a | 66 | 45 |
| E38b | 59 | 41 |
| E39 | 53 | 54 |
| E40 | 50 | 53 |
| cis-E41a | 50 | 38 |
| cis-E41b | 41 | 38 |
| trans-E42a | 26 | 26 |
| trans-E42b | 37 | 36 |
| E43a | 42 | 40 |
| E43b | 34 | 35 |
| E44 | 63 | 42 |
| E45 | 33 | 30 |
| E46 | 34 | 31 |
| E47a | 96 | 99 |
| E47b | 41 | 45 |
| E48 | 69 | 60 |
| E49 | 49 | 43 |
| E50 | 49 | 42 |
| cis-E52a | 59 | 58 |
| cis-E52b | 71 | 70 |
| trans-E53a | 24 | 24 |
| trans-E53b | 33 | 35 |
| E54 | 39 | 31 |
| E55 | 69 | 54 |
| E57 | 65 | 52 |
| E58 | 32 | 32 |
| E59 | 42 | 41 |
| E60 | 21 | 20 |
| trans-E61a | 39 | 41 |
| trans-E61b | 32 | 32 |
| E62 | 22 | 15 |
| E63 | 31 | 27 |
| E64a | 41 | 39 |
| E64b | 35 | 36 |
| E65 | 90 | 93 |
| E66 | 74 | 79 |
| E67 | 33 | 33 |
| E68 | 36 | 27 |
| E69 | 28 | 23 |
| trans-E70a | 29 | 34 |
| trans-E70b | 36 | 42 |
| E71 | 79 | 79 |
| E72 | 33 | 32 |
| E73 | 64 | 64 |
| E74 | 26 | 21 |
| E75 | 65 | 60 |
| E76 | 49 | 36 |
| E77 | 27 | 22 |
| E78 | 43 | 41 |
| E79 | 20 | 14 |
| E80 | 37 | 27 |
| E81 | 25 | 24 |
| E82 | 20 | 15 |
| E83 | 30 | 26 |
| E84 | 38 | 37 |
| E85 | 37 | 24 |
| E86 | 61 | 44 |

### Erk Phosphorylation Assay

ERK phosphorylation assays are used to examine the potency with which compounds inhibit the SOS1-mediated signal transduction in a KRAS mutant human cancer cell line in vitro. This demonstrates the molecular mode of action of compounds by interfering with the RAS-family protein signal transduction cascade. Low IC₅₀ values are indicative of high potency of the SOS1 inhibitor compounds in this assay setting. It is observed that SOS1 inhibitor compounds demonstrate an inhibitory effect on ERK phosphorylation in a KRAS mutant human cancer cell line, thus confirming the molecular mode of action of the SOS1 inhibitor compounds on RAS-family protein signal transduction.

ERK phosphorylation assays are performed using the following human cell line:
NCI-H358 SOS2 KO (Hofmann, Gmachl, Ramharter et al, Cancer Discov. 2021, 11(1):142-15): human lung cancer with a KRAS G12C mutation;

### Materials Used:

RPMI-1640 Medium (ATCC@A10491-01™)
DMEM Medium (Sigma Aldrich #D6429)
Fetal Bovine Serum (FBS) from HyClone (SH30084-03)
384 plates from Greiner Bio-One (781182)
Proxiplate™ 384 from PerkinElmer Inc. (6008280)
AlphaLISA SureFire Ultra p-ERK1/2 (Thr202/Tyr204) Assay Kit (ALSU-PERK-A10K)
Acceptor Mix: Protein A Acceptor Beads from PerkinElmer (6760137M)
Donor Mix: AlphaScreen Streptavidin-coated Donor Beads from PerkinElmer (6760002)
Human EGF 100 μg/mL Peprotech (PNr.: AF-100-15)
Complete Mini, Proteaseinhibitor Cocktail Tablets, Roche #11836170001

### Assay Setup:

NCI-H358 SOS2 KO are seeded at 50000 cells per well in 60 μL of DMEM with 2% FBS, in Greiner TC 384 plates. The cells are incubated overnight in an incubator at 37° C. and 5% CO₂ in a humidified atmosphere. 60 nL compound solution (10 mM DMSO stock solution) is then added using a Beckman Coulter Labcyte Echo 550 device. After 50 min incubation in the aforementioned incubator, 5 nl EGF are added to each well for a final concentration of approx. 8 ng/mL using a Beckman Coulter Labcyte Echo 550 device and cells are incubated for another 10-15 minutes before lysis. The medium is removed, and the cells are lysed by addition of 20 μL of 1.6-fold lysis buffer from the AlphaLISA SureFire Ultra pERK1/2 (Thr202/Tyr204) Assay Kit with added protease inhibitors. After 20 minutes of incubation at rt with shaking, 6 μL of each lysate sample is transferred to a 384-well Proxiplate and analyzed for pERK (Thr202/Tyr204) with the AlphaLISA SureFire Ultra pERK1/2 (Thr202/Tyr204) Assay Kit. 3 μL Acceptor Mix and 3 μL Donor Mix are added under subdued light and incubated for 2 h at rt in the dark, before the signal is measured on a Perkin Elmer Envision plate reader using 384 AlphaScreen settings for Proxiplates. Data are fitted by iterative calculation with variable hill slope. The sigmoidal curve slope is fitted using a default fitting curve to ascertain IC₅₀ values. A minimal efficacy of 35% normalized pathway modulation is set as threshold for curve fitting and consideration of resulting IC₅₀ values.

Table 2: Erk Phosphorylation assay. The value indicates an average value of at least two measurements.

| Example | pERK |
|---|---|
| Number | IC50 [nM] |
| E1 | 118 |
| E2 | 92 |
| E3 | 155 |
| E4 | 336 |
| E5 | 324 |
| E6 | 247 |
| E7 | 246 |
| E8 | 188 |
| E10 | 212 |
| cis-E11a | 173 |
| cis-E11b | 145 |
| E12 | 110 |
| E13 | 288 |
| E14 | 490 |
| E15 | 554 |
| cis-E16a | 320 |
| cis-E16b | 316 |
| E17a | 94 |
| E17b | 201 |
| E18 | 54 |
| E19 | 222 |
| E20 | 146 |
| cis-E21a | 216 |
| cis-E21b | 242 |
| E22 | 187 |
| E24 | 325 |
| E25 | 282 |
| E26 | 147 |
| E27 | 73 |
| E28 | 186 |
| cis-E29 | 261 |
| E30 | 296 |
| E31 | 153 |
| E32 | 270 |
| E33 | 326 |
| E34 | 62 |
| E35 | 160 |
| E36 | 144 |
| E37 | 152 |
| E38a | 112 |
| E38b | 217 |
| E39 | 251 |
| E40 | 277 |
| cis-E41a | 463 |
| cis-E41b | 367 |
| trans-E42a | 183 |
| trans-E42b | 153 |
| E43a | 46 |
| E43b | 101 |
| E44 | 58 |
| E45 | 139 |
| E46 | 162 |
| E47a | 162 |
| E47b | 198 |
| E48 | 82 |
| E49 | 250 |
| E50 | 220 |
| cis-E52a | 115 |
| cis-E52b | 40 |
| trans-E53a | 143 |
| trans-E53b | 287 |
| E54 | 186 |
| E55 | 397 |
| E57 | 149 |
| E58 | 122 |
| E59 | 166 |
| E60 | 290 |
| trans-E61a | 391 |
| trans-E61b | 138 |
| E62 | 315 |
| E63 | 89 |
| E64a | 198 |
| E64b | 366 |
| E65 | 165 |
| E66 | 152 |
| E67 | 46 |
| E68 | 116 |
| E69 | 218 |
| trans-E70a | 259 |
| trans-E70b | 169 |
| E71 | 196 |
| E72 | 92 |
| E73 | 136 |
| E74 | 206 |
| E75 | 408 |
| E76 | 230 |
| E77 | 202 |
| E78 | 223 |
| E79 | 106 |
| E80 | 213 |
| E81 | 258 |
| E82 | 90 |
| E83 | 132 |
| E84 | 174 |
| E85 | 158 |
| E86 | 174 |

### Metabolic Stability in Human Hepatocytes

The metabolic degradation of a test compound is performed with hepatocytes in suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 50% human serum) to obtain a final cell density of 1.0×10⁶ cells/mL. Following a preincubation in a cell culture incubator (37° C., 10% CO₂), test compound dissolved in DMSO is mixed with the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation. The supernatant is transferred to a deep-well plate and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T_{1/2}).

In vitro intrinsic clearance (CLᵢₙₜ) is calculated from in vitro T_{1/2} and scaled to whole liver using a hepatocellularity of 120×10⁶ cells/g liver, a human liver per body weight of 25.7 g liver/kg as well as in vitro incubation parameters, applying the following equation:

CL_INTRINSIC_IN VIVO[mL/min/kg]=(CL_INTRINSIC[μL/min/10⁶cells]×hepatocellularity[10⁶cells/g liver]×liver factor[g/kg body weight])/1000

Hepatic in vivo blood clearance (CL) is predicted according to the well-stirred liver model considering an average liver blood flow (QH) of 20.7 mL/min/kg:

CL[mL/min/kg]=CL_INTRINSIC_IN VIVO[mL/min/kg]×hepatic blood flow[mL/min/kg]/(CL_INTRINSIC_IN VIVO[mL/min/kg]+hepatic blood flow[mL/min/kg])

Results are expressed as percentage of hepatic blood flow:

QH[%]=CL[mL/min/kg]/hepatic blood flow[mL/min/kg])

Table 3: Hepatic clearance expressed as percentage of liver blood flow (QH [%]) of exemplified compounds. Data obtained with the disclosed assay for a selection of compounds (I) according to the invention. At least one measurement otherwise average values of at least two measurements.

| | Hepatic clearance - |
|---|---|
| Example | QH[%] in human |
| Number | hepatocytes |
| E1 | 17 |
| E2 | 15 |
| E3 | 24 |
| E4 | 20 |
| E5 | 16 |
| E6 | 16 |
| E7 | 31 |
| E8 | 18 |
| E10 | 29 |
| cis-E11a | 38 |
| cis-E11b | 27 |
| E12 | 12 |
| E13 | 21 |
| E14 | 31 |
| E15 | 31 |
| cis-E16a | 32 |
| cis-E16b | 24 |
| E17a | 13 |
| E17b | 30 |
| E18 | 17 |
| E19 | 19 |
| E20 | 19 |
| cis-E21a | 25 |
| cis-E21b | 38 |
| E22 | 19 |
| E24 | 22 |
| E25 | 17 |
| E26 | 8 |
| E27 | 9 |
| E28 | 23 |
| cis-E29 | 24 |
| E30 | 18 |
| E31 | 13 |
| E32 | 23 |
| E33 | 15 |
| E34 | 10 |
| E35 | 6 |
| E36 | 10 |
| E37 | 25 |
| E38a | 10 |
| E38b | 11 |
| E39 | 43 |
| E40 | 22 |
| cis-E41a | 37 |
| cis-E41b | 26 |
| trans-E42a | 18 |
| trans-E42b | 16 |
| E43a | 25 |
| E43b | 25 |
| E44 | 16 |
| E45 | <4 |
| E46 | 9 |
| E47a | 30 |
| E47b | 28 |
| E48 | 27 |
| E49 | 23 |
| E50 | 15 |
| cis-E52a | 7 |
| cis-E52b | 14 |
| trans-E53a | 45 |
| trans-E53b | 28 |
| E54 | 12 |
| E55 | <4 |
| E57 | 15 |
| E58 | 15 |
| E59 | 15 |
| E60 | 26 |
| trans-E61a | 40 |
| trans-E61b | 30 |
| E62 | 9 |
| E63 | 23 |
| E64a | 16 |
| E64b | 23 |
| E65 | 38 |
| E66 | 32 |
| E67 | 10 |
| E68 | 12 |
| E69 | <4 |
| trans-E70a | 25 |
| trans-E70b | 19 |
| E71 | 7 |
| E72 | 21 |
| E73 | 24 |
| E74 | 8 |
| E75 | 9 |
| E76 | 20 |
| E77 | <4 |
| E78 | 5 |
| E79 | 8 |
| E80 | <4 |
| E81 | 15 |
| E82 | 5 |
| E83 | 11 |
| E84 | 10 |
| E85 | 14 |
| E86 | 15 |

### Cytochrome P450 Isoenzyme Inhibition Assays

The inhibition of the conversion of a specific substrate to its metabolite is assayed at 37° C. with human liver microsomes and used to determine the inhibition of cytochrome P450 isoenzymes. For the following cytochrome P450 isoenzymes, these substrates and metabolic reactions are monitored: P450 3A4: hydroxylation of Midazolam (MDZ).

The final incubation volume contains TRIS buffer (0.1 M), MgCl₂ (5 mM), a certain concentration of human liver microsomes dependent on the P450 isoenzyme measured (P450 3A4: 0.1 mg/ml) and a certain concentration of the individual substrate for each isoenzyme (P450 3A4: Midazolam 5 μM).

The effect of the test compound is determined at five different concentrations in duplicate (e.g. highest concentration 50 μM with subsequent serial 1:4 dilutions) or without test compound (high control). Following a short preincubation period, reactions are started with the cofactor (NADPH, 1 mM) and stopped by cooling the incubation down to 8° C. and subsequently by addition of one volume of ACN. An internal standard solution—usually the stable isotope of the formed metabolite—is added after quenching of incubations. Peak area analyte (=metabolite formed) and internal standard is determined by LC-MS/MS. The resulting peak area ratio analyte to internal standard in these incubations is compared to a control activity containing no test compound. Within each of the assay runs, the IC₅₀ of a positive control inhibitor dependent on the P450 isoenzyme measured (P450 3A4: ketoconazole) is determined. The assay results are plotted against compound concentrations to calculated IC₅₀ values (half maximal inhibitory concentrations) for inhibitory compounds utilizing Software IDBS E-WorkBook.

Table 4: Inhibition of P450 isoenzyme 3A⁴. Data obtained with the disclosed assay for a selection of compounds (I) according to the invention with Midazolam as substrate. At least one measurement otherwise average values of at least two measurements.

| Example | CYP3A4 inhibition |
|---|---|
| Number | IC50 [μM] |
| E1 | >50 |
| E2 | >50 |
| E3 | >50 |
| E4 | >50 |
| E5 | >50 |
| E6 | >50 |
| E7 | >50 |
| E8 | >50 |
| E10 | >50 |
| cis-E11a | >50 |
| cis-E11b | >50 |
| E12 | >50 |
| E13 | >50 |
| E14 | >50 |
| E15 | >50 |
| cis-E16a | >50 |
| cis-E16b | >50 |
| E17a | >50 |
| E17b | >50 |
| E18 | >50 |
| E19 | >50 |
| E20 | >50 |
| cis-E21a | >50 |
| cis-E21b | >50 |
| E22 | >50 |
| E24 | >50 |
| E25 | >50 |
| E26 | >50 |
| E27 | >50 |
| E28 | >50 |
| cis-E29 | >50 |
| E30 | >50 |
| E31 | >50 |
| E32 | >50 |
| E33 | >50 |
| E34 | >50 |
| E35 | >50 |
| E36 | >50 |
| E37 | >50 |
| E38a | >50 |
| E38b | >50 |
| E39 | >50 |
| E40 | >50 |
| cis-E41a | >50 |
| cis-E41b | >50 |
| trans-E42a | >50 |
| trans-E42b | >50 |
| E43a | >50 |
| E43b | >50 |
| E44 | >50 |
| E45 | >50 |
| E46 | >50 |
| E47a | >50 |
| E47b | >50 |
| E48 | >50 |
| E49 | >50 |
| E50 | >50 |
| cis-E52a | >50 |
| cis-E52b | >50 |
| trans-E53a | >50 |
| trans-E53b | >50 |
| E54 | >50 |
| E55 | >50 |
| E57 | >50 |
| E58 | >50 |
| E59 | >50 |
| E60 | >50 |
| trans-E61a | >50 |
| trans-E61b | >50 |
| E62 | >50 |
| E63 | >50 |
| E64a | >50 |
| E64b | >50 |
| E65 | >50 |
| E66 | >50 |
| E67 | >50 |
| E68 | >50 |
| E69 | >50 |
| trans-E70a | >50 |
| trans-E70b | >50 |
| E71 | >50 |
| E72 | >50 |
| E73 | >50 |
| E74 | >50 |
| E75 | >50 |
| E76 | >50 |
| E77 | >50 |
| E78 | >50 |
| E79 | >50 |
| E80 | >50 |
| E81 | >50 |
| E82 | >50 |
| E83 | >50 |
| E84 | >50 |
| E85 | >50 |
| E86 | >50 |

Mechanism-Based Inhibition of CYP3A4 assay (MBI 3A4): The mechanism-based inhibition towards CYP3A4 is assayed in human liver microsomes with midazolam as substrate. The test compounds and water control (wells w/o test compound) are preincubated in presence of NADPH (1 mM) with human liver microsomes (0.2 mg/mL) at a concentration of 0, 5 and 25 μM for 0, 10 and 30 min. After preincubation, the incubate is diluted 1:10 (to 0.02 mg/mL) and the substrate midazolam (15 μM) is added for the main incubation (10 min). The main incubation is quenched with ACN and the formation of hydroxy-midazolam is quantified via LC/MS-MS. The formation of hydroxy-midazolam from the 30 min preincubation relative to the formation from the 0 min preincubation is used as a readout. Values of less than 100% mean that the substrate midazolam is metabolized to a lower extent upon 30 min preincubation compared to 0 min preincubation. In general, low effects upon 30 min preincubation are desired (corresponding to values close to 100%/not different to the values determined with water control).

Table 5: Mechanism-based inhibition of P450 isoenzyme 3A4 with midazolam as substrate. Data obtained with the disclosed assay for a selection of compounds (I) according to the invention. At least one measurement otherwise average values of at least two measurements.

| Example | MBI inhibition % ctrl. |
|---|---|
| Number | [%] with MDZ |
| E1 | 98 |
| E2 | 95 |
| E3 | 94 |
| E4 | 99 |
| E5 | 103 |
| E6 | 102 |
| E7 | 106 |
| E8 | 97 |
| E10 | 97 |
| E12 | 79 |
| cis-E16a | 92 |
| cis-E16b | 87 |
| E17b | 98 |
| E18 | 99 |
| E19 | 102 |
| E20 | 94 |
| cis-E21a | 102 |
| cis-E21b | 96 |
| E22 | 100 |
| E24 | 99 |
| E25 | 91 |
| E26 | 92 |
| E27 | 90 |
| E28 | 90 |
| cis-E29 | 95 |
| E30 | 103 |
| E31 | 91 |
| E32 | 92 |
| E34 | 106 |
| E35 | 94 |
| E36 | 96 |
| E37 | 90 |
| E38a | 94 |
| E38b | 86 |
| E39 | 75 |
| E40 | 75 |
| cis-E41b | 95 |
| trans-E42a | 101 |
| trans-E42b | 94 |
| E43a | 91 |
| E43b | 94 |
| E44 | 95 |
| E45 | 97 |
| E46 | 98 |
| E47a | 97 |
| E47b | 88 |
| E48 | 92 |
| E50 | 83 |
| cis-E52b | 98 |
| trans-E53b | 91 |
| E54 | 85 |
| E55 | 83 |
| E57 | 106 |
| E58 | 96 |
| E59 | 99 |
| E60 | 90 |
| trans-E61a | 93 |
| trans-E61b | 101 |
| E62 | 98 |
| E63 | 94 |
| E64a | 95 |
| E64b | 76 |
| E65 | 102 |
| E66 | 81 |
| E67 | 95 |
| E68 | 91 |
| E69 | 85 |
| trans-E70b | 107 |
| E71 | 94 |
| E72 | 98 |
| E73 | 103 |
| E74 | 89 |
| E75 | 99 |
| E76 | 84 |
| E77 | 88 |
| E78 | 103 |
| E79 | 90 |
| E80 | 88 |
| E81 | 93 |
| E82 | 92 |
| E83 | 86 |
| E84 | 103 |
| E85 | 102 |
| E86 | 96 |

1. A compound of formula (I) 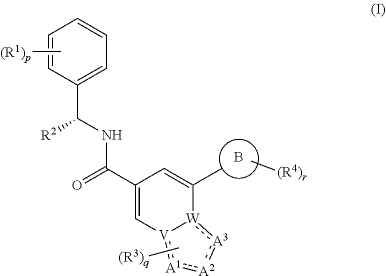
wherein
each R¹ is independently selected from the group consisting of C₁₋₆alkyl, C₁₋₆haloalkyl and halogen;
p denotes 1, 2 or 3;
R² is H, Me or Et;
V is nitrogen (—N═) or carbon 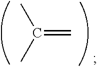
W is nitrogen (—N═) or carbon 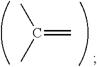
at least one of V and/or W is nitrogen (—N═);
A¹, A² and A³ are each independently selected from nitrogen (—N═) or carbon (═CH— or ═CR³—)
and [symbol] is a single or double bond;
each R³, if present, is independently selected from the group consisting of C₁₋₆alkyl, C₁₋₆alkoxy, halogen and C₁₋₆haloalkyl;
q denotes 0, 1 or 2;
ring system B is selected from C₃₋₁₀cycloalkyl, C₄₋₁₀cycloalkenyl, 4-13 membered heterocyclyl,
r denotes 0, 1, 2, 3 or 4;
each R⁴, if present, is independently selected from the group consisting of R⁵ and R⁶;
each R⁵ is independently selected from the group consisting of —OR⁶, —NR⁶R⁶, halogen, —CN, —C(═O)R⁶, —C(═O)OR⁶, —C(═O)NR⁶R⁶, —NHC(═O)OR⁶ and the bivalent substituent ═O;
each R⁶ is independently selected from the group consisting of hydrogen, C₁₋₆alkyl, C₁₋₆alkoxy, C₁₋₆haloalkyl, C₃₋₁₀cycloalkyl, C₄₋₁₀cycloalkenyl, 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl and 4-11 membered heterocyclyl, are each independently optionally substituted with one or more, identical or different R⁷ and/or R⁸;
each R⁷ is independently selected from the group consisting of —OH, —NH₂, —NHR⁸, —NR⁸R⁸, halogen, —CN, and C₁₋₆alkoxy;
each R⁸ is independently selected from the group consisting of C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, are each independently optionally substituted with one or more, identical or different R⁹;
each R⁹ is —OH, halogen or C₁₋₆alkoxy;
or a salt thereof.

2. The compound or salt according to claim 1, wherein R² is Me.

3. The compound or salt according to claim 1, wherein A¹, A², A³, V and W form a triazole.

4. The compound or salt according to claim 1, wherein the ring 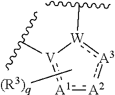
is selected from the group consisting of 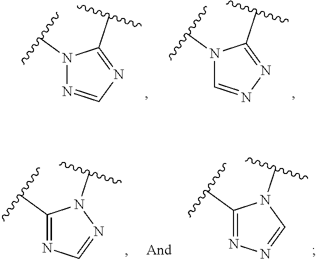 [SMILES: *c1ncnn1* ; *c1nncn1* ; *c1ncnn1* ; *c1nncn1*]
and is optionally substituted with R³.

5. The compound or salt according to claim 1, wherein each R³, if present, is C₁₋₆alkyl.

6. The compound or salt according to claim 1, wherein p is 2 and R¹ is independently selected from the group consisting of C₁₋₆alkyl, C₁₋₆haloalkyl and halogen.

7. The compound or salt according to claim 1, wherein R¹ is independently selected from the group consisting of Me, —CFH₂, —CF₂H, —CF₃, —CFMeH, —CFMe₂, —CF₂Me, and F.

8. The compound or salt according to claim 1, wherein ring system B is selected from C₃₋₁₀cycloalkyl, and C₄₋₁₀cycloalkenyl; and wherein the C₃₋₁₀cycloalkyl, and C₄₋₁₀cycloalkenyl is optionally and independently substituted with r, identical or different R⁴.

9. The compound or salt according to claim 1, wherein ring system B is a 4-13 membered heterocyclyl; and the 4-13 membered heterocyclyl is optionally and independently substituted with r, identical or different R⁴.

10. The compound or salt according to claim 1, wherein ring system B is a 5-6 membered heterocyclyl; and the 5-6 membered heterocyclyl is optionally and independently substituted with r, identical or different R⁴.

11. The compound or salt according to claim 1, wherein ring system B is selected from the group consisting of 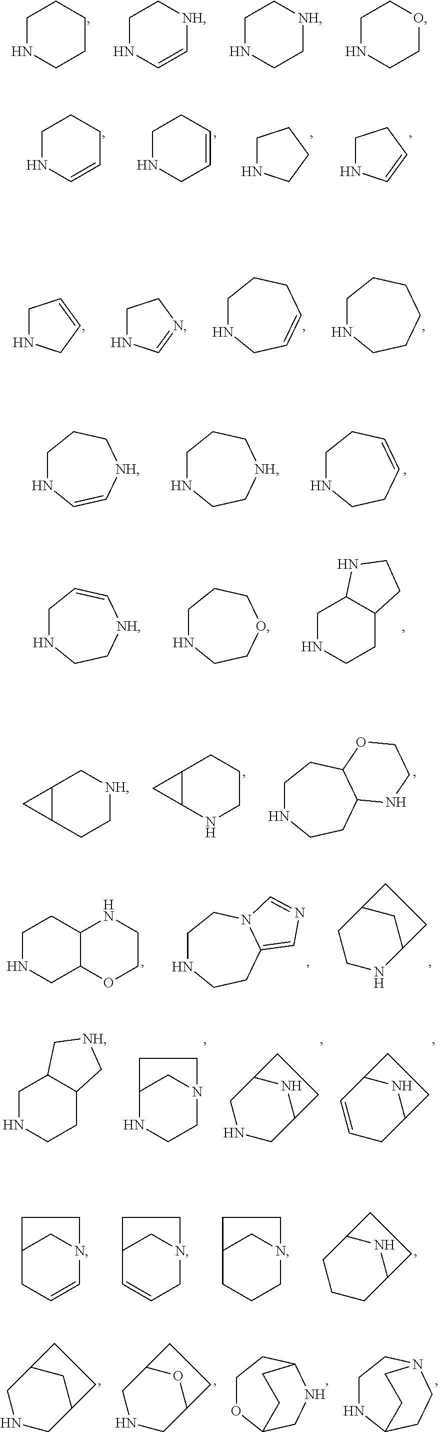 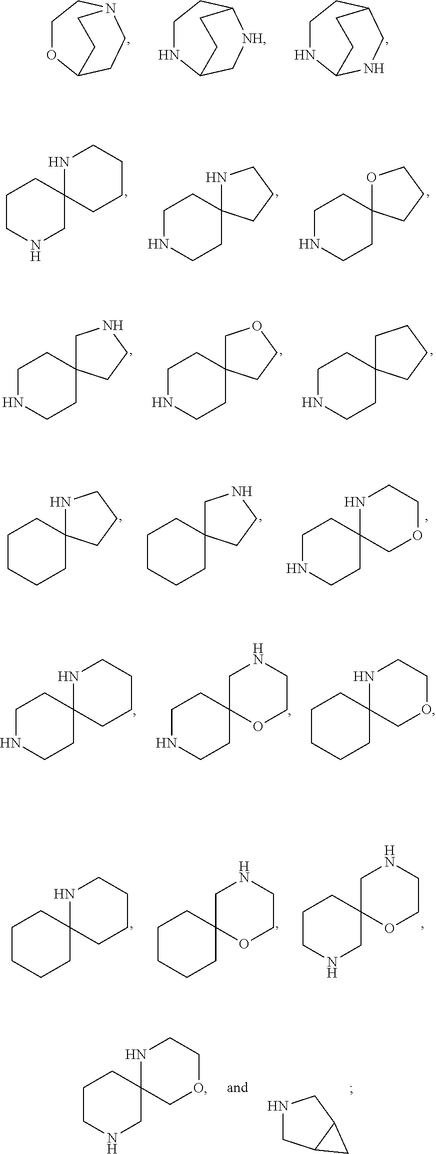
wherein ring system B can be attached to the compound of formula (I) and to R⁴, if present, at any ring position by removal of a hydrogen atom.

12. The compound or salt according to claim 1, wherein each R⁴, if present, is independently selected from the group consisting of R⁵ and R⁶;
each R⁵ is independently selected from the group consisting of —OR⁶, —NR⁶R⁶, halogen, and —CN;
each R⁶ is independently selected from the group consisting of hydrogen, C₁₋₆alkyl, C₁₋₆alkoxy, C₁₋₆haloalkyl, C₃₋₁₀cycloalkyl, C₄₋₁₀cycloalkenyl, 4-11 membered heterocyclyl, wherein the C₁₋₆alkyl, C₁₋₆alkoxy, C₁₋₆haloalkyl, C₃₋₁₀cycloalkyl,
C₄₋₁₀cycloalkenyl and 4-11 membered heterocyclyl, are each independently optionally substituted with one or more, identical or different R⁷ and/or R⁸;
each R⁷ is independently selected from the group consisting of —OH, —NH₂, —NHR⁸, —NR⁸R⁸, halogen, —CN, and C₁₋₆alkoxy;
each R⁸ is independently selected from the group consisting of C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, wherein the C₁₋₆alkyl, C₁₋₆alkoxy, C₁₋₆haloalkyl, C₃₋₁₀cycloalkyl, C₄₋₁₀cycloalkenyl, and 4-11 membered heterocyclyl, are each independently optionally substituted with one or more, identical or different R⁹;
each R⁹ is —OH, halogen or C₁₋₆alkoxy.

13. The compound or salt according to claim 1, wherein each R⁴, if present, is independently selected from the group consisting of R⁵ and R⁶;
each R⁵ is independently selected from the group consisting of —OR⁶, —NR⁶R⁶, halogen, and —CN;
each R⁶ is independently selected from the group consisting of hydrogen, C₁₋₆alkyl, C₃₋₁₀cycloalkyl, 4-11 membered heterocyclyl, wherein the C₁₋₆alkyl, C₃₋₁₀cycloalkyl and 4-11 membered heterocyclyl, are each independently optionally substituted with one or more, identical or different R⁷ and/or R⁸;
each R⁷ is independently selected from the group consisting of —OH, halogen, and C₁₋₆alkoxy;
each R⁸ is independently selected from the group consisting of C₁₋₆alkyl, C₃₋₁₀cycloalkyl, and 4-11 membered heterocyclyl, wherein the C₁₋₆alkyl, C₃₋₁₀cycloalkyl, and 4-11 membered heterocyclyl, are each independently optionally substituted with one or more, identical or different R⁹;
each R⁹ is —OH.

14. The compound or salt according to claim 1, wherein each R⁴, if present, is independently selected from the group consisting of

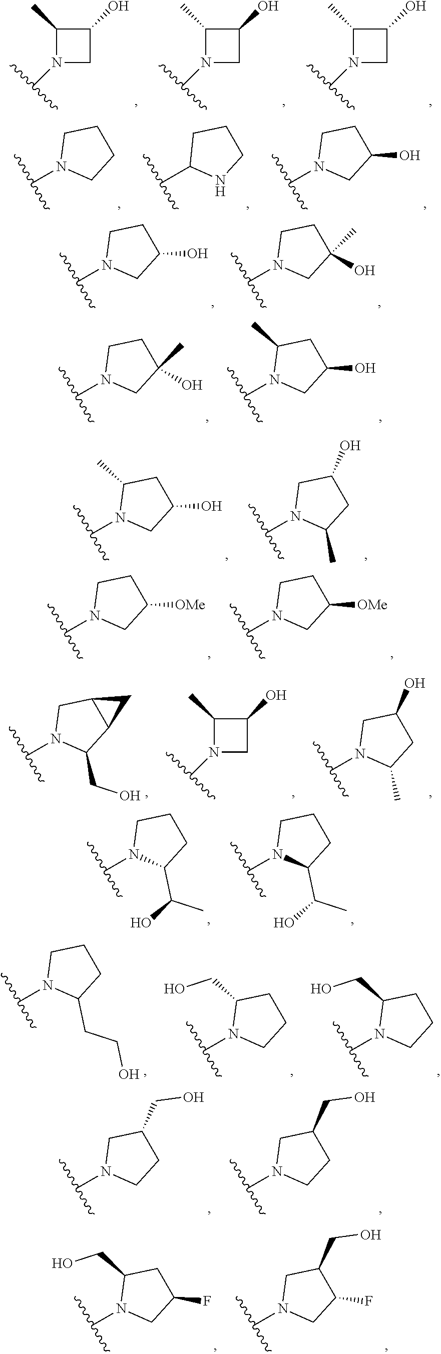

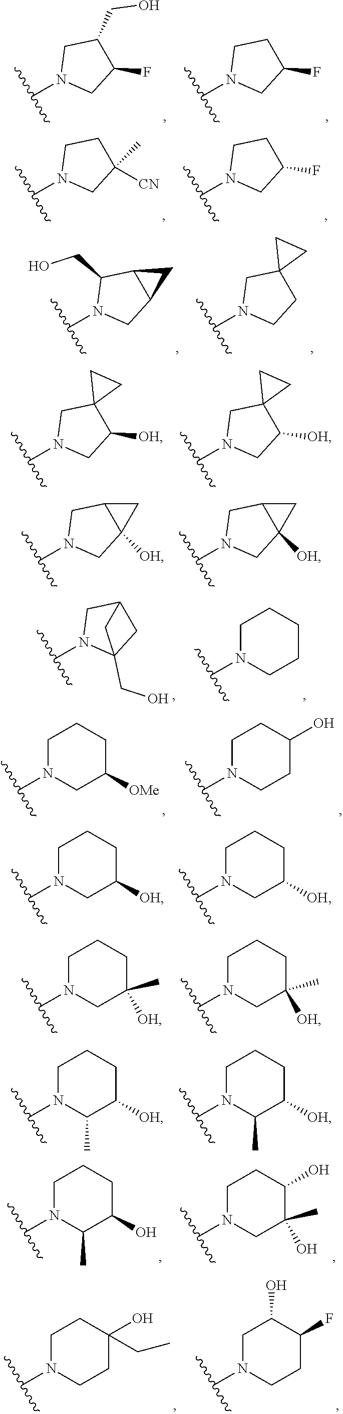

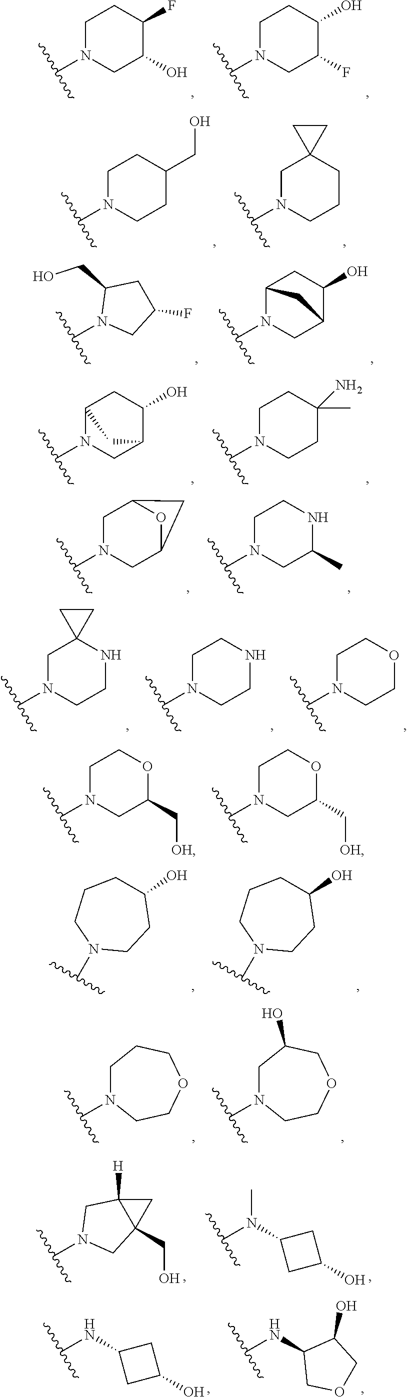

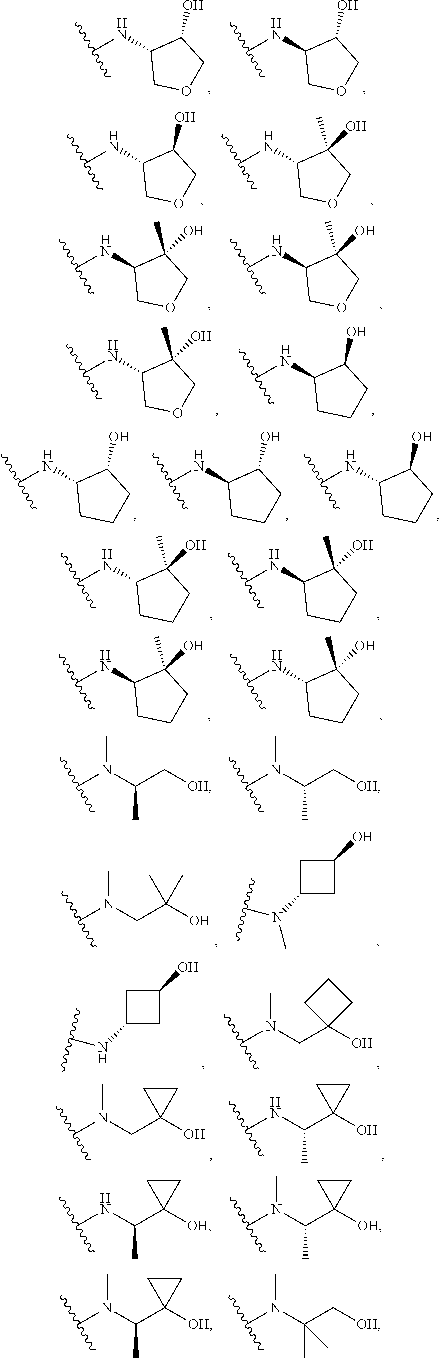

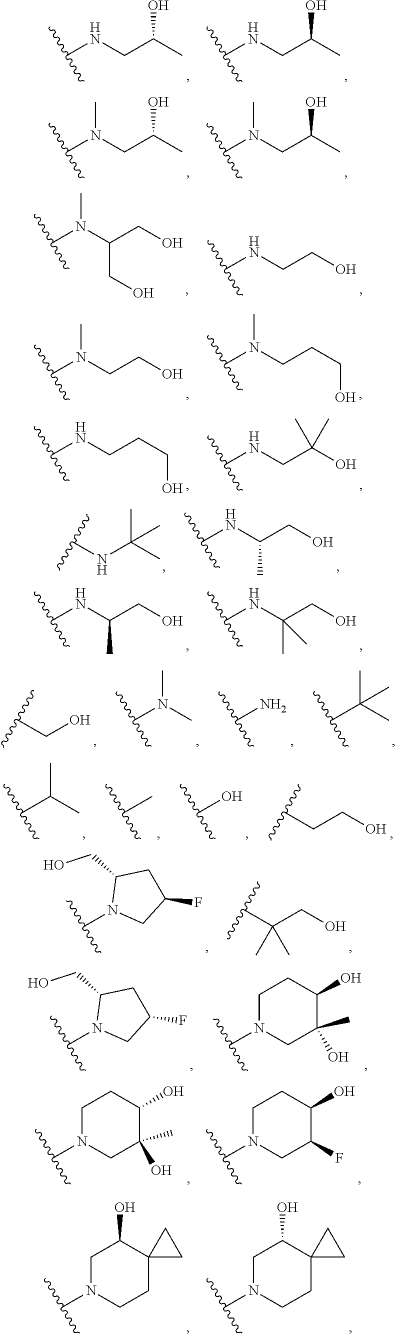

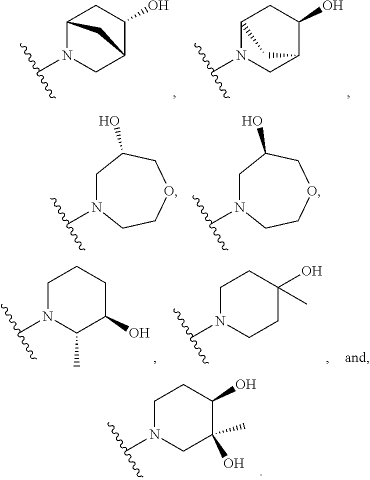

15. A compound selected from the group consisting of: 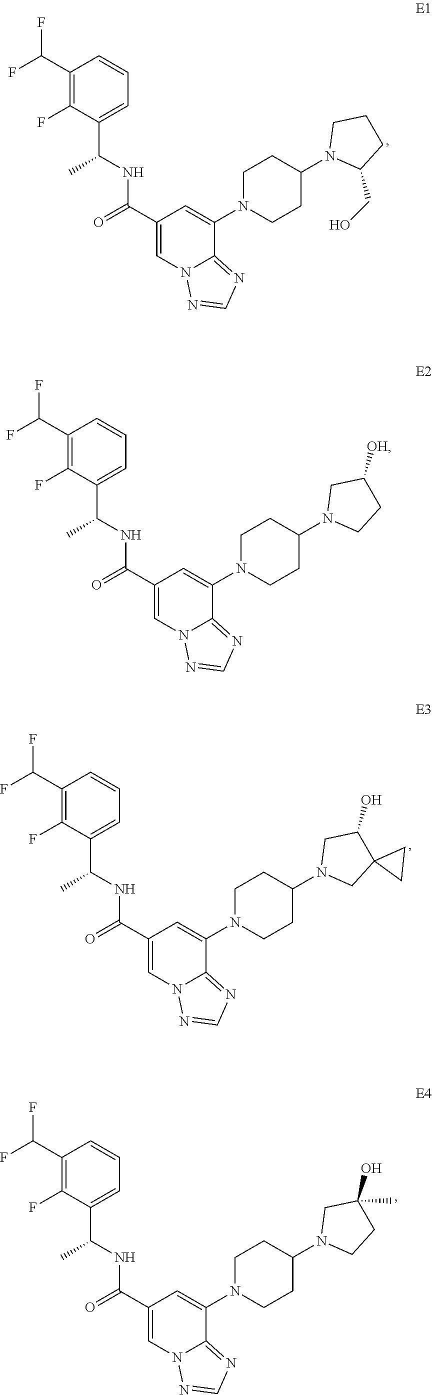 [SMILES: C[C@@H](NC(=O)c1cc(N2CCC(N3CCC[C@@H]3CO)CC2)c2ncnn2c1)c1cccc(C(F)F)c1F ; C[C@@H](NC(=O)c1cc(N2CCC(N3CC[C@@H](O)C3)CC2)c2ncnn2c1)c1cccc(C(F)F)c1F ; C[C@@H](NC(=O)c1cc(N2CCC(N3C[C@H](O)C4(CC4)C3)CC2)c2ncnn2c1)c1cccc(C(F)F)c1F ; C[C@@H](NC(=O)c1cc(N2CCC(N3CC[C@](C)(O)C3)CC2)c2ncnn2c1)c1cccc(C(F)F)c1F] 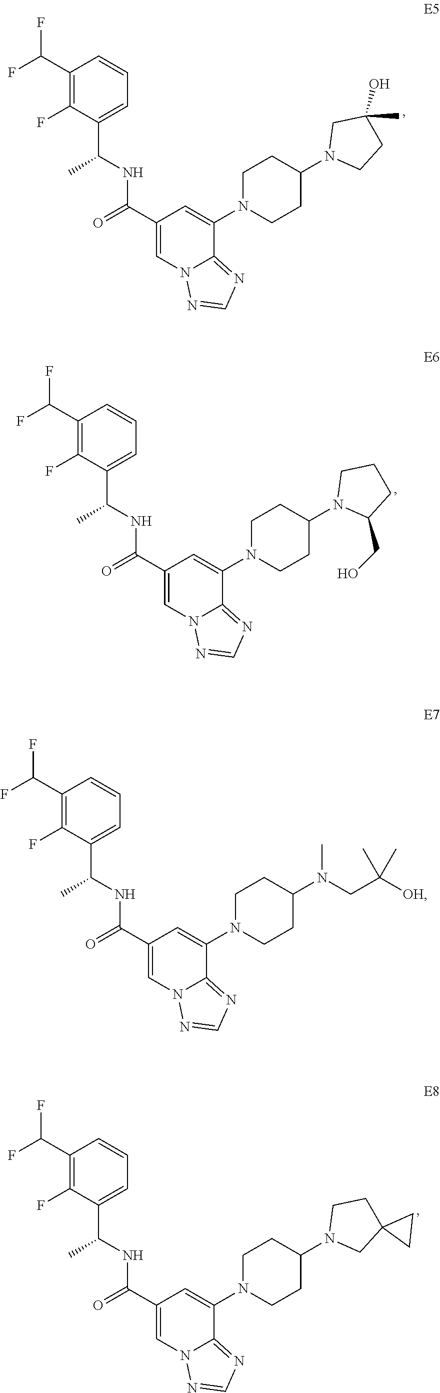 [SMILES: C[C@@H](NC(=O)c1cc(N2CCC(N3CC[C@@](C)(O)C3)CC2)c2ncnn2c1)c1cccc(C(F)F)c1F ; C[C@@H](NC(=O)c1cc(N2CCC(N3CCC[C@H]3CO)CC2)c2ncnn2c1)c1cccc(C(F)F)c1F ; C[C@@H](NC(=O)c1cc(N2CCC(N(C)CC(C)(C)O)CC2)c2ncnn2c1)c1cccc(C(F)F)c1F ; C[C@@H](NC(=O)c1cc(N2CCC(N3CCC4(CC4)C3)CC2)c2ncnn2c1)c1cccc(C(F)F)c1F] 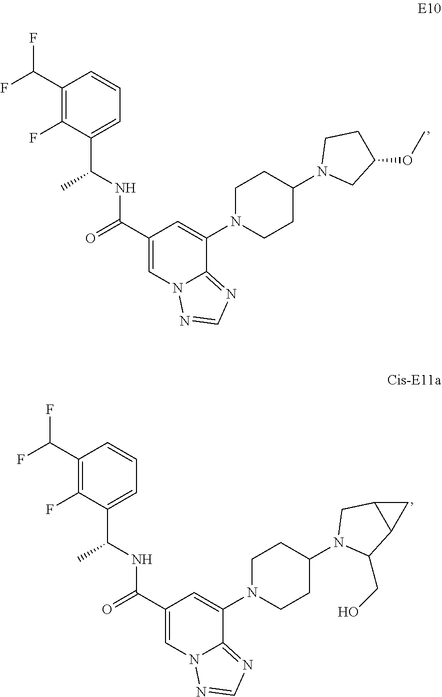 [SMILES: CO[C@H]1CCN(C2CCN(c3cc(C(=O)N[C@H](C)c4cccc(C(F)F)c4F)cn4ncnc34)CC2)C1 ; C[C@@H](NC(=O)c1cc(N2CCC(N3CC4CC4C3CO)CC2)c2ncnn2c1)c1cccc(C(F)F)c1F] 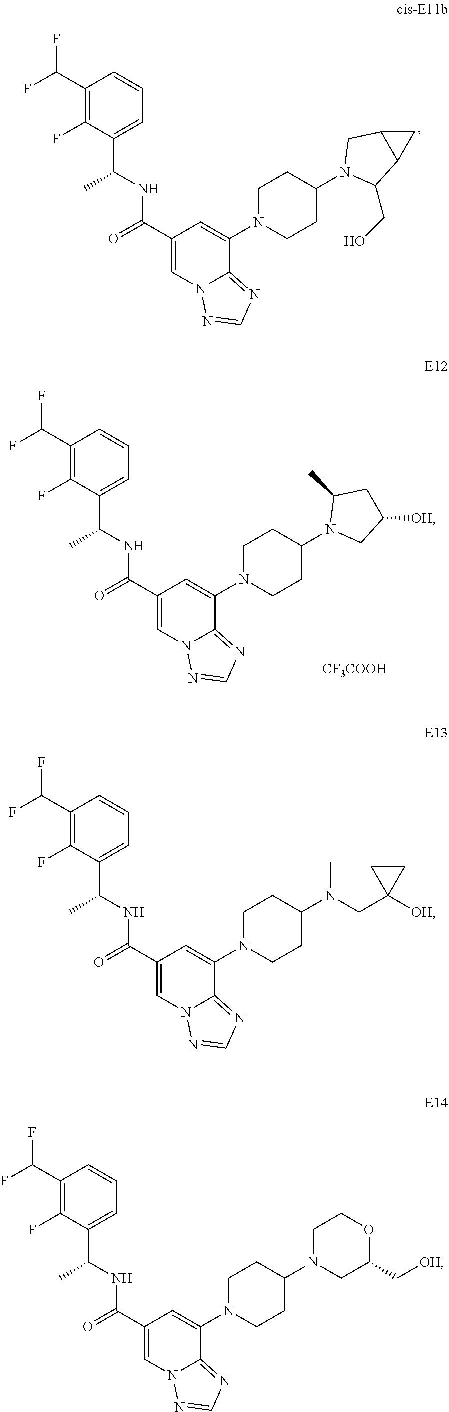 [SMILES: C[C@@H](NC(=O)c1cc(N2CCC(N3CC4CC4C3CO)CC2)c2ncnn2c1)c1cccc(C(F)F)c1F ; C[C@@H](NC(=O)c1cc(N2CCC(N3C[C@@H](O)C[C@@H]3C)CC2)c2ncnn2c1)c1cccc(C(F)F)c1F ; "CF3COOH" ; C[C@@H](NC(=O)c1cc(N2CCC(N(C)CC3(O)CC3)CC2)c2ncnn2c1)c1cccc(C(F)F)c1F ; C[C@@H](NC(=O)c1cc(N2CCC(N3CCO[C@H](CO)C3)CC2)c2ncnn2c1)c1cccc(C(F)F)c1F] 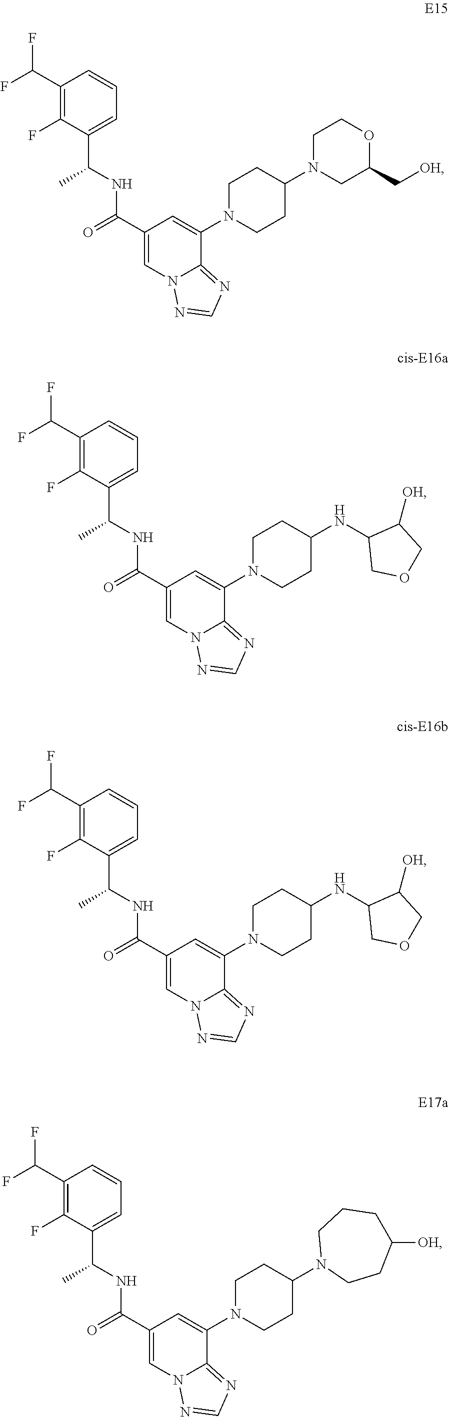 [SMILES: C[C@@H](NC(=O)c1cc(N2CCC(N3CCO[C@@H](CO)C3)CC2)c2ncnn2c1)c1cccc(C(F)F)c1F ; C[C@@H](NC(=O)c1cc(N2CCC(NC3COCC3O)CC2)c2ncnn2c1)c1cccc(C(F)F)c1F ; C[C@@H](NC(=O)c1cc(N2CCC(NC3COCC3O)CC2)c2ncnn2c1)c1cccc(C(F)F)c1F ; C[C@@H](NC(=O)c1cc(N2CCC(N3CCCC(O)CC3)CC2)c2ncnn2c1)c1cccc(C(F)F)c1F] 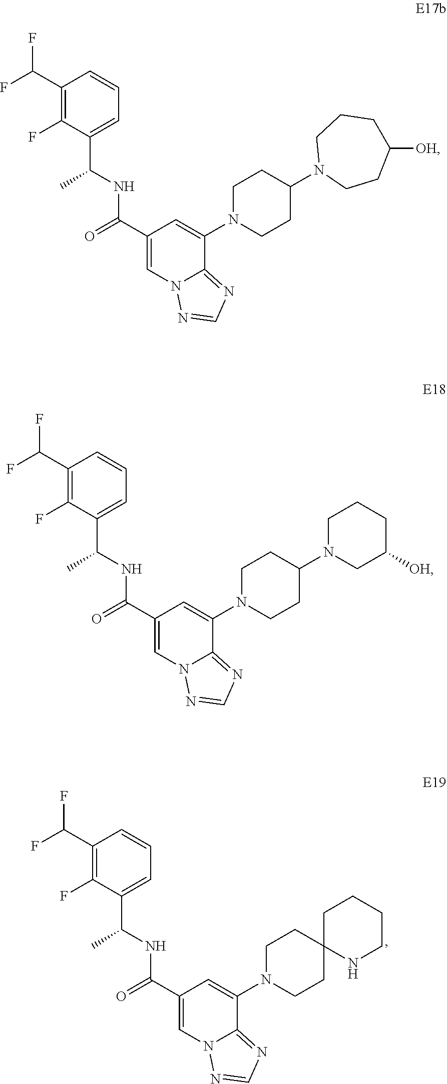 [SMILES: C[C@@H](NC(=O)c1cc(N2CCC(N3CCCC(O)CC3)CC2)c2ncnn2c1)c1cccc(C(F)F)c1F ; C[C@@H](NC(=O)c1cc(N2CCC(N3CCC[C@H](O)C3)CC2)c2ncnn2c1)c1cccc(C(F)F)c1F ; C[C@@H](NC(=O)c1cc(N2CCC3(CCCCN3)CC2)c2ncnn2c1)c1cccc(C(F)F)c1F] 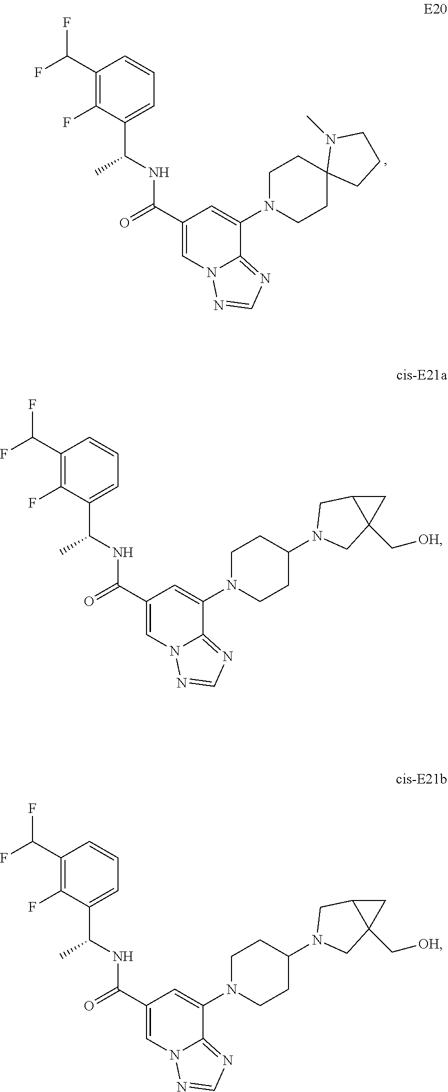 [SMILES: C[C@@H](NC(=O)c1cc(N2CCC3(CCCN3C)CC2)c2ncnn2c1)c1cccc(C(F)F)c1F ; C[C@@H](NC(=O)c1cc(N2CCC(N3CC4CC4(CO)C3)CC2)c2ncnn2c1)c1cccc(C(F)F)c1F ; C[C@@H](NC(=O)c1cc(N2CCC(N3CC4CC4(CO)C3)CC2)c2ncnn2c1)c1cccc(C(F)F)c1F] 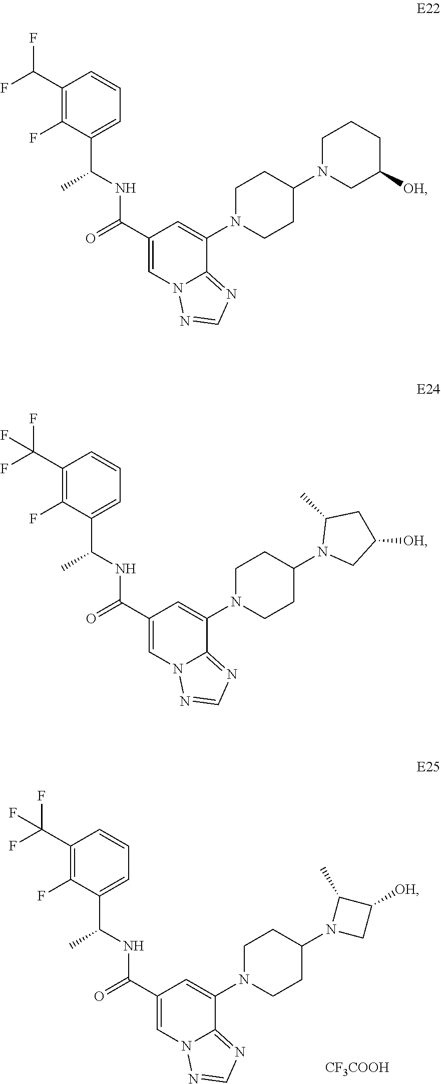 [SMILES: C[C@@H](NC(=O)c1cc(N2CCC(N3CCC[C@@H](O)C3)CC2)c2ncnn2c1)c1cccc(C(F)F)c1F ; C[C@@H]1C[C@H](O)CN1C1CCN(c2cc(C(=O)N[C@H](C)c3cccc(C(F)(F)F)c3F)cn3ncnc23)CC1 ; C[C@@H]1[C@H](O)CN1C1CCN(c2cc(C(=O)N[C@H](C)c3cccc(C(F)(F)F)c3F)cn3ncnc23)CC1 ; "CF3COOH"] 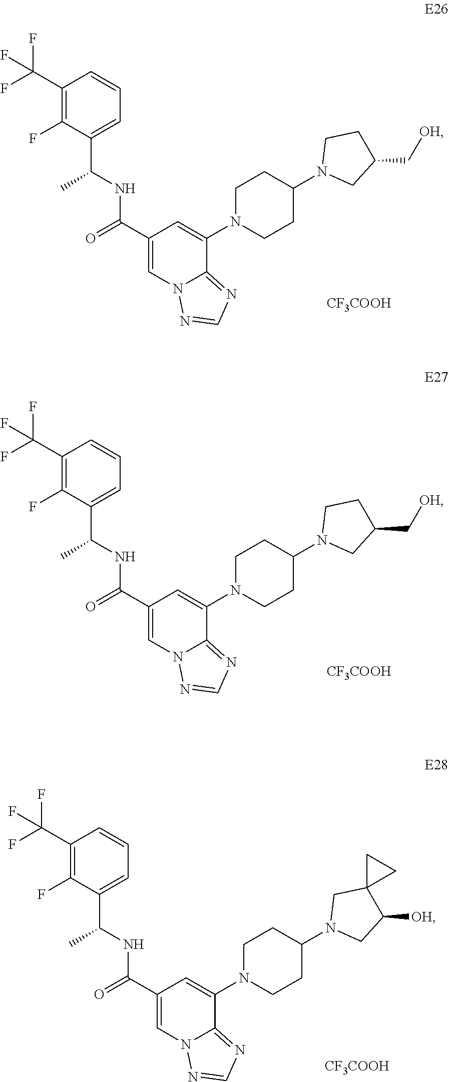 [SMILES: C[C@@H](NC(=O)c1cc(N2CCC(N3CC[C@H](CO)C3)CC2)c2ncnn2c1)c1cccc(C(F)(F)F)c1F ; "CF3COOH" ; C[C@@H](NC(=O)c1cc(N2CCC(N3CC[C@@H](CO)C3)CC2)c2ncnn2c1)c1cccc(C(F)(F)F)c1F ; "CF3COOH" ; C[C@@H](NC(=O)c1cc(N2CCC(N3C[C@H](O)C4(CC4)C3)CC2)c2ncnn2c1)c1cccc(C(F)(F)F)c1F ; "CF3COOH"] 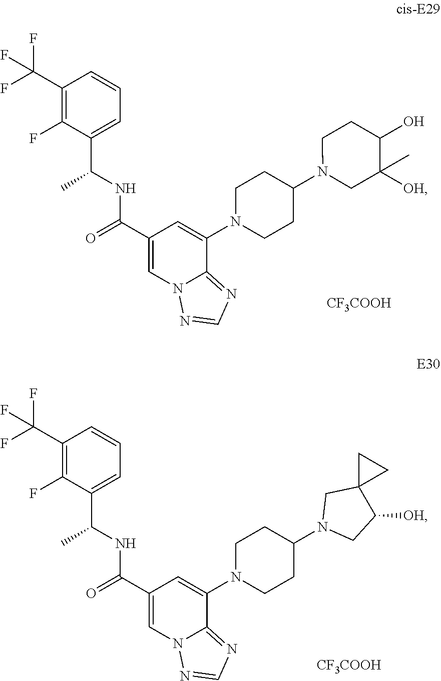 [SMILES: C[C@@H](NC(=O)c1cc(N2CCC(N3CCC(O)C(C)(O)C3)CC2)c2ncnn2c1)c1cccc(C(F)(F)F)c1F ; "CF3COOH" ; C[C@@H](NC(=O)c1cc(N2CCC(N3C[C@@H](O)C4(CC4)C3)CC2)c2ncnn2c1)c1cccc(C(F)(F)F)c1F ; "CF3COOH"] 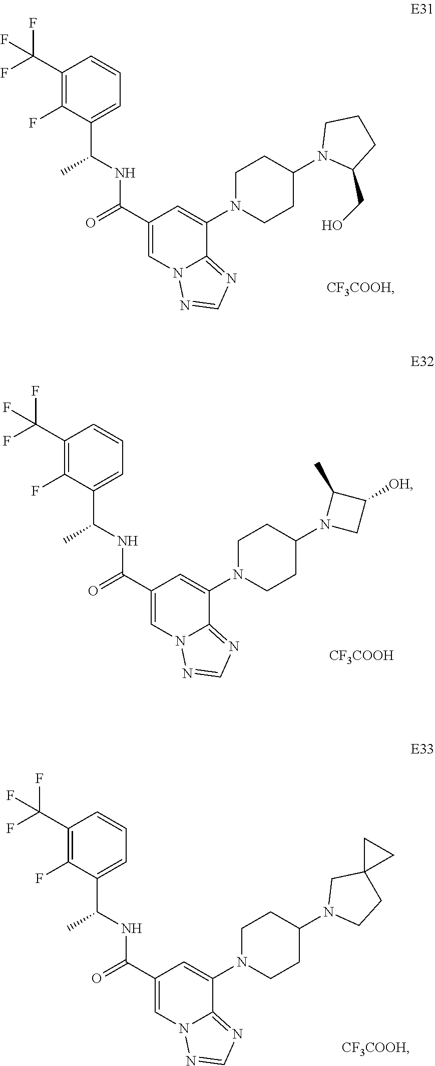 [SMILES: C[C@@H](NC(=O)c1cc(N2CCC(N3CCC[C@H]3CO)CC2)c2ncnn2c1)c1cccc(C(F)(F)F)c1F ; "CF3COOH," ; C[C@@H](NC(=O)c1cc(N2CCC(N3C[C@@H](O)[C@@H]3C)CC2)c2ncnn2c1)c1cccc(C(F)(F)F)c1F ; "CF3COOH" ; C[C@@H](NC(=O)c1cc(N2CCC(N3CCC4(CC4)C3)CC2)c2ncnn2c1)c1cccc(C(F)(F)F)c1F ; "CF3COOH,"] 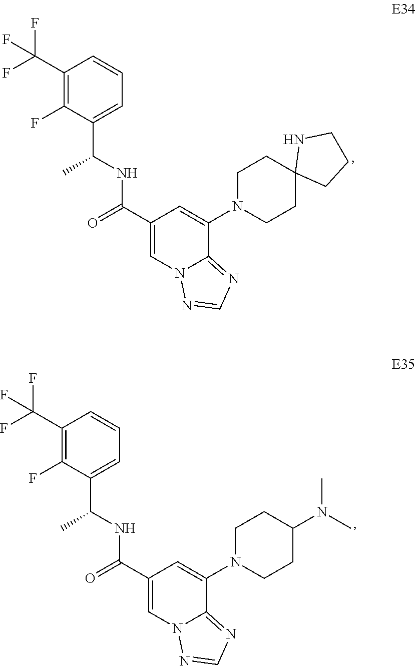 [SMILES: C[C@@H](NC(=O)c1cc(N2CCC3(CCCN3)CC2)c2ncnn2c1)c1cccc(C(F)(F)F)c1F ; C[C@@H](NC(=O)c1cc(N2CCC(N(C)C)CC2)c2ncnn2c1)c1cccc(C(F)(F)F)c1F] 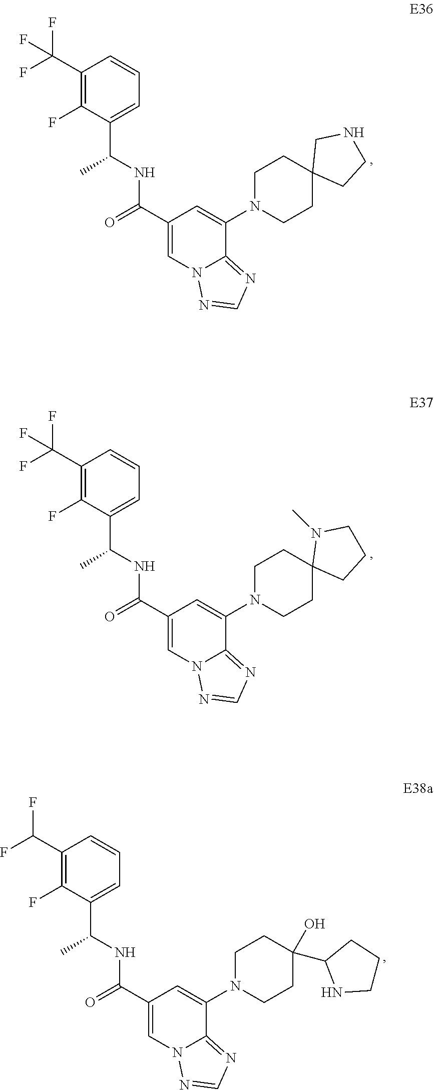 [SMILES: C[C@@H](NC(=O)c1cc(N2CCC3(CCNC3)CC2)c2ncnn2c1)c1cccc(C(F)(F)F)c1F ; C[C@@H](NC(=O)c1cc(N2CCC3(CCCN3C)CC2)c2ncnn2c1)c1cccc(C(F)(F)F)c1F ; C[C@@H](NC(=O)c1cc(N2CCC(O)(C3CCCN3)CC2)c2ncnn2c1)c1cccc(C(F)F)c1F] 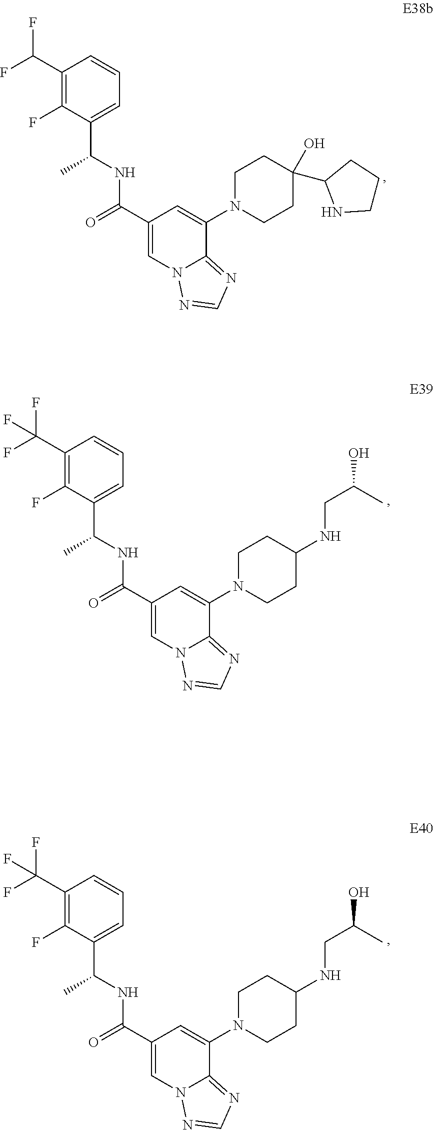 [SMILES: C[C@@H](NC(=O)c1cc(N2CCC(O)(C3CCCN3)CC2)c2ncnn2c1)c1cccc(C(F)F)c1F ; C[C@@H](O)CNC1CCN(c2cc(C(=O)N[C@H](C)c3cccc(C(F)(F)F)c3F)cn3ncnc23)CC1 ; C[C@H](O)CNC1CCN(c2cc(C(=O)N[C@H](C)c3cccc(C(F)(F)F)c3F)cn3ncnc23)CC1] 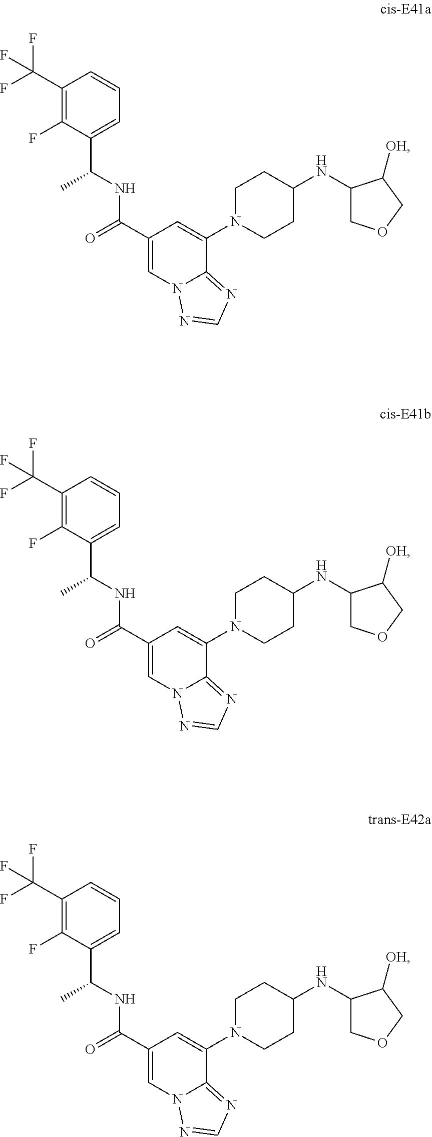 [SMILES: C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(NC3COCC3O)CC2)c2ncnn2c1)c1cccc(C(F)(F)F)c1F] 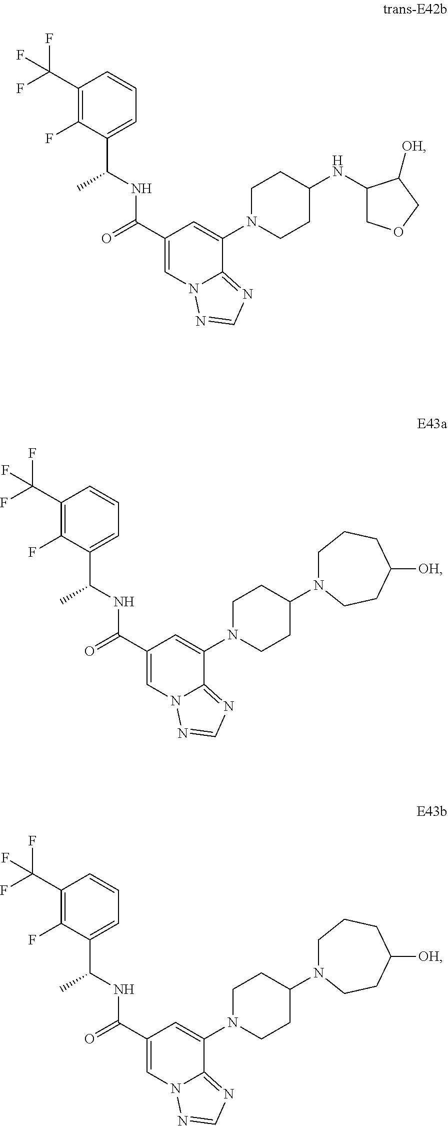 [SMILES: C[C@@H](NC(=O)c1cc(N2CCC(NC3COCC3O)CC2)c2ncnn2c1)c1cccc(C(F)(F)F)c1F ; C[C@@H](NC(=O)c1cc(N2CCC(N3CCCC(O)CC3)CC2)c2ncnn2c1)c1cccc(C(F)(F)F)c1F ; C[C@@H](NC(=O)c1cc(N2CCC(N3CCCC(O)CC3)CC2)c2ncnn2c1)c1cccc(C(F)(F)F)c1F] 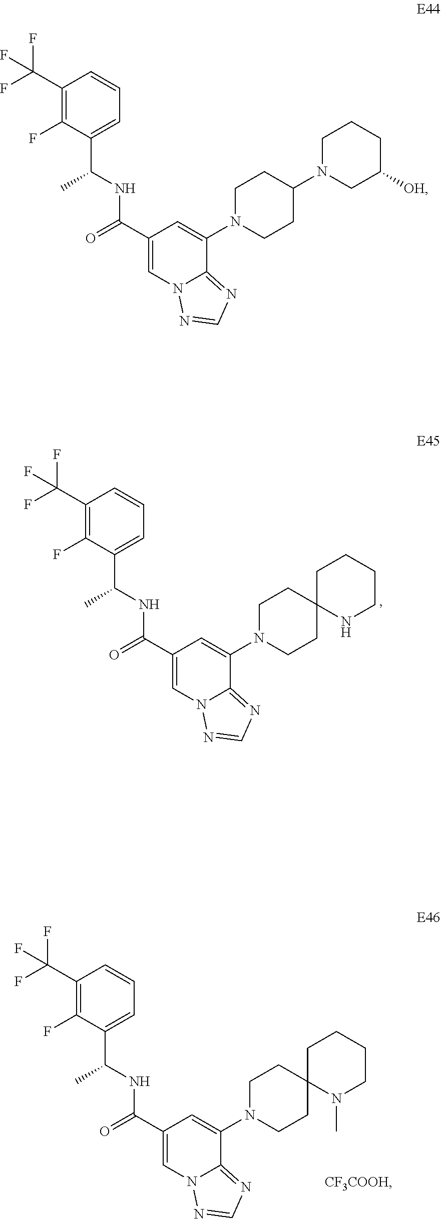 [SMILES: C[C@@H](NC(=O)c1cc(N2CCC(N3CCC[C@H](O)C3)CC2)c2ncnn2c1)c1cccc(C(F)(F)F)c1F ; C[C@@H](NC(=O)c1cc(N2CCC3(CCCCN3)CC2)c2ncnn2c1)c1cccc(C(F)(F)F)c1F ; C[C@@H](NC(=O)c1cc(N2CCC3(CCCCN3C)CC2)c2ncnn2c1)c1cccc(C(F)(F)F)c1F] 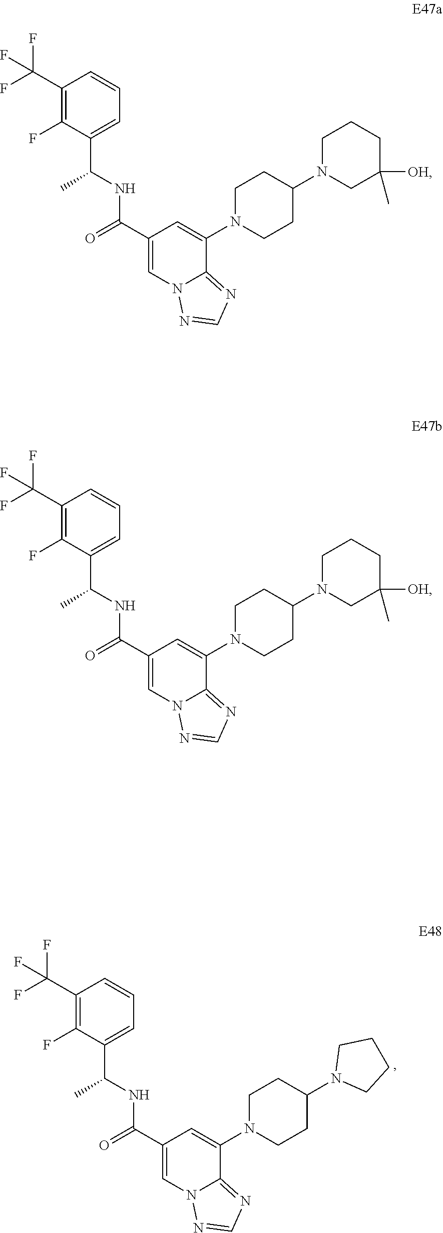 [SMILES: C[C@@H](NC(=O)c1cc(N2CCC(N3CCCC(C)(O)C3)CC2)c2ncnn2c1)c1cccc(C(F)(F)F)c1F ; C[C@@H](NC(=O)c1cc(N2CCC(N3CCCC(C)(O)C3)CC2)c2ncnn2c1)c1cccc(C(F)(F)F)c1F ; C[C@@H](NC(=O)c1cc(N2CCC(N3CCCC3)CC2)c2ncnn2c1)c1cccc(C(F)(F)F)c1F] 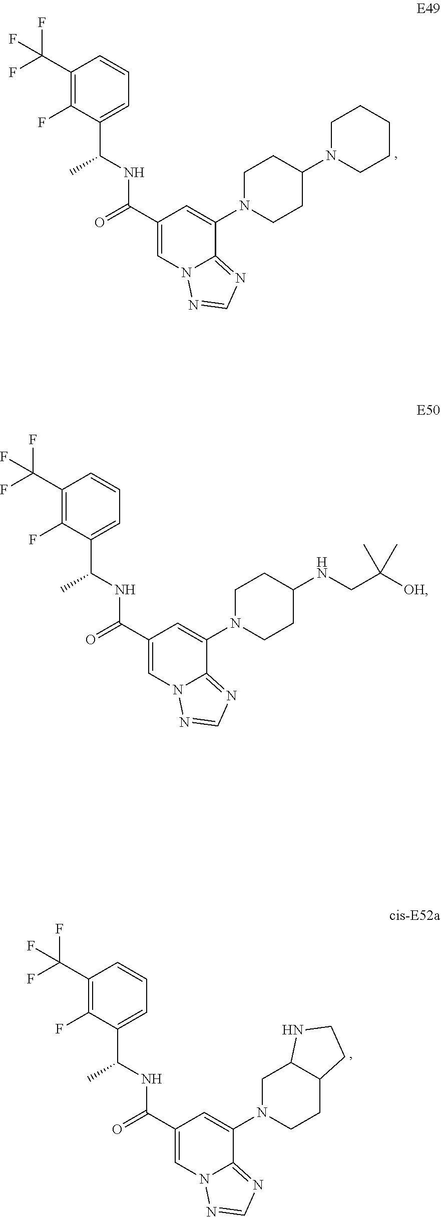 [SMILES: C[C@@H](NC(=O)c1cc(N2CCC(N3CCCCC3)CC2)c2ncnn2c1)c1cccc(C(F)(F)F)c1F ; C[C@@H](NC(=O)c1cc(N2CCC(NCC(C)(C)O)CC2)c2ncnn2c1)c1cccc(C(F)(F)F)c1F ; C[C@@H](NC(=O)c1cc(N2CCC3CCNC3C2)c2ncnn2c1)c1cccc(C(F)(F)F)c1F] 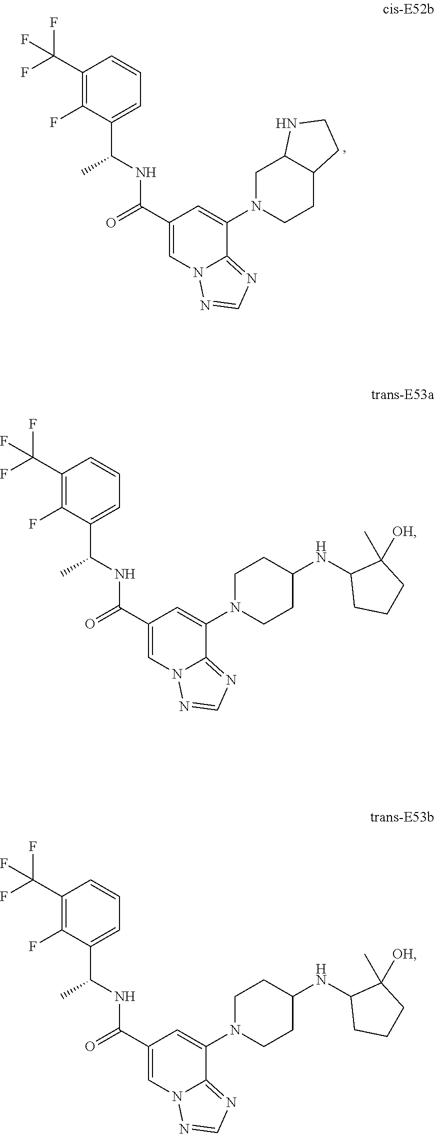 [SMILES: C[C@@H](NC(=O)c1cc(N2CCC3CCNC3C2)c2ncnn2c1)c1cccc(C(F)(F)F)c1F ; C[C@@H](NC(=O)c1cc(N2CCC(NC3CCCC3(C)O)CC2)c2ncnn2c1)c1cccc(C(F)(F)F)c1F ; C[C@@H](NC(=O)c1cc(N2CCC(NC3CCCC3(C)O)CC2)c2ncnn2c1)c1cccc(C(F)(F)F)c1F] 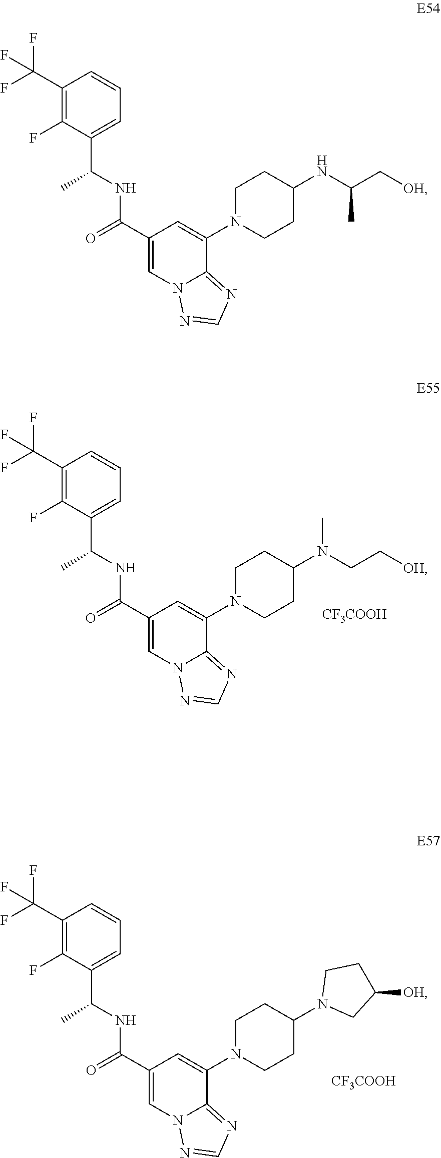 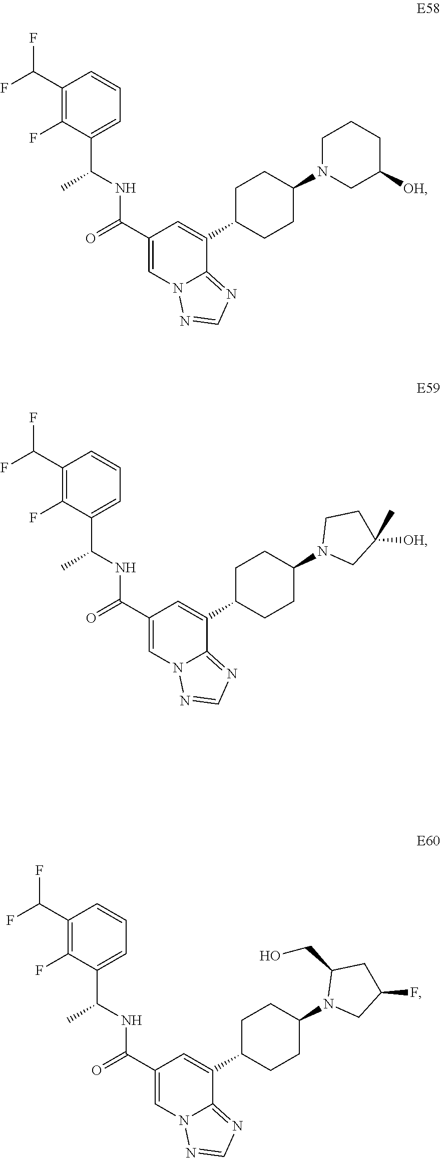 [SMILES: C[C@@H](NC(=O)c1cc([C@H]2CC[C@H](N3CCC[C@@H](O)C3)CC2)c2ncnn2c1)c1cccc(C(F)F)c1F ; C[C@@H](NC(=O)c1cc([C@H]2CC[C@H](N3CC[C@](C)(O)C3)CC2)c2ncnn2c1)c1cccc(C(F)F)c1F ; C[C@@H](NC(=O)c1cc([C@H]2CC[C@H](N3C[C@H](F)C[C@@H]3CO)CC2)c2ncnn2c1)c1cccc(C(F)F)c1F] 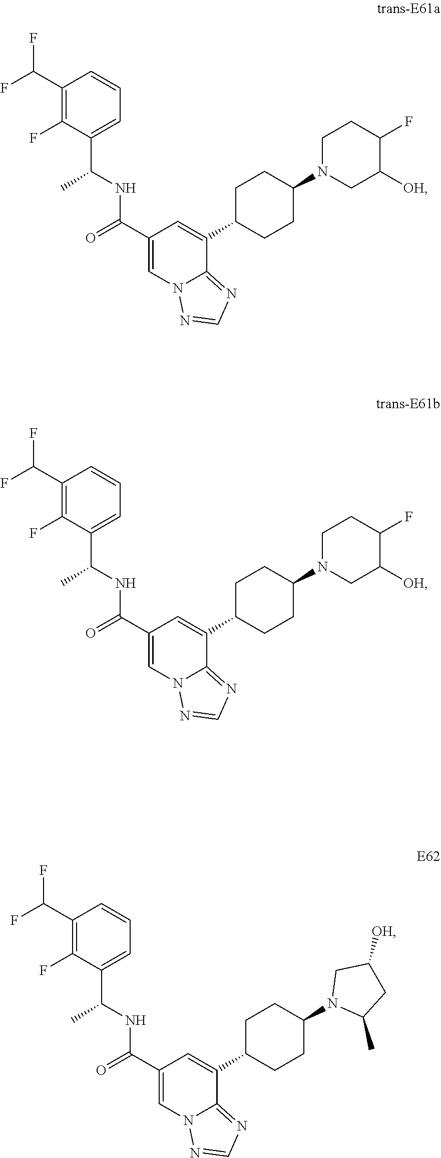 [SMILES: C[C@@H](NC(=O)c1cc([C@H]2CC[C@H](N3CCC(F)C(O)C3)CC2)c2ncnn2c1)c1cccc(C(F)F)c1F ; C[C@@H](NC(=O)c1cc([C@H]2CC[C@H](N3CCC(F)C(O)C3)CC2)c2ncnn2c1)c1cccc(C(F)F)c1F ; C[C@@H]1C[C@@H](O)CN1[C@H]1CC[C@H](c2cc(C(=O)N[C@H](C)c3cccc(C(F)F)c3F)cn3ncnc23)CC1] 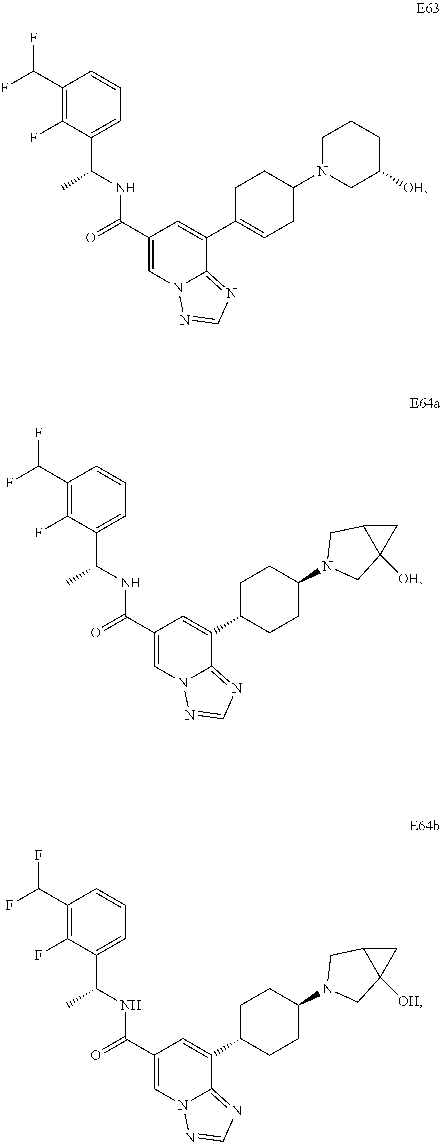 [SMILES: C[C@@H](NC(=O)c1cc(C2=CCC(N3CCC[C@H](O)C3)CC2)c2ncnn2c1)c1cccc(C(F)F)c1F ; C[C@@H](NC(=O)c1cc([C@H]2CC[C@H](N3CC4CC4(O)C3)CC2)c2ncnn2c1)c1cccc(C(F)F)c1F ; C[C@@H](NC(=O)c1cc([C@H]2CC[C@H](N3CC4CC4(O)C3)CC2)c2ncnn2c1)c1cccc(C(F)F)c1F] 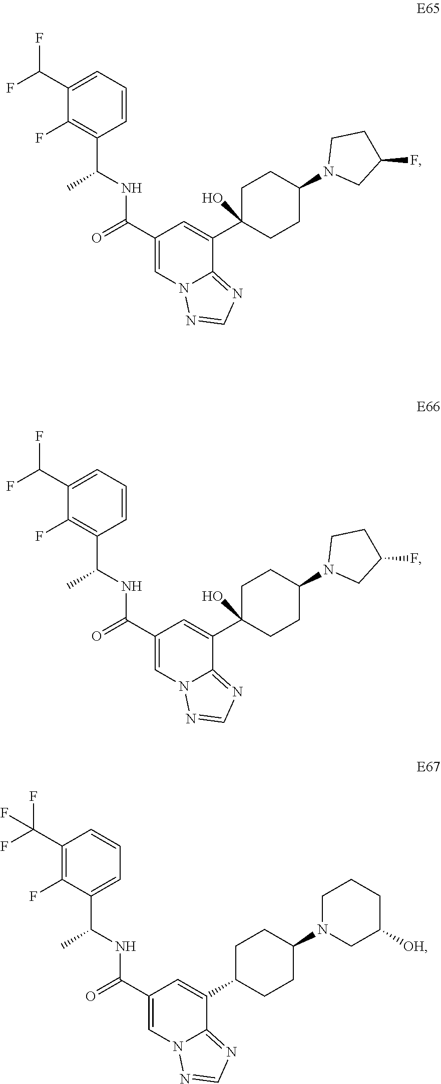 [SMILES: C[C@@H](NC(=O)c1cc([C@]2(O)CC[C@@H](N3CC[C@@H](F)C3)CC2)c2ncnn2c1)c1cccc(C(F)F)c1F ; C[C@@H](NC(=O)c1cc([C@]2(O)CC[C@@H](N3CC[C@H](F)C3)CC2)c2ncnn2c1)c1cccc(C(F)F)c1F ; C[C@@H](NC(=O)c1cc([C@H]2CC[C@H](N3CCC[C@H](O)C3)CC2)c2ncnn2c1)c1cccc(C(F)(F)F)c1F] 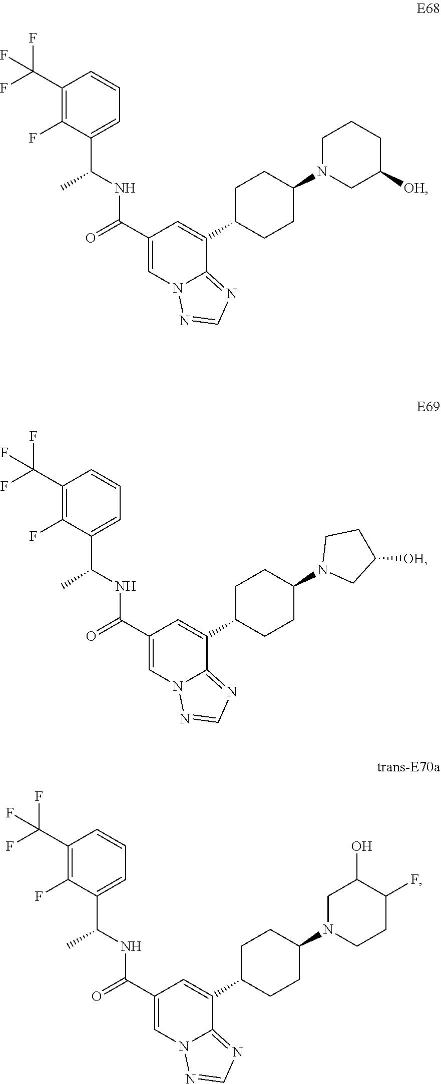 [SMILES: C[C@@H](NC(=O)c1cc([C@H]2CC[C@H](N3CCC[C@@H](O)C3)CC2)c2ncnn2c1)c1cccc(C(F)(F)F)c1F ; C[C@@H](NC(=O)c1cc([C@H]2CC[C@H](N3CC[C@H](O)C3)CC2)c2ncnn2c1)c1cccc(C(F)(F)F)c1F ; C[C@@H](NC(=O)c1cc([C@H]2CC[C@H](N3CCC(F)C(O)C3)CC2)c2ncnn2c1)c1cccc(C(F)(F)F)c1F] 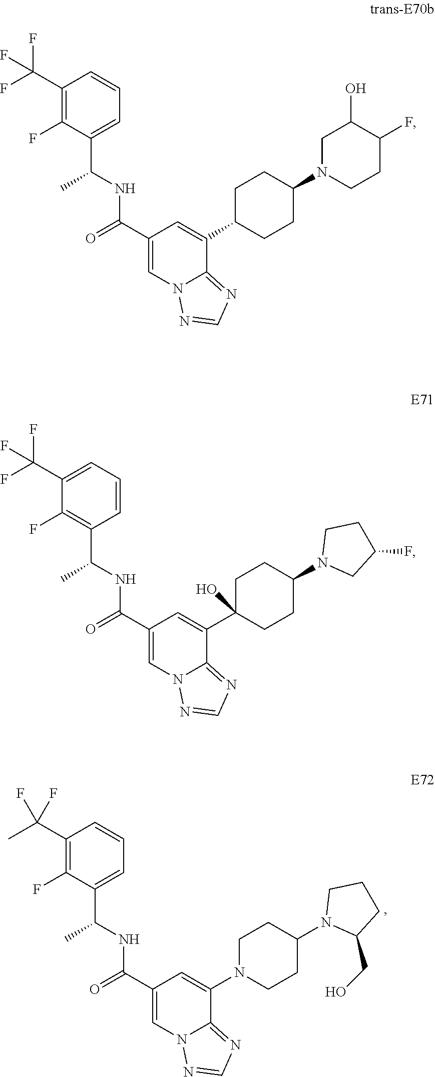 [SMILES: C[C@@H](NC(=O)c1cc([C@H]2CC[C@H](N3CCC(F)C(O)C3)CC2)c2ncnn2c1)c1cccc(C(F)(F)F)c1F ; C[C@@H](NC(=O)c1cc([C@]2(O)CC[C@@H](N3CC[C@H](F)C3)CC2)c2ncnn2c1)c1cccc(C(F)(F)F)c1F ; C[C@@H](NC(=O)c1cc(N2CCC(N3CCC[C@H]3CO)CC2)c2ncnn2c1)c1cccc(C(C)(F)F)c1F] 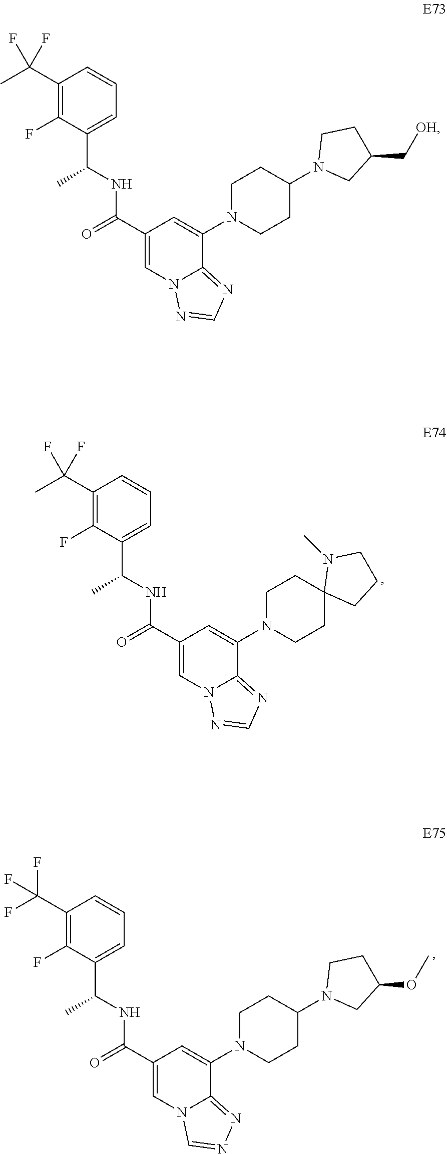 [SMILES: C[C@@H](NC(=O)c1cc(N2CCC(N3CC[C@@H](CO)C3)CC2)c2ncnn2c1)c1cccc(C(C)(F)F)c1F ; C[C@@H](NC(=O)c1cc(N2CCC3(CCCN3C)CC2)c2ncnn2c1)c1cccc(C(C)(F)F)c1F ; CO[C@@H]1CCN(C2CCN(c3cc(C(=O)N[C@H](C)c4cccc(C(F)(F)F)c4F)cn4cnnc34)CC2)C1] 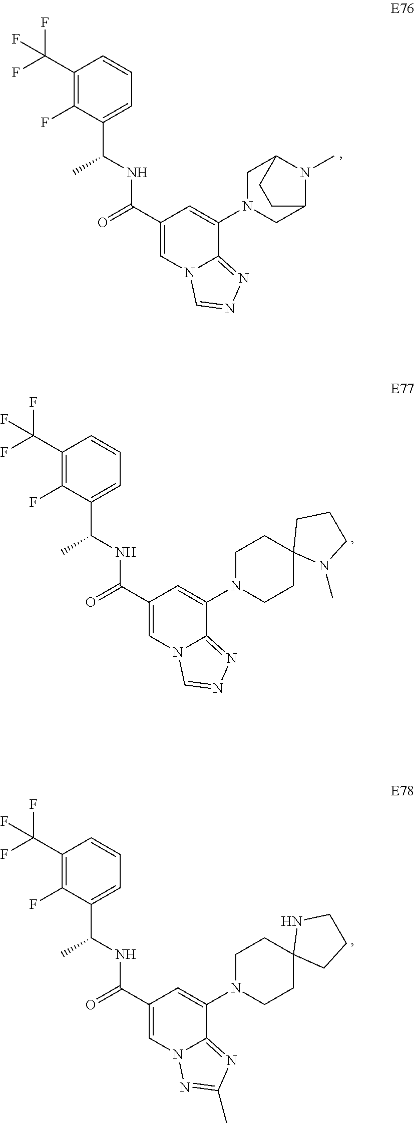 [SMILES: C[C@@H](NC(=O)c1cc(N2CC3CCC(C2)N3C)c2nncn2c1)c1cccc(C(F)(F)F)c1F ; C[C@@H](NC(=O)c1cc(N2CCC3(CCCN3C)CC2)c2nncn2c1)c1cccc(C(F)(F)F)c1F ; Cc1nc2c(N3CCC4(CCCN4)CC3)cc(C(=O)N[C@H](C)c3cccc(C(F)(F)F)c3F)cn2n1] 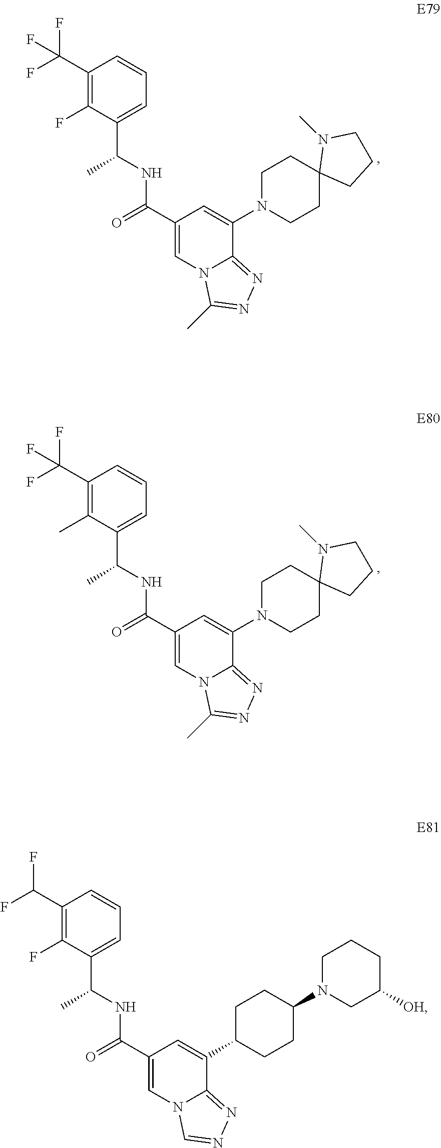 [SMILES: Cc1nnc2c(N3CCC4(CCCN4C)CC3)cc(C(=O)N[C@H](C)c3cccc(C(F)(F)F)c3F)cn12 ; Cc1c([C@@H](C)NC(=O)c2cc(N3CCC4(CCCN4C)CC3)c3nnc(C)n3c2)cccc1C(F)(F)F ; C[C@@H](NC(=O)c1cc([C@H]2CC[C@H](N3CCC[C@H](O)C3)CC2)c2nncn2c1)c1cccc(C(F)F)c1F] 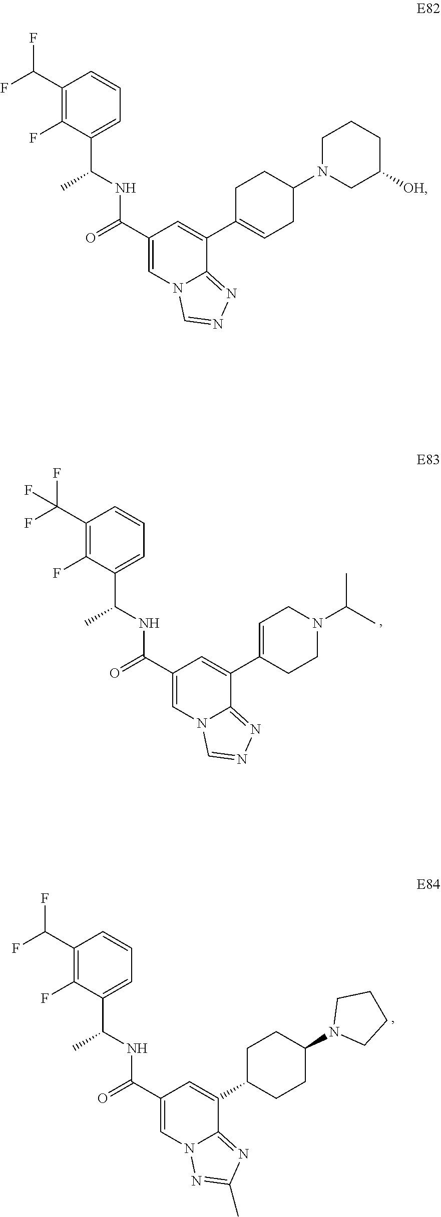 [SMILES: C[C@@H](NC(=O)c1cc(C2=CCC(N3CCC[C@H](O)C3)CC2)c2nncn2c1)c1cccc(C(F)F)c1F ; CC(C)N1CC=C(c2cc(C(=O)N[C@H](C)c3cccc(C(F)(F)F)c3F)cn3cnnc23)CC1 ; Cc1nc2c([C@H]3CC[C@H](N4CCCC4)CC3)cc(C(=O)N[C@H](C)c3cccc(C(F)F)c3F)cn2n1] 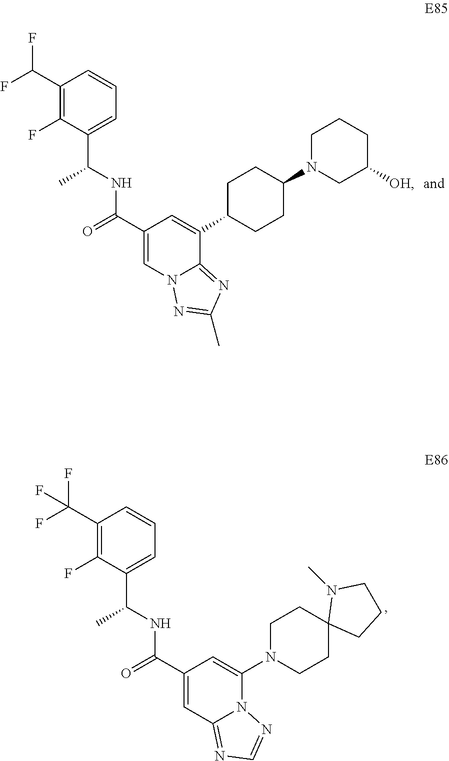 [SMILES: Cc1nc2c([C@H]3CC[C@H](N4CCC[C@H](O)C4)CC3)cc(C(=O)N[C@H](C)c3cccc(C(F)F)c3F)cn2n1 ; C[C@@H](NC(=O)c1cc(N2CCC3(CCCN3C)CC2)n2ncnc2c1)c1cccc(C(F)(F)F)c1F]
or a pharmaceutical acceptable salt thereof.

16. A method for the treatment and/or prevention of cancer comprising administering a therapeutically effective amount of a compound of formula (I) according to claim 1, or a pharmaceutically acceptable salt thereof, to a human being.

17. The method according to claim 16, wherein said compound or salt is administered in combination with a therapeutically effective amount of at least one other pharmacologically active substance.

18. The method according to claim 16, wherein the cancer is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, gastroesophageal cancer, chronic lymphocytic leukaemia, hepatocellular cancer, breast cancer, ovarian cancer, prostate cancer, glioblastoma, renal cancer and sarcoma.

19. A pharmaceutical composition comprising a compound of formula (I) according to claim 1, or a pharmaceutically acceptable salt thereof, and one or more pharmaceutically acceptable excipient(s).
